(19) United States
(12) Patent Application Publication
(10) Pub. No.: US 2025/0154152 A1
(43) Pub. Date: May 15, 2025

(54) INHIBITORS OF NAEGLERIA FOWLERI

(71) Applicants: Wisconsin Alumni Research Foundation, Madison, WI (US); Clemson University, Clemson, SC (US)

(72) Inventors: Jennifer GOLDEN, Waunakee, WI (US); Matthew LISH, Madison, WI (US); James MORRIS, Clemson, SC (US)

(21) Appl. No.: 18/945,316

(22) Filed: Nov. 12, 2024

Related U.S. Application Data
(60) Provisional application No. 63/598,939, filed on Nov. 14, 2023.

(51) Int. Cl. C07D 471/14; A61K 31/517; A61K 31/519; A61K 31/5377; A61K 31/5383; A61P 33/04; C07D 401/04; C07D 401/14; C07D 405/14; C07D 471/04; C07D 471/22; C07D 498/14

(52) U.S. Cl. CPC C07D 471/14; A61K 31/517; A61K 31/519; A61K 31/5377; A61K 31/5383; A61P 33/04; C07D 401/04; C07D 401/14; C07D 405/14; C07D 471/04; C07D 471/22; C07D 498/14

(57) ABSTRACT

The present technology provides compounds of Formulas I and II (as defined herein), pharmaceutical compositions including such compounds and methods of treatment using such compositions.

### CROSS-REFERENCE TO RELATED APPLICATIONS

This application claims the benefit of and priority to U.S. Provisional Appl. No. 63/598,939, filed Nov. 14, 2023, the contents of which are incorporated herein by reference in their entirety for any and all purposes.

### U.S. GOVERNMENT RIGHTS

This invention was made with government support under AI161232 awarded by the National Institutes of Health. The government has certain rights in the invention.

### FIELD OF THE TECHNOLOGY

The present invention relates generally to the field of compounds, compositions comprising the compounds, and methods of using the compounds as inhibitors of Naegleria fowleri and other amebic parasites.

### BACKGROUND

Humans can encounter the N. fowleri parasite when swimming in warm, fresh-water lakes or rivers, contaminated community pools, or by using non-sterile water with at-home medical devices. When the parasite is introduced in the nasal passages, it penetrates the cribriform plate and invades and destroys brain tissue. Severe brain infection, known as Primary Amoebic Meningoencephalitis (PAM), is associated with a 97% mortality rate in humans. An aggressive treatment regimen often involves using a combination of drugs such as miltefosine, amphotericin B, fluconazole, rifampin or others. However, additional therapeutic options with an improved safety margin and greater efficacy would be desirable.

### SUMMARY OF THE INVENTION

In one aspect, the present technology provides compounds of Formula I,

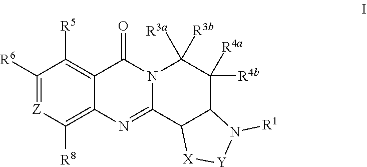

a stereoisomer of thereof, a pharmaceutically acceptable salt of the compound, or a pharmaceutically acceptable salt of the stereoisomer;
wherein,
   X is CH₂, O, or NR²;
   Y is CH₂ or CH₂CH₂, provided that when Y is CH₂, X is CH₂;
   Z is N or CR⁷;
   R¹ is H, C(O)R⁹, or a substituted or unsubstituted alkyl, cycloalkyl, cycloalkylalkyl, alkenyl, aralkyl, heterocyclylalkyl, or heteroarylalkyl group;
   R² is H or an unsubstituted alkyl group;
   R^{3a}, R^{3b}, R^{4a}, and R^{4b} are independently selected from the group consisting of H and a substituted or unsubstituted alkyl group;
   R⁵ and R⁸ are independently selected from the group consisting of H, OR¹⁰, halogen and a substituted or unsubstituted alkyl, alkenyl, aryl, aralkyl, or heteroaryl group;
   R⁶ and R⁷ are independently selected from the group consisting of H, halogen, CN, NO₂, C(O)R¹¹, C(O)OR¹¹, C(O)NR¹¹R¹², OR¹², and a substituted or unsubstituted alkyl, aryl or heteroaryl group;
   R⁹ is substituted or unsubstituted alkyl, cycloalkyl, cycloalkylalkyl, alkenyl, aryl, aralkyl, heterocyclyl, heterocyclylalkyl, heteroaryl, or heteroarylalkyl group; and
   R¹⁰, R¹¹, and R¹² at each occurrence are independently H or an unsubstituted alkyl group.

In another aspect, the present technology provides compounds of Formula II,

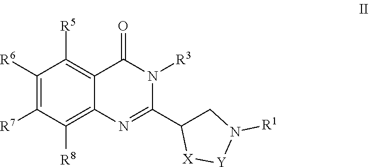

a stereoisomer of thereof, a pharmaceutically acceptable salt of the compound, or a pharmaceutically acceptable salt of the stereoisomer;
wherein,
   X is CH₂, O, or NR²;
   Y is CH₂ or CH₂CH₂, provided that when Y is CH₂, X is CH₂;
   R¹ is H, C(O)R⁹, or a substituted or unsubstituted alkyl, cycloalkyl, cycloalkylalkyl, alkenyl, aralkyl, heterocyclylalkyl, or heteroarylalkyl group;
   R² is H or an unsubstituted alkyl group;
   R³ is selected from the group consisting of H and a substituted or unsubstituted alkyl or alkenyl group;
   R⁵ and R⁸ are independently selected from the group consisting of H, OR¹⁰, and a substituted or unsubstituted alkyl, alkenyl or aralkyl group;
   R⁶ and R⁷ are independently selected from the group consisting of H, halogen, CN, OR¹⁰, and a substituted or unsubstituted aryl or heteroaryl group;
   R⁹ is substituted or unsubstituted alkyl, cycloalkyl, cycloalkylalkyl, alkenyl, aryl, aralkyl, heterocyclyl, heterocyclylalkyl, heteroaryl, or heteroarylalkyl group; and
   R¹⁰ at each occurrence is independently H or an unsubstituted alkyl group.

In another aspect, the present technology provides pharmaceutical compositions including any compound described herein and a pharmaceutically acceptable excipient and/or carrier.

In still another aspect, there are provided methods of treatment comprising administering an effective amount of any compound described herein to a subject suffering from or at risk of suffering from an infection of Naegleria fowleri.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The following terms are used throughout as defined below. All other terms and phrases used herein have their ordinary meanings as one of skill in the art would understand.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt %” would be understood to mean “9 wt % to 11 wt %.” It is to be understood that when “about” precedes a term, the term is to be construed as disclosing “about” the term as well as the term without modification by “about”—for example, “about 10 wt %” discloses “9 wt % to 11 wt %” as well as disclosing “10 wt %.”

The phrase “and/or” as used in the present disclosure will be understood to mean any one of the recited members individually or a combination of any two or more thereof—for example, “A, B, and/or C” would mean “A or B or C; A and B; A and C; B and C; or the combination of A, B, and C.”

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to one or more hydrogen atom contained therein are replaced by one or more bonds to one or more atoms other than hydrogen or carbon, or are replaced by a group of atoms. Substituted groups also include groups in which one or more bonds to a carbon(s) or hydrogen(s) atom are replaced by one or more bonds, including double or triple bonds, to a heteroatom. Unless otherwise specified, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; sulfonamides; sulfates; phosphates; amines; N-oxides; hydrazines; hydrazides; hydrazones; azides (—N₃); amides; ureas; amidines; guanidines; enamines; imides; imines; nitro groups (—NO₂); nitriles (—CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having (unless indicated otherwise) from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, amidinealkyl, guanidinealkyl, alkoxyalkyl, carboxyalkyl, and the like.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)=CH₂, among others. Representative substituted alkenyl groups may be mono-substituted or substituted more than once, such as, but not limited to, mono-, di- or tri-substituted with substituents such as those listed above for alkyl.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic carbon-containing ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, oxadiazolonyl (including 1,2,4-oxazol-5(4H)-one-3-yl), isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridinyl or morpholinyl groups, which are 2-, 3-, 4-, 5-, or 6-substituted, or disubstituted with various substituents such as those listed above.

Heteroaryl groups are aromatic carbon-containing ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent alkenyl groups are alkenylene groups, and so forth. Substituted groups having a single point of attachment to a compound or polymer of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

[The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, cycloalkyl, aryl, aralkyl, heterocyclylalkyl or heterocyclyl group as defined herein. Amido groups therefore include but are not limited to carbamoyl groups (—C(O)NH₂) (also referred to as “carboxamido groups”) and formamido groups (—NHC(O)H). In some embodiments, the amide is —NR⁷¹C(O)—(C₁₋₅ alkyl) and the group is termed “alkanoylamino.”

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, cycloalkyl, aryl, aralkyl, heterocyclylalkyl or heterocyclyl group as defined herein. In some embodiments, the amine is NH₂, alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino. It will be understood that amines may exist in protonated forms in certain aqueous solutions or mixtures and are examples of charged functional groups herein.

The term “carboxyl” or “carboxylate” as used herein refers to a —COOH group or its ionized salt form. As such, it will be understood that carboxyl groups are examples of charged functional groups herein.

The term “ester” as used herein refers to —COOR⁷⁰ and —C(O)O-G groups. R⁷⁰ is a substituted or unsubstituted alkyl, cycloalkyl, alkenyl, aryl, aralkyl, heterocyclylalkyl or heterocyclyl group as defined herein. G is a carboxylate protecting group. As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3^{rd} Edition, 1999). Which can be added or removed using the procedures set forth therein and which is hereby incorporated by reference in its entirety and for any and all purposes as if fully set forth herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, aryl aralkyl, heterocyclyl or heterocyclylalkyl group as defined herein. It will be understood that guanidines may exist in protonated forms in certain aqueous solutions or mixtures and are examples of charged functional groups herein.

The term “hydroxyl” as used herein can refer to —OH or its ionized form, —O⁻. A “hydroxyalkyl” group is a hydroxyl-substituted alkyl group, such as HO—CH₂—.

The term “imidazolyl” as used herein refers to an imidazole group or the salt thereof. An imidazolyl may be protonated in certain aqueous solutions or mixtures and is then termed an “imidazolate.”

The term “phosphate” as used herein refers to —OPO₃H₂ or any of its ionized salt forms, —OPO₃HR⁸⁴ or —OPO₃R⁸⁴R⁸⁵ wherein R⁸⁴ and R¹⁵ are independently a positive counterion, e.g., Na⁺, K⁺, ammonium, etc. As such, it will be understood that phosphates are examples of charged functional groups herein.

term “pyridinyl” refers to a pyridine group or a salt thereof. A pyridinyl may be protonated in certain aqueous solutions or mixtures and is then termed a “pyridinium group”.

The term “sulfate” as used herein refers to —OSO₃H or its ionized salt form, —OSO₃R⁸⁶ wherein R⁸⁶ is a positive counterion, e.g., Na⁺, K⁺, ammonium, etc. As such, it will be understood that sulfates are examples of charged functional groups herein.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, and R⁸² are each independently a substituted or unsubstituted alkyl, cycloalkyl, alkenyl, aryl aralkyl, heterocyclyl or heterocyclylalkyl group as defined herein. In some embodiments the sulfide is an alkylthio group, —S-alkyl. R⁸³ includes H or, when the sulfonyl is ionized (i.e., as a sulfonate), a positive counterion, e.g., Na⁺, K⁺, ammonium or the like. As such, it will be understood that sulfonyls are examples of charged functional groups herein.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

“Treating” within the context of the instant technology, means alleviation, in whole or in part, of symptoms associated with a disorder or disease, or slowing, inhibition or halting of further progression or worsening of those symptoms, or prevention or prophylaxis of the disease or disorder in a subject at risk for developing the disease or disorder. For example, within the context of treating a parasitic infection such as an amebic infection (e.g., Naegleria fowleri), successful treatment may include reduction or eradication of the parasite, from the body; clinical benefit; an alleviation of symptoms, such as a reduction or elimination of rash, itching, chafing, burning, throat thrush, redness, soreness, fever, cough, night sweats, weight loss, wheezing, and shortness of breath.

As used herein, a “therapeutically effective amount” of a compound of the present technology refers to an amount of the compound that alleviates, in whole or in part, symptoms associated with a disorder or disease, or slows or halts of further progression or worsening of those symptoms, or prevents or provides prophylaxis for the disease or disorder in a subject at risk for developing the disease or disorder. Those skilled in the art are readily able to determine a therapeutically effective amount. For example, one way of assessing a therapeutically effective amount for a particular disease state is by simply administering a compound of the present technology to a patient in increasing amounts until progression of the disease state is decreased or stopped or reversed. An “effective amount” of a compound of the present technology also refers to an amount of the compound that, for example, reduces a population of a parasite, such as Naegleria fowleri, where the population may be outside a subject (e.g., in media in a container).

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereoisomeric or geometric isomeric forms, it should be understood that the technology encompasses any tautomeric, conformational isomeric, stereoisomeric and/or geometric isomeric forms of the compounds having one or more of the utilities described herein, as well as mixtures of these various different forms.

Stereoisomers of compounds (also known as optical isomers) include all chiral, diastereomeric, and racemic forms of a structure, unless the specific stereochemistry is expressly indicated. Thus, compounds disclosed herein include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, imines may exhibit the following isomeric forms, which are referred to as tautomers of each other:

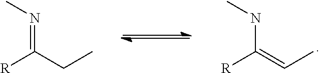

Because of the limits of representing compounds by structural formulas, it is to be understood that all chemical formulas of the compounds described herein represent all tautomeric forms of compounds and are within the scope of the present technology.

In an aspect, the present technology provides compounds of Formula I,

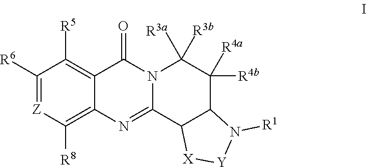

a stereoisomer of thereof, a pharmaceutically acceptable salt of the compound, or a pharmaceutically acceptable salt of the stereoisomer;
wherein,
   X is CH₂, O, or NR²;
   Y is CH₂ or CH₂CH₂, provided that when Y is CH₂, X is CH₂;
   Z is N or CR⁷;
   R¹ is H, C(O)R⁹, or a substituted or unsubstituted alkyl, cycloalkyl, cycloalkylalkyl, alkenyl, aralkyl, heterocyclylalkyl, or heteroarylalkyl group;
   R² is H or an unsubstituted alkyl group;
   R^{3a}, R^{3b}, R^{4a}, and R^{4b} are independently selected from the group consisting of H and a substituted or unsubstituted alkyl group;
   R⁵ and R⁸ are independently selected from the group consisting of H, OR¹⁰, halogen and a substituted or unsubstituted alkyl, alkenyl, aryl, aralkyl, or heteroaryl group;
   R⁶ and R⁷ are independently selected from the group consisting of H, halogen, CN, NO₂, C(O)R¹¹, C(O)OR¹¹, C(O)NR¹¹R¹², OR¹², and a substituted or unsubstituted alkyl, aryl or heteroaryl group;
   R⁹ is substituted or unsubstituted alkyl, cycloalkyl, cycloalkylalkyl, alkenyl, aryl, aralkyl, heterocyclyl, heterocyclylalkyl, heteroaryl, or heteroarylalkyl group; and
   R¹⁰, R¹¹, and R¹² at each occurrence are independently H or an unsubstituted alkyl group.

In any embodiments of the compounds of Formula I, X may be CH₂. In any embodiments, X may be O. In any embodiments, X may be NR².

In any embodiments of the compounds of Formula I, Y may be CH₂CH₂. In any embodiments, Y may be CH₂.

In any embodiments of the compounds of Formula I, Z may be N. In any embodiments of the compounds of Formula I, Z may be CR⁷ and the compound has the Formula IA:

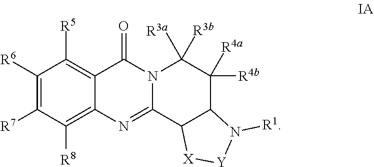

Various groups in Formula I may be substituted as defined herein. For example, substituted alkyl, cycloalkyl, cycloalkylalkyl, alkenyl, aryl, aralkyl, heterocyclyl, heterocyclylalkyl, heteroaryl, and heteroarylalkyl groups (such as in one or more of R¹, R^{3a}, R^{3b}R^{4a}, R^{4b}, R⁵, R⁶, R⁷, R⁸, and R⁹) may be substituted with one or more substituents (e.g., 1, 2, 3, 4, 5, 6) selected from the group consisting of halogen, hydroxyl, alkoxy, alkenoxy, aryloxy, aralkyloxy, heterocyclyl, heterocyclylalkyl, heterocyclyloxy, heterocyclylalkoxy, oxo, carboxyl, ester, urethane, oximes, hydroxylamine, alkoxyamine, aralkoxyamine, thiol, sulfide, sulfoxide, sulfone, sulfonyl, sulfonamide, sulfate, phosphate, amine, N-oxide, hydrazine, hydrazide, hydrazone, azide, amide, urea, amidine, guanidine, enamine, imide, imine, nitro, and nitrile groups, and further wherein the cycloalkylalkyl, aralkyl, heterocyclylalkyl, and heteroarylalkyl groups are also optionally substituted with one or more (e.g., 1, 2, 3, 4, 5, 6) of an unsubstituted alkyl or alkenyl group, a haloalkyl group, or a haloalkenyl group. In any embodiments the substituted alkyl, cycloalkyl, cycloalkylalkyl, alkenyl, aryl, aralkyl, heterocyclyl, heterocyclylalkyl, heteroaryl, and heteroarylalkyl groups may be substituted with 1, 2, or 3 substituents. In any embodiments of the compounds of Formula I, the substituents may be selected from the group consisting of F, Cl, Br, OH, NR₂, oxo, C₁₋₆ alkoxy, C₁₋₆ alkyl, CN, halogen, and C₁₋₆ haloalkyl group, wherein R is H or an unsubstituted C₁₋₃ alkyl group.

In any embodiments of the compounds of Formula I, R¹ may be selected from H, C(O)R⁹, or a substituted or unsubstituted alkyl, cycloalkylalkyl, aralkyl, heterocyclylalkyl, or heteroarylalkyl group. In any embodiments, R¹ may be selected from H or an unsubstituted alkyl, cycloalkylalkyl, aralkyl, or heterocyclylalkyl group. In any embodiments, R¹ is selected from H, C(O)R⁹, or a methyl, ethyl, isopropyl, s-butyl, t-butyl, n-pentyl, neopentyl, cyclopropylmethyl, cyclobutylmethyl, cyclopentylmethyl, cyclohexylmethyl, benzyl, pyridinylmethyl, pyridazinylmethyl, morpholinylethyl, oxetanylmethyl, tetrahydropyranylmethyl, 3,5-dimethoxybenzyl, 4-cyano-benzyl, tetrahydropyranylmethyl, pyridin-2-yl-methyl, 6-methylpyridin-2-yl-methyl, 4-t-butyl-benzyl, 4-chlorobenzyl, 4-trifluoromethylbenzyl, or 2,4-dichlorobenzyl group, wherein R⁹ is selected from substituted or unsubstituted alkyl, cycloalkyl, aryl, heterocyclyl, or heteroaryl group. In any embodiments, R¹ may be C(O)R⁹, and R⁹ is selected from a C₁₋₆ alkyl group optionally substituted with a N(CH₃)₂, phenyl, tetrahydropyranyl, or morpholinyl group.

In any embodiments of the compounds of Formula I, R² may be H or an unsubstituted C₁₋₆ alkyl group.

In any embodiments of the compounds of Formula I, R^{3a}, R^{3b}, R^{4a}, and R^{4b} may be independently selected from the group consisting of H and a substituted or unsubstituted C₁₋₆ alkyl group. In any embodiments, R^{3a}, R^{3b}, R^{4a}, and R^{4b} may be independently selected from the group consisting of H and an unsubstituted C₁₋₆ alkyl group. In any embodiments, R^{3a} and R^{3b} are both H. In any embodiments, R^{4a} and R^{4b} are both H. In any embodiments, R^{3a}, R^{3b}, R^{4a}, and R^{4b} are all H.

In any embodiments of the compounds of Formula I, R⁵ and R⁸ may be independently selected from the group consisting of H, halogen, an unsubstituted C₁₋₆ alkyl group, and OR¹⁰, wherein R¹⁰ is an unsubstituted C₁₋₆ alkyl group. In any embodiments, R⁵ and R⁸ may be independently selected from the group consisting of H, Cl, methyl and methoxy.

In any embodiments of compounds of Formula I, R⁶ and R⁷ are not both H. In any embodiments, R⁶ and R⁷ may be independently selected from the group consisting of H, halogen, CN, NO₂, C(O)R¹¹, C(O)OR¹¹, C(O)NR⁹, OR¹², and a substituted or unsubstituted aryl or heteroaryl group. In any embodiments, R⁶ and R⁷ may be independently selected from the group consisting of H, halogen, CN, NO₂, C(O)R¹¹, C(O)OR¹¹, C(O)NR⁹, OR¹², and a substituted or unsubstituted phenyl or pyridinyl group. In any embodiments, R⁶ and R⁷ are independently selected from the group consisting of H, F, Cl, Br, CN, CH₃, CF₃, OCH₃, phenyl, and 2-methylpyridin-4-yl.

In an aspect, the present technology provides compounds of Formula II,

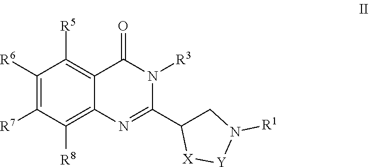

a stereoisomer of thereof, a pharmaceutically acceptable salt of the compound, or a pharmaceutically acceptable salt of the stereoisomer;
wherein,
   X is CH₂, O, or NR²;
   Y is CH₂ or CH₂CH₂, provided that when Y is CH₂, X is CH₂;
   R¹ is H, C(O)R⁹, or a substituted or unsubstituted alkyl, cycloalkyl, cycloalkylalkyl, alkenyl, aralkyl, heterocyclylalkyl, or heteroarylalkyl group;
   R² is H or an unsubstituted alkyl group;
   R³ is selected from the group consisting of H and a substituted or unsubstituted alkyl or alkenyl group;
   R⁵ and R⁸ are independently selected from the group consisting of H, OR¹⁰, and a substituted or unsubstituted alkyl, alkenyl or aralkyl group;
   R⁶ and R⁷ are independently selected from the group consisting of H, halogen, CN, OR¹⁰, and a substituted or unsubstituted aryl or heteroaryl group;
   R⁹ is substituted or unsubstituted alkyl, cycloalkyl, cycloalkylalkyl, alkenyl, aryl, aralkyl, heterocyclyl, heterocyclylalkyl, heteroaryl, or heteroarylalkyl group; and
   R¹⁰ at each occurrence is independently H or an unsubstituted alkyl group.

In any embodiments of the compound of Formula II, X, Y, R¹, R², and R⁵, R⁶, R⁷, R⁸, R⁹, and R¹⁰ may have any of the values disclosed herein for the compounds of Formula I as well as the following values.

In any embodiments of the compound of Formula II, X may be CH₂.

In any embodiments of the compound of Formula II, R¹ may be selected from an unsubstituted C₁₋₆ alkyl, oxetanylmethyl, or tetrahydropyranylmethyl group. In any embodiments of the compounds of Formula II, R³ may be unsubstituted alkyl such as unsubstituted C₁₋₆ alkyl, e.g., CH₃. In any embodiments, R³ may be substituted or unsubstituted aryl, e.g., phenyl. In any embodiments, R⁶ and R⁷ may be independently selected from H or halogen, e.g., Cl.

In another aspect, the present technology provides pharmaceutical compositions including any compound described herein and a pharmaceutically acceptable excipient and/or carrier.

In still another aspect, there are provided methods of treatment comprising administering an effective amount of any compound described herein to a subject suffering from or at risk of suffering of suffering from an infection of Naegleria fowleri. In any embodiments, the subject may be human. In any embodiments, the effective amount of the compound may be 0.001 mg/kg/day to 200 mg/kg/day. In any embodiments, the effective amount of the compound may be 0.01 mg/kg to 100 mg/kg. In any embodiments, the effective amount of the compound may be 0.1 mg/kg to 100 mg/kg. the effective amount of the compound is administered orally.

The instant technology also provides for compositions and medicaments including a compound disclosed herein and a pharmaceutically acceptable carrier. Such compositions may be prepared by mixing one or more compounds of the present technology, pharmaceutically acceptable salts thereof or stereoisomers thereof, with pharmaceutically acceptable carriers, excipients, binders, diluents or the like to treat amebic infections. The compounds and compositions of the present technology may be used to prepare formulations and medicaments that treat a variety of amebic infections, e.g., Naegleria fowleri herein. Such compositions can be in the form of, for example, granules, powders, tablets, capsules, creams, ointments, syrup, suppositories, injections, emulsions, elixirs, suspensions or solutions. The instant compositions can be formulated for various routes of administration, for example, by oral, parenteral, topical, injection, rectal, nasal, vaginal, or via implanted reservoir. Parenteral or systemic administration includes, but is not limited to, subcutaneous, intravenous, intraperitoneally, intramuscular, intrathecal, intracranial, and intracerebroventricular injections. The following dosage forms are given by way of example and should not be construed as limiting the instant technology.

For oral, buccal, and sublingual administration, powders, suspensions, granules, tablets, pills, capsules, gelcaps, and caplets are acceptable as solid dosage forms. These can be prepared, for example, by mixing one or more compounds disclosed herein, or pharmaceutically acceptable salts or stereoisomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions, which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology also may be formulated as a composition for topical administration (e.g., vaginal cream). These formulations may contain various excipients known to those skilled in the art. Suitable excipients may include, but are not limited to, cetyl esters wax, cetyl alcohol, white wax, glyceryl monostearate, propylene glycol monostearate, methyl stearate, benzyl alcohol, sodium lauryl sulfate, glycerin, mineral oil, water, carbomer, ethyl alcohol, acrylate adhesives, polyisobutylene adhesives, and silicone adhesives.

The composition may be in the form of a vaginal cream containing the composition of matter as set forth herein present in a nonliquefying base. The nonliquefying base may contain various inactive ingredients such as, for example, cetyl esters wax, cetyl alcohol, white wax, glyceryl monostearate, propylene glycol monostearate, methyl stearate, benzyl alcohol, sodium lauryl sulfate, glycerin, and mineral oil. Such composition may be formulated similar to PREMARIN® Vaginal Cream made commercially available by Wyeth-Ayerst Laboratories.

Dosage units for rectal administration may be prepared in the form of suppositories which may contain the composition of matter in a mixture with a neutral fat base, or they may be prepared in the form of gelatin-rectal capsules which contain the active substance in a mixture with a vegetable oil or paraffin oil.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. Formulations for inhalation administration contain as excipients, for example, lactose, polyoxyethylene-9-lauryl ether, glycocholate and deoxycholate. Aqueous and nonaqueous aerosols are typically used for delivery of inventive compounds by inhalation.

Ordinarily, an aqueous aerosol is made by formulating an aqueous solution or suspension of the compound together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins such as serum albumin, sorbitan esters, oleic acid, lecithin, amino acids such as glycine, buffers, salts, sugars or sugar alcohols. Aerosols generally are prepared from isotonic solutions. A nonaqueous suspension (e.g., in a fluorocarbon propellant) can also be used to deliver compounds of the present technology.

Aerosols containing compounds for use according to the present technology are conveniently delivered using an inhaler, atomizer, pressurized pack or a nebulizer and a suitable propellant, e.g., without limitation, pressurized dichlorodifluoromethane, trichlorofluoromethane, dichlorotetrafluoroethane, nitrogen, air, or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch. Delivery of aerosols of the present technology using sonic nebulizers is advantageous because nebulizers minimize exposure of the agent to shear, which can result in degradation of the compound.

For nasal administration, the pharmaceutical formulations and medicaments may be a spray, nasal drops or aerosol containing an appropriate solvent(s) and optionally other compounds such as, but not limited to, stabilizers, antimicrobial agents, antioxidants, pH modifiers, surfactants, bioavailability modifiers and combinations of these. For administration in the form of nasal drops, the compounds may be formulated in oily solutions or as a gel. For administration of nasal aerosol, any suitable propellant may be used including compressed air, nitrogen, carbon dioxide, or a hydrocarbon based low boiling solvent.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended-release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant technology.

A therapeutically effective amount of a compound of the present technology may vary depending upon the route of administration and dosage form. Effective amounts of such compounds typically fall in the range of about 0.01 up to about 100 mg/kg/day, or about 0.05 to about 50 mg/kg/day, and more typically in the range of about 0.1 up to 5 mg/kg/day or 10 mg/kg/day. Typically, the compound or compounds of the instant technology are selected to provide a formulation that exhibits a high therapeutic index. The therapeutic index is the dose ratio between toxic and therapeutic effects and can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology is further illustrated by the following examples, which should not be construed as limiting in any way.

### EXAMPLES

### General Procedures

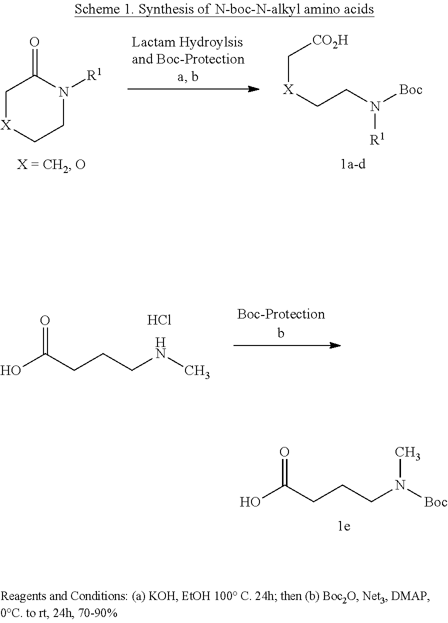

### General Procedure 1: Lactam Hydrolysis and Boc-Protection (Scheme 1)

In an oven-dried 10-20 mL microwave irritation vial, lactam (1 eq.) was suspended in 1.2 M of anhydrous EtOH (R═Me, Et) or MeOH (R═Bn) followed by the addition of KOH (2 eq.). The vial was then sealed, evacuated, backfilled with Ar(g), and heated μW 100° C. for 12 h. The reaction was then transferred to a 250 mL round bottom flask, diluted with 75 mL anhydrous alcohol, followed by the addition of DMAP (2 eq.) and Et₃N (2 eq.). The flask was then flushed with Ar(g) and cooled to 0° C. At 0° C., Boc₂O (2 eq.) in 20 mL of ROH was added dropwise over 10 min. The reaction was stirred at 0° C. for 10 min before being allowed to warm to room temperature and stirred for 24 h. After 24 h, the reaction mixture was filtered, and the solvent was removed under reduced pressure. The crude was then suspended in 1N NaOH (150 mL), washed with DCM (3×150 mL). pH was then adjusted with ice cold 1N HCl to pH=2-3 resulting in a cloudy solution which was then extracted with DCM (3×300 mL). The combined organics were washed with brine, dried over Na₂SO₄, and filtered, then the solvent was evaporated under reduced pressure to afford 1a-d, which didn't require any further purification.

### General Procedure 2: Boc-Protection of 4-(Methylamino)Butyric Acid Hydrochloride (Scheme 1)

4-(Methylamino)butyric acid hydrochloride (1 eq.), DMAP (0.15 eq.), and KOH (2 eq.) were placed in an oven dried 500 mL round bottom flask under Ar (g). Then EtOH (0.2 M) and NEt₃ (2 eq.) were added. The reaction mixture was then cooled to 0° C. followed by the dropwise addition of Boc₂O (2 eq.) in EtOH (2.6 M). The reaction continued to stir at 0° C. for 10 min before allowing it to warm to room temperature and stir for 24 h. After 24 h, the reaction mixture was filtered, and the solvent was removed under reduced pressure. The crude was then suspended in 200 mL 1N NaOH, washed with DCM (2×250 mL). pH was then adjusted with ice cold 1N HCl to pH=2-3 resulting in a cloudy solution which was then extracted with DCM (3×600 mL). The combined organics were washed with brine, dried over Na₂SO₄, and filtered, then the solvent was evaporated under reduced pressure to afford 1e (4.75 g, 84%).

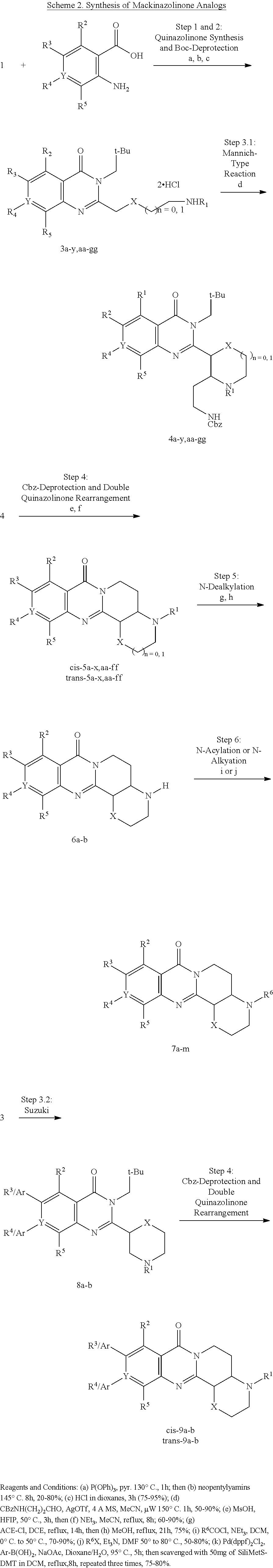

### General Procedure 3: Synthesis of Boc-Protected Quinazolinones (Scheme 2. Step 1)

Substituted 2-amino-benzoic acid (1 eq.), 1 (1.2 eq.), and P(OPh)₃ (2.2 eq.) were suspended in anhydrous pyridine (0.12 M) and heated to 130° C. in a pressure relief vial for 1 h. After 1 h, the reaction was cooled to room temperature and neopentyl amine (2 eq.) was added. The reaction mixture was then heated to 145° C. for 8 h. Solvent was then removed under reduced pressure. Crude was then suspended in EtOAc (80 mL), washed with 10% citric acid, sat. NaHCO₃ (aq), brine, filtered, and dried over Na₂SO₄, and concentrated under reduced pressure. The residue was purified by flash chromatography to afford 2a-y, 2aa-gg.

### General Procedure 4: Synthesis of Bis-Hydrochloride Salts from N-Boc-Protected Quinazolinones (Scheme 2, Step 2)

Boc-protected quinazolinone (1 eq.) was suspended in either 4 N HCl in dioxane (0.16 M) or 3N HCl in MeOH (0.16 M) for 2-3 h after which TLC or LCMS showed consumption of the Boc-protected quinazolinone. Upon completion, the reaction mixture was poured into Et₂O and allowed to stir for 10 min. The bis-HCl salt was then collected by vacuum filtration and dried in a vacuum oven at 60° C. to afford 3a-gg.

### General Procedure 5.1: Synthesis of Mannich Intermediates (Scheme 2, Step 3.1)

Compound 3 (1 eq.), benzyl (3-oxopropyl)carbamate (4 eq.), AgOTf (1 eq.) and activated 4 Å MS were placed in an oven dried 2-5 mL μW vial. The vial was sealed, evacuated, and backfilled with Ar (g) (2×), followed by the addition of degassed anhydrous CH₃CN (0.15 M). The reaction was then heated by μW irradiation at 150° C. for 1 h. The reaction was filtered over celite, diluted with 0.5% NEt₃ in DCM (40 mL), and concentrated under reduced pressure. The residue was then suspended in MeOH (5 mL) and absorbed to a 5 g HF-Mega BE-SCX column (Agilent). After rinsing with 5×20 mL of MeOH, the absorbed crude material was obtained by washing with 7N NH₃ in MeOH (60 mL). After concentration, the crude material was purified by reverse phase flash chromatography to give 4a-gg.

### General Procedure 5.2: Suzuki Cross-Coupling on Mannich Intermediates (Scheme 2, Step 3.2)

Compound 4 was placed in a 20-40 mL oven-dried pressure relief vial with Pd(dppf)₂Cl₂ (0.1 eq.), NaOAc (4.5 eq.) and ArB(OH)₂ (1.5 eq.). The vial was then sealed, evacuated, and backfilled with Ar (g). Degassed anhydrous dioxane/water (3:1) was then added. The vial was heated to 95° C. for 24 h. After, diluted with DCM, washed with H₂O, rinsed with brine, dried over Na₂SO₄, filtered, and concentrated. The residue was then purified by normal phase chromatography to afford 8a-b.

### General Procedure 6: N-Cbz Deprotection and Double Quinazolinone Rearrangement (Scheme 2, Step 4)

Compound 4 or 8 (1 eq.) was suspended in HFIP (0.2 M). MsOH (5 eq.) was then added, and the reaction was heated for 3 h at 50° C. After 3 h, the reaction was cooled to room temperature and quenched with 1 mL of MeOH. Solvent was then removed under reduced pressure by azeotroping three times with chloroform. Then the residue was transferred to a new sealed tube, suspended in CH₃CN (0.08 M) followed by the addition of NEt₃ (15 eq.) The tube was sealed and heated to 80° C. generally for 8 h or until the free amine was fully converted to the cyclized product as monitored by LCMS. Upon completion, solvent was removed under reduced pressure. The residue was then suspended in DCM and rinsed with brine, dried over Na₂SO₄, filtered, and concentrated. The residue was then purified by normal phase chromatography to afford 5a-ff (from 4) or 9a-b (from 8).

### General Procedure 7: N-Demethylation of Trans-Mackinazolinones (Scheme 2, Step 5)

Compound 5 were placed in an oven-dried pressure relief vial. The vial was sealed, evacuated, and backfilled with Ar (g), followed by the addition of DCE (0.2 M). Then ACE-Cl (4 eq.) was added dropwise. The reaction was allowed to stir at room temperature for 10 m, then heated to reflux for 14 h. After solvent and excess ACE-Cl was removed under reduced pressure and then the crude reaction mixture was suspended in anhydrous methanol (0.02 M) and heated to reflux for 21 h. Upon completion, solvent was removed under reduced pressure. The crude reaction mixture was then suspended in 20% IPA/CHCl₃ and washed with sat. NaHCO₃ (aq), dried over Na₂SO₄, filtered, and concentrated. The residue was then purified by normal phase chromatography to afford 6a-b.

### General Procedure 8.1: N-Alkylation of NH-Mackinazolinones (Scheme 2, Step 6)

Compound 6 (1 eq.), alkyl-halide (4 eq.), and Et₃N (6 eq.) were suspended in anhydrous DMF (0.25 M) and heated 50-80° C. until LCMS showed minimal or no starting material. After, the reaction was diluted with 20% IPA/CHCl₃, washed with sat. NaHCO₃ (aq), dried over Na₂SO₄, filtered, and concentrated. The residue was then purified by normal phase chromatography to afford 7.

### General Procedure 8.2: N-Acylation of NH-Mackinazolinones (Scheme 2, Step 6)

Compound 6 (1 eq.) and Et₃N (2 eq.) were suspended in anhydrous DCM (0.25 M) and cooled to 0° C. At 0° C., acyl-chloride (1.2 eq.) was added dropwise. After stirring at 0° C. for 10 m, the reaction was allowed to warm to room temperature and stirred until LCMS showed minimal or no starting material. After, the reaction was diluted with 20% IPA/CHCl₃, washed with sat. NaHCO₃ (aq), dried over Na₂SO₄, filtered, and concentrated. The residue was then purified by normal phase chromatography to afford 7.

### General Procedure 8.3: N-Alkylation of N-Ethanolamine (7j or 7ll)

N-ethanolamine (7J or 7ll) (1 eq.) and Et₃N (2 eq.) were suspended in anhydrous DCE (0.25 M) and cooled to 0° C. At 0° C., MsCl (1.2 eq.) was added dropwise, and then the reaction was allowed to warm to room temperature and stirred for 2 h. After 2 h, amine (6 eq.) was added dropwise. The reaction was allowed to stir at room temperature for 22 h. After, the reaction was diluted with 20% IPA/CHCl₃, washed with sat. NaHCO₃ (aq), water, brine, and dried over Na₂SO₄, filtered, and concentrated. The residue was then purified by normal phase chromatography to afford N-substituted amino ethylene 7.

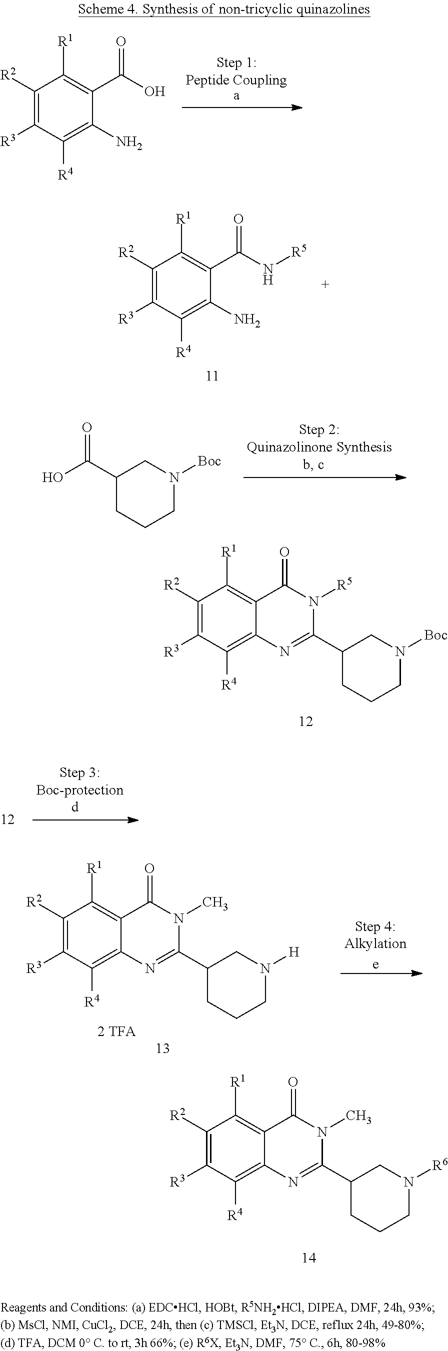

### General Procedure 9a: Synthesis of 2-Amino-N-Substituted Benzamides with Alkylammonium Salts (Scheme 4, Step 1)

Substituted 2-amino-benzoic acid (1 eq.), alkylammonium salt (4.2 eq), EDC*HCl (1.2 eq.), and HOBt (1.3 eq.) were suspended in anhydrous DMF (0.45 M). After stirring at room temperature for 30 minutes, DIPEA (4.2 eq.) was added dropwise. The reaction was stirred at room temperature for 24 h. The reaction was quenched with H₂O and extracted three times with EtOAc. The combined organic layers were then washed with H₂O, sat. NaHCO₃ (aq), and then brine. The combined extracts were dried over Na₂SO₄, filtered, and concentrated to afford 11.

### General Procedure 9b: Synthesis of 2-Amino-N-Substituted Benzamides with Amines (Scheme 4, Step 1)

Substituted 2-amino-benzoic acid (1 eq.), HATU (1.2 eq), and NEt₃ (2 eq.) were suspended in anhydrous DMF (0.25 M). After stirring at room temperature for 10 minutes, amine (1.5 eq.) was added dropwise. The reaction was allowed to stir at room temperature for 18 h. After, the reaction was quenched with H₂O and extracted three times with EtOAc. The combined organics were then washed with H₂O, sat. NaHCO₃ (aq), and brine. The organic extracts were dried over Na₂SO₄, filtered, and concentrated to afford 11.

### General Procedure 10: Synthesis of Boc-Protected Quinazolinones from 2-Amino-N-Substituted Benzamides (Scheme 4, Step 2)

Boc-protected amino acid (1.47 eq.), CuCl₂ (0.5 eq.) were placed in an oven dried 20-40 mL pressure relief vial. The vial was sealed, evacuated, and backfilled with Ar (g) (2×), followed by the addition of anhydrous DCE (0.10 M). The reaction was then cooled to 0° C. At 0° C., NMI (3.4 eq.) was added dropwise, followed by the dropwise addition of MsCl (1.47 eq). The reaction was then allowed to stir at room temperature for 3 h. Then 11 was added and the reaction was allowed to stir at room temperature for an additional 16 h. Upon completion, the reaction was cooled to room temperature. Diluted with DCM, washed with 1 N HCl (aq), sat. NaHCO₃ (aq), and brine. The organic extracts were dried over Na₂SO₄, filtered, and concentrated. The crude reaction mixture was then purified by flash chromatography to afford 12.

### General Procedure 11: Synthesis of Quinazolinone Trifluoroacetic Acid Salts 13 (Scheme 4, Step 3)

To a solution of 12 (1 eq.) in anhydrous DCM (0.10 M) was added TFA (30 eq.) dropwise at 0° C. Then the reaction was allowed to stir at room temperature for 2 h. Upon completion, the reaction mixture was slowly dripped into rapidly stirring anhydrous Et₂O and allowed to stir for 15 min. The product was isolated by vacuum filtration to afford 13.

### General Procedure 12 Alkylation of Quinazolinone Trifluoroacetic Acid Salts (Scheme 4, Step 4)

Compound 13 (1 eq.), alkyl-halide (3 eq.), and Et₃N (6 eq.) were suspended in anhydrous DMF (0.25 M) and heated to 75° C. for 6 h or until LCMS showed consumption of 13. Upon completion, the reaction was diluted with DCM, washed with sat. NaHCO₃ (aq), H₂O, and brine. The organic extracts were dried over Na₂SO₄, filtered, and concentrated. The crude reaction was then purified by flash chromatography to afford 14.

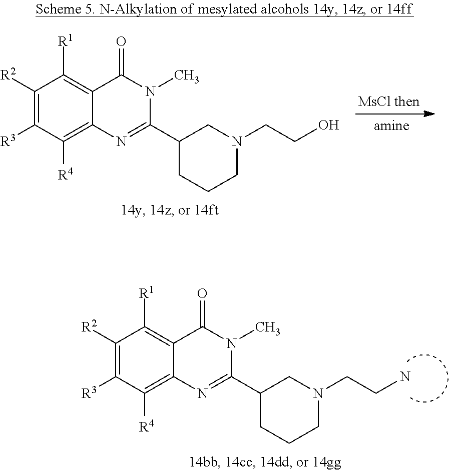

### General Procedure 12b: N-Alkylation of Mesylated Alcohols 14y, 14z, or 14ff to Afford 14bb, 14cc, 14dd, or 14gg

Alkyl alcohol 14y, 14z, or 14ff (1 eq.) and Et₃N (2 eq.) was suspended in anhydrous DCE (0.25 M) and cooled to 0° C. at which MsCl (1.3 eq.) was added dropwise. The reaction warmed to room temperature and was stirred for 2 h. After 2 h, pyrrolidine or morpholine (6 eq.) was added dropwise, and the resulting mixture was stirred at room temperature for 24 h. The reaction was diluted with 20% IPA/CHCl₃, washed with sat. NaHCO₃ (aq), water, brine, and dried over Na₂SO₄, filtered, and concentrated. The residue was then purified by normal phase chromatography to afford N-substituted amino ethylene 14bb, 14cc, 14dd or 14gg, depending on the starting material and amine chosen.

### Example 1: 5-((tert-Butoxycarbonyl)(methyl)amino)pentanoic acid (1a)

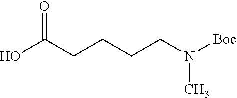 [SMILES: CN(CCCCC(=O)O)C(=O)OC(C)(C)C]

Compound 1a was prepared in accordance with general procedure 1A from 1-methylpiperidine-2-one. 1a was obtained as an off white solid (7.2 g, 71%). ¹H NMR (400 MHz, DMSO) δ 11.99 (s, 1H), 3.16 (s, 2H), 2.74 (s, 3H), 2.29-2.18 (m, 2H), 1.49-1.39 (m, 4H), 1.38 (s, 9H). HRMS (ESI) m/z: [M+Na]⁺ Calcd for C₁₁H₂₁NO₄Na, 254.1363. Found 254.1361.

### Example 2: 5-((tert-butoxycarbonyl)(ethyl)amino)pentanoic acid (1b)

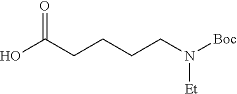 [SMILES: CCN(CCCCC(=O)O)C(=O)OC(C)(C)C]

Compound 1b was prepared in accordance with general procedure 1A from 1-ethylpiperidine-2-one. 1b was obtained as a yellow oil (0.71 g, 79%). ¹H NMR (400 MHz, DMSO) δ 3.11 (q, J=6.8 Hz, 4H), 2.24-2.16 (m, 2H), 1.45 (p, J=3.5 Hz, 4H), 1.38 (s, 9H), 1.02 (td, J=7.2, 2.9 Hz, 3H).

### Example 3: 5-(benzyl(tert-butoxycarbonyl)amino)pentanoic acid (1c)

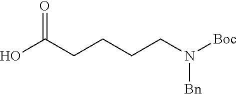 [SMILES: CC(C)(C)OC(=O)N(CCCCC(=O)O)Cc1ccccc1]

Compound 1c was prepared in accordance with general procedure 1A from 1-benzylpiperidine-2-one. 1c was obtained as a yellow oil (0.94 g, 23%). ¹H NMR (400 MHz, DMSO) δ 11.99 (s, 1H), 7.33 (t, J=7.3 Hz, 2H), 7.27-7.20 (m, 3H), 4.36 (s, 2H), 3.21-3.00 (m, 2H), 2.24-2.13 (m, 2H), 1.41 (ddd, J=26.1, 13.9, 8.0 Hz, 13H). HRMS (ESI) m/z: [M+H]⁺ Calcd for C₁₇H₂₅NO₄+Na+ 330.1676. Found 330.1670.

### Example 4: 2-(2-((tert-butoxycarbonyl)(methyl)amino)ethoxy)acetic acid (1d)

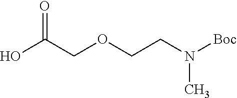 [SMILES: CN(CCOCC(=O)O)C(=O)OC(C)(C)C]

Compound 1d was prepared in accordance with general procedure 1 from 4-methylmorpholin-3-one. 1d obtained as a slight yellow oil (3.9 g, 94%). ¹H NMR (400 MHz, DMSO) δ 12.60 (s, 1H), 4.00 (s, 2H), 3.54 (d, J=11.5 Hz, 2H), 3.31 (t, J=5.7 Hz, 2H), 2.81 (d, J=8.4 Hz, 3H), 1.38 (s, 9H). HRMS (ESI) m/z: [M+Na]+ Calcd for C₁₀H₁₉NO₅Na, 256.1155. Found 256.1151.

### Example 5: 4-((tert-butoxycarbonyl)(methyl)amino)butanoic acid (1e)

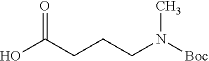

Compound 1e was prepared in accordance with general procedure 2. 1d was obtained as a yellow oil 4.75 g (86%). ¹H NMR (400 MHz, DMSO) δ 12.04 (s, 1H), 3.16 (t, J=7.0 Hz, 2H), 2.74 (s, 3H), 2.16 (t, J=7.2 Hz, 2H), 1.67 (p, J=7.2 Hz, 2H), 1.38 (s, 9H). HRMS (ESI) m/z: [M+Na]+ Calcd for C₁₀H₁₉NO₄Na, 240.1206. Found 240.1204.

### Example 6: Synthesis of (±)-(4aR,13bS)-4-methyl-1,2,3,4,4a,5,6,13b-octahydro-8H-[1,6]naphthyridino[5,6-b]quinazolin-8-one ((±)-trans-5a)) and (±)-(4aR,13bR)-4-methyl-1,2,3,4,4a,5,6,13b-octahydro-8H-[1,6]naphthyridino[5,6-b]quinazolin-8-one ((±)-cis-5a))

Prepared according to Scheme 2.

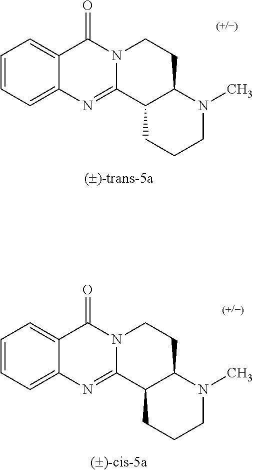 [SMILES: CN1CCC[C@@H]2c3nc4ccccc4c(=O)n3CC[C@H]21 ; CN1CCC[C@H]2c3nc4ccccc4c(=O)n3CC[C@H]21]

Step 1: Synthesis of tert-butyl methyl(4-(3-neopentyl-4-oxo-3,4-dihydroquinazolin-2-yl)butyl)carbamate (2a). Compound 2a was synthesized according to general procedure 3 from anthranilic acid and 1a. Purification by flash chromatography (0-10% EtOAc/DCM) delivered 2a as a colorless oil (1.7 g, 71%). ¹H NMR (400 MHz, CDCl₃) δ 8.22 (dd, J=8.0, 1.5 Hz, 1H), 7.69 (ddd, J=8.4, 7.0, 1.6 Hz, 1H), 7.59 (dd, J=8.2, 1.2 Hz, 1H), 7.41 (ddd, J=8.1, 7.1, 1.2 Hz, 1H), 4.10 (s, 0H), 3.43 (s, 2H), 3.26 (s, 2H), 2.97-2.78 (m, 5H), 1.77 (p, J=7.9 Hz, 2H), 1.63 (p, J=7.2 Hz, 2H), 1.44 (s, 9H), 1.00 (s, 9H). HRMS (ESI) m/z: [M+H]⁺ Calcd for C₂₃H₃₆N₃O₃, 402.2751. Found 402.2754.

Step 2: Synthesis of 2-(4-(methylamino)butyl)-3-neopentylquinazolin-4(3H)-one bis-hydrochloride salt (3a). Compound 3a was synthesized according to general procedure 4 from 2a, which afforded 3a as a white solid (1.25 g, 84%). ¹H NMR (400 MHz, DMSO) δ 9.37-9.14 (m, 2H), 8.21-8.10 (m, 1H), 8.02-7.84 (m, 2H), 7.63 (ddd, J=8.2, 5.8, 2.5 Hz, 1H), 4.12 (s, 2H), 3.17 (t, J=7.3 Hz, 2H), 2.93 (p, J=6.9 Hz, 2H), 2.50 (d, J=10.8 Hz, 4H), 1.98-1.76 (m, 4H), 0.96 (s, 9H). HRMS (ESI) m/z: [M+H]⁺ Calcd for C₁₈H₂₈N₃O, 302.2227. Found 302.2230.

Step 3.1: Synthesis of benzyl (2-(1-methyl-3-(3-neopentyl-4-oxo-3,4-dihydroquinazolin-2-yl)piperidin-2-yl)ethyl)carbamate (4a). Compound 4a was synthesized according to general procedure 5.1 from 3a. Purification by reverse phase flash chromatography (10-80% MeOH/H₂O) which afforded 4a as a white solid (226 mg, 92%). ¹H NMR (400 MHz, CDCl₃) δ 8.21 (ddd, J=7.8, 6.3, 1.7 Hz, 1H), 7.78-7.58 (m, 1.3H), 7.44-7.26 (m, 6.7H), 6.35 (s, 0.4H), 5.19-5.06 (m, 1.7H), 5.06-4.94 (m, 0.6H), 4.77 (d, J=14.2 Hz, 0.3H), 4.52 (s, 0.5H), 3.71-3.52 (m, 1H), 3.35 (s, 0.9H), 3.11 (ddd, J=12.2, 9.5, 3.2 Hz, 1.5H), 2.99-2.69 (m, 2.6H), 2.53 (s, 1.1H), 2.43 (s, 2.9H), 2.25 (dt, J=14.8, 7.4 Hz, 0.4H), 2.02-1.39 (m, 6.3H), 1.36-1.21 (m, 0.7H), 1.00 (d, J=7.9 Hz, 9H), 0.88 (t, J=7.0 Hz, 0.7H). HRMS (ESI) m/z: [M+H]⁺ Calcd for C₂₉H₃₉N₄O₃, 491.3017. Found 491.3022.

Step 4: Synthesis of (±)-(4aR,13bS)-4-methyl-1,2,3,4,4a,5,6,13b-octahydro-8H-[1,6]naphthyridino[5,6-b]quinazolin-8-one ((±)-trans-5a)) and (±)-(4aR,13bR)-4-methyl-1,2,3,4,4a,5,6,13b-octahydro-8H-[1,6]naphthyridino[5,6-b]quinazolin-8-one ((±)-cis-5a)). Both diastereomers of 5a were synthesized according to general procedure 6 starting from 4a. Purified by flash chromatography: 10-50% X/hexanes (X=2% NEt₃ 3:1 EtOAc/EtOH). (±)-trans-5a+(±)-cis-5a: 40.5 mg (75%).

(±)-trans-5a was isolated as a colorless oil (28.9 mg). ¹H NMR (400 MHz, CDCl₃) δ 8.23 (dd, J=8.1, 1.5 Hz, 1H), 7.69 (ddd, J=8.4, 7.0, 1.6 Hz, 1H), 7.61 (dd, J=8.3, 1.2 Hz, 1H), 7.40 (ddd, J=8.1, 7.0, 1.3 Hz, 1H), 4.22-4.06 (m, 2H), 2.96 (ddt, J=11.5, 3.9, 1.9 Hz, 1H), 2.77-2.69 (m, 1H), 2.66 (dd, J=11.1, 3.7 Hz, 1H), 2.41 (dq, J=13.3, 5.5 Hz, 1H), 2.35 (s, 3H), 2.10 (td, J=11.9, 3.2 Hz, 1H), 1.96 (td, J=10.1, 4.8 Hz, 1H), 1.89-1.71 (m, 3H), 1.47 (tdd, J=12.8, 11.3, 4.4 Hz, 1H). HRMS (ESI) m/z: [M+H]⁺ Calcd for C₆H₂₀N₃O, 270.1601. Found 270.1599.

(±)-cis-5a was isolated as a colorless oil (11.6 mg¹H NMR (400 MHz, CDCl₃) δ 8.24 (dd, J=7.9, 1.5 Hz, 1H), 7.69 (ddd, J=8.4, 7.0, 1.6 Hz, 1H), 7.61 (dd, J=8.2, 1.2 Hz, 1H), 7.40 (ddd, J=8.1, 7.0, 1.3 Hz, 1H), 4.17-3.99 (m, 2H), 3.02 (q, J=3.8 Hz, 1H), 2.87-2.72 (m, 2H), 2.58 (dt, J=6.2, 3.2 Hz, 1H), 2.35 (dq, J=14.7, 4.9 Hz, 1H), 2.29 (s, 3H), 2.20 (td, J=10.9, 3.2 Hz, 1H), 1.96 (dddd, J=14.1, 10.5, 6.0, 2.8 Hz, 1H), 1.71-1.52 (m, 3H). HRMS (ESI) m/z: [M+H]⁺ Calcd for C₁₆H₂₀N₃O, 270.1601. Found 270.1600.

### Example 7: Synthesis of (±)-(4aR,13bS)-4,9-dimethyl-1,2,3,4,4a,5,6,13b-octahydro-8H-[1,6]naphthyridino[5,6-b]quinazolin-8-one ((±)-trans-5b) and (±)-(4aR,13bR)-4,9-dimethyl-1,2,3,4,4a,5,6,13b-octahydro-8H-[1,6]naphthyridino[5,6-b]quinazolin-8-one((±)-cis-5b)

Prepared according to Scheme 2.

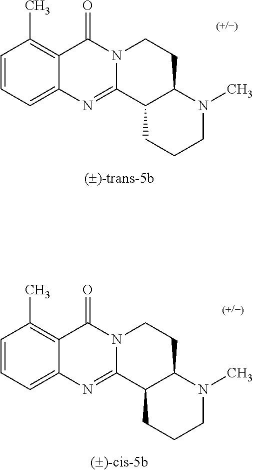 [SMILES: Cc1cccc2nc3n(c(=O)c12)CC[C@@H]1[C@@H]3CCCN1C ; Cc1cccc2nc3n(c(=O)c12)CC[C@@H]1[C@H]3CCCN1C]

Step 1: Synthesis of tert-butyl methyl(4-(5-methyl-3-neopentyl-4-oxo-3,4-dihydroquinazolin-2-yl)butyl)carbamate (2b). Compound 2b was synthesized according to general procedure 3 from 2-amino-6-methyl benzoic acid and 1a. Purification by flash chromatography (5-25% X/hexanes, X=4:1 EtOAc/DCM) delivered 2b as a colorless oil (0.81 g, 59%). ¹H NMR (400 MHz, CDCl₃) δ 7.51 (dd, J=8.1, 7.3 Hz, 1H), 7.43-7.39 (m, 1H), 7.14 (dt, J=7.3, 1.1 Hz, 1H), 4.08 (s, 2H), 3.25 (s, 2H), 2.82 (d, J=4.1 Hz, 8H), 1.75 (p, J=7.4 Hz, 2H), 1.62 (p, J=7.3 Hz, 2H), 1.44 (s, 9H), 0.97 (s, 9H). HRMS (ESI) m/z: [M+H]⁺ Calcd for C₂₄H₃₈N₃O₃, 416.2908. Found 416.2919.

Step 2: Synthesis of 5-methyl-2-(4-(methylamino)butyl)-3-neopentylquinazolin-4(3H)-one bis-hydrochloride salt (3b). Compound 3b was synthesized according to general procedure 4 from 2b, which afforded 3b as a white solid (0.53 g, 78%). ¹H NMR (400 MHz, DMSO) δ 9.23 (q, J=6.1 Hz, 2H), 7.87-7.75 (m, 2H), 7.44 (dt, J=6.7, 1.3 Hz, 1H), 4.10 (s, 2H), 3.19 (t, J=7.3 Hz, 2H), 2.94 (q, J=6.5 Hz, 2H), 2.73 (s, 3H), 2.50 (d, J=10.8 Hz, 4H), 1.92-1.80 (m, 4H), 0.95 (s, 9H). HRMS (ESI) m/z: [M+H]⁺ Calcd for C₁₉H₃₀N₃O, 316.2383. Found 316.2388.

Step 3.1: Synthesis of benzyl (2-(1-methyl-3-(5-methyl-3-neopentyl-4-oxo-3,4-dihydroquinazolin-2-yl)piperidin-2-yl)ethyl)carbamate (4b). Compound 4b was synthesized according to general procedure 5.1 from 3b. Purification by reverse phase flash chromatography (10-80% MeOH/H₂O) afforded 4b a white solid (209 mg, 83%). ¹H NMR (400 MHz, CDCl₃) δ 7.63-7.47 (m, 1H), 7.47-7.22 (m, 5.6H), 7.18-7.07 (m, 1.4H), 6.38 (s, 0.8H), 5.26-4.91 (m, 2H), 4.90-4.38 (m, 1.1H), 3.64-3.23 (m, 2.6H), 3.12-2.98 (m, 0.8H), 2.96-2.67 (m, 5.7H), 2.53 (s, 1.2H), 2.42 (s, 3.2H), 2.01-1.30 (m, 5.4H), 0.99 (d, J=7.9 Hz, 9H). HRMS (ESI) m/z: [M+H]⁺ Calcd for C₃₀H₄₁N₄O₃, 505.3173. Found 505.3188.

Step 4: Synthesis of (±)-(4aR,13bS)-4,9-dimethyl-1,2,3,4,4a,5,6,13b-octahydro-8H-[1,6]naphthyridino[5,6-b]quinazolin-8-one ((±)-trans-5b) and (±)-(4aR,13bR)-4,9-dimethyl-1,2,3,4,4a,5,6,13b-octahydro-8H-[1,6]naphthyridino[5,6-b]quinazolin-8-one((±)-cis-5b). Both diastereomers of 5b were synthesized according to general procedure 6 starting from 4b. Purified by flash chromatography: 10-45% X/hexanes (X=2% NEt₃ 3:1 EtOAc/EtOH). (±)-trans-5b+(±)-cis-5b: 47.5 mg (84%).

(±)-trans-5b was isolated as an off white solid (27 mg). ¹H NMR (400 MHz, CDCl₃) δ 7.56-7.47 (m, 1H), 7.44 (dd, J=8.2, 1.4 Hz, 1H), 7.15 (d, J=7.2 Hz, 1H), 4.15 (ddd, J=14.4, 6.1, 4.9 Hz, 1H), 4.00 (ddd, J=14.8, 9.6, 5.9 Hz, 1H), 2.96 (ddt, J=11.7, 4.1, 2.0 Hz, 1H), 2.85 (s, 3H), 2.74 (dd, J=13.4, 3.7 Hz, 1H), 2.64 (td, J=11.1, 3.7 Hz, 1H), 2.43 (dq, J=13.4, 5.0 Hz, 1H), 2.36 (s, 3H), 2.10 (td, J=11.9, 3.2 Hz, 1H), 1.95 (td, J=10.3, 4.5 Hz, 1H), 1.91-1.71 (m, 3H), 1.43 (tdd, J=13.1, 11.6, 4.5 Hz, 1H). HRMS (ESI) m/z: [M+H]⁺ Calcd for C₁₇H₂₂N₃O, 284.1757. Found 284.1750.

(±)-cis-10b was isolated as a light orange solid (21 mg). ¹H NMR (400 MHz, CDCl₃) δ 7.55-7.47 (m, 1H), 7.43 (dd, J=8.2, 1.3 Hz, 1H), 7.13 (dt, J=7.2, 1.1 Hz, 1H), 4.00 (qdt, J=14.3, 10.5, 4.7 Hz, 2H), 3.00 (p, J=3.4 Hz, 1H), 2.85 (s, 3H), 2.76 (dq, J=11.8, 3.3 Hz, 2H), 2.57 (dt, J=6.3, 3.1 Hz, 1H), 2.34 (dq, J=14.8, 5.0 Hz, 1H), 2.29 (s, 3H), 2.20 (td, J=10.9, 3.4 Hz, 1H), 1.94 (ddd, J=19.7, 10.1, 6.7 Hz, 1H), 1.71-1.44 (m, 3H). HRMS (ESI) m/z: [M+H]⁺ Calcd for C₁₇H₂₂N₃O, 284.1757. Found 284.1754.

### Example 8: Synthesis of (±)-((4aR,13bS)-9-chloro-4-methyl-1,2,3,4,4a,5,6,13b-octahydro-8H-[1,6]naphthyridino[5,6-b]quinazolin-8-one (((±)-trans-5c) and (±)-(4aR,13bR)-9-chloro-4-methyl-1,2,3,4,4a,5,6,13b-octahydro-8H-[1,6]naphthyridino[5,6-b]quinazolin-8-one (((±)-trans-5c)

Prepared according to Scheme 2.

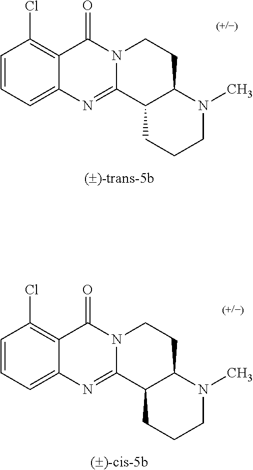 [SMILES: CN1CCC[C@@H]2c3nc4cccc(Cl)c4c(=O)n3CC[C@H]21 ; CN1CCC[C@H]2c3nc4cccc(Cl)c4c(=O)n3CC[C@H]21]

Step 1: Synthesis of tert-butyl (4-(5-chloro-3-neopentyl-4-oxo-3,4-dihydroquinazolin-2-yl)butyl)(methyl)carbamate (2c). Compound 2c was synthesized according to general procedure 3 from 2-amino-6-chloro-benzoic acid and 1a. Purification by flash chromatography (5-25% X/hexanes, X=4:1 EtOAc/DCM) delivered 2b as a yellow oil (1.25 g, 70%). ¹H NMR (400 MHz, CDCl₃) δ 7.56-7.45 (m, 2H), 7.39 (dd, J=7.5, 1.5 Hz, 1H), 4.61-3.62 (m, 2H), 3.25 (t, J=7.1 Hz, 2H), 2.84 (d, J=6.1 Hz, 5H), 1.76 (t, J=7.8 Hz, 2H), 1.68-1.57 (m, 2H), 1.44 (s, 9H), 0.99 (s, 9H). HRMS (ESI) m/z: [M+H]⁺ Calcd for C₂₃H₃₅ClN₃O₃, 436.2361. Found 436.2353.

Step 2: Synthesis of 5-chloro-2-(4-(methylamino)butyl)-3-neopentylquinazolin-4(3H)-one bis-hydrochloride salt. Compound 3c was synthesized according to general procedure 4 from 2c, which afforded 3c as a white solid (0.90 g, 84%). ¹H NMR (400 MHz, DMSO) δ 9.14 (d, J=7.6 Hz, 2H), 7.83-7.71 (m, 2H), 7.58 (dd, J=7.5, 1.5 Hz, 1H), 4.06 (s, 2H), 3.03 (t, J=7.0 Hz, 2H), 2.90 (p, J=6.7 Hz, 2H), 2.50-2.45 (m, 4H), 1.80 (dt, J=10.3, 6.3 Hz, 4H), 0.93 (s, 9H). HRMS (ESI) m/z: [M+H]⁺ Calcd for C₁₈H₂₇ClN₃O, 336.1837. Found 336.1837.

Step 3.1: Synthesis of benzyl (2-(3-(5-chloro-3-neopentyl-4-oxo-3,4-dihydroquinazolin-2-yl)-1-methylpiperidin-2-yl)ethyl)carbamate (4c). Compound 4c was synthesized according to general procedure 5.1 from 3a. Purification by reverse phase flash chromatography (10-90% MeOH/H₂O) which afforded 4c as an off-white solid (0.44 g, 69%). 4c was used directly in the next step without analysis.

Step 4: Synthesis of (±)-((4aR,13bS)-9-chloro-4-methyl-1,2,3,4,4a,5,6,13b-octahydro-8H-[1,6]naphthyridino[5,6-b]quinazolin-8-one (((±)-trans-5c) and (±)-(4aR,13bR)-9-chloro-4-methyl-1,2,3,4,4a,5,6,13b-octahydro-8H-[1,6]naphthyridino[5,6-b]quinazolin-8-one (((±)-trans-5c). Both diastereomers of 5c were synthesized according to general procedure 6 starting from 4c. Purified by flash chromatography 10-60% X/hexanes (X=2% NEt₃ 3:1 EtOAc/EtOH). (±)-trans-5c+(±)-cis-5c: 138 mg (76%).

(±)-trans-5c was isolated as a light brown solid (46 mg). ¹H NMR (400 MHz, CDCl₃) δ 7.56-7.50 (m, 2H), 7.40 (dd, J=6.4, 2.6 Hz, 1H), 4.16 (ddd, J=14.5, 6.1, 4.8 Hz, 1H), 4.02 (ddd, J=14.9, 9.6, 5.9 Hz, 1H), 2.96 (ddt, J=11.7, 4.1, 1.9 Hz, 1H), 2.77-2.69 (m, 1H), 2.65 (td, J=11.1, 3.7 Hz, 1H), 2.44 (ddt, J=13.4, 5.9, 4.7 Hz, 1H), 2.36 (s, 3H), 2.10 (td, J=12.0, 3.2 Hz, 1H), 1.96 (dt, J=10.4, 5.2 Hz, 1H), 1.91-1.70 (m, 3H), 1.43 (tdd, J=13.0, 11.5, 4.4 Hz, 1H). HRMS (ESI) m/z: [M+H]⁺ Calcd for C₁₆H₁₉ClN₃O, 304.1211. Found 304.1200.

(±)-cis-5c was isolated as a light brown solid (92 mg). ¹H NMR (400 MHz, CDCl₃) δ 7.55-7.45 (m, 2H), 7.37 (dd, J=6.6, 2.4 Hz, 1H), 4.07 (ddd, J=14.2, 5.8, 3.9 Hz, 1H), 3.95 (ddd, J=14.2, 11.2, 5.0 Hz, 1H), 2.97 (q, J=3.6 Hz, 1H), 2.84-2.72 (m, 2H), 2.53 (dt, J=6.0, 3.1 Hz, 1H), 2.35 (dtd, J=14.4, 5.1, 3.9 Hz, 1H), 2.27 (s, 3H), 2.17 (td, J=10.8, 3.3 Hz, 1H), 1.92 (dddd, J=14.2, 11.2, 5.9, 2.7 Hz, 1H), 1.69-1.47 (m, 3H). HRMS (ESI) m/z: [M+H]⁺ Calcd for C₁₆H₁₉ClN₃O, 304.1211. Found 304.1202.

### Example 9: Synthesis of (±)-(4aR,13bS)-10-chloro-4-methyl-1,2,3,4,4a,5,6,13b-octahydro-8H-[1,6]naphthyridino[5,6-b]quinazolin-8-one ((±)-trans-5d) and (±)-(4aR,13bR)-10-chloro-4-methyl-1,2,3,4,4a,5,6,13b-octahydro-8H-[1,6]naphthyridino[5,6-b]quinazolin-8-one ((±)-cis-5d)

Prepared according to Scheme 2.

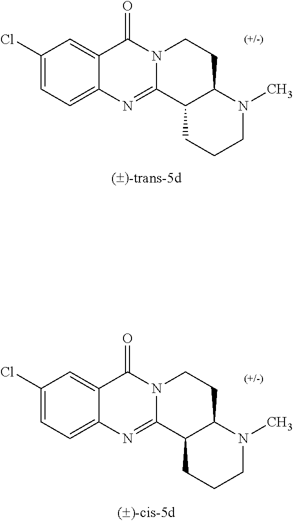 [SMILES: CN1CCC[C@@H]2c3nc4ccc(Cl)cc4c(=O)n3CC[C@H]21 ; CN1CCC[C@H]2c3nc4ccc(Cl)cc4c(=O)n3CC[C@H]21]

Step 1: Synthesis of tert-butyl (4-(6-chloro-3-neopentyl-4-oxo-3,4-dihydroquinazolin-2-yl)butyl)(methyl)carbamate (2d). Compound 2d was synthesized according to general procedure 3 from 2-amino-5-chloro-benzoic acid and 1a. Purification by flash chromatography (5-20% X/hexanes, X=4:1 EtOAc/DCM) delivered 2d as a colorless oil (1.03 g, 79%). ¹H NMR (400 MHz, CDCl₃) δ 8.18 (d, J=2.4 Hz, 1H), 7.62 (dd, J=8.7, 2.5 Hz, 1H), 7.53 (d, J=8.6 Hz, 1H), 4.38-3.90 (m, 2H), 3.26 (t, J=7.0 Hz, 2H), 2.86 (d, J=16.8 Hz, 5H), 1.76 (dd, J=13.8, 6.4 Hz, 2H), 1.69-1.58 (m, 2H), 1.44 (s, 9H), 0.99 (s, 9H). HRMS (ESI) m/z: [M+H]⁺ Calcd for C₂₃H₃₅ClN₃O₃, 436.2361. Found 436.2352.

Step 2: Synthesis of 6-chloro-2-(4-(methylamino)butyl)-3-neopentylquinazolin-4(3H)-one bis-hydrochloride salt (3d). Compound 3d was synthesized according to general procedure 4 from 2d, which afforded 3d as a white solid (1.25 g, 84%). as a white solid (1.02 g, 86%). ¹H NMR (400 MHz, DMSO) δ 9.14 (s, 2H), 8.06 (d, J=2.5 Hz, 1H), 7.89 (dd, J=8.7, 2.5 Hz, 1H), 7.77 (d, J=8.7 Hz, 1H), 4.09 (s, 2H), 3.01 (t, J=7.1 Hz, 2H), 2.96-2.85 (m, 2H), 2.49 (t, J=5.4 Hz, 4H), 1.80 (ddq, J=16.9, 8.7, 5.3, 2.9 Hz, 4H), 0.93 (s, 9H). HRMS (ESI) m/z: [M+H]⁺ Calcd for C₁₈H₂₇ClN₃O, 336.1837. Found 336.1826.

Step 3.1: Synthesis of benzyl (2-(3-(6-chloro-3-neopentyl-4-oxo-3,4-dihydroquinazolin-2-yl)-1-methylpiperidin-2-yl)ethyl)carbamate (4d). Compound 4d was synthesized according to general procedure 5.1 from 3d. Purification by reverse phase flash chromatography (10-75% MeOH/H₂O) afforded 4d a white solid (223 mg, 85%). ¹H NMR (400 MHz, CDCl₃) δ 8.16 (dd, J=10.3, 2.5 Hz, 1H), 7.72-7.51 (m, 1.3H), 7.43-7.28 (m, 5.5H), 7.14 (s, 0.6H), 6.22 (s, 0.6H), 5.11 (q, J=12.1 Hz, 1.8H), 5.00 (d, J=7.9 Hz, 0.5H), 4.74 (d, J=14.2 Hz, 0.3H), 4.48 (s, 0.7H), 3.59 (dt, J=12.3, 3.3 Hz, 0.6H), 3.38-3.31 (m, 1.7H), 3.10 (ddd, J=12.2, 9.5, 3.2 Hz, 0.8H), 3.00-2.68 (m, 2.9H), 2.52 (s, 1H), 2.45-2.29 (m, 3H), 2.28-2.18 (m, 0.3H), 2.00-1.38 (m, 5H), 1.05-0.95 (m, 9H). HRMS (ESI) m/z: [M+H]⁺ Calcd for C₂₉H₃₈ClN₄O₃, 525.2627. Found 525.2639.

Step 4: Synthesis of (±)-(4aR,13bS)-10-chloro-4-methyl-1,2,3,4,4a,5,6,13b-octahydro-8H-[1,6]naphthyridino[5,6-b]quinazolin-8-one ((±)-trans-5d) and (±)-(4aR,13bR)-10-chloro-4-methyl-1,2,3,4,4a,5,6,13b-octahydro-8H-[1,6]naphthyridino[5,6-b]quinazolin-8-one ((±)-cis-5d). Both diastereomers of 5d were synthesized according to general procedure 6 starting from 4d. Purified by flash chromatography: 10-50% X/hexanes (X=2% NEt₃ 3:1 EtOAc/EtOH). (±)-trans-5d+(±)-cis-5d: 52.9 mg (87%).

(±)-trans-5d was isolated as a yellow white solid (37.5 mg). ¹H NMR (400 MHz, CDCl₃) δ 8.17 (d, J=2.4 Hz, 1H), 7.60 (dd, J=8.7, 2.4 Hz, 1H), 7.54 (d, J=8.7 Hz, 1H), 4.20-4.04 (m, 2H), 2.95 (ddt, J=11.5, 4.0, 2.1 Hz, 1H), 2.74-2.61 (m, 2H), 2.41 (dq, J=13.3, 5.4 Hz, 1H), 2.35 (s, 3H), 2.09 (td, J=12.0, 3.1 Hz, 1H), 1.96 (td, J=10.1, 4.8 Hz, 1H), 1.88-1.72 (m, 3H), 1.51-1.40 (m, 1H). HRMS (ESI) m/z: [M+H]⁺ Calcd for C₁₆H₁₉ClN₃O, 304.1211. Found 304.1209.

(±)-cis-5d was isolated as a yellow white solid (15.4 mg). ¹H NMR (400 MHz, CDCl₃) δ 8.20 (d, J=2.4 Hz, 1H), 7.61 (dd, J=8.7, 2.4 Hz, 1H), 7.54 (d, J=8.7 Hz, 1H), 4.12 (ddd, J=14.1, 5.8, 4.1 Hz, 1H), 4.01 (ddd, J=14.1, 11.1, 5.0 Hz, 1H), 2.99 (q, J=3.6 Hz, 1H), 2.87-2.74 (m, 2H), 2.56 (dd, J=5.6, 3.0 Hz, 1H), 2.35 (dq, J=14.5, 4.8 Hz, 1H), 2.28 (s, 3H), 2.19 (td, J=9.8, 8.6, 3.1 Hz, 1H), 1.96 (tdd, J=11.4, 5.9, 2.9 Hz, 1H), 1.67-1.50 (m, 3H). HRMS (ESI) m/z: [M+H]⁺ Calcd for C₁₆H₁₉ClN₃O, 304.1211. Found 304.1209.

### Example 10: Synthesis of (±)-(4aR,13bS)-10-bromo-4-methyl-1,2,3,4,4a,5,6,13b-octahydro-8H-[1,6]naphthyridino[5,6-b]quinazolin-8-one ((±)-trans-5e) and (±)-(4aR,13bR)-10-bromo-4-methyl-1,2,3,4,4a,5,6,13b-octahydro-8H-[1,6]naphthyridino[5,6-b]quinazolin-8-one ((±)-cis-5e)

Prepared according to Scheme 2.

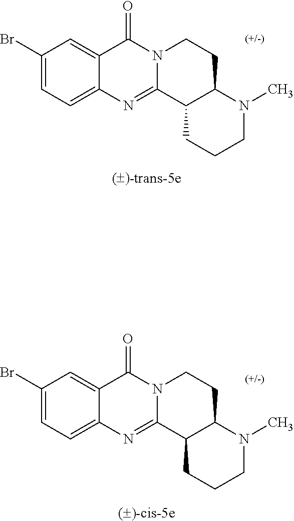 [SMILES: CN1CCC[C@@H]2c3nc4ccc(Br)cc4c(=O)n3CC[C@H]21 ; CN1CCC[C@H]2c3nc4ccc(Br)cc4c(=O)n3CC[C@H]21]

Step 1: Synthesis of tert-butyl (4-(6-bromo-3-neopentyl-4-oxo-3,4-dihydroquinazolin-2-yl)butyl)(methyl)carbamate (2e). Compound 2e was synthesized according to general procedure 3 from 2-amino-5-bromo-benzoic acid and 1a. Purification by flash chromatography (5-15% X/hexanes, X=4:1 EtOAc/DCM) delivered 2e as a colorless oil (0.74 g, 74%). ¹H NMR (400 MHz, CDCl₃) δ 8.35 (d, J=2.3 Hz, 1H), 7.76 (dd, J=8.7, 2.3 Hz, 1H), 7.46 (d, J=8.6 Hz, 1H), 4.08 (s, 2H), 3.26 (t, J=6.9 Hz, 2H), 2.92-2.79 (m, 5H), 1.81-1.73 (m, 2H), 1.68-1.56 (m, 2H), 1.44 (s, 9H), 0.98 (s, 9H). HRMS (ESI) m/z: [M+H]⁺ Calcd for C₂₃H₃₅BrN₃O₃ C₂₃H₃₅BrN₃O₃, 480.1856. Found 480.1844.

Step 2: Synthesis of 6-bromo-2-(4-(methylamino)butyl)-3-neopentylquinazolin-4(3H)-one bis-hydrochloride salt (3e). Compound 3e was synthesized according to general procedure 4 from 2e, which afforded 3d as a white solid (0.67 g, quant.). ¹H NMR (400 MHz, DMSO) δ 9.14 (q, J=6.4, 5.8 Hz, 2H), 8.19 (d, J=2.3 Hz, 1H), 8.00 (dd, J=8.7, 2.4 Hz, 1H), 7.70 (d, J=8.7 Hz, 1H), 6.50 (s, 1H), 4.09 (s, 2H), 3.01 (t, J=7.1 Hz, 2H), 2.90 (q, J=6.6 Hz, 2H), 2.48 (t, J=5.4 Hz, 3H), 1.91-1.68 (m, 4H), 0.93 (s, 9H). HRMS (ESI) m/z: [M+H]⁺ Calcd for C₁₈H₂₇BrN₃O, 380.1332. Found 380.1320.

Step 3.1: Synthesis of benzyl (2-(3-(6-bromo-3-neopentyl-4-oxo-3,4-dihydroquinazolin-2-yl)-1-methylpiperidin-2-yl)ethyl)carbamate (4e). Compound 4e was synthesized according to general procedure 5.1 from 3e. Purification by reverse phase flash chromatography (10-75% MeOH/H₂O) afforded 4e an off-white solid (240 mg, 84%). ¹H NMR (400 MHz, CDCl₃) δ 8.37-8.29 (m, 1H), 7.93-7.79 (m, 0.2H), 7.76 (dd, J=8.6, 2.4 Hz, 0.3H), 7.58 (d, J=8.7 Hz, 0.7H), 7.49 (d, J=8.7 Hz, 0.3H), 7.45-7.25 (m, 5.2H), 6.21 (s, 0.7H), 5.11 (q, J=12.0 Hz, 1.6H), 5.00 (dd, J=12.0, 7.7 Hz, 0.6H), 4.74 (d, J=14.2 Hz, 0.3H), 4.48 (s, 0.8H), 3.58 (dt, J=12.3, 3.2 Hz, 0.6H), 3.37-3.29 (m, 1.6H), 3.09 (ddd, J=12.2, 9.5, 3.2 Hz, 0.8H), 2.98-2.67 (m, 3H), 2.59-2.47 (m, 1.1H), 2.47-2.33 (m, 3H), 2.28-2.16 (m, 0.4H), 2.02-1.89 (m, 0.8H), 1.87-1.64 (m, 5.1H), 1.62-1.33 (m, 0.3H), 1.04-0.95 (m, 9H). HRMS (ESI) m/z: [M+H]⁺ Calcd for C₂₉H₃₈BrN₄O₃, 569.2122.

Step 4: Synthesis of (±)-(4aR,13bS)-10-bromo-4-methyl-1,2,3,4,4a,5,6,13b-octahydro-8H-[1,6]naphthyridino[5,6-b]quinazolin-8-one ((±)-trans-5e) and (±)-(4aR,13bR)-10-bromo-4-methyl-1,2,3,4,4a,5,6,13b-octahydro-8H-[1,6]naphthyridino[5,6-b]quinazolin-8-one ((±)-cis-5e). Both diastereomers of 5e were synthesized according to general procedure 6 starting from 4e. Purified by flash chromatography: 10-50% X/hexanes (X=2% NEt₃ 3:1 EtOAc/EtOH). (±)-trans-5e+(±)-cis-5e: 52.9 mg (87%).

(±)-trans-5e was isolated as a yellow white solid (46.8 mg). ¹H NMR (400 MHz, CDCl₃) δ 8.36 (d, J=2.3 Hz, 1H), 7.76 (dd, J=8.7, 2.3 Hz, 1H), 7.49 (d, J=8.7 Hz, 1H), 4.22-4.05 (m, 2H), 2.97 (tq, J=11.9, 11.5, 2.2 Hz, 1H), 2.77-2.60 (m, 2H), 2.42 (dq, J=13.3, 5.5 Hz, 1H), 2.36 (s, 3H), 2.10 (td, J=12.0, 3.1 Hz, 1H), 2.05-1.92 (m, 1H), 1.91-1.69 (m, 3H), 1.45 (tdd, J=13.0, 11.3, 4.4 Hz, 1H). HRMS (ESI) m/z: [M+H]⁺ Calcd for C₁₆H₁₉BrN₃O, 348.0706. Found 348.0717.

(±)-cis-5e was isolated as a yellow white solid (18.5 mg). ¹H NMR (400 MHz, CDCl₃) δ 8.36 (d, J=2.3 Hz, 1H), 7.75 (dd, J=8.7, 2.3 Hz, 1H), 7.48 (d, J=8.7 Hz, 1H), 4.11 (ddd, J=14.1, 5.9, 4.1 Hz, 1H), 4.01 (ddd, J=14.1, 11.0, 5.0 Hz, 1H), 2.99 (q, J=3.6 Hz, 1H), 2.86-2.73 (m, 2H), 2.56 (dt, J=6.3, 3.1 Hz, 1H), 2.35 (dq, J=14.5, 4.9 Hz, 1H), 2.28 (s, 3H), 2.19 (td, J=10.9, 3.6 Hz, 1H), 1.95 (dddd, J=14.2, 11.0, 5.9, 2.8 Hz, 1H), 1.66-1.52 (m, 3H). HRMS (ESI) m/z: [M+H]⁺ Calcd for C₁₆H₁₉BrN₃O, 348.0706. Found 348.0698.

### Example 11: Synthesis of (±)-(4aR,13bS)-10-fluoro-4-methyl-1,2,3,4,4a,5,6,13b-octahydro-8H-[1,6]naphthyridino[5,6-b]quinazolin-8-one ((±)-trans-5f) and (±)-(4aR,13bR)-10-fluoro-4-methyl-1,2,3,4,4a,5,6,13b-octahydro-8H-[1,6]naphthyridino[5,6-b]quinazolin-8-one ((±)-cis-5f)

Prepared according to Scheme 2.

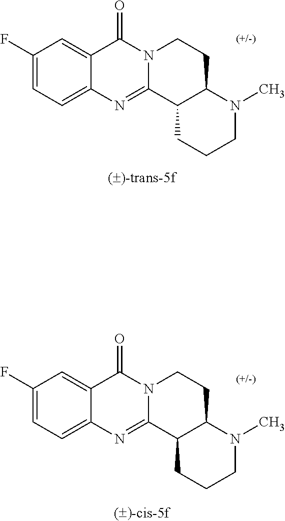 [SMILES: CN1CCC[C@@H]2c3nc4ccc(F)cc4c(=O)n3CC[C@H]21 ; CN1CCC[C@H]2c3nc4ccc(F)cc4c(=O)n3CC[C@H]21]

Step 1: Synthesis of tert-butyl (4-(6-fluoro-3-neopentyl-4-oxo-3,4-dihydroquinazolin-2-yl)butyl)(methyl)carbamate (2f). Compound 2f was synthesized according to general procedure 3 from 2-amino-5-fluoro-benzoic acid and 1a. Purification by flash chromatography (5-20% X/hexanes, X=4:1 EtOAc/DCM) delivered 2f as a colorless oil (1.02 g, 73%). ¹H NMR (400 MHz, CDCl₃) δ 7.85 (dd, J=8.6, 3.0 Hz, 1H), 7.59 (dd, J=8.9, 4.9 Hz, 1H), 7.41 (td, J=8.5, 3.0 Hz, 1H), 4.10 (s, 2H), 3.26 (t, J=7.0 Hz, 2H), 2.86 (d, J=17.3 Hz, 5H), 1.81-1.70 (m, 2H), 1.63 (p, J=6.2, 5.2 Hz, 2H), 1.44 (s, 9H), 0.99 (s, 9H). HRMS (ESI) m/z: [M+H]⁺ Calcd for C₂₃H₃₅FN₃O₃, 420.2657. Found 420.2655.

Step 2: Synthesis of 6-fluoro-2-(4-(methylamino)butyl)-3-neopentylquinazolin-4(3H)-one bis-hydrochloride salt (3f). Compound 3f was synthesized according to general procedure 4 from 2f, which afforded 3f as a white solid (0.86 g, 93%). ¹H NMR (400 MHz, DMSO) δ 9.24-9.06 (m, 2H), 7.90-7.73 (m, 3H), 4.10 (s, 2H), 3.05 (t, J=7.1 Hz, 2H), 2.92 (p, J=6.8 Hz, 2H), 2.50 (d, J=11.1 Hz, 4H), 1.88-1.75 (m, 4H), 0.95 (s, 9H). HRMS (ESI) m/z: [M+H]⁺ Calcd for C₁₈H₂₇FN₃O, 320.2138. Found 320.2135.

Step 3.1: Synthesis of benzyl (2-(3-(6-fluoro-3-neopentyl-4-oxo-3,4-dihydroquinazolin-2-yl)-1-methylpiperidin-2-yl)ethyl)carbamate (4f). Compound 4f was synthesized according to general procedure 5.1 from 3f. Purification by reverse phase flash chromatography (10-70% MeOH/H₂O) afforded 4f a white solid (192 mg, 76%). %). ¹H NMR (400 MHz, CDCl₃) δ 7.88-7.77 (m, 1H), 7.72 (dd, J=9.0, 4.8 Hz, 0.6H), 7.62 (dd, J=8.9, 4.9 Hz, 0.3H), 7.50-7.26 (m, 5.5H), 6.93 (s, 0.5H), 6.29 (s, 0.8H), 5.11 (q, J=12.0 Hz, 1.7H), 5.06-4.94 (m, 0.6H), 4.75 (d, J=14.2 Hz, 0.3H), 4.49 (s, 0.8H), 3.59 (dt, J=12.1, 3.1 Hz, 0.9H), 3.42-3.26 (m, 1.4H), 3.10 (ddd, J=12.2, 9.5, 3.2 Hz, 0.7H), 2.98-2.66 (m, 2.6H), 2.57-2.50 (m, 1H), 2.46-2.32 (m, 2.8H), 2.26-2.18 (m, 0.3H), 1.94 (d, J=13.1 Hz, 1H), 1.88-1.61 (m, 3.8H), 1.60-1.29 (m, 1.2H), 1.05-0.96 (m, 9H). HRMS (ESI) m/z: [M+H]⁺ Calcd for C₂₉H₃₈FN₄O₃, 509.2922. Found 509.2935.

Step 4: Synthesis of (±)-(4aR,13bS)-10-fluoro-4-methyl-1,2,3,4,4a,5,6,13b-octahydro-8H-[1,6]naphthyridino[5,6-b]quinazolin-8-one ((±)-trans-5f) and (±)-(4aR,13bR)-10-fluoro-4-methyl-1,2,3,4,4a,5,6,13b-octahydro-8H-[1,6]naphthyridino[5,6-b]quinazolin-8-one ((±)-cis-5f). Both diastereomers of 5f were synthesized according to general procedure 6 starting from 4f. Purified by flash chromatography: 10-50% X/hexanes (X=2% NEt₃ 3:1 EtOAc/EtOH). (±)-trans-5f+(±)-cis-5f: 48.9 mg (85%).

(±)-trans-5f was isolated as a yellow/white solid (35 mg). ¹H NMR (400 MHz, CDCl₃) δ 7.84 (dd, J=8.5, 3.0 Hz, 1H), 7.61 (dd, J=8.9, 4.8 Hz, 1H), 7.40 (td, J=8.5, 3.0 Hz, 1H), 4.14 (ddd, J=8.4, 5.8, 2.1 Hz, 2H), 2.99 (dt, J=12.8, 3.3 Hz, 1H), 2.70 (ddt, J=10.1, 6.2, 3.5 Hz, 2H), 2.47-2.33 (m, 4H), 2.12 (td, J=11.8, 3.4 Hz, 1H), 2.00 (td, J=10.0, 4.9 Hz, 1H), 1.94-1.71 (m, 3H), 1.46 (dtd, J=14.1, 12.4, 4.6 Hz, 1H). HRMS (ESI) m/z: [M+H]⁺ Calcd for C₁₆H₁₉FN₃O, 288.1507. Found 288.1505.

(±)-cis-5f was isolated as an off white solid (13.9 mg). ¹H NMR (400 MHz, CDCl₃) δ 7.86 (dd, J=8.5, 3.0 Hz, 1H), 7.61 (dd, J=8.9, 4.9 Hz, 1H), 7.40 (td, J=8.5, 3.0 Hz, 1H), 4.17-3.93 (m, 2H), 3.02 (d, J=3.9 Hz, 1H), 2.79 (dq, J=12.0, 4.6, 3.5 Hz, 2H), 2.65-2.52 (m, 1H), 2.42-2.26 (m, 4H), 2.21 (t, J=9.1 Hz, 1H), 1.98 (q, J=13.6, 9.8 Hz, 1H), 1.75-1.50 (m, 3H). HRMS (ESI) m/z: [M+H]⁺ Calcd for C₁₆H₁₉FN₃O, 288.1507. Found 288.1506.

### Example 12: Synthesis of (±)-(4aR,13bS)-10-methoxy-4-methyl-1,2,3,4,4a,5,6,13b-octahydro-8H-[1,6]naphthyridino[5,6-b]quinazolin-8-one ((±)-trans-5g) and (±)-(4aR,13bR)-10-methoxy-4-methyl-1,2,3,4,4a,5,6,13b-octahydro-8H-[1,6]naphthyridino[5,6-b]quinazolin-8-one ((±)-cis-5g)

Prepared according to Scheme 2.

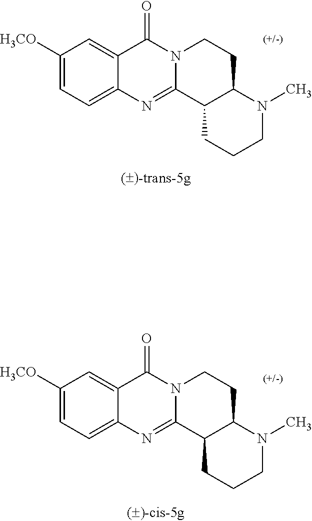 [SMILES: COc1ccc2nc3n(c(=O)c2c1)CC[C@@H]1[C@@H]3CCCN1C ; COc1ccc2nc3n(c(=O)c2c1)CC[C@@H]1[C@H]3CCCN1C]

Step 1: Synthesis of tert-butyl (4-(6-methoxy-3-neopentyl-4-oxo-3,4-dihydroquinazolin-2-yl)butyl)(methyl)carbamate (2g). Compound 2g was synthesized according to general procedure 3 from 2-amino-5-methoxy-benzoic acid and 1a. Purification by flash chromatography (5-20% X/hexanes, X=4:1 EtOAc/DCM) delivered 2g as a yellow oil (0.82 g, 64%). ¹H NMR (400 MHz, CDCl₃) δ 7.59 (d, J=2.9 Hz, 1H), 7.53 (dd, J=8.9, 1.1 Hz, 1H), 7.29 (ddd, J=8.9, 2.9, 1.0 Hz, 1H), 4.46-3.89 (m, 2H), 3.89 (d, J=1.1 Hz, 3H), 3.25 (t, J=7.0 Hz, 2H), 2.92-2.77 (m, 5H), 1.75 (p, J=8.0 Hz, 2H), 1.62 (p, J=7.4 Hz, 2H), 1.44 (d, J=1.0 Hz, 9H), 1.00 (d, J=1.0 Hz, 9H). HRMS (ESI) m/z: [M+H]⁺ Calcd for C₂₄H₃₈N₃O₄, 432.2857. Found 432.2854.

Step 2: Synthesis of 6-methoxy-2-(4-(methylamino)butyl)-3-neopentylquinazolin-4(3H)-one bis-hydrochloride salt (3g). Compound 3g was synthesized according to general procedure 4 from 2g, which afforded 3g as a white solid (0.64 g, 90%). ¹H NMR (400 MHz, DMSO) δ 9.18 (q, J=6.6, 5.7 Hz, 2H), 7.88 (d, J=8.9 Hz, 1H), 7.55 (dd, J=8.9, 3.0 Hz, 1H), 7.52 (d, J=2.8 Hz, 1H), 4.20-4.00 (m, 2H), 3.89 (s, 3H), 3.13 (t, J=7.2 Hz, 2H), 2.91 (q, J=7.0, 6.6 Hz, 2H), 2.52-2.48 (m, 4H), 1.84 (td, J=15.6, 13.8, 7.1 Hz, 4H), 0.96 (s, 9H). HRMS (ESI) m/z: [M+H]⁺ Calcd for C₁₉H₃₀N₃O₂, 332.2333. Found 332.2334.

Step 3.1: Synthesis of benzyl (2-(3-(6-methoxy-3-neopentyl-4-oxo-3,4-dihydroquinazolin-2-yl)-1-methylpiperidin-2-yl)ethyl)carbamate (4g). Compound 4g was synthesized according to general procedure 5.1 from 3g. Purification by reverse phase flash chromatography (10-80% MeOH/H₂O) which afforded 4a as a white solid (226 mg, 92%). Purification by reverse phase flash chromatography (10-70% MeOH/H₂O) afforded 4g an off-white solid (196 mg, 76%). ¹H NMR (400 MHz, CDCl₃) δ 7.68 (d, J=9.0 Hz, 0.7H), 7.57 (dd, J=6.4, 3.1 Hz, 2.1H), 7.46-7.28 (m, 5.9H), 6.87 (d, J=8.8 Hz, 0.6H), 6.44 (s, 0.8H), 5.13 (q, J=12.1 Hz, 1.7H), 5.06-4.95 (m, 0.6H), 4.78 (d, J=14.2 Hz, 0.3H), 4.53 (s, 0.6H), 3.93-3.84 (m, 3.1H), 3.58 (dq, J=11.8, 4.2, 3.3 Hz, 0.6H), 3.41-3.29 (m, 1.7H), 3.09 (ddd, J=12.2, 9.5, 3.2 Hz, 0.8H), 2.98-2.65 (m, 2.8H), 2.51 (s, 1.2H), 2.42 (s, 2.4H), 2.30-2.10 (m, 0.3H), 1.99-1.35 (m, 4.3H), 1.00 (d, J=7.5 Hz, 9H). HRMS (ESI) m/z: [M+H]⁺ Calcd for C₃₀H₄₁N₄O₄, 521.3122. Found 521.3137.

Step 4: Synthesis of (±)-(4aR,13bS)-10-methoxy-4-methyl-1,2,3,4,4a,5,6,13b-octahydro-8H-[1,6]naphthyridino[5,6-b]quinazolin-8-one ((±)-trans-5g) and (±)-(4aR,13bR)-10-methoxy-4-methyl-1,2,3,4,4a,5,6,13b-octahydro-8H-[1,6]naphthyridino[5,6-b]quinazolin-8-one ((±)-cis-5g). Both diastereomers of 5g were synthesized according to general procedure 6 starting from 4g. Purified by flash chromatography: 10-50% X/hexanes (X=2% NEt₃ 3:1 EtOAc/EtOH). (±)-trans-5g+(±)-cis-5g: 49.5 mg (83%).

(±)-trans-5g was isolated as a yellow solid (33 mg). ¹H NMR (400 MHz, CDCl₃) δ 7.58 (d, J=3.0 Hz, 1H), 7.54 (d, J=8.9 Hz, 1H), 7.28 (dd, J=8.9, 3.0 Hz, 1H), 4.13 (tdd, J=14.4, 8.3, 5.7 Hz, 2H), 3.88 (s, 3H), 2.95 (ddt, J=11.6, 4.0, 2.0 Hz, 1H), 2.75-2.62 (m, 2H), 2.41 (dq, J=13.3, 5.5 Hz, 1H), 2.34 (s, 3H), 2.09 (td, J=11.9, 3.2 Hz, 1H), 1.95 (td, J=10.0, 4.8 Hz, 1H), 1.90-1.68 (m, 3H), 1.45 (tdd, J=12.8, 11.2, 4.4 Hz, 1H). HRMS (ESI) m/z: [M+H]⁺ Calcd for C₁₇H₂₂N₃O₂, 300.1707. Found 300.1703.

(±)-cis-5g was isolated as a yellow solid (16.5 mg). ¹H NMR (400 MHz, CDCl₃) δ 7.60 (d, J=3.0 Hz, 1H), 7.54 (d, J=8.9 Hz, 1H), 7.29 (dd, J=8.9, 3.0 Hz, 1H), 4.20-3.99 (m, 2H), 3.90 (s, 3H), 3.00 (q, J=3.8 Hz, 1H), 2.86-2.72 (m, 2H), 2.57 (dt, J=6.2, 3.3 Hz, 1H), 2.34 (dq, J=14.7, 4.9 Hz, 1H), 2.29 (s, 3H), 2.19 (td, J=11.0, 3.1 Hz, 1H), 1.95 (dddd, J=17.0, 10.7, 6.0, 2.8 Hz, 1H), 1.71-1.50 (m, 3H). HRMS (ESI) m/z: [M+H]⁺ Calcd for C₁₇H₂₂N₃O₂, 300.1707. Found 300.1701.

### Example 13: Synthesis of (±)-(4aR,13bS)-4,10-dimethyl-1,2,3,4,4a,5,6,13b-octahydro-8H-[1,6]naphthyridino[5,6-b]quinazolin-8-one ((±)-trans-5h) and (±)-(4aR,13bR)-4,10-dimethyl-1,2,3,4,4a,5,6,13b-octahydro-8H-[1,6]naphthyridino[5,6-b]quinazolin-8-one ((±)-cis-5h)

Prepared according to Scheme 2.

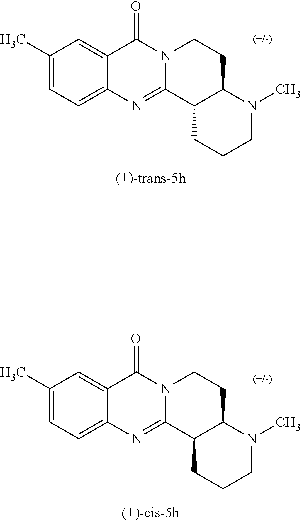

Step 1: Synthesis of tert-butyl methyl(4-(6-methyl-3-neopentyl-4-oxo-3,4-dihydroquinazolin-2-yl)butyl)carbamate (2h). Compound 2h was synthesized according to general procedure 4 from 2-amino-5-methyl-benzoic acid and 1a. Purification by flash chromatography (5-25% X/hexanes, X=4:1 EtOAc/DCM) delivered 2h as a colorless oil (1.1 g, 78%). ¹H NMR (400 MHz, CDCl₃) δ 8.01 (dt, J=1.8, 1.0 Hz, 1H), 7.53-7.46 (m, 2H), 4.46-3.78 (m, 2H), 3.25 (t, J=7.2 Hz, 2H), 2.92-2.78 (m, 5H), 2.45 (s, 3H), 1.75 (p, J=7.7 Hz, 2H), 1.62 (p, J=7.3 Hz, 2H), 1.44 (s, 9H), 0.99 (s, 9H). HRMS (ESI) m/z: [M+H]⁺ Calcd for C₂₄H₃₈N₃O₃, 416.2908. Found 416.2906.

Step 2: Synthesis of 6-methyl-2-(4-(methylamino)butyl)-3-neopentylquinazolin-4(3H)-one bis-hydrochloride salt (3h). Compound 3h was synthesized according to general procedure 4 from 2h, which afforded 3h as a white solid (0.87 g, 89%). ¹H NMR (400 MHz, Acetic) δ 11.37 (s, 5H), 8.10 (d, J=1.8 Hz, 1H), 8.03 (d, J=8.4 Hz, 1H), 7.84 (dd, J=8.5, 1.9 Hz, 1H), 4.43-4.22 (m, 2H), 3.49 (t, J=7.0 Hz, 2H), 3.23 (d, J=5.8 Hz, 2H), 2.79 (s, 3H), 2.53 (s, 3H), 2.14-2.07 (m, 3H), 1.06 (s, 9H). HRMS (ESI) m/z: [M+H]⁺ Calcd for C₁₉H₃₀N₃O, 316.2383. Found 316.2388.

Step 3.1: Synthesis of benzyl (2-(1-methyl-3-(6-methyl-3-neopentyl-4-oxo-3,4-dihydroquinazolin-2-yl)piperidin-2-yl)ethyl)carbamate (4h). Compound 4h was synthesized according to general procedure 5.1 from 3h. Purification by reverse phase flash chromatography (10-80% MeOH/H₂O) afforded 4h a white solid (195 mg, 77%). ¹H NMR (400 MHz, CDCl₃) δ 8.03-7.96 (m, 1H), 7.64 (d, J=8.3 Hz, 0.6H), 7.53-7.48 (m, 0.6H), 7.44-7.26 (m, 5.2H), 7.09 (s, 0.5H), 6.39 (s, 0.7H), 5.12 (q, J=12.1 Hz, 1.7H), 5.04-4.91 (m, 0.6H), 4.76 (d, J=14.2 Hz, 0.3H), 4.51 (s, 0.7H), 3.58 (dt, J=12.6, 3.3 Hz, 0.6H), 3.33 (s, 1.6H), 3.08 (ddd, J=12.3, 9.5, 3.2 Hz, 0.8H), 2.98-2.64 (m, 2.8H), 2.58-2.47 (m, 1.2H), 2.48-2.31 (m, 5.9H), 2.31-2.17 (m, 0.3H), 2.10-1.88 (m, 1.3H), 1.88-1.34 (m, 3.8H), 1.02-0.93 (m, J=8.2 Hz, 9H). HRMS (ESI) m/z: [M+H]⁺ Calcd for C₃₀H₄₁N₄O₃, 505.3173. Found 505.3184.

Step 4: Synthesis of (±)-(4aR,13bS)-4,10-dimethyl-1,2,3,4,4a,5,6,13b-octahydro-8H-[1,6]naphthyridino[5,6-b]quinazolin-8-one ((±)-trans-5h) and (±)-(4aR,13bR)-4,10-dimethyl-1,2,3,4,4a,5,6,13b-octahydro-8H-[1,6]naphthyridino[5,6-b]quinazolin-8-one ((±)-cis-5h). Both diastereomers of 5h were synthesized according to general procedure 6 starting from 4h. Purified by flash chromatography: 10-45% X/hexanes (X=2% NEt₃ 3:1 EtOAc/EtOH). (±)-trans-5h+(±)-cis-5h: 47.5 mg (84%).

(±)-trans-5h was isolated as an off white solid (32.7 mg). ¹H NMR (400 MHz, CDCl₃) δ 8.01 (d, J=2.1 Hz, 1H), 7.50 (t, J=1.4 Hz, 2H), 4.21-4.05 (m, 2H), 2.95 (ddt, J=11.7, 4.0, 2.0 Hz, 1H), 2.77-2.60 (m, 2H), 2.44 (s, 3H), 2.39 (dt, J=13.3, 5.5 Hz, 1H), 2.34 (s, 3H), 2.09 (td, J=11.9, 3.2 Hz, 1H), 1.95 (td, J=10.0, 4.9 Hz, 1H), 1.90-1.69 (m, 3H), 1.46 (tdd, J=12.8, 11.3, 4.4 Hz, 1H). HRMS (ESI) m/z: [M+H]⁺ Calcd for C₁₇H₂₂N₃O, 284.1757. Found 284.1749.

(±)-cis-5h was isolated as an off white solid (32.7 mg). ¹H NMR (400 MHz, CDCl₃) δ 8.03 (q, J=1.2 Hz, 1H), 7.50 (d, J=1.3 Hz, 2H), 4.16-3.98 (m, 2H), 3.01 (q, J=3.9 Hz, 1H), 2.84-2.72 (m, 2H), 2.57 (dt, J=6.3, 3.2 Hz, 1H), 2.46 (d, J=0.7 Hz, 3H), 2.34 (dt, J=14.6, 5.1 Hz, 1H), 2.29 (s, 3H), 2.20 (td, J=10.9, 3.1 Hz, 1H), 1.95 (dddd, J=17.0, 10.4, 6.1, 2.9 Hz, 1H), 1.72-1.49 (m, 3H). HRMS (ESI) m/z: [M+H]⁺ Calcd for C₁₇H₂₂N₃O, 284.1757. Found 284.1757.

### Example 14: Synthesis of (±)-(4aR,13bS)-4-methyl-8-oxo-2,3,4,4a,5,6,8,13b-octahydro-1H-[1,6]naphthyridino[5,6-b]quinazoline-10-carbonitrile ((±)-trans-5i) and (±)-(4aR,13bR)-4-methyl-8-oxo-2,3,4,4a,5,6,8,13b-octahydro-1H-[1,6]naphthyridino[5,6-b]quinazoline-10-carbonitrile ((±)-cis-5i)

Prepared according to Scheme 2.

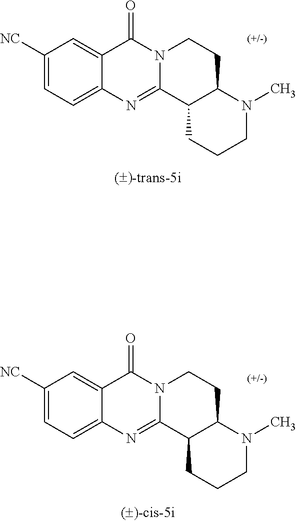 [SMILES: CN1CCC[C@@H]2c3nc4ccc(C#N)cc4c(=O)n3CC[C@H]21 ; CN1CCC[C@H]2c3nc4ccc(C#N)cc4c(=O)n3CC[C@H]21]

Step 1: Synthesis of tert-butyl (4-(6-cyano-3-neopentyl-4-oxo-3,4-dihydroquinazolin-2-yl)butyl)(methyl)carbamate (2i). Compound 2i was synthesized according to general procedure 3 from 2-amino-5-cyano-benzoic acid and 1a. Purification by flash chromatography (5-20% X/hexanes, X=4:1 EtOAc/DCM) delivered 2i a yellow oil (0.48 g, 30%). ¹H NMR (400 MHz, CDCl₃) δ 8.54 (d, J=1.9 Hz, 1H), 7.86 (dd, J=8.4, 2.0 Hz, 1H), 7.65 (d, J=8.4 Hz, 1H), 4.10 (s, 2H), 3.27 (t, J=6.7 Hz, 2H), 2.90 (d, J=8.8 Hz, 2H), 2.84 (s, 3H), 1.84-1.73 (m, 2H), 1.64 (p, J=15.2, 7.3 Hz, 2H), 1.44 (s, 9H), 0.99 (s, 9H). HRMS (ESI) m/z: [M+H]⁺ Calcd for C₂₄H₃₅N₄O₃, 427.2704. Found 427.2700.

Step 2: Synthesis of 2-(4-(methylamino)butyl)-3-neopentyl-4-oxo-3,4-dihydroquinazoline-6-carbonitrile bis-hydrochloride salt (3i). Compound 3i was synthesized according to general procedure 4 from 2i, which afforded 3i as a light tan solid (0.33 g, 84%). ¹H NMR (400 MHz, DMSO) δ 9.15-9.02 (m, 2H), 8.47 (d, J=1.9 Hz, 1H), 8.12 (dd, J=8.5, 2.0 Hz, 1H), 7.72 (d, J=8.5 Hz, 1H), 4.08 (s, 2H), 2.92 (dt, J=19.5, 6.7 Hz, 4H), 2.51-2.47 (m, 3H), 1.84-1.72 (m, 4H), 0.92 (s, 9H). HRMS (ESI) m/z: [M+H]⁺ Calcd for C₁₉H₂₇N₄O, 327.2179. Found 327.2170.

Step 3.1: Synthesis of benzyl (2-(3-(6-cyano-3-neopentyl-4-oxo-3,4-dihydroquinazolin-2-yl)-1-methylpiperidin-2-yl)ethyl)carbamate (4i). Compound 4i was synthesized according to general procedure 5.1 from 3i. Purification by reverse phase flash chromatography (10-75% MeOH/H₂O) afforded 4i an off-white solid (60.9 mg, 19 ¹H NMR (400 MHz, CDCl₃) δ 8.56-8.43 (m, 0.9H), 8.12-7.80 (m, 0.4H), 7.79-7.58 (m, 0.9H), 7.48-7.22 (m, 5.5H), 6.02 (s, 0.5H), 5.16-4.92 (m, 2.1H), 4.74 (d, J=14.2 Hz, 0.3H), 4.45 (s, 0.5H), 3.72-3.55 (m, 1H), 3.42-3.26 (m, 1.4H), 3.14 (ddd, J=12.2, 9.5, 3.2 Hz, 0.7H), 3.02-2.67 (m, 2.6H), 2.61-2.49 (m, 1.2H), 2.48-2.35 (m, 2.7H), 2.33-2.13 (m, 0.4H), 2.01-1.29 (m, 6.4H), 1.04-0.96 (m, 9H), 0.93-0.81 (m, 0.5H). HRMS (ESI) m/z: [M+H]⁺ Calcd for C₃₀H₃₈N₅O₃, 516.2969. Found 516.2957.

Step 4: Synthesis of (±)-(4aR,13bS)-4-methyl-8-oxo-2,3,4,4a,5,6,8,13b-octahydro-1H-[1,6]naphthyridino[5,6-b]quinazoline-10-carbonitrile ((±)-trans-5i) and (±)-(4aR,13bR)-4-methyl-8-oxo-2,3,4,4a,5,6,8,13b-octahydro-1H-[1,6]naphthyridino[5,6-b]quinazoline-10-carbonitrile ((±)-cis-5i). Both diastereomers of 5i were synthesized according to general procedure 6 starting from 4i. Purified by flash chromatography: 15-50% X/hexanes (X=2% NEt₃ 3:1 EtOAc/EtOH). (±)-trans-5i+(±)-cis-5i: 50.4 mg (86%).

(±)-trans-5i was isolated as a white solid (22.7 mg). ¹H NMR (400 MHz, CDCl₃) δ 8.56 (d, J=1.9 Hz, 1H), 7.86 (dd, J=8.5, 2.0 Hz, 1H), 7.69 (d, J=8.5 Hz, 1H), 4.27-4.01 (m, 2H), 2.98 (ddd, J=11.9, 4.2, 2.3 Hz, 1H), 2.79-2.65 (m, 2H), 2.45 (dq, J=13.5, 5.3 Hz, 1H), 2.37 (s, 3H), 2.11 (td, J=12.0, 3.1 Hz, 1H), 1.99 (td, J=10.2, 4.7 Hz, 1H), 1.92-1.71 (m, 3H), 1.46 (tdd, J=12.7, 11.3, 4.2 Hz, 1H). HRMS (ESI) m/z: [M+H]⁺ Calcd for C₁₇H₁₉N₄O, 295.1553. Found 295.1548.

(±)-cis-5i was isolated as a yellow solid (27.7 mg ¹H NMR (400 MHz, CDCl₃) δ 8.55 (d, J=1.9 Hz, 1H), 7.84 (dd, J=8.5, 2.0 Hz, 1H), 7.66 (d, J=8.5 Hz, 1H), 4.14 (ddd, J=14.2, 5.8, 3.7 Hz, 1H), 4.00 (ddd, J=14.1, 11.4, 5.0 Hz, 1H), 3.02 (q, J=3.6 Hz, 1H), 2.87 (dd, J=9.1, 3.6 Hz, 1H), 2.81-2.75 (m, 1H), 2.56 (dt, J=6.1, 3.1 Hz, 1H), 2.38 (dtd, J=14.2, 5.0, 3.7 Hz, 1H), 2.28 (s, 3H), 2.19 (td, J=10.3, 3.8 Hz, 1H), 1.96 (dddd, J=14.4, 11.4, 5.8, 2.7 Hz, 1H), 1.69-1.51 (m, 3H). HRMS (ESI) m/z: [M+H]⁺ Calcd for C₁₇H₁₉N₄O, 295.1553. Found 295.1547.

### Example 15: Synthesis of (±)-(4aR,13bS)-4-methyl-10-(trifluoromethyl)-1,2,3,4,4a,5,6,13b-octahydro-8H-[1,6]naphthyridino[5,6-b]quinazolin-8-one ((±)-trans-5j) and (±)-(4aR,13bR)-4-methyl-10-(trifluoromethyl)-1,2,3,4,4a,5,6,13b-octahydro-8H-[1,6]naphthyridino[5,6-b]quinazolin-8-one ((±)-cis-5j)

Prepared according to Scheme 2.

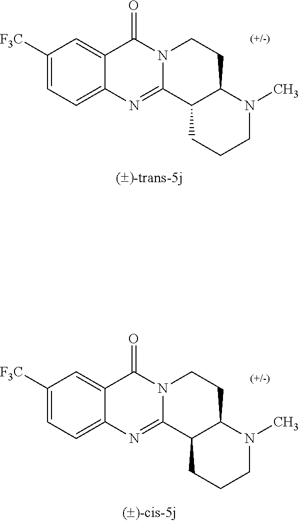 [SMILES: CN1CCC[C@@H]2c3nc4ccc(C(F)(F)F)cc4c(=O)n3CC[C@H]21 ; CN1CCC[C@H]2c3nc4ccc(C(F)(F)F)cc4c(=O)n3CC[C@H]21]

Step 1: Synthesis of tert-butyl methyl(4-(3-neopentyl-4-oxo-6-(trifluoromethyl)-3,4-dihydroquinazolin-2-yl)butyl)carbamate (2j). Compound 2j was synthesized according to general procedure 3 from 2-amino-5-trifluoromethyl-benzoic acid and 1a. Purification by flash chromatography (5-20% X/hexanes, X=4:1 EtOAc/DCM) delivered 2j as a yellow oil (1.16 g, 56%). ¹H NMR (400 MHz, CDCl₃) δ 8.54-8.49 (m, 1H), 7.89 (dd, J=8.6, 2.2 Hz, 1H), 7.69 (d, J=8.5 Hz, 1H), 4.12 (s, 2H), 3.28 (t, J=6.9 Hz, 2H), 2.88 (d, J=29.4 Hz, 5H), 1.79 (d, J=9.8 Hz, 2H), 1.65 (p, J=7.6 Hz, 2H), 1.45 (s, 9H), 1.01 (s, 9H). HRMS (ESI) m/z: [M+H]⁺ Calcd for C₂₄H₃₅F₃N₃O₃, 470.2625. Found 470.2620.

Step 2: Synthesis of 2-(4-(methylamino)butyl)-3-neopentyl-6-(trifluoromethyl)quinazolin-4(3H)-one bis-hydrochloride salt (3j). Compound 3j was synthesized according to general procedure 4 from 2j, which afforded 3j as a white solid (0.71 g, 80%). ¹H NMR (400 MHz, DMSO) δ 8.92 (d, J=7.9 Hz, 2H), 8.16 (d, J=2.2 Hz, 1H), 7.92 (dd, J=8.7, 2.2 Hz, 1H), 7.66 (d, J=8.5 Hz, 1H), 6.02 (s, 1H), 3.93 (s, 2H), 2.81 (t, J=7.1 Hz, 2H), 2.73 (q, J=6.8 Hz, 2H), 2.32 (m, 3H), 1.72-1.53 (m, 4H), 0.76 (s, 9H). HRMS (ESI) m/z: [M+H]⁺ Calcd for C₁₉H₂₇F₃N₃O, 370.2101. Found 370.3709.

Step 3.1: Synthesis of benzyl (2-(1-methyl-3-(3-neopentyl-4-oxo-6-(trifluoromethyl)-3,4-dihydroquinazolin-2-yl)piperidin-2-yl)ethyl)carbamate (4j). Compound 4j was synthesized according to general procedure 5.1 from 3j. Purification by reverse phase flash chromatography (10-90% MeOH/H₂O) which afforded 4j as an off-white solid (319 mg, 76%). 4j was used directly in the next step without analysis.

Step 4: Synthesis of (±)-(4aR,13bS)-4-methyl-10-(trifluoromethyl)-1,2,3,4,4a,5,6,13b-octahydro-8H-[1,6]naphthyridino[5,6-b]quinazolin-8-one ((±)-trans-5j) and (±)-(4aR,13bR)-4-methyl-10-(trifluoromethyl)-1,2,3,4,4a,5,6,13b-octahydro-8H-[1,6]naphthyridino[5,6-b]quinazolin-8-one ((±)-cis-5j). Both diastereomers of 5j were synthesized according to general procedure 6 starting from 4j. Purified by flash chromatography: 5-50% X/hexanes (X=2% NEt₃ 3:1 EtOAc/EtOH). (±)-trans-5j+(±)-cis-5j: 128 mg (79%).

(±)-trans-5j was isolated as a tan solid (79 mg). ¹H NMR (400 MHz, CDCl₃) δ 8.54 (d, J=2.1 Hz, 1H), 7.89 (dd, J=8.6, 2.2 Hz, 1H), 7.73 (d, J=8.6 Hz, 1H), 4.25-4.10 (m, 2H), 3.00 (ddt, J=11.7, 4.1, 1.8 Hz, 1H), 2.73 (qd, J=12.1, 10.8, 4.0 Hz, 2H), 2.46 (dq, J=13.4, 5.4 Hz, 1H), 2.39 (s, 3H), 2.14 (td, J=12.0, 3.2 Hz, 1H), 2.01 (td, J=10.2, 4.7 Hz, 1H), 1.94-1.74 (m, 3H), 1.56-1.43 (m, 1H). HRMS (ESI) m/z: [M+H]⁺ Calcd for C₁₇H₁₉F₃N₃O, 338.1375. Found 338.1471.

(±)-cis-5j was isolated as a tan solid (49 mg). ¹H NMR (400 MHz, CDCl₃) δ 8.53 (d, J=2.1 Hz, 1H), 7.86 (dd, J=8.6, 2.2 Hz, 1H), 7.69 (d, J=8.6 Hz, 1H), 4.14 (ddd, J=14.2, 5.8, 3.9 Hz, 1H), 4.02 (ddd, J=14.0, 11.2, 5.0 Hz, 1H), 3.01 (p, J=4.8, 4.1 Hz, 1H), 2.91-2.73 (m, 2H), 2.56 (dt, J=5.9, 3.1 Hz, 1H), 2.37 (dq, J=14.3, 4.8 Hz, 1H), 2.28 (s, 3H), 2.19 (td, J=10.7, 3.8 Hz, 1H), 1.96 (dddd, J=14.3, 11.3, 5.8, 2.7 Hz, 1H), 1.59 (dddd, J=21.9, 11.5, 8.4, 4.0 Hz, 3H). HRMS (ESI) m/z: [M+H]⁺ Calcd for C₁₇H₁₉F₃N₃O, 338.1375. Found 338.1464.

### Example 16: Synthesis of (±)-(4aR,13bS)-4-methyl-10-(6-methylpyridin-3-yl)-1,2,3,4,4a,5,6,13b-octahydro-8H-[1,6]naphthyridino[5,6-b]quinazolin-8-one ((±)-trans-9a) and (±)-((±)-cis-9a)

Prepared according to Scheme 2.

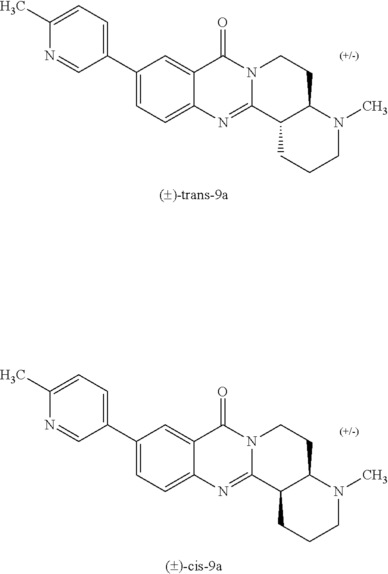

Steps 1-3.1: See example 11 (synthesis of 4d).

Step 3.2: Synthesis of benzyl (2-(1-methyl-3-(6-(6-methylpyridin-3-yl)-3-neopentyl-4-oxo-3,4-dihydroquinazolin-2-yl)piperidin-2-yl)ethyl)carbamate (8a). Compound 8a was synthesized according to general procedure 5.2 from 4d and (6-methylpyridin-3-yl)boronic acid. Purification by flash chromatography (10-60% X/hexanes (X=2% NEt₃ 3:1 EtOAc/EtOH)) and scavenging afforded 8a as an off-white solid (153 mg, 75%). 8a was used directly in the next step without analysis.

Step 4: Synthesis of (±)-(4aR,13bS)-4-methyl-10-(6-methylpyridin-3-yl)-1,2,3,4,4a,5,6,13b-octahydro-8H-[1,6]naphthyridino[5,6-b]quinazolin-8-one ((±)-trans-9a) and (±)-((±)-cis-9a). Both diastereomers of 9a were synthesized according to general procedure 6 starting from 8a. Purified by flash chromatography: 15-50% X/hexanes (X=2% NEt₃ 3:1 EtOAc/EtOH). (±)-trans-9a+(±)-cis-9a: 75.3 mg (79%).

(±)-trans-9a was isolated as a tan solid (22.8 mg). ¹H NMR (400 MHz, CDCl₃) δ 8.77 (d, J=2.3 Hz, 1H), 8.40 (d, J=2.1 Hz, 1H), 7.86 (ddt, J=20.6, 8.0, 2.1 Hz, 2H), 7.67 (dd, J=8.5, 1.8 Hz, 1H), 7.21 (d, J=8.0 Hz, 1H), 4.23-4.06 (m, 2H), 2.94 (ddt, J=11.7, 4.0, 2.0 Hz, 1H), 2.77-2.61 (m, 2H), 2.57 (d, J=1.3 Hz, 3H), 2.47-2.35 (m, 1H), 2.34 (d, J=1.4 Hz, 3H), 2.08 (td, J=11.8, 2.9 Hz, 1H), 2.02-1.91 (m, 1H), 1.88-1.70 (m, 3H), 1.54-1.39 (m, 1H). HRMS (ESI) m/z: [M+H]⁺ Calcd for C₂₂H₂₅N₄O, 361.2023. Found 361.2026.

(±)-cis-9a was isolated as a tan solid (52.5 mg). ¹H NMR (400 MHz, CDCl₃) δ 8.80 (d, J=2.4 Hz, 1H), 8.44 (d, J=2.2 Hz, 1H), 7.88 (ddd, J=15.6, 8.3, 2.4 Hz, 2H), 7.69 (d, J=8.5 Hz, 1H), 7.23 (d, J=8.0 Hz, 1H), 4.19-3.99 (m, 2H), 3.03 (q, J=3.7 Hz, 1H), 2.88-2.73 (m, 2H), 2.60 (s, 4H), 2.36 (dq, J=14.6, 4.9 Hz, 1H), 2.29 (s, 3H), 2.25-2.14 (m, 1H), 1.97 (tdt, J=10.5, 5.7, 2.7 Hz, 1H), 1.62 (dddt, J=19.7, 12.4, 8.1, 3.5 Hz, 3H). HRMS (ESI) m/z: [M+H]⁺ Calcd for C₂₂H₂₅N₄O, 361.2023. Found 361.2023.

### Example 17: Synthesis of (±)-(4aR,13bS)-11-chloro-4-methyl-1,2,3,4,4a,5,6,13b-octahydro-8H-[1,6]naphthyridino[5,6-b]quinazolin-8-one ((±)-trans-5k) and (±)-(4aR,13bR)-11-chloro-4-methyl-1,2,3,4,4a,5,6,13b-octahydro-8H-[1,6]naphthyridino[5,6-b]quinazolin-8-one ((±)-cis-5k)

Prepared according to Scheme 2.

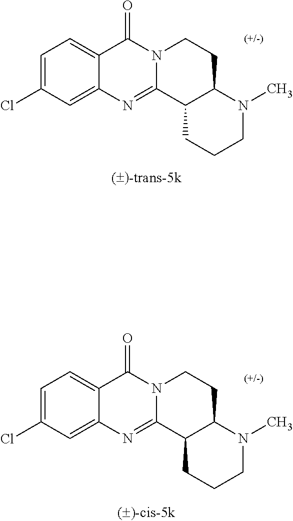 [SMILES: CN1CCC[C@@H]2c3nc4cc(Cl)ccc4c(=O)n3CC[C@H]21 ; CN1CCC[C@H]2c3nc4cc(Cl)ccc4c(=O)n3CC[C@H]21]

Step 1: Synthesis of tert-butyl (4-(7-chloro-3-neopentyl-4-oxo-3,4-dihydroquinazolin-2-yl)butyl)(methyl)carbamate (2k). Compound 2k was synthesized according to general procedure 3 from 2-amino-4-chloro-benzoic acid and 1a. Purification by flash chromatography (5-20% X/hexanes, X=4:1 EtOAc/DCM) delivered 2k as a colorless oil (0.98 g, 63%). ¹H NMR (400 MHz, CDCl₃) δ 8.14 (d, J=8.5 Hz, 1H), 7.59 (d, J=2.0 Hz, 1H), 7.35 (dd, J=8.5, 2.0 Hz, 1H), 4.07 (s, 2H), 3.26 (t, J=7.0 Hz, 2H), 2.92-2.78 (m, 5H), 1.80-1.71 (m, 2H), 1.62 (p, J=7.2 Hz, 2H), 1.44 (s, 9H), 0.98 (s, 9H). HRMS (ESI) m/z: [M+H]⁺ Calcd for C₂₃H₃₅ClN₃O₃, 436.2361. Found 436.2347.

Step 2: Synthesis of 7-chloro-2-(4-(methylamino)butyl)-3-neopentylquinazolin-4(3H)-one bis-hydrochloride salt (3k). Compound 3k was synthesized according to general procedure 4 from 2k, which afforded 3k as a white solid (0.62 g, 97%). ¹H NMR (400 MHz, D₂O) δ 8.08 (d, J=8.7 Hz, 1H), 7.69 (d, J=1.9 Hz, 1H), 7.60 (dd, J=8.7, 1.9 Hz, 1H), 4.20 (s, 2H), 3.25 (d, J=7.2 Hz, 2H), 3.16-3.07 (m, 2H), 2.74 (s, 3H), 2.01-1.79 (m, 4H), 0.97 (s, 9H). HRMS (ESI) m/z: [M+H]⁺ Calcd for C₁₈H₂₇ClN₃O, 336.1837. Found 336.1843.

Step 3.1: Synthesis of benzyl (2-(3-(7-chloro-3-neopentyl-4-oxo-3,4-dihydroquinazolin-2-yl)-1-methylpiperidin-2-yl)ethyl)carbamate (4k). Compound 4k was synthesized according to general procedure 5.1 from 3k. Purification by reverse phase flash chromatography (10-80% MeOH/H₂O) afforded 4k a white solid (219 mg, 84 ¹H NMR (400 MHz, CDCl₃) δ 8.20-8.08 (m, 1H), 7.98-7.80 (m, 0.6H), 7.69-7.58 (m, 0.3H), 7.46-7.23 (m, 6.3H), 6.03 (s, 0.4H), 5.28-5.07 (m, 1.8H), 5.06-4.90 (m, 0.7H), 4.74 (d, J=14.2 Hz, 0.3H), 4.43 (s, 0.4H), 3.58 (ddd, J=12.0, 7.8, 4.0 Hz, 0.7H), 3.38-3.24 (m, 1.4H), 3.18-3.04 (m, 0.8H), 3.00-2.67 (m, 2.8H), 2.61-2.50 (m, 1.3H), 2.49-2.31 (m, 2.9H), 2.31-2.14 (m, 0.4H), 2.05-1.30 (m, 6.2H), 1.03-0.97 (m, 9H). HRMS (ESI) m/z: [M+H]⁺ Calcd for C₂₉H₃₈ClN₄O₃, 525.2627.

Step 4: Synthesis of (±)-(4aR,13bS)-11-chloro-4-methyl-1,2,3,4,4a,5,6,13b-octahydro-8H-[1,6]naphthyridino[5,6-b]quinazolin-8-one ((±)-trans-5k) and (±)-(4aR,13bR)-11-chloro-4-methyl-1,2,3,4,4a,5,6,13b-octahydro-8H-[1,6]naphthyridino[5,6-b]quinazolin-8-one ((±)-cis-5k). Both diastereomers of 5k were synthesized according to general procedure 6 starting from 4k. Purified by flash chromatography: 10-40% X/hexanes (X=2% NEt₃ 3:1 EtOAc/EtOH). (±)-trans-5k+(±)-cis-5k: 53.3 mg (88%).

(±)-trans-5k was isolated as a yellow-white solid (53.3 mg). ¹H NMR (400 MHz, CDCl₃) δ 8.14 (d, J=8.6 Hz, 1H), 7.61 (d, J=2.0 Hz, 1H), 7.34 (dd, J=8.6, 2.0 Hz, 1H), 5.62 (s, 0H), 4.18-4.05 (m, 2H), 3.61 (d, J=7.3 Hz, OH), 2.96 (dp, J=11.5, 1.8 Hz, 1H), 2.74-2.60 (m, 2H), 2.41 (dq, J=13.3, 5.5 Hz, 1H), 2.35 (s, 3H), 2.09 (td, J=12.0, 3.1 Hz, 1H), 1.96 (td, J=10.1, 4.8 Hz, 1H), 1.90-1.68 (m, 3H), 1.52-1.36 (m, 1H). HRMS (ESI) m/z: [M+H]⁺ Calcd for C₁₆H₁₉ClN₃O, 304.1211. Found 304.1209.

(±)-cis-5k was isolated as a yellow-white solid (18.2 mg). ¹H NMR (400 MHz, CDCl₃) δ 8.16 (d, J=8.5 Hz, 1H), 7.61 (d, J=2.0 Hz, 1H), 7.34 (dd, J=8.6, 2.0 Hz, 1H), 4.11 (ddd, J=14.1, 5.8, 3.9 Hz, 1H), 3.99 (ddd, J=14.1, 11.2, 5.0 Hz, 1H), 3.02-2.95 (m, 1H), 2.86-2.73 (m, 2H), 2.54 (dt, J=6.1, 3.2 Hz, 1H), 2.35 (dtd, J=14.3, 5.1, 3.9 Hz, 1H), 2.28 (s, 3H), 2.23-2.12 (m, 1H), 1.94 (dddd, J=14.3, 11.2, 5.8, 2.8 Hz, 1H), 1.71-1.50 (m, 3H). HRMS (ESI) m/z: [M+H]⁺ Calcd for C₁₆H₁₉ClN₃O, 304.1211. Found 304.1209.

### Example 18: Synthesis of (±)-(4aR,13bS)-11-bromo-4-methyl-1,2,3,4,4a,5,6,13b-octahydro-8H-[1,6]naphthyridino[5,6-b]quinazolin-8-one ((±)-trans-51) and (±)-(4aR,13bR)-11-bromo-4-methyl-1,2,3,4,4a,5,6,13b-octahydro-8H-[1,6]naphthyridino[5,6-b]quinazolin-8-one ((±)-cis-51)

Prepared according to Scheme 2.

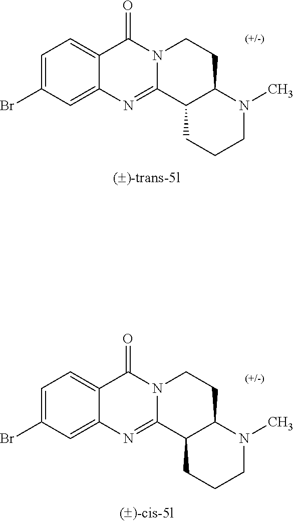 [SMILES: CN1CCC[C@@H]2c3nc4cc(Br)ccc4c(=O)n3CC[C@H]21 ; CN1CCC[C@H]2c3nc4cc(Br)ccc4c(=O)n3CC[C@H]21]

Step 1: Synthesis of tert-butyl (4-(7-bromo-3-neopentyl-4-oxo-3,4-dihydroquinazolin-2-yl)butyl)(methyl)carbamate (21). Compound 21 was synthesized according to general procedure 3 from 2-amino-4-bromo-benzoic acid and 1a. Purification by flash chromatography (5-15% X/hexanes, X=4:1 EtOAc/DCM) delivered 21 as a colorless oil (0.70 g, 70%). %). ¹H NMR (400 MHz, CDCl₃) δ 8.06 (d, J=8.5 Hz, 1H), 7.78 (d, J=1.9 Hz, 1H), 7.50 (dd, J=8.5, 1.9 Hz, 1H), 4.07 (s, 2H), 3.26 (t, J=7.0 Hz, 2H), 2.92-2.78 (m, 5H), 1.82-1.70 (m, 3H), 1.62 (p, J=7.5 Hz, 2H), 1.44 (s, 9H), 0.98 (s, 9H). HRMS (ESI) m/z: [M+H]⁺ Calcd for C₂₃H₃₅BrN₃O₃, 480.1856. Found 480.1838.

Step 2: Synthesis of 7-bromo-2-(4-(methylamino)butyl)-3-neopentylquinazolin-4(3H)-one bis-hydrochloride salt (31). Compound 31 was synthesized according to general procedure 4 from 21, which afforded 31 as a white solid (0.61 g, 98%). ¹H NMR (400 MHz, DMSO) δ 9.14 (q, J=6.2, 5.7 Hz, 2H), 8.02 (d, J=8.5 Hz, 1H), 7.95 (d, J=1.9 Hz, 1H), 7.70 (dd, J=8.5, 1.9 Hz, 1H), 6.59 (s, 1H), 4.08 (s, 2H), 3.01 (t, J=7.0 Hz, 2H), 2.90 (dd, J=9.6, 3.6 Hz, 2H), 2.51-2.47 (m, 3H), 1.86-1.74 (m, 4H), 0.93 (s, 9H). HRMS (ESI) m/z: [M+H]⁺ Calcd for C₁₈H₂₇BrN₃O, 380.1332. Found 380.1318.

Step 3.1: Synthesis of benzyl (2-(3-(7-bromo-3-neopentyl-4-oxo-3,4-dihydroquinazolin-2-yl)-1-methylpiperidin-2-yl)ethyl)carbamate (41). Compound 41 was synthesized according to general procedure 5.1 from 31. Purification by reverse phase flash chromatography (10-75% MeOH/H₂O) afforded 4l a white solid (237 mg, 83%). ¹H NMR (400 MHz, CDCl₃) δ 8.13-7.98 (m, 1.6H), 7.81 (d, J=1.9 Hz, 0.3H), 7.56-7.47 (m, 1H), 7.45-7.38 (m, 1H), 7.38-7.26 (m, 4H), 6.02 (s, 0.85H), 5.27-5.06 (m, 1.7H), 5.06-4.90 (m, 0.6H), 4.74 (d, J=14.3 Hz, 0.3H), 4.43 (s, 0.8H), 3.58 (ddd, J=13.1, 8.1, 4.7 Hz, 0.8H), 3.42-3.20 (m, 1.4H), 3.16-3.04 (m, 0.7H), 2.98-2.68 (m, 2.5H), 2.60-2.50 (m, 1.1H), 2.41 (s, 2.7H), 2.29-2.16 (m, 0.3H), 1.95 (d, J=11.6 Hz, 1H), 1.89-1.50 (m, 4.5H), 1.48-1.23 (m, 0.6H), 1.07-0.92 (m, 9H). HRMS (ESI) m/z: [M+H]⁺ Calcd for C₂₉H₃₈BrN₄O₃, 569.2122. Found 569.2129.

Step 4: Synthesis of (±)-(4aR,13bS)-11-bromo-4-methyl-1,2,3,4,4a,5,6,13b-octahydro-8H-[1,6]naphthyridino[5,6-b]quinazolin-8-one ((±)-trans-51) and (±)-(4aR,13bR)-11-bromo-4-methyl-1,2,3,4,4a,5,6,13b-octahydro-8H-[1,6]naphthyridino[5,6-b]quinazolin-8-one ((±)-cis-51). Both diastereomers of 51 were synthesized according to general procedure 6 starting from 41. Purified by flash chromatography: 10-40% X/hexanes (X=2% NEt₃ 3:1 EtOAc/EtOH). (±)-trans-51+(±)-cis-5l: 60.1 mg (86%).

(±)-trans-51 was isolated as a yellow-white solid (39.9 mg). ¹H NMR (400 MHz, CDCl₃) δ 8.07 (d, J=8.4 Hz, 1H), 7.80 (d, J=1.9 Hz, 1H), 7.50 (dd, J=8.6, 1.9 Hz, 1H), 4.20-4.03 (m, 2H), 2.96 (dq, J=11.7, 2.2 Hz, 1H), 2.75-2.61 (m, 2H), 2.41 (dq, J=13.3, 5.5 Hz, 1H), 2.35 (s, 3H), 2.09 (td, J=12.0, 3.1 Hz, 1H), 1.96 (td, J=10.1, 4.8 Hz, 1H), 1.90-1.68 (m, 3H), 1.44 (qdd, J=12.6, 4.6, 1.7 Hz, 1H). HRMS (ESI) m/z: [M+H]⁺ Calcd for C₁₆H₁₉BrN₃O, 348.0706. Found 348.0716.

(±)-cis-51 was isolated as a yellow-white solid (20.2 mg). ¹H NMR (400 MHz, CDCl₃) δ 8.08 (d, J=8.6 Hz, 1H), 7.80 (d, J=1.9 Hz, 1H), 7.49 (dd, J=8.5, 1.9 Hz, 1H), 4.11 (ddd, J=14.1, 5.8, 3.9 Hz, 1H), 3.99 (ddd, J=14.1, 11.3, 5.0 Hz, 1H), 3.02-2.96 (m, 1H), 2.87-2.73 (m, 2H), 2.54 (dt, J=6.1, 3.1 Hz, 1H), 2.41-2.30 (m, 1H), 2.28 (s, 3H), 2.18 (td, J=11.1, 2.5 Hz, 1H), 1.94 (dddd, J=14.3, 11.3, 5.8, 2.8 Hz, 1H), 1.67-1.59 (m, 1H), 1.57 (ddt, J=16.0, 8.1, 3.5 Hz, 2H). HRMS (ESI) m/z: [M+H]⁺ Calcd for C₁₆H₁₉BrN₃O, 348.0706. Found 348.0705.

### Example 19: Synthesis of (±)-(4aR,13bS)-11-fluoro-4-methyl-1,2,3,4,4a,5,6,13b-octahydro-8H-[1,6]naphthyridino[5,6-b]quinazolin-8-one ((±)-trans-5m) and (±)-(4aR,13bR)-11-fluoro-4-methyl-1,2,3,4,4a,5,6,13b-octahydro-8H-[1,6]naphthyridino[5,6-b]quinazolin-8-one((±)-cis-5m)

Prepared according to Scheme 2.

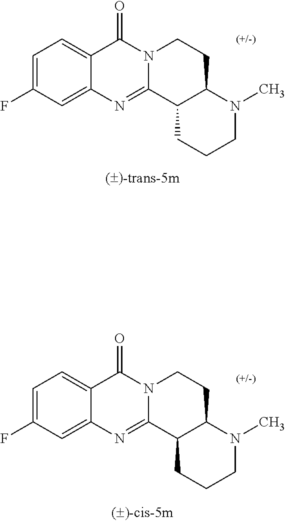 [SMILES: CN1CCC[C@@H]2c3nc4cc(F)ccc4c(=O)n3CC[C@H]21 ; CN1CCC[C@H]2c3nc4cc(F)ccc4c(=O)n3CC[C@H]21]

Step 1: Synthesis of tert-butyl (4-(7-fluoro-3-neopentyl-4-oxo-3,4-dihydroquinazolin-2-yl)butyl)(methyl)carbamate (2m). Compound 2m was synthesized according to general procedure 3 from 2-amino-4-fluoro-benzoic acid and 1a. Purification by flash chromatography (0-5% EtOAc/DCM) delivered 2m as a colorless oil (1.06 g, 71%). ¹H NMR (400 MHz, CDCl₃) δ 8.22 (dd, J=8.9, 6.2 Hz, 1H), 7.22 (dd, J=9.8, 2.5 Hz, 1H), 7.11 (td, J=8.5, 2.5 Hz, 1H), 4.08 (s, 2H), 3.32-3.20 (m, 2H), 2.92-2.79 (m, 5H), 1.81-1.71 (m, 2H), 1.64 (p, J=7.4 Hz, 2H), 1.44 (s, 9H), 0.99 (s, 9H). HRMS (ESI) m/z: [M+H]⁺ Calcd for C₂₃H₃₅FN₃O₃, 420.2647. Found 420.2645.

Step 2: Synthesis of 7-fluoro-2-(4-(methylamino)butyl)-3-neopentylquinazolin-4(3H)-one bis-hydrochloride salt (3m). Compound 3m was synthesized according to general procedure 4 from 2m, which afforded 3m as a white solid (0.86 g, 91%). ¹H NMR (400 MHz, DMSO) δ 13.49 (s, 1H), 9.19 (s, 2H), 8.18 (dd, J=8.9, 6.1 Hz, 1H), 7.53 (dd, J=9.7, 2.5 Hz, 1H), 7.41 (td, J=8.7, 2.5 Hz, 1H), 4.09 (s, 2H), 3.02 (t, J=7.1 Hz, 2H), 2.90 (q, J=6.6 Hz, 2H), 2.48 (d, J=5.5 Hz, 3H), 1.82 (tdd, J=15.3, 8.8, 4.7 Hz, 4H), 0.93 (s, 9H). HRMS (ESI) m/z: [M+H]⁺ Calcd for C₁₈H₂₇FN₃O, 320.2133. Found 320.2121.

Step 3.1: Synthesis of benzyl (2-(3-(7-fluoro-3-neopentyl-4-oxo-3,4-dihydroquinazolin-2-yl)-1-methylpiperidin-2-yl)ethyl)carbamate (4m). Compound 4m was synthesized according to general procedure 5.1 from 3m. Purification by reverse phase flash chromatography (10-80% MeOH/H₂O) afforded 4m a white solid (202 mg, 80%). ¹H NMR (400 MHz, CDCl₃) δ 8.27-8.17 (m, 1H), 7.49-7.22 (m, 6H), 7.17-7.06 (m, 1H), 6.07 (s, 1H), 5.21-5.07 (m, 2H), 5.06-4.95 (m, 1H), 4.75 (d, J=14.2 Hz, OH), 4.44 (s, 1H), 3.58 (ddd, J=13.1, 8.2, 4.7 Hz, 1H), 3.41-3.25 (m, 1H), 3.16-3.05 (m, 1H), 2.97-2.69 (m, 3H), 2.60-2.50 (m, 1H), 2.48-2.32 (m, 2H), 2.28-2.12 (m, 0H), 2.06-1.31 (m, 6H), 1.08-0.93 (m, 9H). HRMS (ESI) m/z: [M+H]+ Calcd for C₂₉H₃₇FN₄O₃ 509.2922. Found 509.2934.

Step 4: Synthesis of (±)-(4aR,13bS)-11-fluoro-4-methyl-1,2,3,4,4a,5,6,13b-octahydro-8H-[1,6]naphthyridino[5,6-b]quinazolin-8-one ((±)-trans-5m) and (±)-(4aR,13bR)-11-fluoro-4-methyl-1,2,3,4,4a,5,6,13b-octahydro-8H-[1,6]naphthyridino[5,6-b]quinazolin-8-one((±)-cis-5m). Both diastereomers of 5m were synthesized according to general procedure 6 starting from 4m. Purified by flash chromatography: 10-50% X/hexanes (X=2% NEt₃ 3:1 EtOAc/EtOH). (±)-trans-5m+(±)-cis-5m: 56.5 mg (98%).

(±)-trans-5m was isolated as a yellow oil (38.7 mg). ¹H NMR (400 MHz, CDCl₃) δ 8.22 (dd, J=8.9, 6.2 Hz, 1H), 7.28-7.20 (m, 1H), 7.11 (td, J=8.5, 2.5 Hz, 1H), 4.21-4.03 (m, 2H), 3.00-2.91 (m, 1H), 2.69 (dt, J=13.8, 11.1 Hz, 2H), 2.41 (dq, J=13.4, 5.5 Hz, 1H), 2.35 (s, 3H), 2.09 (td, J=12.0, 3.1 Hz, 1H), 1.96 (td, J=10.1, 4.8 Hz, 1H), 1.89-1.70 (m, 3H), 1.50-1.37 (m, 1H). HRMS (ESI) m/z: [M+H]⁺ Calcd for C₁₆H₁₉FN₃O, 288.1507. Found 288.1505.

(±)-cis-5m was isolated as an off white solid (17.8 mg). ¹H NMR (400 MHz, CDCl₃) δ 8.24 (dd, J=8.9, 6.2 Hz, 1H), 7.27-7.22 (m, 1H), 7.11 (td, J=8.6, 2.5 Hz, 1H), 4.11 (ddd, J=14.0, 5.9, 4.1 Hz, 1H), 4.01 (ddd, J=14.1, 11.1, 5.0 Hz, 1H), 3.00 (q, J=3.5, 3.0 Hz, 1H), 2.86-2.74 (m, 2H), 2.56 (dt, J=6.2, 3.2 Hz, 1H), 2.35 (dtd, J=14.5, 5.1, 4.1 Hz, 1H), 2.28 (s, 3H), 2.23-2.15 (m, 1H), 1.95 (dddd, J=14.3, 11.0, 5.9, 2.8 Hz, 1H), 1.67-1.53 (m, 3H). HRMS (ESI) m/z: [M+H]⁺ Calcd for C₁₆H₁₉FN₃O, 288.1507. Found 288.1502.

### Example 20: Synthesis of (±)-(4aR,13bS)-4-methyl-11-(trifluoromethyl)-1,2,3,4,4a,5,6,13b-octahydro-8H-[1,6]naphthyridino[5,6-b]quinazolin-8-one ((±)-trans-5n) and (±)-(4aR,13bR)-4-methyl-11-(trifluoromethyl)-1,2,3,4,4a,5,6,13b-octahydro-8H-[1,6]naphthyridino[5,6-b]quinazolin-8-one ((±)-cis-5n)

Prepared according to Scheme 2.

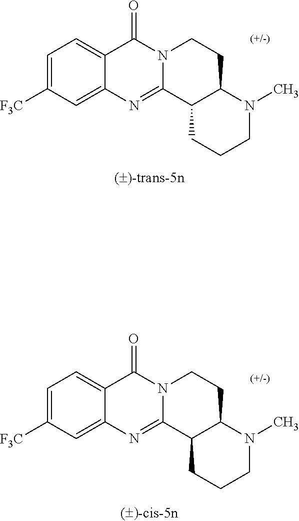 [SMILES: CN1CCC[C@@H]2c3nc4cc(C(F)(F)F)ccc4c(=O)n3CC[C@H]21 ; CN1CCC[C@H]2c3nc4cc(C(F)(F)F)ccc4c(=O)n3CC[C@H]21]

Step 1: Synthesis of tert-butyl methyl(4-(3-neopentyl-4-oxo-7-(trifluoromethyl)-3,4-dihydroquinazolin-2-yl)butyl)carbamate (2n). Compound 2n was synthesized according to general procedure 3 from 2-amino-4-trifluoromethyl-benzoic acid and 1a. Purification by flash chromatography (5-15% X/hexanes, X=4:1 EtOAc/DCM) delivered 2n as a yellow-orange oil (0.92 g, 88%). ¹H NMR (400 MHz, CDCl₃) δ 8.36-8.30 (m, 1H), 7.89 (dd, J=1.8, 0.9 Hz, 1H), 7.60 (dd, J=8.3, 1.7 Hz, 1H), 4.81-3.57 (m, 2H), 3.27 (t, J=7.0 Hz, 2H), 2.91 (t, J=7.5 Hz, 2H), 2.84 (s, 3H), 1.85-1.74 (m, 2H), 1.64 (p, J=8.8, 8.0 Hz, 2H), 1.45 (s, 9H), 1.00 (s, 9H). HRMS (ESI) m/z: [M+H]⁺ Calcd for C₂₄H₃₅F₃N₃O₃, 470.2625. Found 470.2608.

Step 2: Synthesis of 2-(4-(methylamino)butyl)-3-neopentyl-7-(trifluoromethyl)quinazolin-4(3H)-one bis-hydrochloride salt (3n). Compound 3n was synthesized according to general procedure 4 from 2n, which afforded 3n as a white solid (0.76 g, 94%). ¹H NMR (400 MHz, DMSO) δ 9.14 (s, 2H), 8.30 (dt, J=8.3, 0.8 Hz, 1H), 7.99 (dd, J=1.8, 1.0 Hz, 1H), 7.80 (dd, J=8.4, 1.8 Hz, 1H), 4.11 (s, 2H), 3.00 (t, J=7.0 Hz, 2H), 2.95-2.85 (m, 2H), 2.51-2.48 (m, 4H), 1.88-1.71 (m, 4H), 0.93 (s, 9H). HRMS (ESI) m/z: [M+H]⁺ Calcd for C₁₉H₂₇F₃N₃O, 370.2101. Found 370.2106

Step 3.1: Synthesis of benzyl (2-(1-methyl-3-(3-neopentyl-4-oxo-7-(trifluoromethyl)-3,4-dihydroquinazolin-2-yl)piperidin-2-yl)ethyl)carbamate (4n). Compound 4n was synthesized according to general procedure 5.1 from 3n. Purification by reverse phase flash chromatography (10-80% MeOH/H₂O) afforded 4n a white solid (232 mg, 83%). %). ¹H NMR (400 MHz, CDCl₃) δ 8.37-8.303 (m, 1H), 8.10 (s, 0.6H), 7.96-7.87 (m, 0.3H), 7.66-7.55 (m, 1H), 7.43-7.23 (m, 5H), 5.84 (s, 0.7H), 5.25-4.93 (m, 2.2H), 4.77 (d, J=14.2 Hz, 0.3H), 4.40 (s, 0.7H), 3.68-3.55 (m, 0.6H), 3.38-3.24 (m, 1.3H), 3.19-3.07 (m, 0.7H), 3.02-2.65 (m, 2.6H), 2.61-2.20 (m, 4.4H), 2.03-1.46 (m, 6H), 1.44-1.29 (m, 0.4H), 1.04-0.94 (m, 9H). HRMS (ESI) m/z: [M+H]⁺ Calcd for C₃₀H₃₈F₃N₄O₃, 559.2891. Found 559.2890.

Step 4: Synthesis of (±)-(4aR,13bS)-4-methyl-11-(trifluoromethyl)-1,2,3,4,4a,5,6,13b-octahydro-8H-[1,6]naphthyridino[5,6-b]quinazolin-8-one ((±)-trans-5n) and (±)-(4aR,13bR)-4-methyl-11-(trifluoromethyl)-1,2,3,4,4a,5,6,13b-octahydro-8H-[1,6]naphthyridino[5,6-b]quinazolin-8-one ((±)-cis-5n). Both diastereomers of 5n were synthesized according to general procedure 6 starting from 4n. Purified by flash chromatography: 10-40% X/hexanes (X=2% NEt₃ 3:1 EtOAc/EtOH). (±)-trans-5n+(±)-cis-5n: 63.0 mg (93%).

(±)-trans-5n was isolated as an off white solid (39.7 mg). ¹H NMR (400 MHz, CDCl₃) δ 8.33 (d, J=8.3 Hz, 1H), 7.91 (d, J=1.7 Hz, 1H), 7.59 (dd, J=8.4, 1.7 Hz, 1H), 4.20-4.06 (m, 2H), 2.96 (dq, J=11.9, 2.1 Hz, 1H), 2.77-2.65 (m, 2H), 2.43 (dq, J=13.3, 5.5 Hz, 1H), 2.36 (s, 3H), 2.10 (td, J=12.0, 3.1 Hz, 1H), 1.98 (td, J=10.2, 4.9 Hz, 1H), 1.92-1.70 (m, 3H), 1.46 (tdd, J=14.3, 12.1, 4.9 Hz, 1H). HRMS (ESI) m/z: [M+H]⁺ Calcd for C₁₇H₁₉F₃N₃O, 338.1475. Found 338.1475.

(±)-cis-5n was isolated as an off white solid (23.3 mg). ¹H NMR (400 MHz, CDCl₃) δ 8.35 (d, J=8.3 Hz, 1H), 7.93-7.86 (m, 1H), 7.59 (dd, J=8.4, 1.7 Hz, 1H), 4.15 (ddd, J=14.1, 5.8, 3.8 Hz, 1H), 4.02 (ddd, J=14.1, 11.4, 5.0 Hz, 1H), 3.05-2.97 (m, 1H), 2.94-2.82 (m, 1H), 2.79 (ddd, J=12.3, 4.1, 2.4 Hz, 1H), 2.56 (dt, J=6.0, 3.1 Hz, 1H), 2.38 (dtd, J=14.2, 5.0, 3.7 Hz, 1H), 2.28 (s, 3H), 2.19 (td, J=10.9, 3.3 Hz, 1H), 1.96 (dddd, J=14.4, 11.4, 5.8, 2.8 Hz, 1H), 1.71-1.48 (m, 3H). HRMS (ESI) m/z: [M+H]⁺ Calcd for C₁₇H₁₉F₃N₃O, 338.1475. Found 338.1475.

### Example 21: Synthesis of (±)-(4aR,13bS)-11-methoxy-4-methyl-1,2,3,4,4a,5,6,13b-octahydro-8H-[1,6]naphthyridino[5,6-b]quinazolin-8-one ((±)-trans-5o) and (±)-(4aR,13bR)-11-methoxy-4-methyl-1,2,3,4,4a,5,6,13b-octahydro-8H-[1,6]naphthyridino[5,6-b]quinazolin-8-one ((±)-cis-5o)

Prepared according to Scheme 2.

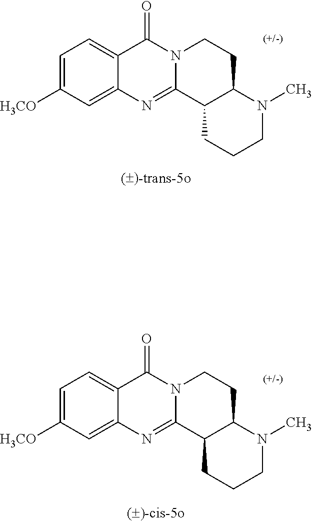 [SMILES: COc1ccc2c(=O)n3c(nc2c1)[C@H]1CCCN(C)[C@@H]1CC3 ; COc1ccc2c(=O)n3c(nc2c1)[C@@H]1CCCN(C)[C@@H]1CC3]

Step 1: Synthesis of tert-butyl (4-(7-methoxy-3-neopentyl-4-oxo-3,4-dihydroquinazolin-2-yl)butyl)(methyl)carbamate (20). Compound 20 was synthesized according to general procedure 3 from 2-amino-4-methoxy-benzoic acid and 1a. Purification by flash chromatography (0-5% EtOAc/DCM) delivered 20 as a colorless oil (0.98 g, 63 ¹H NMR (400 MHz, CDCl₃) δ 8.10 (d, J=9.5 Hz, 1H), 7.00-6.93 (m, 2H), 4.34-3.65 (m, 5H), 3.33-3.16 (m, 2H), 2.92-2.69 (m, 5H), 1.75 (p, J=8.0 Hz, 2H), 1.62 (p, J=6.9 Hz, 2H), 1.43 (s, 9H), 0.98 (s, 9H). HRMS (ESI) m/z: [M+H]⁺ Calcd for C₂₄H₃₈N₃O₄, 432.2857. Found 432.2845.

Step 2: Synthesis of 7-methoxy-2-(4-(methylamino)butyl)-3-neopentylquinazolin-4(3H)-one bis-hydrochloride salt (3o). Compound 3o was synthesized according to general procedure 4 from 2o, which afforded 3o as a white solid (0.77 g, 87%). ¹H NMR (400 MHz, DMSO) δ 9.25-9.07 (m, 2H), 8.05 (d, J=8.9 Hz, 1H), 7.35 (d, J=2.4 Hz, 1H), 7.18 (dd, J=8.9, 2.5 Hz, 1H), 4.18-3.96 (m, 2H), 3.90 (s, 3H), 3.11 (t, J=7.3 Hz, 2H), 2.91 (q, J=6.6 Hz, 2H), 2.50 (d, J=10.8 Hz, 4H), 1.91-1.76 (m, 4H), 0.95 (s, 9H). HRMS (ESI) m/z: [M+H]⁺ Calcd for C₁₉H₃₀N₃O₂, 332.2333. Found 332.2324.

Step 3.1: Synthesis of benzyl (2-(3-(7-methoxy-3-neopentyl-4-oxo-3,4-dihydroquinazolin-2-yl)-1-methylpiperidin-2-yl)ethyl)carbamate (4o). Compound 4o was synthesized according to general procedure 5.1 from 3o. Purification by reverse phase flash chromatography (10-75% MeOH/H₂O) afforded 4o an white solid (216 mg, 83%). ¹H NMR (400 MHz, CDCl₃) δ 7.68 (d, J=9.0 Hz, 0.7H), 7.63-7.50 (m, 2.3H), 7.46-7.27 (m, 5.7H), 6.87 (d, J=8.8 Hz, 0.6H), 6.44 (s, 0.8H), 5.20-5.05 (m, 1.7H), 5.06-4.95 (m, 0.6H), 4.77 (d, J=14.2 Hz, 0.3H), 4.53 (s, 0.6H), 3.93-3.84 (m, 3.1H), 3.68-3.52 (m, 0.7H), 3.41-3.28 (m, 1.7H), 3.09 (ddd, J=12.2, 9.5, 3.2 Hz, 0.8H), 2.96-2.67 (m, 2.8H), 2.58-2.48 (m, 1.2H), 2.46-2.31 (m, 2.4H), 2.30-2.07 (m, 0.3H), 1.98-1.34 (m, 4.4H), 1.08-0.96 (m, 9H). HRMS (ESI) m/z: [M+H]⁺ Calcd for C₃₀H₄₁N₄O₄, 521.3122. Found 521.3136.

Step 4: Synthesis of (±)-(4aR,13bS)-11-methoxy-4-methyl-1,2,3,4,4a,5,6,13b-octahydro-8H-[1,6]naphthyridino[5,6-b]quinazolin-8-one ((±)-trans-5o) and (±)-(4aR,13bR)-11-methoxy-4-methyl-1,2,3,4,4a,5,6,13b-octahydro-8H-[1,6]naphthyridino[5,6-b]quinazolin-8-one ((±)-cis-5o). Both diastereomers of 5o were synthesized according to general procedure 6 starting from 4o. 5o required an additional 16 h of heating at 80° C. for cyclization to be completed. The diastereomers were purified by flash chromatography: 10-50% X/hexanes (X=2% NEt₃ 3:1 EtOAc/EtOH). (±)-trans-5o+(±)-cis-5o: 52.8 mg (88%).

(±)-trans-5o was isolated as a yellow solid (30.9 mg). ¹H NMR (400 MHz, CDCl₃) δ 8.12 (dt, J=9.2, 1.2 Hz, 1H), 7.00-6.95 (m, 2H), 4.18-4.04 (m, 2H), 3.88 (s, 3H), 2.95 (ddd, J=11.6, 4.0, 2.1 Hz, 1H), 2.75-2.61 (m, 2H), 2.40 (dq, J=13.3, 5.4 Hz, 1H), 2.34 (s, 3H), 2.09 (td, J=11.9, 3.2 Hz, 1H), 1.95 (td, J=10.1, 4.8 Hz, 1H), 1.88-1.71 (m, 3H), 1.46 (tdd, J=12.7, 11.3, 4.4 Hz, 1H). HRMS (ESI) m/z: [M+H]⁺ Calcd for C₁₇H₂₁N₃O₂, 322.1526. Found 322.1526.

(±)-cis-5o was isolated as a yellow solid (21.9 mg). ¹H NMR (400 MHz, CDCl₃) δ 8.15-8.10 (m, 1H), 6.99-6.94 (m, 2H), 4.12-3.98 (m, 2H), 3.90 (s, 3H), 3.00 (q, J=3.9 Hz, 1H), 2.76 (dt, J=11.9, 3.6 Hz, 2H), 2.58 (dt, J=6.3, 3.2 Hz, 1H), 2.33 (dt, J=14.5, 5.1 Hz, 1H), 2.29 (s, 3H), 2.20 (td, J=10.6, 3.4 Hz, 1H), 1.99-1.89 (m, 1H), 1.71-1.52 (m, 3H). HRMS (ESI) m/z: [M+H]⁺ Calcd for C₁₇H₂₁N₃O₂, 322.1526. Found 322.1523.

### Example 22: Synthesis of (±)-(4aR,13bS)-4-methyl-1,2,3,4,4a,5,6,13b-octahydro-8H-pyrido[3′,4′:4,5]pyrimido[2,1-f][1,6]naphthyridin-8-one ((±)-trans-5p) and (±)-(4aR,13bR)-4-methyl-1,2,3,4,4a,5,6,13b-octahydro-8H-pyrido[3′,4′:4,5]pyrimido[2,1-f][1,6]naphthyridin-8-one ((±)-cis-5p)

Prepared according to Scheme 2.

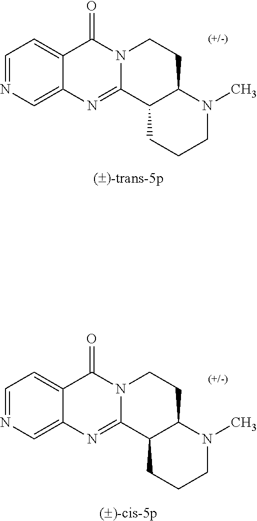 [SMILES: CN1CCC[C@@H]2c3nc4cnccc4c(=O)n3CC[C@H]21 ; CN1CCC[C@H]2c3nc4cnccc4c(=O)n3CC[C@H]21]

Step 1: Synthesis of tert-butyl methyl(4-(3-neopentyl-4-oxo-3,4-dihydropyrido[3,4-d]pyrimidin-2-yl)butyl)carbamate (2p). Compound 2p was synthesized according to general procedure 3 from 3-aminoisonicotinic acid and 1a. Purification by flash chromatography (5-50% X/hexanes, X=4:1 EtOAc/DCM) delivered 2p as a colorless oil (0.38 g, 22%). ¹H NMR (400 MHz, CDCl₃) δ 9.03 (d, J=0.9 Hz, 1H), 8.60 (d, J=5.2 Hz, 1H), 7.96 (dd, J=5.3, 0.9 Hz, 1H), 4.09 (s, 2H), 3.34-3.21 (m, 2H), 2.98-2.88 (m, 2H), 2.83 (s, 3H), 1.85-1.74 (m, 2H), 1.63 (p, J=9.7, 8.5 Hz, 2H), 1.44 (s, 9H), 0.99 (s, 9H). HRMS (ESI) m/z: [M+H]⁺ Calcd for C₂₂H₃₅N₄O₃, 403.2704. Found 403.2709.

Step 2: Synthesis of 2-(4-(methylamino)butyl)-3-neopentylpyrido[3,4-d]pyrimidin-4(3H)-one bis-hydrochloride salt (3p). Compound 3p was synthesized according to general procedure 4 from 2p, which afforded 3p as a light tan solid (0.50 g, 87%). ¹H NMR (400 MHz, DMSO) δ 10.51 (s, 1H), 9.31-9.16 (m, 3H), 8.69 (d, J=5.5 Hz, 1H), 8.16-8.10 (m, 1H), 4.10 (s, 2H), 2.99 (t, J=6.8 Hz, 2H), 2.90 (p, J=6.8 Hz, 2H), 2.48 (d, J=5.5 Hz, 3H), 1.90-1.74 (m, 4H), 0.93 (s, 9H). HRMS (ESI) m/z: [M+H]⁺ Calcd for C₁₇H₂₇N₄O, 303.2179. Found 303.2183.

Step 3.1: Synthesis of benzyl (2-(1-methyl-3-(3-neopentyl-4-oxo-3,4-dihydropyrido[3,4-d]pyrimidin-2-yl)piperidin-2-yl)ethyl)carbamate (4p). Compound 4p was synthesized according to general procedure 5.1 from 3p. Purification by reverse phase flash chromatography (10-60% MeOH/H₂O) afforded 4p a slight-yellow solid (167 mg, 68%). ¹H NMR (400 MHz, CDCl₃) δ 9.17 (s, 0.6H), 9.10-9.03 (m, 0.3H), 8.70-8.52 (m, 1H), 7.96 (dd, J=8.2, 5.3 Hz, 1H), 7.43-7.23 (m, 5H), 5.73 (s, 0.5H), 5.04 (dq, J=41.2, 12.5 Hz, 2.3H), 4.76 (d, J=14.2 Hz, 0.3H), 4.34 (s, 0.6H), 3.67-3.57 (m, 0.6H), 3.37-3.23 (m, 1.5H), 3.20-3.08 (m, 0.8H), 3.02-2.82 (m, 2.2H), 2.73 (t, J=12.7 Hz, 0.3H), 2.62-2.45 (m, 1.7H), 2.45-2.17 (m, 2.5H), 1.97 (d, J=11.7 Hz, 1H), 1.91-1.48 (m, 4.8H), 1.49-1.27 (m, 1H), 1.05-0.96 (m, 9H). HRMS (ESI) m/z: [M+H]⁺ Calcd for C₂₈H₃₈N₅O₃, 492.2969. Found 492.2976.

Step 4: Synthesis of (±)-(4aR,13bS)-4-methyl-1,2,3,4,4a,5,6,13b-octahydro-8H-pyrido[3′,4′:4,5]pyrimido[2,1-f][1,6]naphthyridin-8-one ((±)-trans-5p) and (±)-(4aR,13bR)-4-methyl-1,2,3,4,4a,5,6,13b-octahydro-8H-pyrido[3′,4′:4,5]pyrimido[2,1-f][1,6]naphthyridin-8-one ((±)-cis-5p). Both diastereomers of 5p were synthesized according to general procedure 6 starting from 4p. Purified by flash chromatography: 10-50% X/hexanes (X=2% NEt₃ 3:1 EtOAc/EtOH). (±)-trans-5p+(±)-cis-5p: 38.3 mg (71%).

(±)-trans-5p was isolated as a tan solid (23.7 mg). ¹H NMR (400 MHz, CDCl₃) δ 9.07 (s, 1H), 8.61 (d, J=5.2 Hz, 1H), 7.99 (dt, J=5.3, 0.9 Hz, 1H), 4.21-4.12 (m, 2H), 3.02-2.93 (m, 1H), 2.78-2.67 (m, 2H), 2.43 (dq, J=13.3, 5.5 Hz, 1H), 2.36 (s, 3H), 2.14-2.07 (m, 1H), 1.98 (dt, J=10.3, 5.0 Hz, 1H), 1.91-1.73 (m, 3H), 1.49 (tdd, J=12.9, 11.2, 3.9 Hz, 1H). HRMS (ESI) m/z: [M+H]⁺ Calcd for C₁₅H₁₉N₄O, 271.1553.

(±)-cis-5p was isolated as a tan solid (14.6 mg). ¹H NMR (400 MHz, CDCl₃) δ 9.05 (d, J=1.0 Hz, 1H), 8.60 (d, J=5.2 Hz, 1H), 7.99 (dd, J=5.3, 0.9 Hz, 1H), 4.19-4.11 (m, 1H), 4.04-3.95 (m, 1H), 3.06-2.99 (m, 1H), 2.90 (td, J=5.6, 3.0 Hz, 1H), 2.79 (ddd, J=12.4, 4.1, 2.3 Hz, 1H), 2.55 (dt, J=6.1, 3.2 Hz, 1H), 2.38 (dtd, J=14.2, 5.1, 3.8 Hz, 1H), 2.28 (s, 3H), 2.24-2.13 (m, 1H), 1.96 (dddd, J=14.4, 11.4, 5.8, 2.8 Hz, 1H), 1.72-1.51 (m, 2H), 1.04-0.88 (m, 1H). HRMS (ESI) m/z: [M+H]⁺ Calcd for HRMS (ESI) m/z: [M+H]⁺ Calcd for C₁₅H₁₉N₄O, 271.1553. Found 271.1553.

### Example 23: Synthesis of (±)-(4aR,13bS)-10,11-dichloro-4-methyl-1,2,3,4,4a,5,6,13b-octahydro-8H-[1,6]naphthyridino[5,6-b]quinazolin-8-one ((±)-trans-5q) and (±)-(4aR,13bR)-10,11-dichloro-4-methyl-1,2,3,4,4a,5,6,13b-octahydro-8H-[1,6]naphthyridino[5,6-b]quinazolin-8-one ((±)-cis-5q)

Prepared according to Scheme 2.

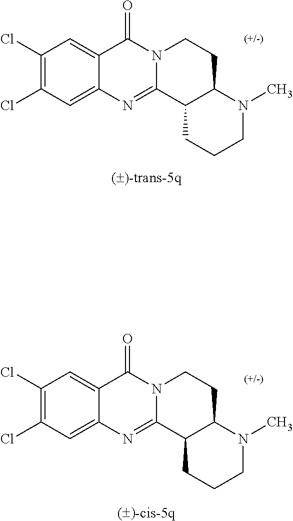 [SMILES: CN1CCC[C@@H]2c3nc4cc(Cl)c(Cl)cc4c(=O)n3CC[C@H]21 ; CN1CCC[C@H]2c3nc4cc(Cl)c(Cl)cc4c(=O)n3CC[C@H]21]

Step 1: Synthesis of tert-butyl (4-(6,7-dichloro-3-neopentyl-4-oxo-3,4-dihydroquinazolin-2-yl)butyl)(methyl)carbamate (2q). Compound 2q was synthesized according to general procedure 3 from 2-amino-4,5-dichloro-benzoic acid and 1a. Purification by flash chromatography (0-25% X/hexanes, X=4:1 EtOAc/DCM) delivered 2q as a colorless oil (0.98 g, 54%). ¹H NMR (400 MHz, CDCl₃) δ 8.27 (s, 1H), 7.70 (s, 1H), 4.06 (s, 2H), 3.27-3.22 (m, 2H), 2.88-2.82 (m, 5H), 1.78-1.73 (m, 2H), 1.66-1.58 (m, 2H), 1.44 (s, 9H), 0.98 (s, 9H). HRMS (ESI) m/z: [M+H]⁺ Calcd for C₂₃H₃₄Cl₂N₃O₃, 470.1972. Found 470.1995.

Step 2: Synthesis of 6,7-dichloro-2-(4-(methylamino)butyl)-3-neopentylquinazolin-4(3H)-one bis-hydrochloride salt (3q). Compound 3q was synthesized according to general procedure 4 from 2q, which afforded 3q as a white solid (0.66 g, 91%). ¹H NMR (400 MHz, DMSO) δ 8.95 (s, 2H), 8.21 (s, 1H), 7.91 (s, 1H), 6.03 (s, 1H), 4.15-3.98 (m, 2H), 2.92 (dt, J=12.4, 6.4 Hz, 4H), 2.51 (d, J=2.0 Hz, 3H), 1.86-1.69 (m, 4H), 0.93 (s, 9H). HRMS (ESI) m/z: [M+H]⁺ Calcd for C₁₈H₂₆Cl₂N₃O, 370.1447. Found 370.1442.

Step 3.1: Synthesis of benzyl (2-(3-(6,7-dichloro-3-neopentyl-4-oxo-3,4-dihydroquinazolin-2-yl)-1-methylpiperidin-2-yl)ethyl)carbamate (4q). Compound 4q was synthesized according to general procedure 5.1 from 3q. Purification by reverse phase flash chromatography (10-90% MeOH/H₂O) which afforded 4q as an off-white solid (449 mg, 92%). 4q was used directly in the next step without analysis.

Step 4: Synthesis of (±)-(4aR,13bS)-10,11-dichloro-4-methyl-1,2,3,4,4a,5,6,13b-octahydro-8H-[1,6]naphthyridino[5,6-b]quinazolin-8-one ((±)-trans-5q) and (±)-(4aR,13bR)-10,11-dichloro-4-methyl-1,2,3,4,4a,5,6,13b-octahydro-8H-[1,6]naphthyridino[5,6-b]quinazolin-8-one ((±)-cis-5q). Both diastereomers of 5q were synthesized according to general procedure 6 starting from 4q Purified by flash chromatography: 15-50% X/hexanes (X=2% NEt₃ 3:1 EtOAc/EtOH). (±)-trans-5q+(±)-cis-5q 165 mg (81%).

(±)-trans-5q was isolated as a tan solid (105 mg). ¹H NMR (400 MHz, CDCl₃) δ 8.29 (s, 1H), 7.75 (s, 1H), 4.14 (ddt, J=8.9, 5.9, 2.8 Hz, 2H), 3.03-2.94 (m, 1H), 2.68 (td, J=11.5, 10.7, 3.6 Hz, 2H), 2.44 (dq, J=13.4, 5.6 Hz, 1H), 2.37 (s, 3H), 2.12 (td, J=12.0, 3.1 Hz, 1H), 1.99 (td, J=10.1, 4.9 Hz, 1H), 1.94-1.70 (m, 3H), 1.45 (dtd, J=14.4, 12.4, 4.3 Hz, 1H). HRMS (ESI) m/z: [M+H]⁺ Calcd for C₁₆H₁₈Cl₂N₃O, 338.0821. Found 338.0817.

(±)-cis-5q was isolated as a yellow solid (60 mg). ¹H NMR (400 MHz, CDCl₃) δ 8.27 (s, 1H), 7.72 (s, 1H), 4.11 (ddd, J=14.2, 5.8, 3.8 Hz, 1H), 3.97 (ddd, J=14.1, 11.4, 5.0 Hz, 1H), 2.97 (q, J=3.5 Hz, 1H), 2.86-2.73 (m, 2H), 2.53 (dt, J=5.9, 3.2 Hz, 1H), 2.35 (dq, J=14.2, 4.7 Hz, 1H), 2.27 (s, 3H), 2.17 (td, J=10.0, 9.4, 3.4 Hz, 1H), 1.94 (dddd, J=14.4, 11.4, 5.8, 2.8 Hz, 1H), 1.57 (dddq, J=17.9, 10.4, 8.0, 3.7 Hz, 3H). HRMS (ESI) m/z: [M+H]⁺ Calcd for C₁₆H₁₈Cl₂N₃O, 338.0821. Found 338.0809.

### Example 24: Synthesis of (±)-(4aR,13bS)-10,11-dimethoxy-4-methyl-1,2,3,4,4a,5,6,13b-octahydro-8H-[1,6]naphthyridino[5,6-b]quinazolin-8-one ((±)-trans-5r) and (±)-(4aR,13bR)-10,11-dimethoxy-4-methyl-1,2,3,4,4a,5,6,13b-octahydro-8H-[1,6]naphthyridino[5,6-b]quinazolin-8-one ((±)-cis-5r)

Prepared according to Scheme 2.

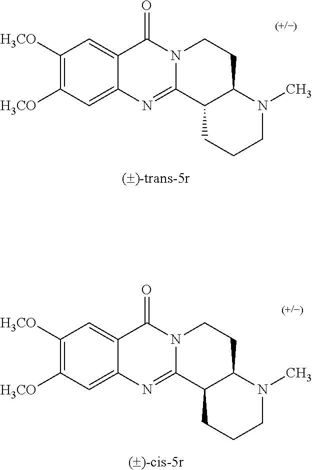 [SMILES: COc1cc2nc3n(c(=O)c2cc1OC)CC[C@@H]1[C@@H]3CCCN1C ; COc1cc2nc3n(c(=O)c2cc1OC)CC[C@@H]1[C@H]3CCCN1C]

Step 1: Synthesis of tert-butyl (4-(6,7-dimethoxy-3-neopentyl-4-oxo-3,4-dihydroquinazolin-2-yl)butyl)(methyl)carbamate (2r). Compound 2r was synthesized according to general procedure 3 from 2-amino-4,5-dimethoxy-benzoic acid and 1a. Purification by flash chromatography (5-50% X/hexanes, X=4:1 EtOAc/DCM) delivered 2r as a yellow oil (0.89 g, 69%). ¹H NMR (400 MHz, CDCl₃) δ 7.54 (s, 1H), 7.00 (s, 1H), 4.33-3.72 (m, 8H), 3.32-3.17 (m, 2H), 2.92-2.77 (m, 5H), 1.79-1.68 (m, 2H), 1.67-1.55 (m, 2H), 1.42 (s, 9H), 0.98 (s, 9H). HRMS (ESI) m/z: [M+H]⁺ Calcd for C₂₅H₄₀N₃O₅, 462.2962. Found 462.2958.

Step 2: Synthesis of 6,7-dimethoxy-2-(4-(methylamino)butyl)-3-neopentylquinazolin-4(3H)-one bis-hydrochloride salt (3r). Compound 3r was synthesized according to general procedure 4 from 2r, which afforded 3r as a white solid (0.75 g, 94%). ¹H NMR (400 MHz, DMSO) δ 9.15 (s, 2H), 7.43 (d, J=14.4 Hz, 2H), 4.11 (s, 2H), 3.90 (d, J=6.8 Hz, 6H), 3.12 (t, J=7.2 Hz, 2H), 2.98-2.79 (m, 2H), 2.49 (s, 4H), 1.84 (dp, J=20.9, 7.4 Hz, 4H), 0.95 (s, 9H). HRMS (ESI) m/z: [M+H]⁺ Calcd for C₂₀H₃₂N₃O₃, 362.2438. Found 362.2446.

Step 3.1: Synthesis of benzyl (2-(3-(6,7-dimethoxy-3-neopentyl-4-oxo-3,4-dihydroquinazolin-2-yl)-1-methylpiperidin-2-yl)ethyl)carbamate (4r). Compound 4r was synthesized according to general procedure 5.1 from 3r. Purification by reverse phase flash chromatography (10-65% MeOH/H₂O) afforded 4r an off-white solid (154 mg, 56 ¹H NMR (400 MHz, CDCl₃) δ 7.55 (d, J=5.1 Hz, 1.2H), 7.38-7.24 (m, 5.2H), 7.09 (s, 0.5H), 7.02 (s, 0.3H), 5.94 (s, 0.3H), 5.22-5.05 (m, 1.7H), 5.05-4.91 (m, 0.6H), 4.78 (d, J=14.2 Hz, 0.5H), 4.09 (s, 0.6H), 4.02-3.93 (m, 4.2H), 3.76 (s, 1.6H), 3.58 (dq, J=9.8, 3.1, 2.5 Hz, 0.8H), 3.33 (qd, J=13.6, 12.8, 6.6 Hz, 2.6H), 3.11 (t, J=9.9 Hz, 1.2H), 3.01-2.66 (m, 2.7H), 2.61-2.32 (m, 0.4H), 2.31-2.16 (m, 1H), 2.09-1.54 (m, 4.4H), 1.43-1.34 (m, 0.4H), 1.01 (d, J=10.7 Hz, 9H). HRMS (ESI) m/z: [M+H]⁺ Calcd for C₃₁H₄₃N₄O₅, 551.3228.

Step 4: Synthesis of (±)-(4aR,13bS)-10,11-dimethoxy-4-methyl-1,2,3,4,4a,5,6,13b-octahydro-8H-[1,6]naphthyridino[5,6-b]quinazolin-8-one ((±)-trans-5r) and (±)-(4aR,13bR)-10,11-dimethoxy-4-methyl-1,2,3,4,4a,5,6,13b-octahydro-8H-[1,6]naphthyridino[5,6-b]quinazolin-8-one ((±)-cis-5r). Both diastereomers of 5r were synthesized according to general procedure 6 starting from 4r. 5r required an additional 12 h of heating at 100° C. for cyclization to be completed. Purified by flash chromatography: 10-60% X/hexanes (X=2% NEt₃ 3:1 EtOAc/EtOH). (±)-trans-5r+(±)-cis-5r: 51.1 mg (76%).

(±)-trans-5r was isolated as a light-yellow solid (30.3 mg). ¹H NMR (400 MHz, CDCl₃) δ 7.54 (s, 1H), 7.01 (s, 1H), 4.22-4.06 (m, 2H), 3.96 (d, J=2.4 Hz, 6H), 2.95 (ddt, J=11.6, 4.0, 1.9 Hz, 1H), 2.74-2.61 (m, 2H), 2.40 (dq, J=13.1, 5.4 Hz, 1H), 2.34 (s, 3H), 2.09 (td, J=11.9, 3.2 Hz, 1H), 1.96 (dt, J=10.2, 5.1 Hz, 1H), 1.88-1.73 (m, 3H), 1.45 (tdd, J=12.8, 11.1, 4.1 Hz, 1H). HRMS (ESI) m/z: [M+H]⁺ Calcd for C₁₈H₂₄N₃O₃, 330.1812. Found 330.1817.

(±)-cis-5r was isolated as a yellow solid (20.8 mg ¹H NMR (400 MHz, CDCl₃) δ 7.56 (s, 1H), 7.01 (s, 1H), 4.09 (tdd, J=15.1, 8.1, 4.7 Hz, 2H), 3.98 (d, J=1.9 Hz, 6H), 3.00 (q, J=3.8 Hz, 1H), 2.76 (dt, J=11.3, 3.5 Hz, 2H), 2.58 (dt, J=6.4, 3.2 Hz, 1H), 2.34 (dt, J=14.5, 5.2 Hz, 1H), 2.29 (s, 3H), 2.25-2.16 (m, 1H), 2.01-1.89 (m, 1H), 1.71-1.52 (m, 3H). HRMS (ESI) m/z: [M+H]⁺ Calcd for C₁₈H₂₄N₃O₃, 330.1812. Found 330.1813.

### Example 25: Synthesis of (±)-(4aR,13bS)-10-chloro-4-methyl-11-phenyl-1,2,3,4,4a,5,6,13b-octahydro-8H-[1,6]naphthyridino[5,6-b]quinazolin-8-one ((±)-trans-9b) and (±)-(4aR,13bR)-10-chloro-4-methyl-11-phenyl-1,2,3,4,4a,5,6,13b-octahydro-8H-[1,6]naphthyridino[5,6-b]quinazolin-8-one ((±)-cis-9b)

Prepared according to Scheme 2.

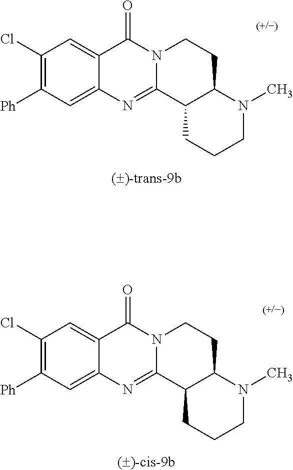 [SMILES: CN1CCC[C@@H]2c3nc4cc(-c5ccccc5)c(Cl)cc4c(=O)n3CC[C@H]21 ; CN1CCC[C@H]2c3nc4cc(-c5ccccc5)c(Cl)cc4c(=O)n3CC[C@H]21]

Step 1: Synthesis of tert-butyl (4-(7-bromo-6-chloro-3-neopentyl-4-oxo-3,4-dihydroquinazolin-2-yl)butyl)(methyl)carbamate (2s). Compound 2s was synthesized according to general procedure 3 from 2-amino-4-bromo-5-chloro-benzoic acid and 1a. Purification by flash chromatography (0-5% EtOAc/DCM) delivered 2s as an orange oil (1.14 g, 50%). ¹H NMR (400 MHz, CDCl₃) δ 8.25 (s, 1H), 7.90 (s, 1H), 4.14-3.95 (m, 2H), 3.26 (t, J=6.9 Hz, 2H), 2.85 (d, J=10.3 Hz, 5H), 1.79-1.73 (m, 2H), 1.62 (p, J=6.9 Hz, 2H), 1.44 (d, J=0.7 Hz, 9H), 0.97 (d, J=0.7 Hz, 9H). HRMS (ESI) m/z: [M+H]⁺ Calcd for C₂₃H₃₄BrClN₃O₃, 514.1467. Found 514.1459.

Step 2: Synthesis of 7-bromo-6-chloro-2-(4-(methylamino)butyl)-3-neopentylquinazolin-4(3H)-one bis-hydrochloride salt (3s). Compound 3s was synthesized according to general procedure 4 from 2s, which afforded 3s as a yellowish white solid (0.61 g, 67%). ¹H NMR (400 MHz, DMSO) δ 9.11 (s, 2H), 8.17 (s, 1H), 8.05 (s, 1H), 6.79 (s, 1H), 4.06 (s, 2H), 2.92 (dt, J=20.8, 6.5 Hz, 4H), 2.49 (d, J=1.5 Hz, 3H), 1.77 (dq, J=14.2, 7.8, 7.4 Hz, 4H), 0.92 (s, 9H). HRMS (ESI) m/z: [M+H]⁺ Calcd for C₁₈H₂₆BrClN₃O, 414.0942. Found 414.0930.

Step 3.1a: Synthesis of benzyl (2-(3-(7-bromo-6-chloro-3-neopentyl-4-oxo-3,4-dihydroquinazolin-2-yl)-1-methylpiperidin-2-yl)ethyl)carbamate (4s). Compound 4s was synthesized according to general procedure 5.1 from 3s. Purification by reverse phase flash chromatography (10-90% MeOH/H₂O) which afforded 4s as a yellowish-white solid (611 mg, 67%). 4s was used directly in the next step without analysis.

Step 3.1b: Synthesis of Suzuki benzyl (2-(3-(6-chloro-3-neopentyl-4-oxo-7-phenyl-3,4-dihydroquinazolin-2-yl)-1-methylpiperidin-2-yl)ethyl)carbamate (8b) Compound 8b was synthesized according to general procedure 5.2 from 4s. Purification by flash chromatography (3-30% X/hexanes (X=2% NEt₃ 3:1 EtOAc/EtOH)) and scavenging afforded 8b as a tan solid (173 mg, 82%). 8b was used directly in the next step without analysis.

Step 4: Synthesis of (±)-(4aR,13bS)-10-chloro-4-methyl-11-phenyl-1,2,3,4,4a,5,6,13b-octahydro-8H-[1,6]naphthyridino[5,6-b]quinazolin-8-one ((±)-trans-9b) and (±)-(4aR,13bR)-10-chloro-4-methyl-11-phenyl-1,2,3,4,4a,5,6,13b-octahydro-8H-[1,6]naphthyridino[5,6-b]quinazolin-8-one ((±)-cis-9b). Both diastereomers of 9b were synthesized according to general procedure 6 starting from 8b. Purified by flash chromatography: 10-60% X/hexanes (X=2% NEt₃ 3:1 EtOAc/EtOH). (±)-trans-9b+(±)-cis-9b=89 mg (81%).

(±)-trans-9b was isolated as a tan solid (57 mg). ¹H NMR (400 MHz, CDCl₃) δ 8.31 (s, 1H), 7.62 (s, 1H), 7.51-7.40 (m, 4H), 7.44-7.36 (m, 1H), 4.22-4.08 (m, 2H), 3.02-2.92 (m, 1H), 2.75-2.63 (m, 2H), 2.43 (dq, J=13.4, 5.5 Hz, 1H), 2.37 (s, 3H), 2.11 (td, J=11.9, 3.2 Hz, 1H), 1.99 (td, J=10.0, 4.9 Hz, 1H), 1.92-1.81 (m, 2H), 1.77 (qt, J=13.2, 3.3 Hz, 1H), 1.53-1.38 (m, 1H). HRMS (ESI) m/z: [M+H]⁺ Calcd for C₂₂H₂₃ClN₃O, 380.1524. Found 380.1523.

(±)-cis-9b was isolated as a waxy yellow solid (32 mg). ¹H NMR (400 MHz, CDCl₃) δ 8.32 (s, 1H), 7.61 (s, 1H), 7.50-7.39 (m, 5H), 4.09 (q, J=9.8, 7.7 Hz, 2H), 3.04 (s, 1H), 2.80 (d, J=10.7 Hz, 2H), 2.62 (t, J=5.8 Hz, 1H), 2.42-2.18 (m, 5H), 1.99 (q, J=10.9, 7.2 Hz, 1H), 1.61 (dd, J=27.4, 12.9 Hz, 3H). HRMS (ESI) m/z: [M+H]⁺ Calcd for C₂₂H₂₃ClN₃O, 380.1524. Found 380.1522.

### Example 26: Synthesis of (±)-(4aR,13bS)-11-chloro-4-methyl-10-(trifluoromethyl)-1,2,3,4,4a,5,6,13b-octahydro-8H-[1,6]naphthyridino[5,6-b]quinazolin-8-one ((±)-trans-5s) and (±)-(4aR,13bR)-11-chloro-4-methyl-10-(trifluoromethyl)-1,2,3,4,4a,5,6,13b-octahydro-8H-[1,6]naphthyridino[5,6-b]quinazolin-8-one ((±)-cis-5s)

Prepared according to Scheme 2.

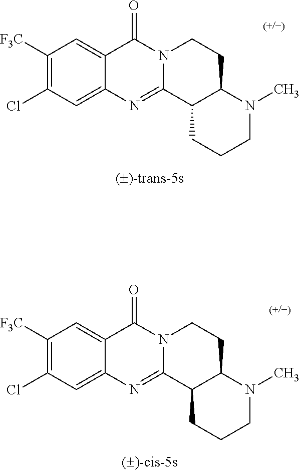 [SMILES: CN1CCC[C@@H]2c3nc4cc(Cl)c(C(F)(F)F)cc4c(=O)n3CC[C@H]21 ; CN1CCC[C@H]2c3nc4cc(Cl)c(C(F)(F)F)cc4c(=O)n3CC[C@H]21]

Step 1: Synthesis of tert-butyl (4-(7-chloro-3-neopentyl-4-oxo-6-(trifluoromethyl)-3,4-dihydroquinazolin-2-yl)butyl)(methyl)carbamate (2t). Compound 2t was synthesized according to general procedure 3 from 2-amino-4-chloro-5-trifluoromethyl-benzoic acid and 1a. Purification by flash chromatography (5-20% X/hexanes, X=4:1 EtOAc/DCM) delivered 2t as an orange oil (1.34 g, 32%). ¹H NMR (400 MHz, CDCl₃) δ 8.54 (s, 1H), 7.72 (s, 1H), 4.36-3.92 (m, 2H), 3.27 (t, J=6.9 Hz, 2H), 2.86 (d, J=18.7 Hz, 5H), 1.77 (d, J=11.5 Hz, 2H), 1.68-1.58 (m, 2H), 1.45 (s, 9H), 0.99 (s, 9H). HRMS (ESI) m/z: [M+H]⁺ Calcd for C₂₄H₃₄ClF₃N₃O₃, 504.2235. Found 504.2265.

Step 2: Synthesis of 7-chloro-2-(4-(methylamino)butyl)-3-neopentyl-6-(trifluoromethyl)quinazolin-4(3H)-one bis-hydrochloride salt (3t). Compound 3t was synthesized according to general procedure 4 from 2t, which afforded 3t as a white solid (1.01 g, 86%). ¹H NMR (400 MHz, DMSO) δ 9.19 (s, 2H), 8.36 (s, 1H), 7.92 (s, 1H), 4.08 (s, 2H), 2.93 (dt, J=29.4, 6.4 Hz, 4H), 2.48 (s, 4H), 1.79 (td, J=10.6, 5.4 Hz, 4H), 0.92 (s, 9H). HRMS (ESI) m/z: [M+H]⁺ Calcd for C₁₉H₂₆ClF₃N₃O, 404.1711. Found 404.1730.

Step 3.1: Synthesis of benzyl (2-(3-(7-chloro-3-neopentyl-4-oxo-6-(trifluoromethyl)-3,4-dihydroquinazolin-2-yl)-1-methylpiperidin-2-yl)ethyl)carbamate (4t). Compound 4t was synthesized according to general procedure 5.1 from 3t. Purification by reverse phase flash chromatography (10-80% MeOH/H₂O) which afforded 4t as a yellow solid (872 mg, 92%). 4t was used directly in the next step without analysis.

Step 4: Synthesis of (±)-(4aR,13bS)-11-chloro-4-methyl-10-(trifluoromethyl)-1,2,3,4,4a,5,6,13b-octahydro-8H-[1,6]naphthyridino[5,6-b]quinazolin-8-one ((±)-trans-5s) and (±)-(4aR,13bR)-11-chloro-4-methyl-10-(trifluoromethyl)-1,2,3,4,4a,5,6,13b-octahydro-8H-[1,6]naphthyridino[5,6-b]quinazolin-8-one ((±)-cis-5s). Both diastereomers of 5s were synthesized according to general procedure 6 starting from 4t. Purified by flash chromatography: 15-50% X/hexanes (X=2% NEt₃ 3:1 EtOAc/EtOH). (±)-trans-5s+(±)-cis-5s: 411 mg (85%).

(±)-trans-5s was isolated as a yellow solid (214 mg). ¹H NMR (400 MHz, CDCl₃) δ 8.54 (s, 1H), 7.74 (s, 1H), 4.22-4.12 (m, 1H), 4.15-4.05 (m, 1H), 2.97 (dq, J=11.7, 2.0 Hz, 1H), 2.69 (tt, J=10.4, 3.8 Hz, 2H), 2.44 (dq, J=13.3, 5.4 Hz, 1H), 2.36 (s, 3H), 2.11 (td, J=12.0, 3.1 Hz, 1H), 1.99 (td, J=10.0, 4.8 Hz, 1H), 1.93-1.69 (m, 3H), 1.52-1.33 (m, 1H). HRMS (ESI) m/z: [M+H]⁺ Calcd for C₁₇H₁₈ClF₃N₃O, 372.1066. Found 372.1099.

(±)-cis-5s was isolated as a yellow solid (197 mg). ¹H NMR (400 MHz, CDCl₃) δ 8.56 (s, 1H), 7.74 (s, 1H), 4.14 (ddd, J=14.2, 5.8, 3.6 Hz, 1H), 3.98 (ddd, J=14.1, 11.6, 5.0 Hz, 1H), 3.00 (q, J=3.6 Hz, 1H), 2.91-2.82 (m, 1H), 2.82-2.74 (m, 1H), 2.54 (dt, J=5.9, 3.1 Hz, 1H), 2.38 (dtd, J=14.0, 5.0, 3.5 Hz, 1H), 2.27 (s, 3H), 2.21-2.12 (m, 1H), 1.95 (dddd, J=14.4, 11.6, 5.8, 2.7 Hz, 1H), 1.58 (dddd, J=15.4, 13.3, 7.3, 4.3 Hz, 3H). HRMS (ESI) m/z: [M+H]⁺ Calcd for C₁₉H₂₆ClF₃N₃O, 404.1711. Found 404.1730.

### Example 27: Synthesis of (±)-(4aR,13bS)-10-chloro-12-methoxy-4-methyl-1,2,3,4,4a,5,6,13b-octahydro-8H-[1,6]naphthyridino[5,6-b]quinazolin-8-one ((±)-trans-5t) and (±)-(4aR,13bR)-10-chloro-12-methoxy-4-methyl-1,2,3,4,4a,5,6,13b-octahydro-8H-[1,6]naphthyridino[5,6-b]quinazolin-8-one ((±)-cis-5t)

Prepared according to Scheme 2.

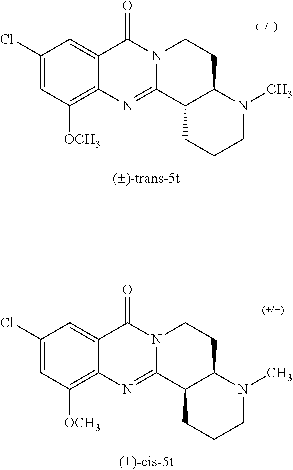 [SMILES: COc1cc(Cl)cc2c(=O)n3c(nc12)[C@H]1CCCN(C)[C@@H]1CC3 ; COc1cc(Cl)cc2c(=O)n3c(nc12)[C@@H]1CCCN(C)[C@@H]1CC3]

Step 1: Synthesis of tert-butyl (4-(6-chloro-8-methoxy-3-neopentyl-4-oxo-3,4-dihydroquinazolin-2-yl)butyl)(methyl)carbamate (2u). Compound 2u was synthesized according to general procedure 3 from 2-amino-3-methoxy-4-chloro benzoic acid and 1a. Purification by flash chromatography (5-20% X/hexanes, X=4:1 EtOAc/DCM) delivered 2u as a yellow oil (1.91 g, 83%). ¹H NMR (400 MHz, CDCl₃) δ 7.77 (d, J=2.2 Hz, 1H), 7.07 (d, J=2.3 Hz, 1H), 4.41-3.88 (m, 0H), 4.40-3.75 (m, 5H), 3.22 (s, 2H), 2.94 (dd, J=14.2, 6.6 Hz, 2H), 2.81 (s, 3H), 1.71 (d, J=7.7 Hz, 2H), 1.61 (q, J=7.2 Hz, 2H), 1.42 (s, 9H), 0.96 (s, 9H). HRMS (ESI) m/z: [M+H]⁺ Calcd for C₂₄H₃₇ClN₃O₄, 466.2467. Found 466.2477.

Step 2: Synthesis of 6-chloro-8-methoxy-2-(4-(methylamino)butyl)-3-neopentylquinazolin-4(3H)-one bis-hydrochloride salt (3u). Compound 3u was synthesized according to general procedure 4 from 2u, which afforded 3u as a white solid (1.36 g, 81%). ¹H NMR (400 MHz, DMSO) δ 9.26-9.16 (m, 2H), 8.17 (s, 2H), 7.57 (d, J=2.2 Hz, 1H), 7.38 (d, J=2.2 Hz, 1H), 4.07 (s, 2H), 3.94 (s, 3H), 2.91 (dt, J=21.8, 6.2 Hz, 4H), 2.47 (m, 2H), 1.76 (p, J=3.2 Hz, 4H), 0.90 (s, 9H). HRMS (ESI) m/z: [M+H]⁺ Calcd for C₁₉H₂₉ClN₃O₂, 366.1942. Found 366.1932.

Step 3.1: Synthesis of benzyl (2-(3-(6-chloro-8-methoxy-3-neopentyl-4-oxo-3,4-dihydroquinazolin-2-yl)-1-methylpiperidin-2-yl)ethyl)carbamate (4u). Compound 4u was synthesized according to general procedure 5.1 from 3u. Purification by reverse phase flash chromatography (10-90% MeOH/H₂O) which afforded 4u as an off-white solid (754 mg, 85%). ¹H NMR (400 MHz, CDCl₃) δ 7.75 (dd, J=3.5, 2.2 Hz, 1H), 7.35-7.23 (m, 4H), 7.24 (dd, J=4.6, 2.4 Hz, 1H), 6.94-6.89 (m, 1H), 5.93 (s, 1H), 5.14 (s, 1H), 5.03-4.91 (m, 2H), 4.48 (s, 1H), 3.92 (s, 1H), 3.75 (s, 2H), 3.67-3.53 (m, 0H), 3.37 (s, 2H), 3.05 (ddd, J=11.9, 9.3, 3.3 Hz, 1H), 2.97-2.86 (m, 2H), 2.84 (s, 1H), 2.48 (s, 1H), 2.39 (s, 2H), 2.33 (s, 2H), 1.96-1.88 (m, 1H), 1.83-1.64 (m, 2H), 1.60 (d, J=16.8 Hz, 1H), 0.96 (d, J=7.9 Hz, 9H). HRMS (ESI) m/z: [M+H]⁺ Calcd for C₃₀H₄₀ClN₄O₄, 555.2733. Found 555.2744.

Step 4: Synthesis of (±)-(4aR,13bS)-10-chloro-12-methoxy-4-methyl-1,2,3,4,4a,5,6,13b-octahydro-8H-[1,6]naphthyridino[5,6-b]quinazolin-8-one ((±)-trans-5t) and (±)-(4aR,13bR)-10-chloro-12-methoxy-4-methyl-1,2,3,4,4a,5,6,13b-octahydro-8H-[1,6]naphthyridino[5,6-b]quinazolin-8-one ((±)-cis-5t). Both diastereomers of 5t were synthesized according to general procedure 6 starting from 4u. Purified by flash chromatography: 15-50% X/hexanes (X=2% NEt₃ 3:1 EtOAc/EtOH). (±)-trans-5t+(±)-cis-5t=262 mg (79%).

(±)-trans-5t was isolated as a yellowish white solid (204 mg). ¹H NMR (400 MHz, CDCl₃) δ 7.79 (d, J=2.2 Hz, 1H), 7.07 (d, J=2.3 Hz, 1H), 4.28 (ddd, J=14.6, 6.0, 4.2 Hz, 1H), 3.98 (s, 4H), 2.96 (ddt, J=11.6, 3.9, 1.9 Hz, 1H), 2.81 (ddt, J=13.2, 4.0, 2.4 Hz, 1H), 2.73 (ddd, J=11.5, 10.5, 3.7 Hz, 1H), 2.46 (ddt, J=13.8, 5.8, 4.2 Hz, 1H), 2.36 (s, 3H), 2.11 (td, J=11.9, 3.4 Hz, 1H), 1.98 (td, J=10.4, 4.1 Hz, 1H), 1.91-1.70 (m, 3H), 1.47 (tdd, J=13.1, 11.6, 4.5 Hz, 1H). HRMS (ESI) m/z: [M+H]⁺ Calcd for C₁₇H₂₁ClN₃O₂, 334.1317. Found 334.1332.

(±)-cis-5t was isolated as a tan yellowish white solid (58 mg). ¹H NMR (400 MHz, CDCl₃) δ 7.79 (d, J=2.2 Hz, 1H), 7.06 (d, J=2.3 Hz, 1H), 4.11 (ddd, J=14.2, 5.8, 3.9 Hz, 1H), 4.05-3.93 (m, 4H), 3.10 (q, J=4.2 Hz, 1H), 2.77 (ddt, J=19.4, 11.2, 3.3 Hz, 2H), 2.57 (p, J=2.8 Hz, 1H), 2.37 (dq, J=14.5, 4.8 Hz, 1H), 2.29 (s, 3H), 2.20 (dt, J=11.4, 6.8 Hz, 1H), 1.91 (dddd, J=13.8, 11.0, 5.7, 2.2 Hz, 1H), 1.72-1.62 (m, 1H), 1.57 (dq, J=10.0, 5.4, 4.6 Hz, 2H). HRMS (ESI) m/z: [M+H]⁺ Calcd for C₁₇H₂₁ClN₃O₂, 334.1317. Found 334.1326.

### Example 28: Synthesis of (±)-(4aR,13bS)-12-methoxy-4-methyl-1,2,3,4,4a,5,6,13b-octahydro-8H-[1,6]naphthyridino[5,6-b]quinazolin-8-one ((±)-trans-5u) and (±)-(4aR,13bR)-12-methoxy-4-methyl-1,2,3,4,4a,5,6,13b-octahydro-8H-[1,6]naphthyridino[5,6-b]quinazolin-8-one((±)-cis-5u)

Prepared according to Scheme 2.

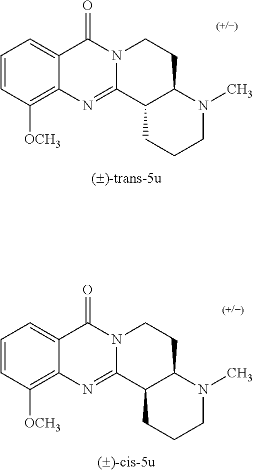 [SMILES: COc1cccc2c(=O)n3c(nc12)[C@H]1CCCN(C)[C@@H]1CC3 ; COc1cccc2c(=O)n3c(nc12)[C@@H]1CCCN(C)[C@@H]1CC3]

Step 1: Synthesis of tert-butyl (4-(8-methoxy-3-neopentyl-4-oxo-3,4-dihydroquinazolin-2-yl)butyl)(methyl)carbamate (2v). Compound 2v was synthesized according to general procedure 3 from 2-amino-3-methoxy-benzoic acid and 1a. Purification by flash chromatography (5-50% X/hexanes, X=4:1 EtOAc/DCM) delivered 2v as a yellow oil (0.97 g, 58%). ¹H NMR (400 MHz, CDCl₃) δ 7.80 (dd, J=8.0, 1.3 Hz, 1H), 7.34 (t, J=8.0 Hz, 1H), 7.14 (dd, J=8.1, 1.3 Hz, 1H), 4.50-3.57 (m, 5H), 3.22 (s, 2H), 2.96 (t, J=7.8 Hz, 2H), 2.81 (s, 3H), 1.73 (p, J=7.5 Hz, 2H), 1.67-1.54 (m, 2H), 1.42 (s, 9H), 0.97 (s, 9H). HRMS (ESI) m/z: [M+H]⁺ Calcd for C₂₄H₃₇N₃O₄, 432.2857. Found 432.2855.

Step 2: Synthesis of 8-methoxy-2-(4-(methylamino)butyl)-3-neopentylquinazolin-4(3H)-one bis-hydrochloride salt (3v). Compound 3v was synthesized according to general procedure 4 from 2v, which afforded 3v as a white solid (0.75 g, 88%). ¹H NMR (400 MHz, DMSO) δ 9.25 (s, 2H), 8.37 (s, 1H), 7.68 (dd, J=7.7, 1.5 Hz, 1H), 7.53-7.40 (m, 2H), 4.15-4.05 (m, 1H), 3.96 (s, 3H), 3.04 (d, J=6.7 Hz, 2H), 2.90 (q, J=6.4 Hz, 2H), 2.50 (d, J=11.2 Hz, 4H), 1.84-1.75 (m, 4H), 0.94 (s, 9H). HRMS (ESI) m/z: [M+H]⁺ Calcd for C₁₉H₃₀N₃O₂, 332.2333. Found 332.2333.

Step 3.1: Synthesis of benzyl (2-(3-(8-methoxy-3-neopentyl-4-oxo-3,4-dihydroquinazolin-2-yl)-1-methylpiperidin-2-yl)ethyl)carbamate (4v). Compound 4v was synthesized according to general procedure 5.1 from 3v. Purification by reverse phase flash chromatography (10-75% MeOH/H₂O) afforded 4v a white solid (247 mg, 94%). ¹H NMR (400 MHz, CDCl₃) δ 7.87-7.73 (m, 1H), 7.37-7.22 (m, 6.4H), 7.14 (d, J=7.9 Hz, 0.3H), 7.04 (d, J=8.0 Hz, 0.6H), 6.07 (s, 0.5H), 5.14-4.92 (m, 2.1H), 4.55 (s, 0.6H), 4.08-3.92 (m, 1H), 3.82 (s, 1.9H), 3.68-3.27 (m, 2.4H), 3.08 (ddd, J=11.8, 9.3, 3.3 Hz, 0.8H), 3.01-2.80 (m, 2.2H), 2.76-2.63 (m, 0.3H), 2.60-2.22 (m, 4H.3), 2.09-1.29 (m, 6.2H), 1.01-0.97 (m, 9H). HRMS (ESI) m/z: [M+H]⁺ Calcd for C₃₀H₄₁N₄O₄, 521.3122. Found 521.3122.

Step 4: Synthesis of (±)-(4aR,13bS)-12-methoxy-4-methyl-1,2,3,4,4a,5,6,13b-octahydro-8H-[1,6]naphthyridino[5,6-b]quinazolin-8-one ((±)-trans-5u) and (±)-(4aR,13bR)-12-methoxy-4-methyl-1,2,3,4,4a,5,6,13b-octahydro-8H-[1,6]naphthyridino[5,6-b]quinazolin-8-one((±)-cis-5u). Both diastereomers of 5u were synthesized according to general procedure 6 starting from 4v. Purified by flash chromatography: 10-60% X/hexanes (X=2% NEt₃ 3:1 EtOAc/EtOH). (±)-trans-5u+(±)-cis-5u: 47.6 mg (79%).

(±)-trans-5u was isolated as an off white solid (36.1 mg). ¹H NMR (400 MHz, CDCl₃) δ 7.81 (dd, J=8.1, 1.2 Hz, 1H), 7.33 (t, J=8.0 Hz, 1H), 7.12 (dd, J=8.0, 1.3 Hz, 1H), 4.28 (ddd, J=14.5, 6.0, 4.2 Hz, 1H), 4.01-3.91 (m, 4H), 2.95 (dp, J=11.5, 2.0 Hz, 1H), 2.89-2.78 (m, 1H), 2.73 (ddd, J=11.6, 10.6, 3.7 Hz, 1H), 2.45 (ddt, J=13.8, 5.7, 4.2 Hz, 1H), 2.35 (s, 3H), 2.10 (td, J=11.8, 3.4 Hz, 1H), 1.97 (td, J=10.5, 4.2 Hz, 1H), 1.90-1.79 (m, 2H), 1.79-1.69 (m, 1H), 1.48 (tdd, J=13.2, 11.7, 4.6 Hz, 1H). HRMS (ESI) m/z: [M+H]⁺ Calcd for C₁₇H₂₂N₃O₂, 300.1707. Found 300.1706.

(±)-cis-5u was isolated as an off white solid (11.5 mg). ¹H NMR (400 MHz, CDCl₃) δ 7.84 (dd, J=8.1, 1.3 Hz, 1H), 7.33 (t, J=8.0 Hz, 1H), 7.13 (dd, J=8.0, 1.3 Hz, 1H), 4.18-4.01 (m, 2H), 3.98 (s, 3H), 3.14 (q, J=4.3 Hz, 1H), 2.86-2.70 (m, 2H), 2.59 (dt, J=6.1, 2.9 Hz, 1H), 2.45-2.32 (m, 1H), 2.30 (s, 3H), 2.27-2.14 (m, 1H), 1.92 (dddd, J=14.0, 10.9, 5.9, 2.5 Hz, 1H), 1.69 (dddd, J=12.8, 10.6, 7.1, 4.2 Hz, 1H), 1.59 (hept, J=3.7 Hz, 2H). HRMS (ESI) m/z: [M+H]⁺ Calcd for C₁₇H₂₂N₃O₂, 300.1707. Found 300.1705.

### Example 29: Synthesis of (±)-(4aR,13bS)-4-methyl-3,4,4a,5,6,13b-hexahydro-[1,4]oxazino[2′,3′:3,4]pyrido[2,1-b]quinazolin-8(2H)-one ((±)-trans-5v) and (±)-(4aR,13bR)-4-methyl-3,4,4a,5,6,13b-hexahydro-[1,4]oxazino[2′,3′:3,4]pyrido[2,1-b]quinazolin-8(2H)-one ((±)-cis-5v)

Prepared according to Scheme 2.

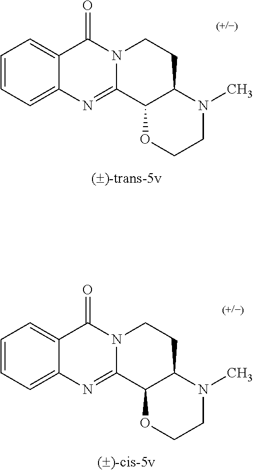 [SMILES: CN1CCO[C@@H]2c3nc4ccccc4c(=O)n3CC[C@H]21 ; CN1CCO[C@H]2c3nc4ccccc4c(=O)n3CC[C@H]21]

Step 1: Synthesis of tert-butyl methyl(4-(8-methyl-3-neopentyl-4-oxo-3,4-dihydroquinazolin-2-yl)butyl)carbamate (2w). Compound 2w was synthesized according to general procedure 3 from anthranilic acid and 1d. Purification by flash chromatography (10-25% X/hexanes, X=4:1 EtOAc/DCM) delivered S22 as a colorless oil (0.76 g, 40%). ¹H NMR (400 MHz, CDCl₃) δ 8.25 (dd, J=8.0, 1.5 Hz, 1H), 7.73 (ddd, J=8.4, 7.0, 1.5 Hz, 1H), 7.66 (dd, J=8.2, 1.3 Hz, 1H), 7.47 (ddd, J=8.2, 6.9, 1.3 Hz, 1H), 4.68 (s, 2H), 4.44-4.12 (m, 2H), 3.61 (d, J=11.2 Hz, 2H), 3.39 (s, 2H), 2.86 (s, 3H), 1.41 (s, 9H), 0.99 (s, 9H). HRMS (ESI) m/z: [M+H]⁺ Calcd for C₂₂H₃₄N₃O₄, 404.2544. Found 404.2556.

Step 2: Synthesis of 2-((2-(methylamino)ethoxy)methyl)-3-neopentylquinazolin-4(3H)-one bis-hydrochloride salt (3w). Compound 3w was synthesized according to general procedure 4 from 2w, which afforded 3w as a grey-white solid (0.52 g, 82%). ¹H NMR (400 MHz, DMSO) δ 9.51 (d, J=6.1 Hz, 3H), 8.25-8.13 (m, 2H), 7.94 (ddd, J=8.4, 7.2, 1.5 Hz, 1H), 7.65 (ddd, J=8.1, 7.2, 1.1 Hz, 1H), 4.93 (s, 2H), 4.06 (s, 2H), 3.95 (t, J=5.0 Hz, 2H), 3.16 (p, J=5.4 Hz, 2H), 2.55 (t, J=5.4 Hz, 3H), 0.96 (s, 9H). HRMS (ESI) m/z: [M+H]⁺ Calcd for C₁₇H₂₆N₃O₂, 304.2020. Found 304.2030.

Step 3.1: Synthesis of benzyl (2-(4-methyl-2-(3-neopentyl-4-oxo-3,4-dihydroquinazolin-2-yl)morpholin-3-yl)ethyl)carbamate (4w). Compound 4w was synthesized according to general procedure 5.1 from 3w. Purification by reverse phase flash chromatography (10-80% MeOH/H₂O) afforded 4w an off-white solid (204 mg, 83%). ¹H NMR (400 MHz, CDCl₃) δ 8.29-8.18 (m, 1H), 8.06 (s, 0.1H), 7.89-7.72 (m, 0.9H), 7.68-7.57 (m, 0.3H), 7.50-7.26 (m, 6.8H), 6.37 (s, 0.5H), 5.21-4.95 (m, 2.4H), 4.62-4.43 (m, 1.5H), 4.04 (dd, J=11.4, 3.8 Hz, 0.3H), 3.93 (dd, J=11.2, 3.3 Hz, 0.8H), 3.89-3.65 (m, 1.8H), 3.56-3.32 (m, 1.5H), 3.13-2.76 (m, 1.8H), 2.70 (d, J=12.1 Hz, 0.7H), 2.60-2.31 (m, 4.2H), 2.31-1.92 (m, 0.7H), 1.91-1.61 (m, 1.3H), 1.08-0.95 (m, 9H). HRMS (ESI) m/z: [M+H]⁺ Calcd for C₂₈H₃₆N₄O₄, 493.2809. Found 493.2828.

Step 4: Synthesis of (±)-(4aR,13bS)-4-methyl-3,4,4a,5,6,13b-hexahydro-[1,4]oxazino[2′,3′:3,4]pyrido[2,1-b]quinazolin-8(2H)-one ((±)-trans-5v) and (±)-(4aR,13bR)-4-methyl-3,4,4a,5,6,13b-hexahydro-[1,4]oxazino[2′,3′:3,4]pyrido[2,1-b]quinazolin-8(2H)-one ((±)-cis-5v). Both diastereomers of 5v were synthesized according to general procedure 6 starting from 4w. Purified by flash chromatography: 10-75% X/hexanes (X=2% NEt₃ 3:1 EtOAc/EtOH). (±)-trans-5v+(±)-cis-5v: 53.5 mg (98%).

(±)-trans-5v was isolated as a yellow oil (38.5 mg). ¹H NMR (400 MHz, CDCl₃) δ 8.24 (dd, J=8.0, 1.6 Hz, 1H), 7.83 (dt, J=8.2, 0.8 Hz, 1H), 7.71 (ddd, J=8.4, 7.1, 1.6 Hz, 1H), 7.44 (ddd, J=8.1, 7.1, 1.2 Hz, 1H), 4.32 (d, J=10.1 Hz, 1H), 4.22 (ddd, J=11.6, 3.4, 1.3 Hz, 1H), 4.19-4.06 (m, 2H), 3.96 (td, J=11.7, 2.5 Hz, 1H), 2.78 (ddd, J=12.0, 2.5, 1.3 Hz, 1H), 2.53-2.38 (m, 2H), 2.36 (s, 3H), 2.24 (td, J=10.0, 5.2 Hz, 1H), 1.84 (dddd, J=13.7, 10.0, 8.8, 6.8 Hz, 1H). HRMS (ESI) m/z: [M+H]⁺ Calcd for C₁₅H₁₈N₃O₂, 272.1394. Found 272.1397.

(±)-cis-5v was isolated as a yellow-white solid (15 mg). ¹H NMR (400 MHz, CDCl₃) δ 8.26 (dd, J=8.0, 1.5 Hz, 1H), 7.79 (dd, J=8.3, 1.2 Hz, 1H), 7.72 (ddd, J=8.3, 6.9, 1.6 Hz, 1H), 7.45 (ddd, J=8.0, 6.9, 1.3 Hz, 1H), 4.69 (d, J=3.7 Hz, 1H), 4.12 (ddd, J=14.0, 5.6, 4.4 Hz, 1H), 3.99 (ddd, J=14.4, 10.7, 4.8 Hz, 1H), 3.88 (ddd, J=12.1, 9.4, 2.9 Hz, 1H), 3.79 (dt, J=11.6, 3.6 Hz, 1H), 2.81 (dt, J=6.3, 3.1 Hz, 1H), 2.65 (dt, J=11.8, 3.3 Hz, 1H), 2.55-2.36 (m, 2H), 2.34 (s, 3H), 1.95 (dddd, J=14.6, 10.6, 5.6, 2.7 Hz, 1H). HRMS (ESI) m/z: [M+H]⁺ Calcd for C₁₅H₁₈N₃O₂, 272.1394. Found 272.1397.

### Example 30: Synthesis of (±)-(4aR,13bS)-10-chloro-4-methyl-3,4,4a,5,6,13b-hexahydro-[1,4]oxazino[2′,3′:3,4]pyrido[2,1-b]quinazolin-8(2H)-one ((±)-trans-5w) and (±)-(4aR,13bR)-10-chloro-4-methyl-3,4,4a,5,6,13b-hexahydro-[1,4]oxazino[2′,3′:3,4]pyrido[2,1-b]quinazolin-8(2H)-one ((±)-cis-5w)

Prepared according to Scheme 2.

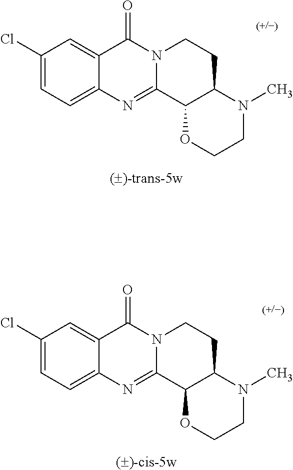 [SMILES: CN1CCO[C@@H]2c3nc4ccc(Cl)cc4c(=O)n3CC[C@H]21 ; CN1CCO[C@H]2c3nc4ccc(Cl)cc4c(=O)n3CC[C@H]21]

Step 1: Synthesis of tert-butyl (2-((6-chloro-3-neopentyl-4-oxo-3,4-dihydroquinazolin-2-yl)methoxy)ethyl)(methyl)carbamate (2×). Compound 2x was synthesized according to general procedure 3 from 2-amino-5-chloro-benzoic acid and 1d. Purification by flash chromatography (1-20% EtOAc/DCM) delivered 2x as a slight yellow oil (548.3 mg, 41%). ¹H NMR (400 MHz, Chloroform-d) δ 8.22 (d, J=2.3 Hz, 1H), 7.67 (dd, J=8.7, 2.4 Hz, 1H), 7.61 (d, J=8.7 Hz, 1H), 4.67 (s, 2H), 4.21 (s, 2H), 3.63 (s, 2H), 3.40 (s, 2H), 2.87 (s, 3H), 1.42 (s, 9H), 0.99 (s, 9H). HRMS (ESI) m/z: [M+H]⁺ Calcd for C₂₂H₃₃ClN₃O₄: 438.2154; Found: 438.2148.

Step 2: Synthesis of 6-chloro-2-((2-(methylamino)ethoxy)methyl)-3-neopentylquinazolin-4(3H)-one bis hydrochloride salt (3x). Compound 3x was synthesized according to general procedure 4 from 2x, which afforded 3x as a white solid (392.5 mg, 79%). ¹H NMR (400 MHz, DMSO-d6) δ 9.23 (s, 2H), 8.09 (d, J=2.2 Hz, 1H), 7.93-7.86 (m, 2H), 6.48 (s, 1H), 4.78 (s, 2H), 4.06 (s, 2H), 3.87 (t, J=5.0 Hz, 2H), 3.12 (t, J=5.6 Hz, 2H), 2.55 (t, J=5.4 Hz, 3H), 0.94 (s, 9H). HRMS (ESI) m/z: [M+H]⁺ Calcd for C₁₇H₂₄ClN₃O₂: 338.1630; Found: 338.1629.

Step 3.1: Synthesis of benzyl (2-(2-(6-chloro-3-neopentyl-4-oxo-3,4-dihydroquinazolin-2-yl)-4-methylmorpholin-3-yl)ethyl)carbamate (4x). Compound 4x was synthesized according to general procedure 5.1 from 3x. Purification by reverse phase flash chromatography (10-75% MeOH/H₂O) which afforded 4x as a white solid (356 mg, 74%). 4t was used directly in the next step without analysis.

Step 4: Synthesis of (±)-(4aR,13bS)-10-chloro-4-methyl-3,4,4a,5,6,13b-hexahydro-[1,4]oxazino[2′,3′:3,4]pyrido[2,1-b]quinazolin-8(2H)-one ((±)-trans-5w) and (±)-(4aR,13bR)-10-chloro-4-methyl-3,4,4a,5,6,13b-hexahydro-[1,4]oxazino[2′,3′:3,4]pyrido[2,1-b]quinazolin-8(2H)-one ((±)-cis-5w). Both diastereomers of 5w were synthesized according to general procedure 6 starting from 4x. Purified by flash chromatography: 10-60% X/hexanes (X=2% NEt₃ 3:1 EtOAc/EtOH). (±)-trans-5w+(±)-cis-5w: 127.6 mg (62%).

(±)-trans-5w was isolated as an off white solid (89.1 mg). ¹H NMR (400 MHz, Chloroform-d) δ 8.20 (d, J=2.4 Hz, 1H), 7.77 (d, J=8.7 Hz, 1H), 7.65 (dd, J=8.7, 2.5 Hz, 1H), 4.32 (d, J=10.1 Hz, 1H), 4.22 (ddd, J=11.6, 3.5, 1.3 Hz, 1H), 4.17-4.11 (m, 2H), 3.96 (td, J=11.7, 2.5 Hz, 1H), 2.79 (ddd, J=12.0, 2.6, 1.3 Hz, 1H), 2.49-2.41 (m, 2H), 2.37 (s, 3H), 2.24 (td, J=10.1, 5.2 Hz, 1H), 1.85 (dddd, J=13.8, 10.1, 8.7, 7.0 Hz, 1H). HRMS (ESI) m/z: [M+H]⁺ Calcd for C₁₅H₁₇N₃O₂Cl⁺: 306.1004; Found: 306.1003.

(±)-cis-5w was isolated as an off white solid (38.5 mg). ¹H NMR (400 MHz, Chloroform-d) δ 8.22 (d, J=2.5 Hz, 1H), 7.73 (d, J=8.7 Hz, 1H), 7.66 (dd, J=8.7, 2.4 Hz, 1H), 4.68 (d, J=3.7 Hz, 1H), 4.12 (ddd, J=14.1, 5.6, 4.2 Hz, 1H), 3.98 (ddd, J=14.1, 10.9, 4.8 Hz, 1H), 3.90-3.75 (m, 2H), 2.81 (dt, J=6.2, 3.1 Hz, 1H), 2.66 (dt, J=11.8, 3.2 Hz, 1H), 2.54-2.37 (m, 2H), 2.34 (s, 3H), 1.95 (dddd, J=14.8, 10.9, 5.6, 2.7 Hz, 1H). HRMS (ESI) m/z: [M+H]⁺ Calcd for C₁₅H₁₇N₃O₂Cl⁺: 306.1004; Found: 306.1005.

### Example 31: Synthesis of (±)-(4aR,13bS)-4-methyl-10-(trifluoromethyl)-3,4,4a,5,6,13b-hexahydro-[1,4]oxazino[2′,3′:3,4]pyrido[2,1-b]quinazolin-8(2H)-one ((±)-trans-5x) and (±)-(4aR,13bR)-4-methyl-10-(trifluoromethyl)-3,4,4a,5,6,13b-hexahydro-[1,4]oxazino[2′,3′:3,4]pyrido[2,1-b]quinazolin-8(2H)-one ((±)-cis-5x)

Prepared according to Scheme 2.

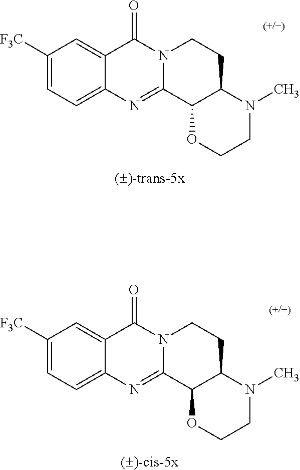 [SMILES: CN1CCO[C@@H]2c3nc4ccc(C(F)(F)F)cc4c(=O)n3CC[C@H]21 ; CN1CCO[C@H]2c3nc4ccc(C(F)(F)F)cc4c(=O)n3CC[C@H]21]

Step 1: Synthesis of tert-butyl methyl(2-((3-neopentyl-4-oxo-6-(trifluoromethyl)-3,4-dihydroquinazolin-2-yl)methoxy)ethyl)carbamate (2y). Compound 2y was synthesized according to general procedure 3 from 2-amino-5-trifluoromethyl-benzoic acid and 1d. Purification by flash chromatography (1-10% EtOAc/DCM) delivered 2y as a yellow oil (0.81 g, 39%). ¹H NMR (400 MHz, CDCl₃) δ 8.58-8.52 (m, 1H), 7.92 (dd, J=8.6, 2.1 Hz, 1H), 7.77 (d, J=8.5 Hz, 1H), 4.70 (s, 2H), 4.32-4.14 (m, 2H), 3.64 (s, 2H), 3.40 (s, 2H), 2.87 (s, 3H), 1.41 (s, 9H), 1.00 (s, 9H). HRMS (ESI) m/z: [M+H]⁺ Calcd for C₂₃H₃₃F₃N₃O₄, 472.2418. Found 472.2425.

Step 2: Synthesis of 2-((2-(methylamino)ethoxy)methyl)-3-neopentyl-6-(trifluoromethyl)quinazolin-4(3H)-one bis-hydrochloride salt (3y). Compound 3y was synthesized according to general procedure 4 from 2y, which afforded 3y as an off-white solid (0.56 g, 78%). ¹H NMR (400 MHz, DMSO) δ 11.77 (s, 1H), 9.39-9.28 (m, 2H), 8.37 (d, J=2.2 Hz, 1H), 8.16 (dd, J=8.6, 2.2 Hz, 1H), 8.04 (d, J=8.6 Hz, 1H), 4.82 (s, 2H), 4.08 (s, 2H), 3.90 (t, J=5.1 Hz, 2H), 3.13 (p, J=5.4 Hz, 2H), 2.55 (t, J=5.4 Hz, 3H), 0.94 (s, 9H). HRMS (ESI) m/z: [M+H]⁺ Calcd for C₁₈H₂₆F₃N₃O₂, 372.1893. Found 372.1887.

Step 3.1: Synthesis of benzyl (2-(4-methyl-2-(3-neopentyl-4-oxo-6-(trifluoromethyl)-3,4-dihydroquinazolin-2-yl)morpholin-3-yl)ethyl)carbamate (4y). Compound 4y was synthesized according to general procedure 5.1 from 3y. Purification by reverse phase flash chromatography (10-85% MeOH/H₂O) which afforded 4y as a light yellow solid (527 mg, 84%). 4y was used directly in the next step without analysis.

Step 4: Synthesis of (±)-(4aR,13bS)-4-methyl-10-(trifluoromethyl)-3,4,4a,5,6,13b-hexahydro-[1,4]oxazino[2′,3′:3,4]pyrido[2,1-b]quinazolin-8(2H)-one ((±)-trans-5x) and (±)-(4aR,13bR)-4-methyl-10-(trifluoromethyl)-3,4,4a,5,6,13b-hexahydro-[1,4]oxazino[2′,3′:3,4]pyrido[2,1-b]quinazolin-8(2H)-one ((±)-cis-5x). Both diastereomers of 5x were synthesized according to general procedure 6 starting from 4y. Purified by flash chromatography: 15-50% X/hexanes (X=2% NEt₃ 3:1 EtOAc/EtOH). (±)-trans-5x+(±)-cis-5x: 180 mg (79%).

(±)-trans-5x was isolated as a yellow solid (128 mg). ¹H NMR (400 MHz, CDCl₃) δ 8.54 (dd, J=2.0, 1.0 Hz, 1H), 7.96-7.88 (m, 2H), 4.35 (d, J=10.1 Hz, 1H), 4.28-4.08 (m, 3H), 3.97 (td, J=11.7, 2.5 Hz, 1H), 2.80 (ddd, J=12.0, 2.5, 1.3 Hz, 1H), 2.55-2.40 (m, 2H), 2.38 (s, 3H), 2.27 (td, J=10.1, 5.2 Hz, 1H), 1.88 (dddd, J=13.7, 9.9, 8.8, 6.7 Hz, 1H). HRMS (ESI) m/z: [M+H]⁺ Calcd for C₁₆H₁₇F₃N₃O₂, 340.1267. Found 340.1270.

(±)-cis-5x was isolated as a brownish yellow solid (52 mg). ¹H NMR (400 MHz, CDCl₃) δ 8.55 (d, J=2.0 Hz, 1H), 7.96-7.86 (m, 2H), 4.71 (d, J=3.7 Hz, 1H), 4.15 (ddd, J=14.1, 5.6, 3.9 Hz, 1H), 3.98 (ddd, J=14.0, 11.1, 4.7 Hz, 1H), 3.91-3.75 (m, 2H), 2.81 (dt, J=6.2, 3.0 Hz, 1H), 2.66 (dt, J=11.8, 3.0 Hz, 1H), 2.55-2.41 (m, 2H), 2.34 (s, 3H), 1.97 (dddd, J=14.4, 11.2, 5.6, 2.6 Hz, 1H). HRMS (ESI) m/z: [M+H]⁺ Calcd for C₁₆H₁₇F₃N₃O₂, 340.1267. Found 340.1267.

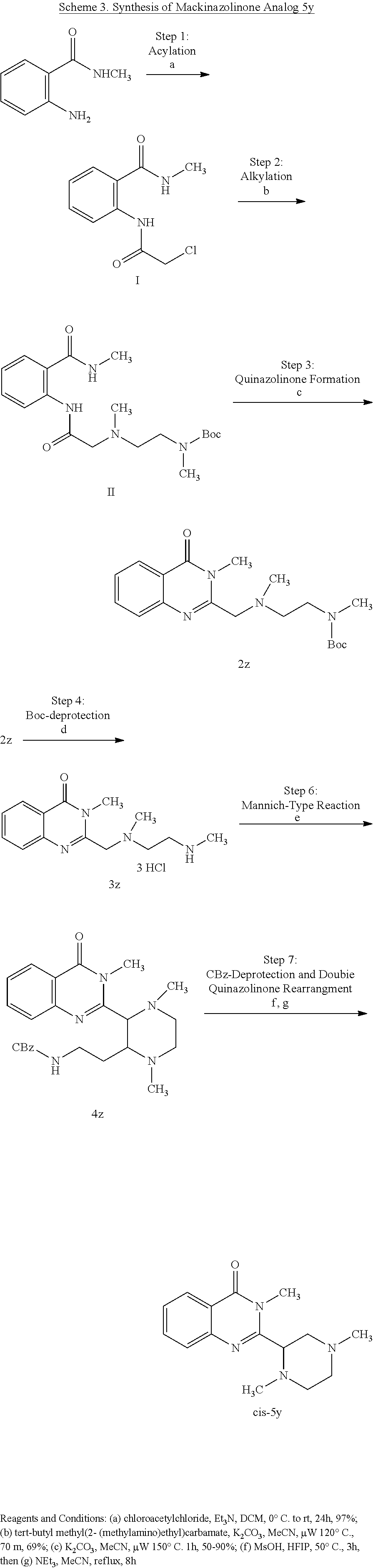

### Example 32: Synthesis of (±)-(4aR,13bR)-1,4-dimethyl-1,2,3,4,4a,5,6,13b-octahydro-8H-pyrazino[2′,3′:3,4]pyrido[2,1-b]quinazolin-8-one ((±)-cis-5y)

Prepared according to Scheme 3.

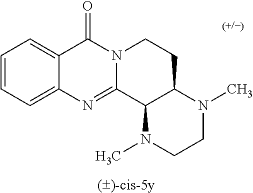 [SMILES: CN1CCN(C)[C@H]2c3nc4ccccc4c(=O)n3CC[C@H]21]

Step 1: Synthesis of 2-(2-chloroacetamido)-N-methylbenzamide (I). 2-amino-N-methylbenzamide (1 eq.) was placed in an oven dried round bottom flask under Ar (g). Anhydrous DCM (0.25 M) and NEt₃ (1.2 eq.) were then added. The reaction was cooled to 0° C. followed by the dropwise addition of chloroacetyl chloride over 10 min. After the reaction continued to stir for an additional 20 min at 0° C. before allowing to reach room temperature and stir for 24 h. After, the reaction was diluted with DCM (200 mL) and washed with 10% citric acid, sat. NaHCO₃ (aq), brine, dried over Na₂SO₄, filtered, and concentrated under reduced pressure to afford 2-(2-chloroacetamido)-N-methylbenzamide as a light brown solid (3.66 g, 97%). ¹H NMR (400 MHz, DMSO) δ 12.03 (s, 1H), 8.75 (q, J=4.5 Hz, 1H), 8.43 (dd, J=8.4, 1.2 Hz, 1H), 7.75 (dd, J=7.9, 1.5 Hz, 1H), 7.52 (ddd, J=8.6, 7.4, 1.5 Hz, 1H), 7.19 (td, J=7.6, 1.2 Hz, 1H), 4.40 (s, 2H), 2.80 (d, J=4.5 Hz, 3H). HRMS (ESI) m/z: [M+H]⁺ Calcd for C₁₀H₁₂ClN₂O₂, 227.0582. Found 227.0576.

Step 2: Synthesis of tert-butyl methyl(2-(methyl(2-((2-(methylcarbamoyl)phenyl)amino)-2-oxoethyl)amino)ethyl)carbamate (II): I (1 eq.) and K₂CO₃ (3 eq.) were placed in a 10-20 mL microwave irradiation vial. Sealed, evacuated, and backfilled with Ar(g). MeCN (0.25 M) and CH₃NH(CH₂)₂NCH₃Boc (1.2 eq.) were then added. The vial was then heated μW to 120° C. for 70 min. After the reaction was filtered and concentrated. Purified by Flash chromatography 0-1% MeOH/DCM to afford tert-butyl methyl(2-(methyl(2-((2-(methylcarbamoyl)phenyl)amino)-2-oxoethyl)amino)ethyl)carbamate as a light orange solid (1.14 g, 69%). ¹H NMR (400 MHz, CDCl₃) δ 11.67-11.30 (m, 1H), 8.53 (s, 1H), 7.42 (d, J=7.7 Hz, 2H), 7.03 (td, J=7.6, 1.2 Hz, 1H), 6.43 (s, 1H), 3.41 (d, J=8.8 Hz, 2H), 3.19 (s, 2H), 2.96 (d, J=4.8 Hz, 3H), 2.87 (s, 3H), 2.66 (q, J=8.7, 6.9 Hz, 2H), 2.42 (s, 3H), 1.40 (s, 9H). HRMS (ESI) m/z: [M+H]⁺ Calcd for C₁₉H₃₁N₄O₄, 379.2340. Found 379.2341.

Step 3: Synthesis of tert-butyl methyl(2-(methyl((3-methyl-4-oxo-3,4-dihydroquinazolin-2-yl)methyl)amino)ethyl)carbamate (2z). II (1 eq.) and K₂CO₃ (3 eq.) were placed in a 10-20 mL microwave irradiation vial. Sealed, evacuated, and backfilled with Ar(g). Added MeCN (0.25 M) and heated μW 150° C. for 8 h. After, the reaction was filtered and concentrated under reduced pressure. Purified by flash chromatography 2-25% X/hexanes (X=4:1 EtOAc/DCM) to afford 2z as a yellow oil (0.64 g, 76%). ¹H NMR (400 MHz, CDCl₃) δ 8.26 (dd, J=8.0, 1.5 Hz, 1H), 7.71 (ddd, J=8.5, 7.0, 1.5 Hz, 1H), 7.64 (dd, J=8.2, 1.3 Hz, 1H), 7.45 (ddd, J=8.1, 6.9, 1.3 Hz, 1H), 3.74 (s, 3H), 3.67 (s, 2H), 3.32 (d, J=22.2 Hz, 2H), 2.75 (d, J=18.5 Hz, 3H), 2.64 (s, 2H), 2.35 (s, 3H), 1.38 (d, J=18.2 Hz, 9H). HRMS (ESI) m/z: [M+H]⁺ Calcd for C₁₄H₂₁N₄O, 361.2234. Found 361.2220.

Step 4: Synthesis of 3-methyl-2-((methyl(2-(methylamino)ethyl)amino)methyl)quinazolin-4(3H)-one tris-hydrochloride salt (3z). Compound 3z was synthesized according to general procedure 4 from 2z, which afforded 3z as a white solid (1.25 g, 84%). as a tan solid (0.60g, quant.). ¹H NMR (400 MHz, DMSO) δ 9.86 (s, 2H), 8.15 (dd, J=8.0, 1.4 Hz, 1H), 7.87 (ddd, J=8.4, 7.0, 1.5 Hz, 1H), 7.80 (dd, J=8.2, 1.2 Hz, 1H), 7.57 (ddd, J=8.2, 7.0, 1.3 Hz, 1H), 4.91 (s, 2H), 3.74 (d, J=6.3 Hz, 3H), 3.58-3.37 (m, 6H), 3.07 (s, 3H), 2.64-2.56 (m, 3H). HRMS (ESI) m/z: [M+H]⁺ Calcd for C₁₄H₂₁N₄O 261.1710. Found 261.1718.

Step 5: Synthesis of benzyl (2-(1,4-dimethyl-3-(3-methyl-4-oxo-3,4-dihydroquinazolin-2-yl)piperazin-2-yl)ethyl)carbamate (4z). Compound 4z was synthesized according to general procedure 5.1 from 3z. Purification by reverse phase flash chromatography (10-50% MeOH/H₂O) afforded 4z an off-white solid (92.3 mg, 41%). ¹H NMR (500 MHz, CDCl₃) δ 8.24 (d, J=8.1 Hz, 1H), 7.82 (d, J=8.1 Hz, 1H), 7.71 (t, J=7.8 Hz, 1H), 7.43 (t, J=7.6 Hz, 1H), 7.32-7.19 (m, 5H), 5.02-4.88 (m, 2H), 4.74 (s, 1H), 3.70 (s, 3H), 3.04-2.76 (m, 6H), 2.52-2.44 (m, 5H), 2.29 (s, 3H), 2.10 (s, 1H), 1.91 (s, 1H). HRMS (ESI) m/z: [M+H]⁺ Calcd for C₂₅H₃₂N₅O₃, 450.2500. Found 450.2508.

Step 6: Synthesis of (±)-(4aR,13bR)-1,4-dimethyl-1,2,3,4,4a,5,6,13b-octahydro-8H-pyrazino[2′,3′:3,4]pyrido[2,1-b]quinazolin-8-one ((±)-cis-5y). (±)-cis-5y were synthesized according to general procedure 6 starting from 4z. Purified by flash chromatography: 10-50% X/hexanes (X=2% NEt₃ 3:1 EtOAc/EtOH). (±)-cis-5y was isolated as a brown-orange oil (12 mg, 43%). ¹H NMR (400 MHz, CDCl₃) δ 8.28 (dd, J=7.9, 1.4 Hz, 1H), 7.78-7.63 (m, 2H), 7.46 (ddd, J=8.2, 6.5, 1.8 Hz, 1H), 4.56 (s, 1H), 4.24 (ddd, J=13.4, 10.4, 6.0 Hz, 1H), 3.55 (d, J=3.5 Hz, 1H), 3.09 (dq, J=8.6, 4.3, 3.6 Hz, 1H), 2.99 (dt, J=11.6, 3.8 Hz, 1H), 2.75 (ddd, J=11.2, 8.3, 3.1 Hz, 1H), 2.57-2.40 (m, 5H), 2.35 (s, 3H), 2.21 (dtd, J=14.8, 5.8, 3.6 Hz, 1H), 1.89 (ddt, J=14.5, 10.2, 7.2 Hz, 1H). HRMS (ESI) m/z: [M+H]⁺ Calcd for C₁₆H₂₁N₄O, 285.1710. Found 285.1715.

### Example 33: Synthesis of (±)-(4aR,13bS)-4-ethyl-1,2,3,4,4a,5,6,13b-octahydro-8H-[1,6]naphthyridino[5,6-b]quinazolin-8-one ((±)-trans-5z) and (±)-(4aR,13bR)-4-ethyl-1,2,3,4,4a,5,6,13b-octahydro-8H-[1,6]naphthyridino[5,6-b]quinazolin-8-one ((±)-cis-5z)

Prepared according to Scheme 2.

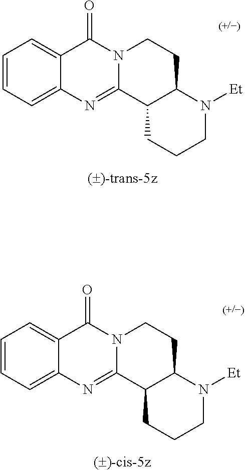 [SMILES: CCN1CCC[C@@H]2c3nc4ccccc4c(=O)n3CC[C@H]21 ; CCN1CCC[C@H]2c3nc4ccccc4c(=O)n3CC[C@H]21]

Step 1: Synthesis of tert-butyl ethyl(4-(3-neopentyl-4-oxo-3,4-dihydroquinazolin-2-yl)butyl)carbamate (2aa). Compound 2aa was synthesized according to general procedure 3 from anthranilic acid and 1b. Purification by flash chromatography (10-30% X/hexanes, X=4:1 EtOAc/DCM) delivered 2aa as a colorless oil (0.76 g, 73%). ¹H NMR (400 MHz, CDCl₃) δ 8.22 (dd, J=8.0, 1.5 Hz, 1H), 7.69 (ddd, J=8.5, 7.0, 1.6 Hz, 1H), 7.59 (dd, J=8.2, 1.2 Hz, 1H), 7.41 (ddd, J=8.1, 7.0, 1.2 Hz, 1H), 4.10 (s, 2H), 3.29-3.13 (m, 4H), 2.89 (t, J=7.5 Hz, 2H), 1.83-1.71 (m, 2H), 1.64 (p, J=8.3, 7.9 Hz, 2H), 1.44 (s, 9H), 1.08 (t, J=7.1 Hz, 3H), 1.00 (s, 9H). HRMS (ESI) m/z: [M+H]⁺ Calcd for C₂₄H₃₈N₃O₃, 416.2908. Found 416.2895

Step 2: Synthesis of 2-(4-(ethylamino)butyl)-3-neopentylquinazolin-4(3H)-one bis-hydrochloride salt (3aa). Compound 3aa was synthesized according to general procedure 4 from 2aa, which afforded 3aa as an off white solid (0.57 g, 81%). ¹H NMR (400 MHz, DMSO) δ 9.16 (s, 2H), 8.16 (dd, J=8.0, 1.3 Hz, 1H), 7.98-7.86 (m, 2H), 7.62 (ddd, J=8.2, 6.7, 1.5 Hz, 1H), 4.25-3.98 (m, 2H), 3.15 (t, J=7.3 Hz, 2H), 3.00-2.80 (m, 5H), 1.96-1.75 (m, 4H), 1.22 (t, J=7.2 Hz, 3H), 0.96 (s, 9H). HRMS (ESI) m/z: [M+H]⁺ Calcd for C₁₉H₃₀N₃O, 316.2383. Found 316.2382.

Step 3.1: Synthesis of benzyl (2-(1-ethyl-3-(3-neopentyl-4-oxo-3,4-dihydroquinazolin-2-yl)piperidin-2-yl)ethyl)carbamate (4aa). Compound 4aa was synthesized according to general procedure 5.1 from 3aa. Purification by reverse phase flash chromatography (10-75% MeOH/H₂O) afforded 4aa an off-white solid (205 mg, 81%). ¹H NMR (400 MHz, CDCl₃) δ 8.25-8.14 (m, 1H), 7.90 (s, 0.1H), 7.77-7.64 (m, 0.9H), 7.64-7.54 (m, 0.2H), 7.47-7.23 (m, 7.1H), 6.21 (s, 0.6H), 5.58 (s, 0.1H), 5.19-4.95 (m, 2.1H), 4.78 (d, J=14.2 Hz, 0.2H), 4.40 (s, 0.5H), 3.76 (s, 0.2H), 3.61-3.48 (m, 0.3H), 3.40-3.01 (m, 4.6H), 3.01-2.54 (m, 2.7H), 2.52-2.29 (m, 1H), 1.96 (d, J=11.5 Hz, 1.4H), 1.86-1.44 (m, 4.7H), 1.18-1.04 (m, 3H), 1.03-0.97 (m, 9H). HRMS (ESI) m/z: [M+H]⁺ Calcd for C₃₀H₄₁N₄O₃, 505.3173. Found 505.3179.

Step 4: Synthesis of (±)-(4aR,13bS)-4-ethyl-1,2,3,4,4a,5,6,13b-octahydro-8H-[1,6]naphthyridino[5,6-b]quinazolin-8-one ((±)-trans-5z) and (±)-(4aR,13bR)-4-ethyl-1,2,3,4,4a,5,6,13b-octahydro-8H-[1,6]naphthyridino[5,6-b]quinazolin-8-one ((±)-cis-5z). Both diastereomers of 5z were synthesized according to general procedure 6 starting from 4aa. Purified by flash chromatography: 10-50% X/hexanes (X=2% NEt₃ 3:1 EtOAc/EtOH). (±)-trans-5z+(±)-cis-5z: 51.4 mg (91%).

(±)-trans-5z was isolated as an off white solid (40.2 mg).). ¹H NMR (400 MHz, CDCl₃) δ 8.23 (dd, J=8.1, 1.5 Hz, 1H), 7.69 (ddd, J=8.4, 7.0, 1.6 Hz, 1H), 7.62 (dd, J=8.2, 1.2 Hz, 1H), 7.40 (ddd, J=8.1, 7.0, 1.3 Hz, 1H), 4.25-4.07 (m, 2H), 3.05 (dq, J=11.4, 2.5, 2.1 Hz, 1H), 2.92 (dq, J=13.2, 7.3 Hz, 1H), 2.76-2.64 (m, 2H), 2.56-2.37 (m, 2H), 2.24 (ddd, J=10.5, 9.4, 5.1 Hz, 1H), 2.14 (td, J=12.0, 2.9 Hz, 1H), 1.92-1.79 (m, 2H), 1.73 (tdd, J=16.9, 8.5, 4.8 Hz, 1H), 1.47 (dtd, J=14.4, 12.7, 12.0, 3.7 Hz, 1H), 1.06 (t, J=7.2 Hz, 3H). HRMS (ESI) m/z: [M+H]⁺ Calcd for C₁₇H₂₂N₃O, 284.1757. Found. 284.1758.

(±)-cis-5z was isolated as an off white solid (11.2 mg). ¹H NMR (400 MHz, CDCl₃) δ 8.25 (dd, J=8.0, 1.5 Hz, 1H), 7.70 (ddd, J=8.5, 6.9, 1.6 Hz, 1H), 7.61 (dd, J=8.2, 1.2 Hz, 1H), 7.41 (ddd, J=8.2, 6.9, 1.2 Hz, 1H), 4.14 (ddd, J=14.3, 9.2, 5.2 Hz, 1H), 4.02 (dt, J=14.1, 5.8 Hz, 1H), 3.09 (dt, J=6.0, 4.3 Hz, 1H), 2.98 (dt, J=7.0, 3.2 Hz, 1H), 2.80-2.70 (m, 2H), 2.56 (ddd, J=22.5, 13.4, 6.5 Hz, 2H), 2.43-2.29 (m, 2H), 1.95 (dddd, J=14.4, 8.8, 5.6, 2.8 Hz, 1H), 1.81-1.69 (m, 1H), 1.68-1.60 (m, 2H), 1.05 (t, J=7.2 Hz, 3H). HRMS (ESI) m/z: [M+H]⁺ Calcd for C₁₇H₂₂N₃O, 284.1757. Found. 284.1765.

### Example 34: Synthesis of (±)-(4aR,13bS)-4-benzyl-1,2,3,4,4a,5,6,13b-octahydro-8H-[1,6]naphthyridino[5,6-b]quinazolin-8-one ((±)-trans-5aa) and (±)-(4aR,13bR)-4-benzyl-1,2,3,4,4a,5,6,13b-octahydro-8H-[1,6]naphthyridino[5,6-b]quinazolin-8-one((±)-cis-5a)

Prepared according to Scheme 2.

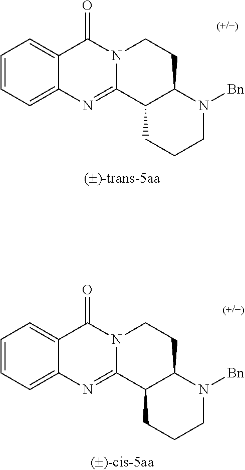 [SMILES: O=c1c2ccccc2nc2n1CC[C@@H]1[C@@H]2CCCN1Cc1ccccc1 ; O=c1c2ccccc2nc2n1CC[C@@H]1[C@H]2CCCN1Cc1ccccc1]

Step 1: Synthesis of tert-butyl benzyl(4-(3-neopentyl-4-oxo-3,4-dihydroquinazolin-2-yl)butyl)carbamate (2bb). Compound 2bb was synthesized according to general procedure 3 from anthranilic acid and 1c. Purification by flash chromatography (5-20% X/hexanes, X=4:1 EtOAc/DCM) delivered 2bb as a colorless oil (0.86 g, 51%). ¹H NMR (400 MHz, CDCl₃) δ 8.23 (dd, J=8.0, 1.5 Hz, 1H), 7.69 (ddd, J=8.5, 7.0, 1.5 Hz, 1H), 7.59 (dd, J=8.3, 1.1 Hz, 1H), 7.41 (ddd, J=8.2, 7.0, 1.2 Hz, 1H), 7.30-7.19 (m, 5H), 4.52-4.35 (m, 2H), 4.07 (s, 2H), 3.23 (d, J=33.0 Hz, 2H), 2.85 (s, 2H), 1.73 (p, J=6.7 Hz, 2H), 1.68-1.54 (m, 2H), 1.45 (s, 9H), 0.98 (s, 9H). HRMS (ESI) m/z: [M+H]⁺ Calcd for C₂₉H₄₀N₃O₃, 478.3064. Found 478.3060.

Step 2: Synthesis of 2-(4-(benzylamino)butyl)-3-neopentylquinazolin-4(3H)-one bis-hydrochloride salt (3bb). Compound 3bb was synthesized according to general procedure 4 from 2bb, which afforded 3bb as a white solid (0.72 g, 95%). ¹H NMR (400 MHz, DMSO) δ 9.62 (s, 2H), 8.15 (dd, J=8.0, 1.3 Hz, 1H), 8.00-7.84 (m, 2H), 7.62 (ddd, J=7.4, 4.5, 1.8 Hz, 3H), 7.39 (qd, J=4.7, 1.7 Hz, 3H), 4.12 (t, J=5.8 Hz, 4H), 3.13 (t, J=7.0 Hz, 2H), 2.93 (p, J=6.9, 6.3 Hz, 2H), 1.94-1.80 (m, 4H), 0.95 (s, 9H). HRMS (ESI) m/z: [M+H]⁺ Calcd for C₂₄H₃₂N₃O, 378.2540. Found 378.2546.

Step 3.1: Synthesis of benzyl (2-(1-benzyl-3-(3-neopentyl-4-oxo-3,4-dihydroquinazolin-2-yl)piperidin-2-yl)ethyl)carbamate (4bb). Compound 4bb was synthesized according to general procedure 5.1 from 3bb. Purification by reverse phase flash chromatography (10-80% MeOH/H₂O) afforded 4bb a slight-yellow solid (145 mg, 51%). ¹H NMR (400 MHz, CDCl₃) δ 8.28-8.14 (m, 1H), 7.93-7.83 (m, 0.1H), 7.78-7.56 (m, 1.2H), 7.53-7.13 (m, 11.6H), 6.34-5.79 (m, 0.5H), 5.25-4.96 (m, 2H), 4.68-4.31 (m, 0.8H), 3.97 (dd, J=42.8, 12.1 Hz, 0.8H), 3.68-3.12 (m, 5.7H), 2.90 (d, J=13.1 Hz, 1.2H), 2.80-2.58 (m, 0.2H), 2.38 (s, 1H), 2.08-1.62 (m, 5H) 1.60-1.37 (m, 1H), 1.06-1.02 (m, 9H), 1.01-0.96 (m, 0.3H). HRMS (ESI) m/z: [M+H]⁺ Calcd for C₃₅H₄₃N₄O₃, 567.3330. Found 567.3338.

Step 4: Synthesis of (±)-(4aR,13bS)-4-benzyl-1,2,3,4,4a,5,6,13b-octahydro-8H-[1,6]naphthyridino[5,6-b]quinazolin-8-one ((±)-trans-5aa) and (±)-(4aR,13bR)-4-benzyl-1,2,3,4,4a,5,6,13b-octahydro-8H-[1,6]naphthyridino[5,6-b]quinazolin-8-one ((±)-cis-5a). Both diastereomers of 5aa were synthesized according to general procedure 6 starting from 4bb. Purified by flash chromatography: 5-60% EtOAc/Hexanes. (±)-trans-5aa+(±)-cis-5aa: 37.0 mg (54%).

(±)-trans-5aa was isolated as an off white solid (30.0 mg). ¹H NMR (400 MHz, CDCl₃) δ 8.26 (dd, J=8.0, 1.5 Hz, 1H), 7.71 (ddd, J=8.4, 6.9, 1.6 Hz, 1H), 7.64 (dd, J=8.3, 1.3 Hz, 1H), 7.43 (ddd, J=8.1, 6.9, 1.3 Hz, 1H), 7.33 (d, J=4.4 Hz, 4H), 7.29-7.24 (m, 1H), 4.32 (ddd, J=14.3, 8.0, 6.3 Hz, 1H), 4.19-4.04 (m, 2H), 3.23 (d, J=13.6 Hz, 1H), 2.94 (ddd, J=12.1, 4.8, 2.4 Hz, 1H), 2.78 (td, J=11.1, 3.7 Hz, 1H), 2.73-2.65 (m, 1H), 2.54 (dq, J=13.5, 6.2 Hz, 1H), 2.30 (ddd, J=10.6, 9.1, 5.4 Hz, 1H), 2.08-1.91 (m, 2H), 1.82-1.75 (m, 1H), 1.67 (qt, J=12.8, 3.6 Hz, 1H), 1.51 (tdd, J=13.1, 11.4, 4.1 Hz, 1H). HRMS (ESI) m/z: [M+H]⁺ Calcd for C₂₂H₂₄N₃O, 346.1914. Found 346.1917.

(±)-cis-5aa was isolated as an off white solid (7.0 mg). ¹H NMR (400 MHz, CDCl₃) δ 8.27 (dd, J=8.0, 1.5 Hz, 1H), 7.71 (ddd, J=8.4, 7.0, 1.6 Hz, 1H), 7.62 (dd, J=8.4, 1.2 Hz, 1H), 7.42 (ddd, J=8.1, 7.0, 1.2 Hz, 1H), 7.28 (d, J=4.7 Hz, 4H), 7.22 (ddd, J=8.7, 5.2, 3.4 Hz, 1H), 4.28 (ddd, J=14.0, 8.8, 5.1 Hz, 1H), 4.00 (dd, J=14.0, 4.7 Hz, 2H), 3.36 (d, J=13.4 Hz, 1H), 3.15 (d, J=5.7 Hz, 1H), 3.00 (q, J=3.9, 2.9 Hz, 1H), 2.69 (dt, J=10.8, 5.0 Hz, 1H), 2.60-2.42 (m, 2H), 2.23 (dd, J=11.6, 5.9 Hz, 1H), 2.06 (dd, J=8.5, 5.9 Hz, 1H), 1.82 (d, J=7.3 Hz, 1H), 1.58 (t, J=5.8 Hz, 2H). HRMS (ESI) m/z: [M+H]⁺ Calcd for C₂₂H₂₄N₃O, 346.1914. Found 346.1915.

### Example 35: Synthesis of (±)-(4aR,13bS)-4,12-dimethyl-1,2,3,4,4a,5,6,13b-octahydro-8H-[1,6]naphthyridino[5,6-b]quinazolin-8-one ((±)-trans-5bb) and (±)-(4aR,13bR)-4,12-dimethyl-1,2,3,4,4a,5,6,13b-octahydro-8H-[1,6]naphthyridino[5,6-b]quinazolin-8-one((±)-cis-5bb)

Prepared according to Scheme 2.

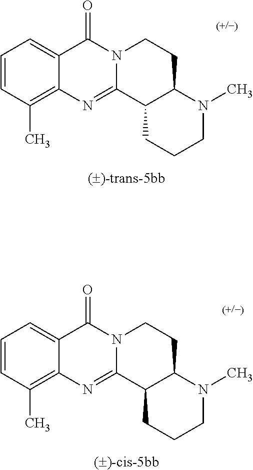 [SMILES: Cc1cccc2c(=O)n3c(nc12)[C@H]1CCCN(C)[C@@H]1CC3 ; Cc1cccc2c(=O)n3c(nc12)[C@@H]1CCCN(C)[C@@H]1CC3]

Step 1: Synthesis of tert-butyl methyl(4-(8-methyl-3-neopentyl-4-oxo-3,4-dihydroquinazolin-2-yl)butyl)carbamate (2cc). Compound 2cc was synthesized according to general procedure 3 from 2-amino-3-methyl-benzoic acid and 1a. Purification by flash chromatography (5-20% X/hexanes, X=4:1 EtOAc/DCM) delivered 2cc as a colorless oil (1.18 g, 80%). ¹H NMR (400 MHz, CDCl₃) δ 8.07 (dd, J=8.2, 1.6 Hz, 1H), 7.54 (dt, J=7.2, 1.3 Hz, 1H), 7.29 (t, J=7.6 Hz, 1H), 4.08 (s, 2H), 3.28 (t, J=7.2 Hz, 2H), 2.89 (t, J=7.3 Hz, 2H), 2.85 (s, 4H), 2.58 (s, 3H), 1.84 (p, J=7.4 Hz, 2H), 1.67 (td, J=16.3, 14.7, 8.8 Hz, 2H), 1.45 (s, 9H), 1.00 (s, 9H). HRMS (ESI) m/z: [M+H]⁺ Calcd for C₂₄H₃₈N₃O₃, 416.2908. Found 416.2892.

Step 2: Synthesis of 8-methyl-2-(4-(methylamino)butyl)-3-neopentylquinazolin-4(3H)-one bis-hydrochloride salt (3cc). Compound 3cc was synthesized according to general procedure 4 from 2cc, which afforded 3cc as a white solid (0.81 g, 75%). ¹H NMR (400 MHz, DMSO) δ 9.13 (s, 2H), 7.92 (ddd, J=7.9, 1.6, 0.7 Hz, 1H), 7.64 (ddd, J=7.2, 1.6, 0.9 Hz, 1H), 7.35 (t, J=7.6 Hz, 1H), 4.07 (s, 2H), 2.92 (dt, J=17.0, 7.1 Hz, 4H), 2.54 (s, 3H), 2.49 (t, J=5.5 Hz, 4H), 1.93-1.69 (m, 4H), 0.92 (s, 9H). HRMS (ESI) m/z: [M+H]⁺ Calcd for C₁₉H₃₀N₃O, 316.2383. Found 316.2389.

Step 3.1: Synthesis of benzyl (2-(1-methyl-3-(8-methyl-3-neopentyl-4-oxo-3,4-dihydroquinazolin-2-yl)piperidin-2-yl)ethyl)carbamate (4cc). Compound 4cc was synthesized according to general procedure 5.1 from 3cc Purification by reverse phase flash chromatography (10-75% MeOH/H₂O) afforded 4cc a white solid (72.7 mg, 29%). ¹H NMR (400 MHz, CDCl₃) δ 8.10-8.02 (m, 1H), 7.56-7.47 (m, 1H), 7.40-7.22 (m, 6H), 5.28-4.93 (m, 3H), 4.77 (d, J=14.2 Hz, 0.2H), 4.13 (s, 1.1H), 3.66-3.53 (m, 0.5H), 3.34 (s, 1.8H), 3.19-3.07 (m, 0.9H), 3.03-2.74 (m, 2.2H), 2.66-2.51 (m, 4.8H), 2.47-2.26 (m, 2.6H), 2.00 (d, J=10.8 Hz, 1H), 1.91-1.41 (m, 5H), 1.05-0.97 (m, 9H).

Step 4: Synthesis of (±)-(4aR,13bS)-4,12-dimethyl-1,2,3,4,4a,5,6,13b-octahydro-8H-[1,6]naphthyridino[5,6-b]quinazolin-8-one ((±)-trans-5bb) and (±)-(4aR,13bR)-4,12-dimethyl-1,2,3,4,4a,5,6,13b-octahydro-8H-[1,6]naphthyridino[5,6-b]quinazolin-8-one((±)-cis-5bb). Both diastereomers of 5bb were synthesized according to general procedure 6 starting from 4cc. Purified by flash chromatography: 10-40% X/hexanes (X=2% NEt₃ 3:1 EtOAc/EtOH). (±)-trans-5bb+(±)-cis-5bb: 21.2 mg (75%).

(±)-trans-5bb was isolated as a yellow solid (13.7 mg). ¹H NMR (400 MHz, CDCl₃) δ 8.13-8.07 (m, 1H), 7.54 (d, J=7.2 Hz, 1H), 7.30 (t, J=7.6 Hz, 1H), 4.23-4.04 (m, 2H), 2.97 (dq, J=11.6, 2.1 Hz, 1H), 2.84-2.77 (m, 1H), 2.69 (td, J=11.1, 3.7 Hz, 1H), 2.58 (s, 3H), 2.50-2.40 (m, 1H), 2.37 (s, 3H), 2.12 (td, J=11.9, 3.3 Hz, 1H), 1.98 (td, J=10.2, 4.7 Hz, 1H), 1.90-1.75 (m, 3H), 1.49 (tdd, J=13.3, 11.6, 4.5 Hz, 1H). HRMS (ESI) m/z: [M+H]⁺ Calcd for C₁₇H₂₂N₃O, 284.1757. Found 284.1747.

(±)-cis-5bb was isolated as a yellow oil (7.5 mg). ¹H NMR (500 MHz, cdcl₃) δ 8.08 (dd, J=7.9, 1.6 Hz, 1H), 7.52 (d, J=7.2 Hz, 1H), 7.27 (t, J=7.6 Hz, 1H), 4.12 (ddd, J=14.0, 5.8, 4.1 Hz, 1H), 4.02 (ddd, J=14.1, 11.1, 5.0 Hz, 1H), 3.02-2.97 (m, 1H), 2.97-2.90 (m, 1H), 2.78 (dd, J=10.1, 5.4 Hz, 1H), 2.57 (s, 3H), 2.54-2.49 (m, 1H), 2.32 (dt, J=14.3, 4.7 Hz, 1H), 2.26 (s, 3H), 2.16 (td, J=11.5, 2.9 Hz, 1H), 1.96 (dddd, J=14.3, 11.1, 5.8, 3.1 Hz, 1H), 1.71 (tq, J=12.1, 4.4, 3.9 Hz, 1H), 1.56 (dddd, J=22.9, 13.2, 7.7, 3.9 Hz, 2H). HRMS (ESI) m/z: [M+H]⁺ Calcd for C₁₇H₂₂N₃O, 284.1757. Found 284.1756.

### Example 36: Synthesis of (±)-(3aR,12bR)-3-methyl-2,3,3a,4,5,12b-hexahydropyrrolo[3′,2′:3,4]pyrido[2,1-b]quinazolin-7(1H)-one((±)-cis-5cc)

Prepared according to Scheme 2.

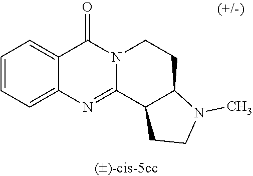 [SMILES: CN1CC[C@H]2c3nc4ccccc4c(=O)n3CC[C@H]21]

Step 1: Synthesis of tert-butyl methyl(3-(3-neopentyl-4-oxo-3,4-dihydroquinazolin-2-yl)propyl)carbamate (2dd). Compound 2dd was synthesized according to general procedure 3 from anthranilic acid and 1e. Purification by flash chromatography (5-20% X/hexanes, X=4:1 EtOAc/DCM) delivered 2dd as a yellow oil (0.97 g, 69%). ¹H NMR (400 MHz, CDCl₃) δ 8.22 (dd, J=8.0, 1.5 Hz, 1H), 7.70 (td, J=7.6, 6.9, 1.5 Hz, 1H), 7.59 (dd, J=8.2, 1.1 Hz, 1H), 7.46-7.37 (m, 1H), 4.07 (s, 2H), 3.36 (t, J=6.8 Hz, 2H), 2.97-2.76 (m, 5H), 2.02 (p, J=7.0 Hz, 2H), 1.44 (s, 9H), 0.99 (s, 9H). HRMS (ESI) m/z: [M+H]⁺ Calcd for C₂₂H₃₄N₃O₃, 388.2595. Found 388.2594.

Step 2: Synthesis of 2-(3-(methylamino)propyl)-3-neopentylquinazolin-4(3H)-one bis-hydrochloride salt (3dd). Compound 3dd was synthesized according to general procedure 4 from 2dd, which afforded 3dd as a white solid (0.81 g, 92%). ¹H NMR (400 MHz, DMSO) δ 9.46-9.22 (m, 2H), 8.14 (dd, J=8.0, 1.4 Hz, 1H), 7.91 (ddd, J=8.4, 7.0, 1.5 Hz, 1H), 7.87-7.77 (m, 1H), 7.59 (ddd, J=8.1, 7.0, 1.2 Hz, 1H), 4.13 (s, 2H), 3.22 (t, J=7.7 Hz, 2H), 3.11-2.99 (m, 2H), 2.53 (d, J=5.4 Hz, 4H), 2.18 (p, J=7.3 Hz, 2H), 0.95 (s, 9H). HRMS (ESI) m/z: [M+H]⁺ Calcd for C₁₇H₂₆N₃O, 288.2070. Found 288.2078.

Step 3.1: Synthesis of benzyl (2-(1-methyl-3-(3-neopentyl-4-oxo-3,4-dihydroquinazolin-2-yl)pyrrolidin-2-yl)ethyl)carbamate (4dd). Compound 4dd was synthesized according to general procedure 5.1 from 3dd. Purification by reverse phase flash chromatography (10-70% MeOH/H₂O) afforded 4dd an waxy yellow solid (194 mg, 82 ¹H NMR (400 MHz, CDCl₃) δ 8.22 (dt, J=8.0, 2.0 Hz, 1H), 7.73-7.62 (m, 1.1H), 7.49 (t, J=7.6 Hz, 0.7H), 7.45-7.27 (m, 5.9H), 6.31 (s, 0.6H), 6.06 (s, 0.1H), 5.42 (s, 0.2H), 5.12 (s, 1.5H), 5.03 (s, 0.4H), 4.75 (d, J=14.3 Hz, 0.2H), 4.65-4.42 (m, 0.7H), 3.80 (q, J=9.3 Hz, 0.3H), 3.73-3.34 (m, 2.6H), 3.33-3.00 (m, 3H), 2.99-2.76 (m, 0.4H), 2.51-2.31 (m, 4.7H), 2.13-1.39 (m, 1H), 1.77-1.55 (m, 1.5H), 1.46-1.34 (m, 0.2H), 1.18-1.06 (m, 0.2H), 1.03-0.93 (m, 9H), 0.92-0.78 (m, 0.4H). HRMS (ESI) m/z: [M+H]⁺ Calcd for C₂₈H₃₇N₄O₃, 477.2860. Found 477.2884.

Step 4: Synthesis of (±)-(3aR,12bR)-3-methyl-2,3,3a,4,5,12b-hexahydropyrrolo[3′,2′:3,4]pyrido[2,1-b]quinazolin-7(1H)-one((±)-cis-5cc). (±)-cis-5cc was synthesized according to general procedure 6 starting from 4dd with the following modification: instead of heating at 80° C. for 8 h, the reaction was heated to 150° C. for 4h. Purified by flash chromatography: 10-50% X/hexanes (X=2% NEt₃ 3:1 EtOAc/EtOH).

(±)-cis-5cc was isolated as a waxy-yellow solid (17.9 mg, 35%). ¹H NMR (400 MHz, CDCl₃) δ 8.25 (dd, J=8.1, 1.6 Hz, 1H), 7.70 (ddd, J=8.5, 7.1, 1.6 Hz, 1H), 7.60 (dd, J=8.3, 1.1 Hz, 1H), 7.41 (ddd, J=8.2, 7.1, 1.2 Hz, 1H), 4.55 (dddd, J=13.6, 4.7, 3.6, 1.2 Hz, 1H), 3.85 (ddd, J=13.8, 11.3, 2.8 Hz, 1H), 3.56 (q, J=9.2 Hz, 1H), 3.07 (dd, J=8.5, 6.8 Hz, 1H), 2.78-2.73 (m, 1H), 2.43-2.30 (m, 5H), 2.03-1.91 (m, 2H), 1.77 (dddd, J=14.5, 11.3, 4.5, 3.6 Hz, 1H). HRMS (ESI) m/z: [M+H]⁺ Calcd for C₁₅H₁₈N₃O, 256.1444. Found 256.1444.

### Example 37: Synthesis of (±)-(3aR,12bR)-11-methoxy-3-methyl-2,3,3a,4,5,12b-hexahydropyrrolo[3′,2′:3,4]pyrido[2,1-b]quinazolin-7(1H)-one ((±)-cis-5dd)

Prepared according to Scheme 2.

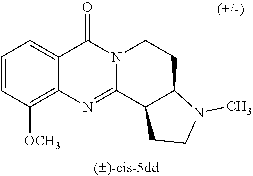 [SMILES: COc1cccc2c(=O)n3c(nc12)[C@@H]1CCN(C)[C@@H]1CC3]

Step 1: Synthesis of tert-butyl (3-(8-methoxy-3-neopentyl-4-oxo-3,4-dihydroquinazolin-2-yl)propyl)(methyl)carbamate (2ee). Compound 2ee was synthesized according to general procedure 3 from 2-amino-3-methoxy benzoic acid and 1e. Purification by flash chromatography (10-45% X/hexanes, X=4:1 EtOAc/DCM) delivered 2ee as a light-yellow solid (0.96 g, 57%). %). ¹H NMR (400 MHz, CDCl₃) δ 7.80 (dd, J=8.1, 1.3 Hz, 1H), 7.34 (t, J=8.0 Hz, 1H), 7.14 (dd, J=7.9, 1.3 Hz, 1H), 3.99 (s, 5H), 3.34 (s, 2H), 2.99-2.81 (m, 5H), 2.06-1.91 (m, 2H), 1.43 (s, 9H), 0.97 (s, 9H). HRMS (ESI) m/z: [M+H]⁺ Calcd for C₂₃H₃₆N₃O₄, 418.2706. Found 418.2715.

Step 2: Synthesis of 8-methoxy-2-(3-(methylamino)propyl)-3-neopentylquinazolin-4(3H)-one bis-hydrochloride salt (2ee). Compound 3ee was synthesized according to general procedure 4 from 2ee, which afforded 3ee as a white solid (0.89 g, 96%). 1H NMR (400 MHz, DMSO) δ 9.50-9.39 (m, 2H), 8.47 (s, 1H), 7.66 (dd, J=7.8, 1.5 Hz, 1H), 7.48-7.36 (m, 2H), 4.09 (s, 2H), 3.95 (s, 3H), 3.12 (t, J=7.1 Hz, 2H), 3.03 (q, J=6.4 Hz, 2H), 2.54 (t, J=5.4 Hz, 3H), 2.09 (p, J=7.0 Hz, 2H), 0.93 (s, 9H). HRMS (ESI) m/z: [M+H]⁺ Calcd for C₁₈H₂₈N₃O₂, 318.2176. Found 318.2169.

Step 3.1: benzyl (2-(3-(8-methoxy-3-neopentyl-4-oxo-3,4-dihydroquinazolin-2-yl)-1-methylpyrrolidin-2-yl)ethyl)carbamate (4ee). Compound 4ee was synthesized according to general procedure 5.1 from 3ee. Purification by reverse phase flash chromatography (10-70% MeOH/H₂O) afforded 4ee an off-white solid (186 mg, 74%). ¹H NMR (400 MHz, CDCl₃) δ 7.85-7.78 (m, 1H), 7.42-7.28 (m, 5.3H), 7.16 (dd, J=8.1, 1.3 Hz, 0.2H), 7.09 (d, J=8.0 Hz, 0.7H), 7.02-6.91 (m, 0.8H), 5.41 (s, 0.2H), 5.17 (s, 1.5H), 5.03 (s, 0.4H), 4.70 (d, J=14.2 Hz, 0.9H), 4.16 (s, 0.3H), 3.98 (s, 0.6H), 3.88-3.72 (m, 2.4H), 3.73-3.57 (m, 1H), 3.50 (d, J=14.2 Hz, 0.9H), 3.41-3.30 (m, 1.6H), 3.26 (ddd, J=9.4, 7.3, 1.9 Hz, 0.2H), 3.16-3.02 (m, 1.1H), 2.99-2.74 (m, 1H), 2.51-2.09 (m, 6.1H), 1.59-1.47 (m, 0.8H), 1.46-1.30 (m, 1.6H), 1.02-0.92 (m, 9H). HRMS (ESI) m/z: [M+H]⁺ Calcd for C₂₉H₃₉N₄O₄, 507.2966. Found 507.2966.

Step 4: Synthesis of (±)-(3aR,12bR)-11-methoxy-3-methyl-2,3,3a,4,5,12b-hexahydropyrrolo[3′,2′:3,4]pyrido[2,1-b]quinazolin-7(1H)-one ((±)-cis-5dd). (±)-cis-5dd was synthesized according to general procedure 6 starting from 4ee with the following modification: instead of heating at 80° C. for 8 h, the reaction was heated to 150° C. for 4h. Purified by flash chromatography: 5-40% X/hexanes (4:1 EtOAc/DCM).

(±)-cis-5dd was isolated as a yellow-orange oil (23 mg, 40 ¹H NMR (400 MHz, CDCl₃) δ 7.83 (dd, J=8.1, 1.2 Hz, 1H), 7.34 (t, J=8.0 Hz, 1H), 7.14 (dd, J=7.9, 1.3 Hz, 1H), 4.64 (dtd, J=13.8, 3.8, 1.2 Hz, 1H), 3.98 (s, 3H), 3.81-3.67 (m, 2H), 3.09-3.04 (m, 1H), 2.79 (d, J=9.7 Hz, 1H), 2.48-2.29 (m, 5H), 2.01-1.82 (m, 2H), 1.68 (tt, J=14.6, 3.9 Hz, 1H). HRMS (ESI) m/z: [M+H]⁺ Calcd for C₁₆H₂₀N₃O₂, 286.1550. Found 286.1549.

### Example 38: Synthesis of (±)-(3aR,12bR)-9,10-difluoro-3-methyl-2,3,3a,4,5,12b-hexahydropyrrolo[3′,2′:3,4]pyrido[2,1-b]quinazolin-7(1H)-one ((±)-cis-5ee)

Prepared according to Scheme 2.

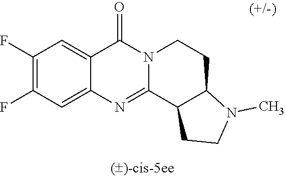 [SMILES: CN1CC[C@H]2c3nc4cc(F)c(F)cc4c(=O)n3CC[C@H]21]

Step 1: Synthesis of tert-butyl (3-(6,7-difluoro-3-neopentyl-4-oxo-3,4-dihydroquinazolin-2-yl)propyl)(methyl)carbamate (2ff). Compound 2ff was synthesized according to general procedure 3 from 2-amino-4,5-difluorobenzoic acid and 2ff. Purification by flash chromatography (10-30% X/hexanes, X=4:1 EtOAc/DCM) delivered S26 as a yellow-white solid (0.83 g, 68%). ¹H NMR (400 MHz, CDCl₃) δ 7.96 (dd, J=10.1, 8.5 Hz, 1H), 7.35 (dd, J=10.8, 7.1 Hz, 1H), 4.04 (s, 2H), 3.35 (t, J=6.8 Hz, 2H), 2.94-2.76 (m, 5H), 2.00 (p, J=7.0 Hz, 2H), 1.43 (s, 9H), 0.97 (s, 9H). HRMS (ESI) m/z: [M+H]⁺ Calcd for C₂₂H₃₂F₂N₃O₃, 424.2406. Found 424.2419.

Step 2: Synthesis of 6,7-difluoro-2-(3-(methylamino)propyl)-3-neopentylquinazolin-4(3H)-one bis-hydrochloride salt (3ff). Compound 3ff was synthesized according to general procedure 4 from 2ff, which afforded 3ff as a white solid (0.58 g, 89%). ¹H NMR (400 MHz, DMSO) δ 9.39-9.25 (m, 2H), 9.06 (s, 1H), 7.99 (dd, J=10.4, 8.6 Hz, 1H), 7.70 (dd, J=11.2, 7.2 Hz, 1H), 4.06 (s, 2H), 3.09-2.94 (m, 4H), 2.51 (t, J=5.6 Hz, 3H), 2.11 (p, J=7.4 Hz, 2H), 0.92 (s, 9H). HRMS (ESI) m/z: [M+H]⁺ Calcd for C₁₇H₂₄F₂N₃O, 324.1882. Found 324.1878.

Step 3.1: Synthesis of benzyl (2-(3-(6,7-difluoro-3-neopentyl-4-oxo-3,4-dihydroquinazolin-2-yl)-1-methylpyrrolidin-2-yl)ethyl)carbamate (4ff). Compound 4ff was synthesized according to general procedure 5.1 from 3ff. Purification by reverse phase flash chromatography (10-75% MeOH/H₂O) afforded 4ff an off-white solid (152 mg, 59%). %). ¹H NMR (400 MHz, CDCl₃) δ 7.89 (dd, J=10.1, 8.5 Hz, 1H), 7.45-7.33 (m, 0.8H), 7.34-7.17 (m, 5H), 5.95 (s, 0.6H), 5.29 (s, 0.2H), 5.09-4.99 (m, 1.6H), 4.96 (s, 0.4H), 4.63 (d, J=14.3 Hz, 0.2H), 4.39 (s, 0.6H), 3.77-3.43 (m, 1H), 3.42-3.24 (m, 1.6H), 3.21-2.92 (m, 2.9H), 2.85-2.66 (m, 0.5H), 2.42-2.21 (m, 4.6H), 2.03-1.93 (m, 0.2H), 1.86 (s, 0.8H), 1.72-1.49 (m, 1.5H), 1.36-1.26 (m, 0.3H), 1.07-0.97 (m, 0.3H), 0.91 (s, 9H), 0.84-0.71 (m, 0.5H). HRMS (ESI) m/z: [M+H]⁺ Calcd for C₂₈H₃₅F₂N₄O₃, 513.2672. Found 513.2680.

Step 4: Synthesis of (±)-(3aR,12bR)-9,10-difluoro-3-methyl-2,3,3a,4,5,12b-hexahydropyrrolo[3′,2′:3,4]pyrido[2,1-b]quinazolin-7(1H)-one ((±)-cis-5ee).

(±)-cis-5ee was synthesized according to general procedure 6 starting from 4ff with the following modification: instead of heating at 80° C. for 8 h, the reaction was heated to 150° C. for 4h. Purified by flash chromatography: 10-30% X/hexanes (X=2% NEt₃ 3:1 EtOAc/EtOH).

(±)-cis-5ee was isolated as an off white solid (23.7 mg, 41%). ¹H NMR (500 MHz, cdcl₃) δ 7.98 (dd, J=10.1, 8.5 Hz, 1H), 7.36 (dd, J=10.9, 7.0 Hz, 1H), 4.48 (dt, J=13.7, 3.7 Hz, 1H), 3.87 (ddd, J=13.8, 11.1, 2.8 Hz, 1H), 3.52 (p, J=9.1 Hz, 1H), 3.08 (t, J=7.6 Hz, 1H), 2.74 (dt, J=8.5, 3.9 Hz, 1H), 2.40-2.32 (m, 5H), 2.00-1.91 (m, 2H), 1.78 (ddt, J=14.7, 11.0, 4.0 Hz, 1H). HRMS (ESI) m/z: [M+H]⁺ Calcd for C₁₅H₁₆F₂N₃O, 292.1256. Found 292.1256.

### Example 39: Synthesis of (±)-(3aR,12bR)-3-benzyl-2,3,3a,4,5,12b-hexahydropyrrolo [3′,2′:3,4]pyrido[2,1-b]quinazolin-7(1H)-one ((±)-cis-5ff)

Prepared according to Scheme 2.

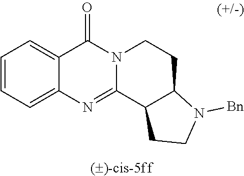 [SMILES: O=c1c2ccccc2nc2n1CC[C@@H]1[C@H]2CCN1Cc1ccccc1]

Step 1: Synthesis of tert-butyl benzyl(3-(3-neopentyl-4-oxo-3,4-dihydroquinazolin-2-yl)propyl)carbamate (2gg). Compound 2gg was synthesized according to general procedure 3 from anthranilic acid and 4-(benzyl(tert-butoxycarbonyl)amino)butanoic acid. Purification by flash chromatography (5-20% X/hexanes, X=4:1 EtOAc/DCM) delivered 2gg as a yellow-white solid (0.96 g, 57%). ¹H NMR (400 MHz, CDCl₃) δ 8.26 (dd, J=8.0, 1.5 Hz, 1H), 7.73 (ddd, J=8.4, 7.1, 1.5 Hz, 1H), 7.62 (dd, J=8.2, 1.1 Hz, 1H), 7.45 (ddd, J=8.2, 7.1, 1.2 Hz, 1H), 7.35-7.23 (m, 5H), 4.59-4.41 (m, 2H), 4.07 (s, 2H), 3.37 (d, J=33.1 Hz, 2H), 3.00-2.72 (m, 2H), 2.16-1.95 (m, 2H), 1.49 (s, 9H), 1.02 (s, 9H). HRMS (ESI) m/z: [M+H]⁺ Calcd for C₂₈H₃₈N₃O₃, 464.2908. Found 464.2889.

Step 2: Synthesis of 2-(3-(benzylamino)propyl)-3-neopentylquinazolin-4(3H)-one bis-hydrochloride salt (3gg). Compound 3gg was synthesized according to general procedure 4 from 2gg, which afforded 3gg as a white solid (0.67 g, 90%). ¹H NMR (400 MHz, DMSO) δ 9.80 (p, J=6.3, 5.9 Hz, 2H), 8.14 (dd, J=8.0, 1.4 Hz, 1H), 7.90 (ddd, J=8.5, 7.1, 1.5 Hz, 1H), 7.82 (d, J=8.1 Hz, 1H), 7.66-7.55 (m, 3H), 7.44-7.35 (m, 3H), 4.14 (q, J=5.4 Hz, 4H), 3.22 (t, J=7.6 Hz, 2H), 3.08 (p, J=7.0 Hz, 2H), 2.26 (p, J=7.4 Hz, 2H), 0.95 (s, 9H). HRMS (ESI) m/z: [M+H]⁺ Calcd for C₂₃H₃₀N₃O, 364.2383. Found 364.2382.

Step 3.1: Synthesis of benzyl (2-(1-benzyl-3-(3-neopentyl-4-oxo-3,4-dihydroquinazolin-2-yl)pyrrolidin-2-yl)ethyl)carbamate (4gg). Compound 4gg was synthesized according to general procedure 5.1 from 3gg. Purification by reverse phase flash chromatography (10-85% MeOH/H₂O) afforded 4gg an off-white solid (57.2 mg, 20%). ¹H NMR (400 MHz, CDCl₃) δ 8.23 (d, J=7.9 Hz, 1H), 7.79-7.53 (m, 2H), 7.46-7.23 (m, 11.2H), 6.40 (s, 0.7H), 5.67 (s, 0.1H), 5.26-4.98 (m, 2H), 4.75 (d, J=14.2 Hz, 0.1H), 4.50 (s, 0.7H), 4.19 (d, J=12.5 Hz, 0.8H), 3.93-3.54 (m, 0.9H), 3.54-3.44 (m, 2H), 3.38-3.14 (m, 1.9H), 2.96-2.88 (m, 1H), 2.68-2.56 (m, 0.1H), 2.43-2.19 (m, 1.7H), 2.00 (s, 0.7H), 1.92-1.56 (m, 2.2H), 1.33-1.23 (m, 1.2H), 1.02-0.95 (m, 9H), 0.91-0.85 (m, 1.1H). HRMS (ESI) m/z: [M+H]⁺ Calcd for C₃₄H₁N₄O₃, 553.3173. Found 553.3150.

Step 4: Synthesis of (±)-(3aR,12bR)-3-benzyl-2,3,3a,4,5,12b-hexahydropyrrolo[3′,2′:3,4]pyrido[2,1-b]quinazolin-7(1H)-one ((±)-cis-5ff). (±)-cis-5ff was synthesized according to general procedure 6 starting from 4gg with the following modification: instead of heating at 80° C. for 8 h, the reaction was heated to 150° C. for 4h. Purified by flash chromatography: 10-50% X/hexanes (X=2% NEt₃ 3:1 EtOAc/EtOH).

(±)-cis-5ff was isolated as an orange oil (17.9 mg, 54%). ¹H NMR (400 MHz, CDCl₃) δ 8.22 (dd, J=8.0, 1.5 Hz, 1H), 7.65 (ddd, J=8.4, 7.0, 1.6 Hz, 1H), 7.55 (dd, J=8.3, 1.2 Hz, 1H), 7.37 (ddd, J=8.2, 7.0, 1.2 Hz, 1H), 7.27 (s, 4H), 7.20 (q, J=4.4 Hz, 1H), 4.63-4.52 (m, 1H), 3.96 (d, J=13.2 Hz, 1H), 3.89 (ddd, J=13.8, 11.6, 2.6 Hz, 1H), 3.57 (q, J=9.4 Hz, 1H), 3.36 (d, J=13.2 Hz, 1H), 3.03 (dt, J=8.7, 3.5 Hz, 1H), 2.93 (dd, J=8.8, 6.9 Hz, 1H), 2.41-2.29 (m, 1H), 2.24 (ddd, J=11.2, 8.9, 5.7 Hz, 1H), 1.96 (ddt, J=14.3, 4.4, 2.8 Hz, 1H), 1.84 (dddd, J=12.1, 10.9, 9.4, 6.9 Hz, 1H), 1.76-1.65 (m, 1H). HRMS (ESI) m/z: [M+H]⁺ Calcd for C₂₁H₂₂N₃O, 354.1577. Found 354.1560.

### Example 40. Synthesis of (±)-(4aR,13bS)-10-chloro-11-methoxy-4-methyl-1,2,3,4,4a,5,6,13b-octahydro-8H-[1,6]naphthyridino[5,6-b]quinazolin-8-one ((±)-trans-5gg) and (±)-(4aR,13bR)-10-chloro-11-methoxy-4-methyl-1,2,3,4,4a,5,6,13b-octahydro-8H-[1,6]naphthyridino[5,6-b]quinazolin-8-one ((±)-cis-5gg)

Prepared according to Scheme 2.

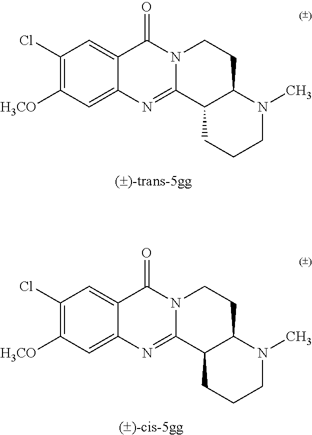 [SMILES: COc1cc2nc3n(c(=O)c2cc1Cl)CC[C@@H]1[C@@H]3CCCN1C ; COc1cc2nc3n(c(=O)c2cc1Cl)CC[C@@H]1[C@H]3CCCN1C]

Step 1: Synthesis of tert-butyl (4-(6-chloro-7-methoxy-3-neopentyl-4-oxo-3,4-dihydroquinazolin-2-yl)butyl)(methyl)carbamate (2hh). Compound 2hh was synthesized according to general procedure 3 from 2-amino-5-chloro-4-methoxybenzoic acid and 1a. Purification by flash chromatography (3-25% X/hexanes, X=4:1 EtOAc/DCM) delivered 2hh as a yellow oil (4.40 g, 86%). ¹H NMR (400 MHz, CDCl₃) δ 8.18 (s, 1H), 7.02 (s, 1H), 4.18-3.95 (m, 4H), 3.32-3.18 (m, 2H), 2.91-2.78 (m, 5H), 1.89-1.68 (m, 3H), 1.67-1.57 (m, 2H), 1.43 (s, 9H), 0.97 (s, 9H). ¹³C NMR (101 MHz, CDCl₃) δ 161.8, 159.7, 155.9, 147.7, 128.4, 128.2, 122.4, 114.6, 107.8, 100.0, 79.4, 56.6, 52.4, 35.8, 34.8, 34.3, 28.8, 28.6, 27.5, 24.9. HRMS (ESI) m/z: [M+H]⁺ Calcd for C₂₄H₃₇ClN₃O₄, 466.2467. Found 466.2469.

Step 2: Synthesis of 6-chloro-7-methoxy-2-(4-(methylamino)butyl)-3-neopentylquinazolin-4(3H)-one bis-hydrochloride salt (3hh). Compound 3hh was synthesized according to general procedure 4 from 2hh, which afforded 3hh as a white solid (3.25 g, 79%). ¹H NMR (400 MHz, DMSO) δ 10.66 (s, 1H), 9.22 (q, J=6.1 Hz, 2H), 8.07 (s, 1H), 7.42 (s, 1H), 4.09 (s, 2H), 4.02 (s, 3H), 3.04 (t, J=7.1 Hz, 2H), 2.93 (p, J=6.9 Hz, 2H), 2.53-2.51 (m, 3H), 1.84 (p, J=7.3 Hz, 4H), 0.95 (s, 9H). ¹³C NMR (101 MHz, DMSO) δ 160.6, 160.0, 159.3, 127.4, 121.5, 113.4, 106.1, 66.4, 57.0, 52.0, 47.6, 34.2, 33.3, 32.2, 28.2, 24.8, 24.0. HRMS (ESI) m/z: [M+H]⁺ Calcd for C₁₉H₂₉ClN₃O₂, 366.1943. Found 366.1932.

Step 3.1: Synthesis of benzyl (2-(3-(6-chloro-7-methoxy-3-neopentyl-4-oxo-3,4-dihydroquinazolin-2-yl)-1-methylpiperidin-2-yl)ethyl)carbamate (4hh). Compound 4hh was synthesized according to general procedure 5.1 from 3hh. Purification by normal phase flash chromatography (3-25% X/hexanes (X=2% NEt₃ 3:1 EtOAc/EtOH)) afforded 4hh as a an off-white solid (4.73 g, 82%). 4hh was used directly in the next step without analysis.

Step 4: Synthesis of (±)-(4aR,13bS)-10-chloro-11-methoxy-4-methyl-1,2,3,4,4a,5,6,13b-octahydro-8H-[1,6]naphthyridino[5,6-b]quinazolin-8-one ((±)-trans-5gg) and (±)-(4aR,13bR)-10-chloro-11-methoxy-4-methyl-1,2,3,4,4a,5,6,13b-octahydro-8H-[1,6]naphthyridino[5,6-b]quinazolin-8-one ((±)-cis-5gg). Both diastereomers of 5gg were synthesized according to general procedure 6 starting from 4hh with the following modification: the cyclization step was carried out at 95° C. for 18 h. Purified by flash chromatography: 10-50% X/hexanes (X=2% NEt₃ 3:1 EtOAc/EtOH). (±)-trans-5gg+(±)-cis-5gg: 2.26 g (80%).

(±)-trans-5gg was isolated as a light yellow-white solid (1.58 g). ¹H NMR (400 MHz, CDCl₃) δ 8.19 (s, 1H), 7.04 (s, 1H), 4.20-4.06 (m, 2H), 3.99 (s, 3H), 2.96 (dq, J=11.7, 2.2 Hz, 1H), 2.73-2.62 (m, 2H), 2.41 (dq, J=13.3, 5.5 Hz, 1H), 2.35 (s, 3H), 2.10 (td, J=12.0, 3.1 Hz, 1H), 1.97 (td, J=10.1, 4.8 Hz, 1H), 1.88-1.73 (m, 3H), 1.46 (qdd, J=12.6, 3.5, 1.4 Hz, 1H). ¹³C NMR (101 MHz, CDCl₃) δ 160.7, 159.6, 156.8, 148.4, 127.7, 122.5, 114.3, 108.0, 62.4, 56.9, 56.6, 44.9, 42.7, 40.4, 27.4, 26.6, 25.2. HRMS (ESI) m/z: [M+H]⁺ Calcd for C₁₇H₂₁ClN₃O₂, 334.1317. Found 334.1313.

(±)-cis-5gg was isolated as a light yellow-white solid (0.68 g). ¹H NMR (400 MHz, CDCl₃) δ 8.20 (s, 1H), 7.03 (s, 1H), 4.08 (ddd, J=14.0, 5.9, 4.3 Hz, 1H), 3.99 (s, 4H), 2.98 (q, J=3.7 Hz, 1H), 2.77 (dq, J=11.8, 4.4, 3.2 Hz, 2H), 2.56 (dt, J=6.2, 3.2 Hz, 1H), 2.33 (dq, J=14.7, 4.9 Hz, 1H), 2.28 (s, 3H), 2.19 (td, J=10.5, 3.6 Hz, 1H), 1.94 (dddd, J=14.1, 10.7, 5.9, 2.8 Hz, 1H), 1.60 (ddq, J=23.5, 12.4, 4.2 Hz, 3H). ¹³C NMR (101 MHz, CDCl₃) δ 161.2, 159.5, 157.2, 148.3, 127.6, 122.2, 114.3, 107.8, 58.0, 56.6, 55.7, 42.9, 42.1, 39.8, 27.0, 24.6, 22.7. HRMS (ESI) m/z: [M+H]⁺ Calcd for C₁₇H₂₁ClN₃O₂, 334.1312. Found 334.1313.

### Example 41. Synthesis of (±)-(3aR,12bR)-9-chloro-3-methyl-2,3,3a,4,5,12b-hexahydropyrrolo[3′,2′:3,4]pyrido[2,1-b]quinazolin-7(1H)-one((±)-cis-5hh)

Prepared according to Scheme 2.

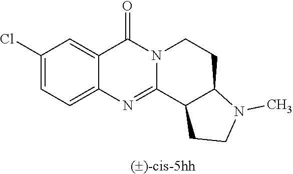 [SMILES: CN1CC[C@H]2c3nc4ccc(Cl)cc4c(=O)n3CC[C@H]21]

Step 1: Synthesis of tert-butyl (3-(6-chloro-3-neopentyl-4-oxo-3,4-dihydroquinazolin-2-yl)propyl)(methyl)carbamate (2ii). Compound 2ii was synthesized according to general procedure 3 from anthranilic acid and 1e. Purification by flash chromatography (5-20% X/hexanes, X=4:1 EtOAc/DCM) delivered 2ii as a yellow oil (4.36 g, 88%). ¹H NMR (400 MHz, CDCl₃) δ 8.18 (d, J=2.4 Hz, 1H), 7.62 (dd, J=8.7, 2.4 Hz, 1H), 7.54 (d, J=8.7 Hz, 1H), 4.06 (s, 2H), 3.36 (t, J=6.8 Hz, 2H), 2.91-2.80 (m, 5H), 2.01 (p, J=7.1 Hz, 2H), 1.44 (s, 9H), 0.98 (s, 9H). ¹³C NMR (101 MHz, CDCl₃) δ 162.2, 145.5, 134.6, 132.1, 128.5, 126.5, 121.8, 79.6, 52.6, 48.4, 47.9, 34.8, 34.3, 32.9, 28.8, 28.6, 27.5, 25.5. HRMS (ESI) m/z: [M+H]⁺ Calcd for C₂₂H₃₃ClN₃O₃, 422.2205. Found 422.2199.

Step 2: Synthesis of 6-chloro-2-(3-(methylamino)propyl)-3-neopentylquinazolin-4(3H)-one bis-hydrochloride salt (3ii). Compound 3ii was synthesized according to general procedure 4 from 2ii, which afforded 3ii as a white solid (3.44 g, 86%). ¹H NMR (400 MHz, DMSO) δ 10.26 (s, 2H), 9.38 (q, J=6.1 Hz, 2H), 8.03 (d, J=2.5 Hz, 1H), 7.86 (dd, J=8.7, 2.5 Hz, 1H), 7.75 (d, J=8.7 Hz, 1H), 4.08 (s, 2H), 3.12 (t, J=7.6 Hz, 2H), 3.07-2.96 (m, 2H), 2.50 (t, J=5.6 Hz, 3H), 2.14 (p, J=7.4 Hz, 2H), 0.92 (s, 9H). ¹³C NMR (101 MHz, DMSO) δ 160.6, 159.1, 142.7, 134.9, 131.2, 127.0, 125.6, 121.1, 52.1, 47.4, 34.3, 32.2, 31.4, 28.2, 23.2. HRMS (ESI) m/z: [M+H]⁺ Calcd for C₁₇H₂₅N₃O, 322.1681. Found 322.1673.

Step 3.1: Synthesis of benzyl (2-(3-(6-chloro-3-neopentyl-4-oxo-3,4-dihydroquinazolin-2-yl)-1-methylpyrrolidin-2-yl)ethyl)carbamate (4ii). Compound 4ii was synthesized according to general procedure 5.1 from 3ii. Purification by normal phase flash chromatography (3-15% X/hexanes (X=2% NEt₃ 3:1 EtOAc/EtOH)) afforded 4ii as a yellow solid (1.63 g, 63%). 4ii was used directly in the next step without analysis.

Step 4: Synthesis of (±)-(3aR,12bR)-9-chloro-3-methyl-2,3,3a,4,5,12b-hexahydropyrrolo[3′,2′:3,4]pyrido[2,1-b]quinazolin-7(1H)-one((±)-cis-5hh). (±)-cis-5hh was synthesized according to general procedure 6 starting from 4ii with the following modification: instead of heating at 80° C. for 8 h, the reaction was heated to 150° C. for 4h. Purified by flash chromatography: 3-20% X/hexanes (X=2% NEt₃ 3:1 EtOAc/EtOH). (±)-cis-5hh was isolated as a yellow solid (186 mg, 55%). ¹H NMR (400 MHz, CDCl₃) δ 8.20 (d, J=2.4 Hz, 1H), 7.62 (dd, J=8.7, 2.4 Hz, 1H), 7.53 (d, J=8.7 Hz, 1H), 4.50 (dddd, J=13.7, 4.9, 3.5, 1.1 Hz, 1H), 3.87 (ddd, J=13.8, 11.1, 2.8 Hz, 1H), 3.54 (q, J=9.1 Hz, 1H), 3.11-3.06 (m, 1H), 2.76 (dt, J=8.6, 3.8 Hz, 1H), 2.42-2.32 (m, 5H), 2.04-1.90 (m, 2H), 1.79 (tt, J=10.6, 4.0 Hz, 1H). ¹³C NMR (101 MHz, CDCl₃) δ 160.9, 157.7, 146.3, 134.6, 131.9, 128.5, 126.2, 121.2, 61.7, 55.9, 44.6, 40.1, 37.3, 31.8, 27.0. HRMS (ESI) m/z: [M+H]⁺ Calcd for C₁₅H₁₇N₃O, 290.1040. Found 290.1047.

### Example 42: Synthesis of (±)-(4aR,13bS)-10-chloro-1,2,3,4,4a,5,6,13b-octahydro-8H-[1,6]naphthyridino[5,6-b]quinazolin-8-one ((±)-trans-6a)

Prepared according to Scheme 2.

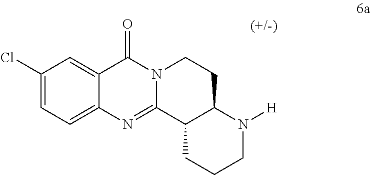 [SMILES: O=c1c2cc(Cl)ccc2nc2n1CC[C@H]1NCCC[C@H]21]

Steps 1-4: See Example 4—Synthesis of trans-5d.

Step 5: Synthesis of (±)-(4aR,13bS)-10-chloro-1,2,3,4,4a,5,6,13b-octahydro-8H-[1,6]naphthyridino[5,6-b]quinazolin-8-one (6a). Compound 6a was synthesized according to general procedure 7 from (±)-trans-5d. Purification by flash chromatography 40-75% X/hexanes (X=2% NEt₃ 3:1 EtOAc/EtOH) afforded 6a as a light yellow solid (54 mg, 58%). ¹H NMR (400 MHz, CDCl₃) δ 8.19 (d, J=2.6 Hz, 1H), 7.68-7.51 (m, 2H), 4.27-4.02 (m, 2H), 3.20-3.06 (m, 1H), 2.71 (tdd, J=12.0, 8.1, 3.3 Hz, 3H), 2.48 (td, J=11.0, 3.6 Hz, 1H), 2.19 (dq, J=10.9, 5.2 Hz, 1H), 1.92-1.57 (m, 4H), 1.50 (td, J=12.3, 11.6, 3.8 Hz, 1H). HRMS (ESI) m/z: [M+H]⁺ Calcd for C₁₅H₁₇ClN₃O, 290.1055. Found 290.1061.

### Example 43: Synthesis of (±)-(4aR,13bS)-10,11-dichloro-1,2,3,4,4a,5,6,13b-octahydro-8H-[1,6]naphthyridino[5,6-b]quinazolin-8-one (6b)

Prepared according to Scheme 2.

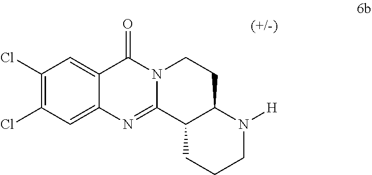 [SMILES: O=c1c2cc(Cl)c(Cl)cc2nc2n1CC[C@H]1NCCC[C@H]21]

Steps 1-4: See Example 18—Synthesis of trans-5q.

Step 5: Synthesis of (±)-(4aR,13bS)-10,11-dichloro-1,2,3,4,4a,5,6,13b-octahydro-8H-[1,6]naphthyridino[5,6-b]quinazolin-8-one (6b). Compound 6b was synthesized according to general procedure 7 from (±)-trans-5q. Purification by flash chromatography 40-75% X/hexanes (X=2% NEt₃ 3:1 EtOAc/EtOH) afforded 6b as a light yellow solid (134 mg, 70%). ¹H NMR (400 MHz, CDCl₃) δ 8.28 (s, 1H), 7.74 (s, 1H), 4.18 (ddd, J=14.4, 6.3, 4.7 Hz, 1H), 4.08 (ddd, J=14.7, 9.5, 5.9 Hz, 1H), 3.15 (ddt, J=11.9, 4.0, 1.9 Hz, 1H), 2.70 (ddt, J=12.1, 9.1, 4.3 Hz, 3H), 2.47 (td, J=11.0, 3.6 Hz, 1H), 2.19 (dq, J=13.6, 5.2 Hz, 1H), 1.85 (ddt, J=16.8, 9.6, 3.2 Hz, 2H), 1.73-1.57 (m, 2H), 1.47 (qd, J=13.2, 3.9 Hz, 1H). HRMS (ESI) m/z: [M+H]⁺ Calcd for C₁₅H₁₆Cl₂N₃O, 324.0665. Found 324.0672.

### Example 44. (±)-(4aR,13bS)-10-chloro-11-methoxy-1,2,3,4,4a,5,6,13b-octahydro-8H-[1,6]naphthyridino[5,6-b]quinazolin-8-one (6c)

Prepared according to Scheme 2.

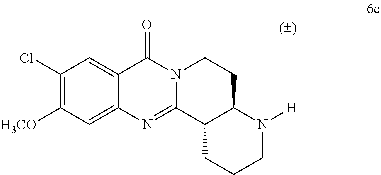 [SMILES: COc1cc2nc3n(c(=O)c2cc1Cl)CC[C@H]1NCCC[C@H]31]

Steps 1-4: See Example 40-synthesis of trans-5gg.

Step 5: Synthesis of (±)-(4aR,13bS)-10-chloro-11-methoxy-1,2,3,4,4a,5,6,13b-octahydro-8H-[1,6]naphthyridino[5,6-b]quinazolin-8-one (6c). Compound 6c was synthesized according to general procedure 7 from (±)-trans-5gg. Purification by flash chromatography 55-80% X/hexanes (X=2% NEt₃ 3:1 EtOAc/EtOH) afforded 6c as a tan solid (583 mg, 61%). ¹H NMR (400 MHz, CDCl₃) δ 8.20 (s, 1H), 7.04 (s, 1H), 4.18 (ddd, J=14.3, 6.2, 4.8 Hz, 1H), 4.07 (ddd, J=14.2, 9.6, 5.8 Hz, 1H), 3.99 (s, 3H), 3.15 (ddt, J=11.9, 3.9, 1.8 Hz, 1H), 2.76-2.65 (m, 3H), 2.47 (td, J=11.0, 3.6 Hz, 1H), 2.24-2.14 (m, 1H), 1.91-1.72 (m, 3H), 1.64 (qt, J=12.9, 4.0 Hz, 1H), 1.49 (tdd, J=13.2, 11.3, 3.8 Hz, 1H). HRMS (ESI) m/z: [M+H]⁺ Calcd for C₁₆H₁₉ClN₃O₂, 320.1160. Found 320.1159.

### Example 45: Synthesis of (±)-(4aR,13bS)-10,11-dichloro-4-isopropyl-1,2,3,4,4a,5,6,13b-octahydro-8H-[1,6]naphthyridino[5,6-b]quinazolin-8-one(7a)

Prepared according to Scheme 2.

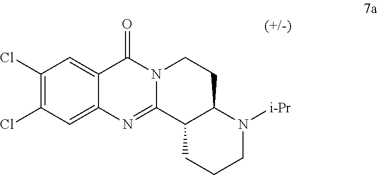 [SMILES: CC(C)N1CCC[C@@H]2c3nc4cc(Cl)c(Cl)cc4c(=O)n3CC[C@H]21]

Steps 1-5: See Example 43—Synthesis of 6b.

Step 6: Synthesis of (±)-(4aR,13bS)-10,11-dichloro-4-isopropyl-1,2,3,4,4a,5,6,13b-octahydro-8H-[1,6]naphthyridino[5,6-b]quinazolin-8-one(7a). Compound 7a was prepared by suspending 6b (1 eq.), isopropyl iodide (9.25 eq.), and Et₃N (4 eq.) in DMF (0.25 M) and heating to 80° C. for 20 h. Purification by flash chromatography 1-20% X/hexanes (X=2% NEt₃ 3:1 EtOAc/EtOH) afforded 7a as a light yellow solid (27 mg, 48%). ¹H NMR (400 MHz, CDCl₃) δ 8.27 (s, 1H), 7.74 (s, 1H), 4.16-4.10 (m, 2H), 3.40 (q, J=6.6 Hz, 1H), 3.02-2.97 (m, 1H), 2.72-2.64 (m, 2H), 2.48-2.39 (m, 2H), 2.11-2.05 (m, 1H), 1.93-1.78 (m, 2H), 1.68-1.58 (m, 1H), 1.47-1.37 (m, 1H), 1.17 (d, J=6.7 Hz, 3H), 0.88 (d, J=6.5 Hz, 3H). HRMS (ESI) m/z: [M+H]⁺ Calcd for C₁₈H₂₂Cl₂N₃O, 366.1134. Found 366.1152.

### Example 46: Synthesis of (±)-(4aR,13bS)-10,11-dichloro-4-((tetrahydro-2H-pyran-4-yl)methyl)-1,2,3,4,4a,5,6,13b-octahydro-8H-[1,6]naphthyridino[5,6-b]quinazolin-8-one (7b)

Prepared according to Scheme 2.

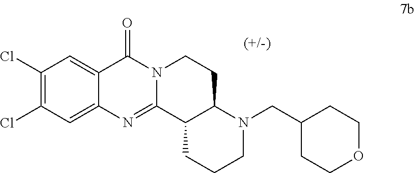

Steps 1-5: See Example 43—Synthesis of 6b.

Step 6: Synthesis of (±)-(4aR,13bS)-10,11-dichloro-4-((tetrahydro-2H-pyran-4-yl)methyl)-1,2,3,4,4a,5,6,13b-octahydro-8H-[1,6]naphthyridino[5,6-b]quinazolin-8-one (7b). Compound 7b was prepared according to general procedure 8.1 by suspending 6b (1 eq.), 4-bromomethyl-tetrahydropyran (4 eq.), and Et₃N (6 eq.) in DMF (0.25 M) and heating to 80° C. for 20 h. Purification by flash chromatography 1-12% X/hexanes (X=2% NEt₃ 3:1 EtOAc/EtOH) afforded 7b as a yellowish tan solid (109 mg, 70%). ¹H NMR (400 MHz, CDCl₃) δ 8.28 (s, 1H), 7.75 (s, 1H), 4.20 (ddd, J=14.4, 8.3, 6.2 Hz, 1H), 4.10-3.94 (m, 3H), 3.39 (tdd, J=11.7, 9.2, 2.2 Hz, 2H), 3.10-3.04 (m, 1H), 2.67-2.57 (m, 3H), 2.38 (dq, J=13.4, 5.9 Hz, 1H), 2.17 (td, J=9.8, 5.2 Hz, 1H), 2.00 (ddd, J=12.5, 10.5, 7.0 Hz, 2H), 1.84-1.56 (m, 6H), 1.50-1.39 (m, 1H), 1.32-1.18 (m, 2H). HRMS (ESI) m/z: [M+H]⁺ Calcd for C₂₁H₂₆Cl₂N₃O, 422.1397. Found 422.1418.

### Example 47: Synthesis of (±)-2-((4aR,13bS)-10,11-dichloro-8-oxo-2,3,5,6,8,13b-hexahydro-1H-[1,6]naphthyridino[5,6-b]quinazolin-4(4aH)-yl)-N,N-dimethylacetamide (7c)

Prepared according to Scheme 2.

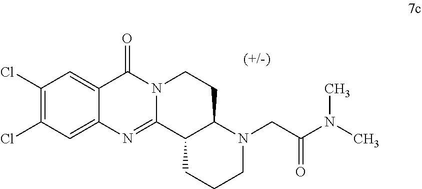

Steps 1-5: See Example 43—Synthesis of 6b.

Step 6: Synthesis of (±)-2-((4aR,13bS)-10,11-dichloro-8-oxo-2,3,5,6,8,13b-hexahydro-1H-[1,6]naphthyridino[5,6-b]quinazolin-4(4aH)-yl)-N,N-dimethylacetamide (7c). Compound 7c was prepared according to general procedure 8.1 by suspending 6b (1 eq.), 2-bromo-N,N-dimethylacetamide (1.4 eq.), and Et₃N (2 eq.) in DMF (0.25 M) and heating to 50° C. for 24 h. Purification by flash chromatography 10-50% X/hexanes (X=2% NEt₃ 3:1 EtOAc/EtOH) afforded 7c as a tan solid (79 mg, 90%). ¹H NMR (400 MHz, CDCl₃) δ 8.26 (s, 1H), 7.72 (s, 1H), 4.22-4.16 (m, 1H), 4.00 (ddd, J=14.5, 9.8, 5.9 Hz, 1H), 3.63 (d, J=15.2 Hz, 1H), 3.27 (d, J=15.2 Hz, 1H), 3.07 (s, 3H), 2.94 (s, 4H), 2.73-2.61 (m, 3H), 2.50 (td, J=11.9, 3.0 Hz, 1H), 2.45-2.38 (m, 1H), 1.86-1.67 (m, 3H), 1.44 (tdd, J=13.6, 11.0, 4.5 Hz, 1H). HRMS (ESI) m/z: [M+H]⁺ Calcd for C₁₉H₂₃Cl₂N₄O₂, 409.1193. Found 409.1221.

### Example 48: Synthesis of (±)-(4aR,13bS)-10,11-dichloro-4-pivaloyl-1,2,3,4,4a,5,6,13b-octahydro-8H-[1,6]naphthyridino[5,6-b]quinazolin-8-one (7d)

Prepared according to Scheme 2.

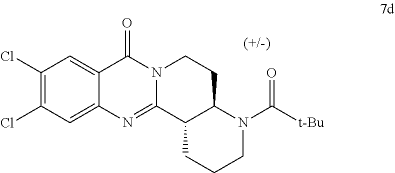

Steps 1-5: See Example 43—Synthesis of 6b.

Step 6: Synthesis of (±)-(4aR,13bS)-10,11-dichloro-4-pivaloyl-1,2,3,4,4a,5,6,13b-octahydro-8H-[1,6]naphthyridino[5,6-b]quinazolin-8-one (7d). Compound 7d was prepared according to general procedure 8.2 from 6b (1 eq.), pivaloyl chloride (1.3 eq) and Et₃N (2 eq.) in DCM (0.25 M). Purification by flash chromatography 5-50% EtOAc/Hexanes afforded 7d as a white solid (77 mg, 87%). ¹H NMR (400 MHz, CDCl₃) δ 8.28 (s, 1H), 7.73 (s, 1H), 4.65 (dt, J=13.9, 5.4 Hz, 1H), 4.09 (ddd, J=13.9, 8.3, 5.4 Hz, 1H), 3.99-3.88 (m, 1H), 3.47-3.31 (m, 2H), 3.08 (td, J=11.3, 3.5 Hz, 1H), 2.67-2.55 (m, 1H), 2.28-2.14 (m, 2H), 2.00-1.89 (m, 1H), 1.80-1.64 (m, 2H), 1.29 (s, 9H). FIRMS (ESI) m/z: [M+H]⁺ Calcd for C₂₀H₂₄Cl₂N₃O₂, 408.1240. Found 408.1052.

### Example 49: Synthesis of (±)-(4aR,13bS)-10,11-dichloro-4-(tetrahydro-2H-pyran-4-carbonyl)-1,2,3,4,4a,5,6,13b-octahydro-8H-[1,6]naphthyridino[5,6-b]quinazolin-8-one (7e)

Prepared according to Scheme 2.

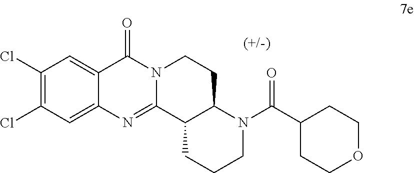

Steps 1-5: See Example 43—Synthesis of 6b.

Step 6: Synthesis of (±)-(4aR,13bS)-10,11-dichloro-4-(tetrahydro-2H-pyran-4-carbonyl)-1,2,3,4,4a,5,6,13b-octahydro-8H-[1,6]naphthyridino[5,6-b]quinazolin-8-one (7e). Compound 7e was prepared according to general procedure 8.2 from 6b (1 eq.), tetrahydro-2H-pyran-4-carbonyl chloride (1.3 eq), and Et₃N (2 eq.) in DCM (0.25 M) and stirring at room temperature for 24h. Purification by flash chromatography 50-100% EtOAc/Hexanes afforded 7e as an off-white solid (78 mg, 90%). ¹H NMR (400 MHz, CDCl₃) δ 8.28 (s, 1H), 7.73 (s, 1H), 4.54 (dt, J=14.1, 6.0 Hz, 1H), 4.12 (dt, J=13.6, 6.5 Hz, 1H), 4.02 (dtd, J=11.2, 4.3, 2.3 Hz, 2H), 3.75 (dt, J=14.2, 4.8 Hz, 1H), 3.52-3.36 (m, 4H), 3.02 (td, J=11.4, 3.8 Hz, 1H), 2.78-2.63 (m, 2H), 2.35 (q, J=6.6 Hz, 2H), 2.02-1.85 (m, 3H), 1.78-1.60 (m, 4H). HRMS (ESI) m/z: [M+H]⁺ Calcd for C₂₁H₂₄Cl₂N₃O₃, 436.1189. Found 436.1184.

### Example 50: Synthesis of (±)-(4aR,13bS)-10,11-dichloro-4-(morpholine-4-carbonyl)-1,2,3,4,4a,5,6,13b-octahydro-8H-[1,6]naphthyridino[5,6-b]quinazolin-8-one (7f)

Prepared according to Scheme 2.

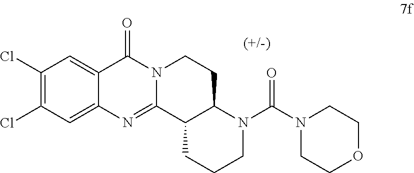

Steps 1-5: See Example 43—Synthesis of trans-6b.

Step 6: Synthesis of (±)-(4aR,13bS)-10,11-dichloro-4-(morpholine-4-carbonyl)-1,2,3,4,4a,5,6,13b-octahydro-8H-[1,6]naphthyridino[5,6-b]quinazolin-8-one (7f). Compound 7f was prepared according to general procedure 8.1 from 6b (1 eq.), morpholine-4-carbonyl chloride (2.6 eq.), and DMAP (0.25 eq) in DCM (0.25 M) and heating to 40° C. for 16h. Purification by flash chromatography 5-40% X/hexanes (X=2% NEt₃ 3:1 EtOAc/EtOH) afforded 7f as an off-white solid (87 mg, 93%).). ¹H NMR (400 MHz, CDCl₃) δ 8.28 (s, 1H), 7.74 (s, 1H), 4.49 (dt, J=14.0, 5.9 Hz, 1H), 4.02 (ddd, J=13.8, 8.3, 5.1 Hz, 1H), 3.68-3.65 (m, 4H), 3.47 (q, J=4.5 Hz, 3H), 3.39-3.33 (m, 1H), 3.27-3.24 (m, 1H), 3.12-2.98 (m, 2H), 2.82 (td, J=12.5, 2.8 Hz, 1H), 2.68-2.59 (m, 1H), 2.37 (dddd, J=13.9, 8.2, 6.9, 5.6 Hz, 1H), 1.93-1.82 (m, 2H), 1.71-1.57 (m, 2H). HRMS (ESI) m/z: [M+H]⁺ Calcd for C₂₀H₂₃Cl₂N₄O₃, 437.1120. Found 437.1132.

### Example 51: Synthesis of (±)-(4aR,13bS)-10,11-dichloro-4-(dimethylglycyl)-1,2,3,4,4a,5,6,13b-octahydro-8H-[1,6]naphthyridino[5,6-b]quinazolin-8-one (7g)

Prepared according to Scheme 2.

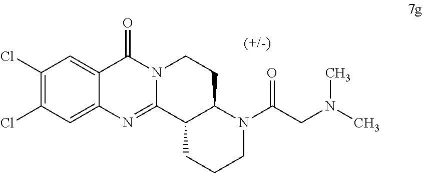

Steps 1-5: See Example 43—Synthesis of 6b.

Step 6: Synthesis of (±)-(4aR,13bS)-10,11-dichloro-4-(dimethylglycyl)-1,2,3,4,4a,5,6,13b-octahydro-8H-[1,6]naphthyridino[5,6-b]quinazolin-8-one (7g). Compound 7g was prepared according to general procedure 8.1 from 6b (1 eq.), dimethylglycinoyl chloride (1.3 eq.), and Et₃N (2 eq.) in DCM (0.25 M) and stirring at room temperature for 23h. Purification by flash chromatography 5-50% X/hexanes (X=2% NEt₃ 3:1 EtOAc/EtOH) afforded 7g as an off-white solid (73 mg, 83%). ¹H NMR (400 MHz, CDCl₃) δ 8.28 (s, 1H), 7.72 (s, 1H), 4.50 (dt, J=14.2, 6.1 Hz, 1H), 4.12 (ddd, J=14.1, 7.3, 5.6 Hz, 1H), 3.81 (dt, J=14.3, 5.4 Hz, 1H), 3.45 (ddt, J=21.9, 7.7, 6.0 Hz, 2H), 3.17-2.95 (m, 3H), 2.65 (ddt, J=8.8, 5.8, 3.2 Hz, 1H), 2.41 (dq, J=7.4, 6.1 Hz, 2H), 2.29 (s, 6H), 2.02-1.93 (m, 1H), 1.71 (tdd, J=14.2, 8.3, 3.9 Hz, 2H). HRMS (ESI) m/z: [M+H]⁺ Calcd for C₁₉H₂₃Cl₂N₄O₃, 409.1193. Found 409.1188.

### Example 52: Synthesis of (±)-(4aR,13bS)-4-benzoyl-10,11-dichloro-1,2,3,4,4a,5,6,13b-octahydro-8H-[1,6]naphthyridino[5,6-b]quinazolin-8-one (7h)

Prepared according to Scheme 2.

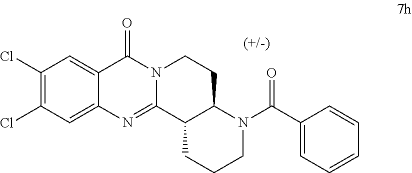

Steps 1-5: See Example 43—Synthesis of 6b.

Step 6: Synthesis of (±)-(4aR,13bS)-4-benzoyl-10,11-dichloro-1,2,3,4,4a,5,6,13b-octahydro-8H-[1,6]naphthyridino[5,6-b]quinazolin-8-one (7h). Compound 7h was prepared according to general procedure 8.2 from 6b (1 eq.), benzoyl chloride (2.6 eq.), Et₃N (2 eq.), and DMAP (0.9 eq.) in DCM (0.25 M) and stirring at room temperature for 48 h. Purification by flash chromatography (5-55% EtOAc/Hexanes) afforded 7h as a white solid (44.5 mg, 48%). ¹H NMR (400 MHz, Chloroform-d) δ 8.33 (s, 1H), 7.78 (s, 1H), 7.56-7.41 (m, 5H), 4.70 (dt, J=13.9, 5.3 Hz, 1H), 4.24 (ddd, J=13.8, 9.0, 4.5 Hz, 1H), 3.84 (dt, J=13.7, 4.2 Hz, 1H), 3.48 (ddd, J=11.1, 8.1, 6.0 Hz, 1H), 3.25 (ddd, J=13.7, 10.8, 2.8 Hz, 2H), 2.71-2.53 (m, 2H), 2.36 (dtd, J=14.0, 8.6, 5.1 Hz, 1H), 1.87-1.71 (m, 2H), 1.64-1.54 (m, 1H). FIRMS (ESI) m/z: [M+H]⁺ Calcd for C₂₂H₂₀Cl₂N₃O₂⁺: 428.0927; Found: 428.0920.

### Example 53: Synthesis of (±)-(4aR,13bS)-10,11-dichloro-4-(2-methoxyethyl)-1,2,3,4,4a,5,6,13b-octahydro-8H-[1,6]naphthyridino[5,6-b]quinazolin-8-one (7i)

Prepared according to Scheme 2.

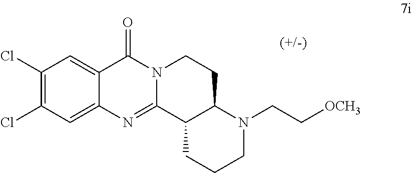 [SMILES: COCCN1CCC[C@@H]2c3nc4cc(Cl)c(Cl)cc4c(=O)n3CC[C@H]21]

Steps 1-5: See Example 43—Synthesis of 6b.

Step 6: Synthesis of (±)-(4aR,13bS)-10,11-dichloro-4-(2-methoxyethyl)-1,2,3,4,4a,5,6,13b-octahydro-8H-[1,6]naphthyridino[5,6-b]quinazolin-8-one (7i). Compound 7i was prepared according to general procedure 8.1 by suspending 6b (1 eq.), 1-bromo-2-methoxyethane (4 eq.), and Et₃N (6 eq.) in DMF (0.25 M) and heating to 50° C. for 22 h. Purification by flash chromatography 3-20% X/hexanes (X=2% NEt₃ 3:1 EtOAc/EtOH) afforded 7i as a yellow solid (62 mg, 75%). ¹H NMR (400 MHz, CDCl₃) δ 8.27 (s, 1H), 7.74 (s, 1H), 4.20 (ddd, J=14.4, 8.2, 6.2 Hz, 1H), 4.05 (dt, J=14.4, 6.2 Hz, 1H), 3.52 (dd, J=6.2, 5.1 Hz, 2H), 3.35 (s, 3H), 3.13-2.99 (m, 2H), 2.76-2.52 (m, 3H), 2.46 (dq, J=13.3, 5.9 Hz, 1H), 2.39-2.18 (m, 2H), 1.91-1.69 (m, 3H), 1.50-1.39 (m, 1H). HRMS (ESI) m/z: [M+H]⁺ Calcd for C₁₈H₂₂Cl₂N₃O₂, 382.1084. Found 382.1070.

### Example 54: Synthesis of (±)-(4aR,13bS)-10,11-dichloro-4-(2-hydroxyethyl)-1,2,3,4,4a,5,6,13b-octahydro-8H-[1,6]naphthyridino[5,6-b]quinazolin-8-one (7j)

Prepared according to Scheme 2.

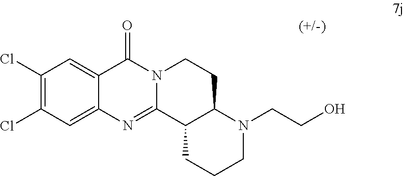 [SMILES: O=c1c2cc(Cl)c(Cl)cc2nc2n1CC[C@@H]1[C@@H]2CCCN1CCO]

Steps 1-5: See Example 43—Synthesis of 6b.

Step 6: Synthesis of (±)-Synthesis of (±)-(4aR,13bS)-10,11-dichloro-4-(2-hydroxyethyl)-1,2,3,4,4a,5,6,13b-octahydro-8H-[1,6]naphthyridino[5,6-b]quinazolin-8-one (7j). Compound 7c was prepared according to general procedure 8.1 by suspending 6b (1 eq.), 2-bromo-ethanol (4 eq.), and Et₃N (6 eq.) in DMF (0.25 M) and heating to 50° C. for 24 h. Purification by flash chromatography 10-60% X/hexanes (X=2% NEt₃ 3:1 EtOAc/EtOH) afforded 7j as a yellow solid (54 mg, 68%). ¹H NMR (400 MHz, CDCl₃) δ 8.29 (s, 1H), 7.76 (s, 1H), 4.29-4.14 (m, 1H), 4.07 (dt, J=14.0, 6.0 Hz, 1H), 3.75 (ddd, J=12.4, 9.6, 3.7 Hz, 1H), 3.66-3.57 (m, 1H), 3.20-3.06 (m, 2H), 2.74-2.63 (m, 2H), 2.54-2.29 (m, 4H), 2.17 (td, J=12.2, 2.7 Hz, 1H), 1.93-1.80 (m, 2H), 1.77-1.67 (m, 1H), 1.59-1.43 (m, 1H). HRMS (ESI) m/z: [M+H]⁺ Calcd for C₁₇H₂₀Cl₂N₃O₂, 368.0909. Found 368.0930.

### Example 55: Synthesis of (±)-(4aR,13bS)-10,11-dichloro-4-(pyridin-4-ylmethyl)-1,2,3,4,4a,5,6,13b-octahydro-8H-[1,6]naphthyridino[5,6-b]quinazolin-8-one (7k)

Prepared according to Scheme 2.

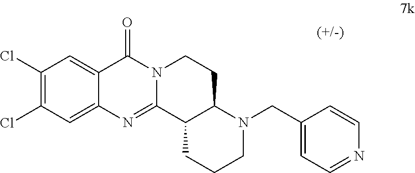 [SMILES: O=c1c2cc(Cl)c(Cl)cc2nc2n1CC[C@@H]1[C@@H]2CCCN1Cc1ccncc1]

Steps 1-5: See Example 43—Synthesis of 6b.

Step 6: Synthesis of (±)-(4aR,13bS)-10,11-dichloro-4-(pyridin-4-ylmethyl)-1,2,3,4,4a,5,6,13b-octahydro-8H-[1,6]naphthyridino[5,6-b]quinazolin-8-one (7k). Compound 7k was prepared according to general procedure 8.1 by suspending 6b (1 eq.), 4-(bromomethyl)-pyridine hydrobromide (4 eq.), and Et₃N (6 eq.) in DMF (0.25 M) and heating to 50° C. for 4 h. Purification by flash chromatography 5-30% X/hexanes (X=2% NEt₃ 3:1 EtOAc/EtOH) afforded 7k as a tan solid (38 mg, 42%). ¹H NMR (400 MHz, CDCl₃) δ 8.57 (d, J=5.1 Hz, 2H), 8.30 (s, 1H), 7.77 (s, 1H), 7.35-7.30 (m, 2H), 4.25 (ddd, J=14.5, 8.3, 6.2 Hz, 1H), 4.15-4.06 (m, 2H), 3.22 (d, J=14.7 Hz, 1H), 2.89 (dq, J=11.7, 2.1 Hz, 1H), 2.81-2.75 (m, 1H), 2.71-2.65 (m, 1H), 2.44 (dt, J=13.2, 5.8 Hz, 1H), 2.35 (ddd, J=10.6, 9.0, 5.3 Hz, 1H), 2.07 (td, J=11.9, 2.9 Hz, 1H), 1.94-1.86 (m, 1H), 1.85-1.78 (m, 1H), 1.70 (qt, J=13.0, 3.7 Hz, 1H), 1.50 (tdd, J=13.2, 11.5, 4.1 Hz, 1H). HRMS (ESI) m/z: [M+H]⁺ Calcd for C₂₁H₂₁Cl₂N₄O, 415.1087. Found 415.1071.

### Example 56: Synthesis of (±)-(4aR,13bS)-10,11-dichloro-4-isobutyl-1,2,3,4,4a,5,6,13b-octahydro-8H-[1,6]naphthyridino[5,6-b]quinazolin-8-one (71)

Prepared according to Scheme 2.

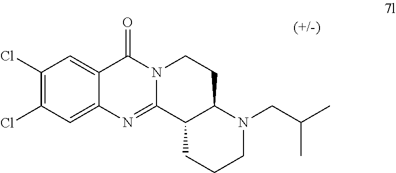 [SMILES: CC(C)CN1CCC[C@@H]2c3nc4cc(Cl)c(Cl)cc4c(=O)n3CC[C@H]21]

Steps 1-5: See Example 43—Synthesis of 6b.

Step 6: Synthesis of (±)-Synthesis of (±)-(4aR,13bS)-10,11-dichloro-4-isobutyl-1,2,3,4,4a,5,6,13b-octahydro-8H-[1,6]naphthyridino[5,6-b]quinazolin-8-one (71). Compound 71 was prepared according to general procedure 8.1 by suspending 6b (1 eq.), isobutylbromide (4 eq.), and Et₃N (6 eq.) in DMF (0.25 M) and heating to 80° C. for 18 h. Purification by flash chromatography 5-25% X/hexanes (X=4:1 EtOAc/DCM) afforded 71 as a yellowish white solid (40 mg, 50%). ¹H NMR (400 MHz, CDCl₃) δ 8.28 (s, 1H), 7.75 (s, 1H), 4.20 (dt, J=14.7, 7.1 Hz, 1H), 4.04 (dt, J=14.4, 6.2 Hz, 1H), 3.07 (d, J=11.4 Hz, 1H), 2.73-2.56 (m, 2H), 2.54-2.33 (m, 2H), 2.15 (q, J=8.9 Hz, 1H), 2.03-1.87 (m, 2H), 1.75 (d, J=37.4 Hz, 4H), 1.45 (qd, J=13.0, 4.0 Hz, 1H), 0.92 (dd, J=11.0, 6.5 Hz, 6H). HRMS (ESI) m/z: [M+H]⁺ Calcd for C₁₉H₂₄Cl₂N₄O, 380.1272. Found 380.1277.

### Example 57: Synthesis of (±)-(4aR,13bS)-10,11-dichloro-4-(oxetan-3-ylmethyl)-1,2,3,4,4a,5,6,13b-octahydro-8H-[1,6]naphthyridino[5,6-b]quinazolin-8-one (7m)

Prepared according to Scheme 2.

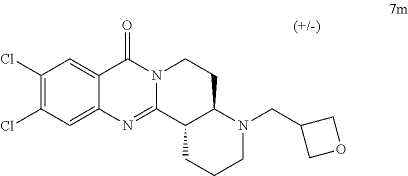 [SMILES: O=c1c2cc(Cl)c(Cl)cc2nc2n1CC[C@@H]1[C@@H]2CCCN1CC1COC1]

Steps 1-5: See Example 43—Synthesis of 6b.

Step 6: Synthesis of (±)-(±)-(4aR,13bS)-10,11-dichloro-4-(oxetan-3-ylmethyl)-1,2,3,4,4a,5,6,13b-octahydro-8H-[1,6]naphthyridino[5,6-b]quinazolin-8-one (7m). Compound 7m was prepared according to general procedure 8.1 by suspending 6b (1 eq.), 3-(bromomethyl)oxetane (4 eq.), and Et₃N (6 eq.) in DMF (0.25 M) and heating to 80° C. for 19 h. Purification by flash chromatography 3-30% X/hexanes (X=2% NEt₃ 3:1 EtOAc/EtOH) afforded 7c as a tan solid (60 mg, 71%). ¹H NMR (400 MHz, CDCl₃) δ 8.27 (s, 1H), 7.73 (s, 1H), 4.81 (q, J=7.7, 7.3 Hz, 2H), 4.44 (t, J=5.9 Hz, 1H), 4.37 (t, J=5.8 Hz, 1H), 4.21 (dt, J=14.6, 7.1 Hz, 1H), 4.07 (dt, J=14.0, 6.2 Hz, 1H), 3.24 (q, J=8.2 Hz, 2H), 2.88 (d, J=11.3 Hz, 1H), 2.64 (d, J=12.3 Hz, 2H), 2.46 (dq, J=19.2, 6.3, 5.8 Hz, 2H), 2.21 (s, 1H), 2.01 (t, J=12.0 Hz, 1H), 1.85 (t, J=14.3 Hz, 2H), 1.67 (d, J=13.3 Hz, 1H), 1.49 (d, J=3.8 Hz, 1H). HRMS (ESI) m/z: [M+H]⁺ Calcd for C₁₉H₂₂Cl₂N₄O₂, 394.1064. Found 394.1070.

### Example 58: (±)-(4aR,13bS)-10,11-dichloro-4-(3,5-dimethoxybenzyl)-1,2,3,4,4a,5,6,13b-octahydro-8H-[1,6]naphthyridino[5,6-b]quinazolin-8-one (7n)

Prepared according to Scheme 2.

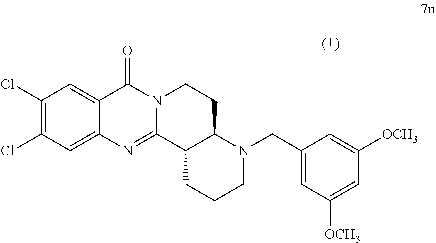 [SMILES: COc1cc(CN2CCC[C@@H]3c4nc5cc(Cl)c(Cl)cc5c(=O)n4CC[C@H]32)cc(OC)c1]

Steps 1-5: See Example 43—Synthesis of 6b.

Step 6: Synthesis of (±)-(4aR,13bS)-10,11-dichloro-4-(3,5-dimethoxybenzyl)-1,2,3,4,4a,5,6,13b-octahydro-8H-[1,6]naphthyridino[5,6-b]quinazolin-8-one (7n). Compound 7n was prepared according to general procedure 8.1 by suspending 6b (1 eq.), 1-(bromomethyl)-3,5-dimethoxybenzene (4 eq.), and Et₃N (6 eq.) in DMF (0.25 M) and heating to 50° C. for 20 h. Purification by flash chromatography 0.5-10% X/hexanes (X=2% NEt₃ 3:1 EtOAc/EtOH) afforded 7n as a light-yellow solid (70.5 mg, 69%). ¹H NMR (400 MHz, CDCl₃) δ 8.29 (d, J=3.5 Hz, 1H), 7.76 (d, J=2.9 Hz, 1H), 6.50 (d, J=2.3 Hz, 2H), 6.36 (t, J=2.4 Hz, 1H), 4.31-4.19 (m, 1H), 4.13-4.02 (m, 2H), 3.79 (s, 6H), 3.17 (d, J=13.8 Hz, 1H), 2.96 (dd, J=9.7, 6.0 Hz, 1H), 2.75 (td, J=11.2, 3.7 Hz, 1H), 2.64 (dd, J=13.5, 3.6 Hz, 1H), 2.49 (dq, J=12.0, 5.9 Hz, 1H), 2.29 (td, J=9.9, 5.3 Hz, 1H), 2.07-1.86 (m, 2H), 1.84-1.59 (m, 2H), 1.47 (qd, J=13.1, 4.1 Hz, 1H). HRMS (ESI) m/z: [M+H]⁺ Calcd for C₂₄H₂₆Cl₂N₃O₃, 474.1346. Found 474.1342.

### Example 59: (±)-(4aR,13bS)-10,11-dichloro-4-(pyridin-2-ylmethyl)-1,2,3,4,4a,5,6,13b-octahydro-8H-[1,6]naphthyridino[5,6-b]quinazolin-8-one (7o)

Prepared according to Scheme 2.

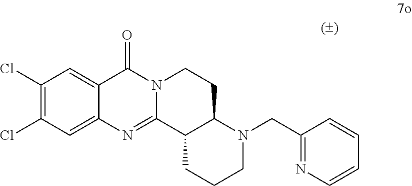 [SMILES: O=c1c2cc(Cl)c(Cl)cc2nc2n1CC[C@@H]1[C@@H]2CCCN1Cc1ccccn1]

Steps 1-5: See Example 43—Synthesis of 6b.

Step 6: Synthesis of (±)-(4aR,13bS)-10,11-dichloro-4-(pyridin-2-ylmethyl)-1,2,3,4,4a,5,6,13b-octahydro-8H-[1,6]naphthyridino[5,6-b]quinazolin-8-one (7o). Compound 7o was prepared according to general procedure 8.1 by suspending 6b (1 eq.), 2-(chloromethyl)pyridine hydrochloride (4 eq.), and Et₃N (6 eq.) in DMF (0.25 M) and heating to 50° C. for 20 h. Purification by flash chromatography 3-30% X/hexanes (X=2% NEt₃ 3:1 EtOAc/EtOH) afforded 7o as an off-white solid (72.3 mg, 81%). ¹H NMR (400 MHz, CDCl₃) δ 8.55 (dd, J=5.0, 1.7 Hz, 1H), 8.27 (s, 1H), 7.73 (s, 1H), 7.65 (td, J=7.7, 1.8 Hz, 1H), 7.41 (d, J=7.8 Hz, 1H), 7.20-7.12 (m, 1H), 4.25-4.03 (m, 3H), 3.56 (d, J=14.4 Hz, 1H), 2.95 (dt, J=11.6, 3.4 Hz, 1H), 2.76 (td, J=11.2, 3.7 Hz, 1H), 2.70-2.54 (m, 2H), 2.37 (td, J=10.0, 5.1 Hz, 1H), 2.18 (td, J=11.8, 3.2 Hz, 1H), 1.97-1.64 (m, 3H), 1.45 (qd, J=12.9, 4.4 Hz, 1H). HRMS (ESI) m/z: [M+H]⁺ Calcd for C₂₁H₂₁Cl₂N₄O, 415.1087. Found 415.1090.

### Example 60: (±)-(4aR,13bS)-10,11-dichloro-4-((6-methylpyridin-2-yl)methyl)-1,2,3,4,4a,5,6,13b-octahydro-8H-[1,6]naphthyridino[5,6-b]quinazolin-8-one (7p)

Prepared according to Scheme 2.

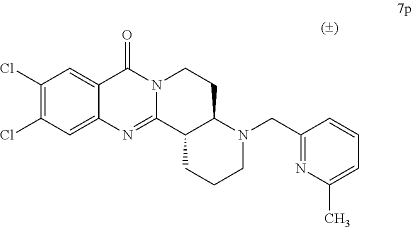 [SMILES: Cc1cccc(CN2CCC[C@@H]3c4nc5cc(Cl)c(Cl)cc5c(=O)n4CC[C@H]32)n1]

Steps 1-5: See Example 43—Synthesis of 6b.

Step 6: Synthesis of (±)-(4aR,13bS)-10,11-dichloro-4-((6-methylpyridin-2-yl)methyl)-1,2,3,4,4a,5,6,13b-octahydro-8H-[1,6]naphthyridino[5,6-b]quinazolin-8-one (7p). Compound 7p was prepared according to general procedure 8.1 by suspending 6b (1 eq.), 2-(bromomethyl)-6-methyl-pyridine (4 eq.), and Et₃N (6 eq.) in DMF (0.25 M) and heating to 50° C. for 20 h. Purification by flash chromatography 3-30% X/hexanes (X=2% NEt₃ 3:1 EtOAc/EtOH) afforded 7p as a tan solid (77.8 mg, 84%). ¹H NMR (400 MHz, CDCl₃) δ 8.28 (s, 1H), 7.74 (s, 1H), 7.54 (t, J=7.7 Hz, 1H), 7.24 (d, J=7.7 Hz, 1H), 7.02 (d, J=7.6 Hz, 1H), 4.13 (dd, J=8.1, 6.0 Hz, 2H), 4.08 (d, J=14.8 Hz, 1H), 3.63 (d, J=14.8 Hz, 1H), 3.02-2.94 (m, 1H), 2.75 (td, J=11.2, 3.8 Hz, 1H), 2.71-2.60 (m, 2H), 2.53 (s, 3H), 2.38 (td, J=10.2, 4.9 Hz, 1H), 2.22 (td, J=11.9, 3.1 Hz, 1H), 1.92-1.68 (m, 3H), 1.44 (qd, J=12.5, 4.6 Hz, 1H). HRMS (ESI) m/z: [M+H]⁺ Calcd for C₂₂H₂₃Cl₂N₄O, 429.1243. Found 429.1248.

### Example 61: (±)-(4aR,13bS)-10,11-dichloro-4-(cyclopropylmethyl)-1,2,3,4,4a,5,6,13b-octahydro-8H-[1,6]naphthyridino[5,6-b]quinazolin-8-one (7q)

Prepared according to Scheme 2.

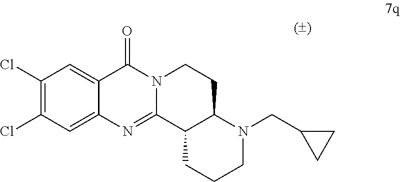 [SMILES: O=c1c2cc(Cl)c(Cl)cc2nc2n1CC[C@@H]1[C@@H]2CCCN1CC1CC1]

Steps 1-5: See Example 43—Synthesis of 6b.

Step 6: Synthesis of (±)-(4aR,13bS)-10,11-dichloro-4-(cyclopropylmethyl)-1,2,3,4,4a,5,6,13b-octahydro-8H-[1,6]naphthyridino[5,6-b]quinazolin-8-one (7q). Compound 7q was prepared according to general procedure 8.1 by suspending 6b (1 eq.), (bromomethyl)cyclopropane (4 eq.), and Et₃N (6 eq.) in DMF (0.25 M) and heating to 80° C. for 20 h. Purification by flash chromatography 0.5-6% X/hexanes (X=2% NEt₃ 3:1 EtOAc/EtOH) afforded 7q as a light-yellow solid (52.3 mg, 65%). ¹H NMR (400 MHz, CDCl₃) δ 8.28 (s, 1H), 7.75 (s, 1H), 4.20 (ddd, J=14.5, 8.3, 6.2 Hz, 1H), 4.06 (dt, J=14.4, 6.1 Hz, 1H), 3.30 (ddt, J=11.9, 4.0, 1.8 Hz, 1H), 2.77 (dd, J=13.4, 6.1 Hz, 1H), 2.74-2.62 (m, 2H), 2.43 (dq, J=13.4, 6.0 Hz, 1H), 2.35-2.19 (m, 3H), 1.91-1.68 (m, 3H), 1.46 (tdd, J=12.9, 11.2, 3.8 Hz, 1H), 0.93-0.82 (m, 1H), 0.55 (dpt, J=8.7, 6.1, 3.0 Hz, 2H), 0.13 (ddp, J=5.3, 4.0, 1.7 Hz, 2H). HRMS (ESI) m/z: [M+H]⁺ Calcd for C₁₉H₂₂Cl₂N₃O, 378.1134. Found 378.1140.

### Example 62: (±)-(4-(((4aR,13bS)-10,11-dichloro-8-oxo-2,3,5,6,8,13b-hexahydro-1H-[1,6]naphthyridino[5,6-b]quinazolin-4(4aH)-yl)methyl)benzonitrile (7r)

Prepared according to Scheme 2.

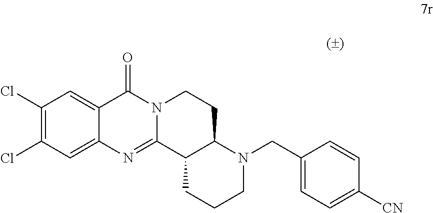 [SMILES: N#Cc1ccc(CN2CCC[C@@H]3c4nc5cc(Cl)c(Cl)cc5c(=O)n4CC[C@H]32)cc1]

Steps 1-5: See Example 43—Synthesis of 6b.

Step 6: Synthesis of (±)-(4-(((4aR,13bS)-10,11-dichloro-8-oxo-2,3,5,6,8,13b-hexahydro-1H-[1,6]naphthyridino[5,6-b]quinazolin-4(4aH)-yl)methyl)benzonitrile (7r). Compound 7r was prepared according to general procedure 8.1 by suspending 6b (1 eq.), 4-(bromomethyl)benzonitrile (4 eq.), and Et₃N (6 eq.) in DMF (0.25 M) and heating to 50° C. for 20 h. Purification by flash chromatography 0.5-10% X/hexanes (X=2% NEt₃ 3:1 EtOAc/EtOH) afforded 7r as a yellow solid (62.8 mg, 66%). ¹H NMR (400 MHz, CDCl₃) δ 8.29 (s, 1H), 7.76 (s, 1H), 7.65-7.59 (m, 2H), 7.48 (d, J=8.1 Hz, 2H), 4.30-4.15 (m, 2H), 4.08 (dt, J=14.4, 6.2 Hz, 1H), 3.23 (d, J=14.5 Hz, 1H), 2.90-2.82 (m, 1H), 2.75 (td, J=11.1, 3.7 Hz, 1H), 2.72-2.62 (m, 1H), 2.45 (dq, J=13.1, 6.0 Hz, 1H), 2.34 (ddd, J=10.6, 9.1, 5.3 Hz, 1H), 2.04 (td, J=11.9, 2.8 Hz, 1H), 1.89 (dtd, J=13.3, 8.6, 6.2 Hz, 1H), 1.83-1.76 (m, 1H), 1.67 (tdd, J=13.0, 9.2, 3.6 Hz, 1H), 1.49 (tdd, J=13.2, 11.5, 4.0 Hz, 1H). FIRMS (ESI) m/z: [M+H]⁺ Calcd for C₂₃H₂₁Cl₂N₄O, 439.1087. Found 439.1089.

### Example 63: (±)-(4aR,13bS)-4-(4-(tert-butyl)benzyl)-10,11-dichloro-1,2,3,4,4a,5,6,13b-octahydro-8H-[1,6]naphthyridino[5,6-b]quinazolin-8-one (7s)

Prepared according to Scheme 2.

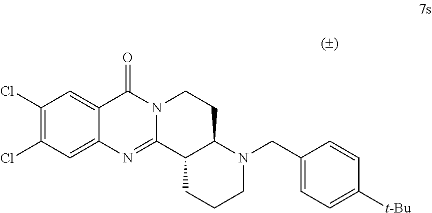 [SMILES: CC(C)(C)c1ccc(CN2CCC[C@@H]3c4nc5cc(Cl)c(Cl)cc5c(=O)n4CC[C@H]32)cc1]

Steps 1-5: See Example 43—Synthesis of 6b.

Step 6: Synthesis of (±)-(4aR,13bS)-4-(4-(tert-butyl)benzyl)-10,11-dichloro-1,2,3,4,4a,5,6,13b-octahydro-8H-[1,6]naphthyridino[5,6-b]quinazolin-8-one (7s). Compound 7s was prepared according to general procedure 8.1 by suspending 6b (1 eq.), 1-(bromomethyl)-4-(tert-butyl)benzene (4 eq.), and Et₃N (6 eq.) in DMF (0.25 M) and heating to 50° C. for 20 h. Purification by flash chromatography 0.5-8% X/hexanes (X=2% NEt₃ 3:1 EtOAc/EtOH) afforded 7s as a yellow solid (88.1 mg, 86%). ¹H NMR (400 MHz, CDCl₃) δ 8.30 (s, 1H), 7.76 (s, 1H), 7.37-7.33 (m, 2H), 7.26-7.23 (m, 2H), 4.30 (ddd, J=14.3, 8.0, 6.3 Hz, 1H), 4.12-4.04 (m, 2H), 3.25 (d, J=13.7 Hz, 1H), 2.98 (dd, J=9.6, 5.8 Hz, 1H), 2.76 (td, J=11.2, 3.7 Hz, 1H), 2.66-2.52 (m, 2H), 2.29 (ddd, J=10.7, 9.0, 5.4 Hz, 1H), 2.00 (qd, J=12.7, 12.3, 4.8 Hz, 2H), 1.84-1.73 (m, 1H), 1.67 (dddd, J=16.7, 13.0, 8.3, 3.6 Hz, 1H), 1.47 (dd, J=12.6, 4.2 Hz, 1H), 1.32 (s, 9H). FIRMS (ESI) m/z: [M+H]⁺ Calcd for C₂₆H₃₀Cl₂N₃O, 470.1760. Found 470.1771.

### Example 64: (±)-(4aR,13bS)-10,11-dichloro-4-(4-chlorobenzyl)-1,2,3,4,4a,5,6,13b-octahydro-8H-[1,6]naphthyridino[5,6-b]quinazolin-8-one (7t)

Prepared according to Scheme 2.

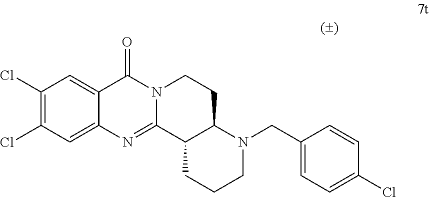 [SMILES: O=c1c2cc(Cl)c(Cl)cc2nc2n1CC[C@@H]1[C@@H]2CCCN1Cc1ccc(Cl)cc1]

Steps 1-5: See Example 43—Synthesis of 6b.

Step 6: Synthesis of (±)-(4aR,13bS)-10,11-dichloro-4-(4-chlorobenzyl)-1,2,3,4,4a,5,6,13b-octahydro-8H-[1,6]naphthyridino[5,6-b]quinazolin-8-one (7t). Compound 7t was prepared according to general procedure 8.1 by suspending 6b (1 eq.), 1-(bromomethyl)-4-chlorobenzene (4 eq.), and Et₃N (6 eq.) in DMF (0.25 M) and heating to 50° C. for 20 h. Purification by flash chromatography 0.5-8% X/hexanes (X=2% NEt₃ 3:1 EtOAc/EtOH) afforded 7t as a yellow solid (72.8 mg, 75%). ¹H NMR (400 MHz, CDCl₃) δ 8.29 (s, 1H), 7.76 (s, 1H), 7.32-7.25 (m, 4H), 4.27 (ddd, J=14.3, 8.0, 6.3 Hz, 1H), 4.08 (ddd, J=14.4, 10.9, 4.7 Hz, 2H), 3.17 (d, J=13.8 Hz, 1H), 2.93-2.85 (m, 1H), 2.75 (td, J=11.1, 3.7 Hz, 1H), 2.67-2.60 (m, 1H), 2.50 (dq, J=13.3, 6.0 Hz, 1H), 2.29 (ddd, J=10.6, 9.1, 5.4 Hz, 1H), 1.95 (dtd, J=22.5, 14.4, 13.2, 5.4 Hz, 2H), 1.82-1.75 (m, 1H), 1.65 (dddd, J=16.7, 13.0, 8.3, 3.7 Hz, 1H), 1.53-1.42 (m, 1H). HRMS (ESI) m/z: [M+H]⁺ Calcd for C₂₂H₂₁Cl₃N₃O, 448.0745. Found 448.0750.

Example 65: (±)-(4aR,13bS)-10,11-dichloro-4-(4-(trifluoromethyl)benzyl)-1,2,3,4,4a,5,6,13b-octahydro-8H-[1,6]naphthyridino[5,6-b]quinazolin-8-one (7u)

Prepared according to Scheme 2.

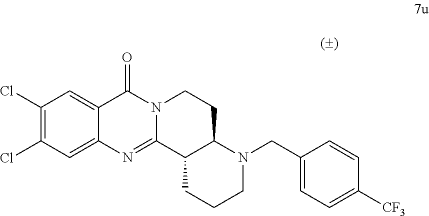 [SMILES: O=c1c2cc(Cl)c(Cl)cc2nc2n1CC[C@@H]1[C@@H]2CCCN1Cc1ccc(C(F)(F)F)cc1]

Steps 1-5: See Example 43—Synthesis of 6b.

Step 6: Synthesis of (±)-((4aR,13bS)-10,11-dichloro-4-(4-(trifluoromethyl)benzyl)-1,2,3,4,4a,5,6,13b-octahydro-8H-[1,6]naphthyridino[5,6-b]quinazolin-8-one (7u). Compound 7u was prepared according to general procedure 8.1 by suspending 6b (1 eq.), 1-(bromomethyl)-4-(trifluoromethyl)benzene (4 eq.), and Et₃N (6 eq.) in DMF (0.25 M) and heating to 50° C. for 20 h. Purification by flash chromatography 0.5-8% X/hexanes (X=2% NEt₃ 3:1 EtOAc/EtOH) afforded 7u as an off-white solid (86.6 mg, 83%). ¹H NMR (400 MHz, CDCl₃) δ 8.30 (s, 1H), 7.77 (s, 1H), 7.59 (d, J=8.0 Hz, 2H), 7.47 (d, J=8.0 Hz, 2H), 4.28 (ddd, J=14.4, 8.1, 6.2 Hz, 1H), 4.19 (d, J=14.1 Hz, 1H), 4.09 (dt, J=14.4, 6.2 Hz, 1H), 3.25 (d, J=14.1 Hz, 1H), 2.92-2.85 (m, 1H), 2.77 (td, J=11.2, 3.6 Hz, 1H), 2.69-2.62 (m, 1H), 2.49 (dq, J=13.3, 6.0 Hz, 1H), 2.33 (td, J=10.7, 10.1, 5.3 Hz, 1H), 2.02 (td, J=11.9, 2.8 Hz, 1H), 1.93 (dq, J=14.8, 8.0 Hz, 1H), 1.83-1.76 (m, 1H), 1.68 (tdd, J=16.4, 10.1, 6.4 Hz, 1H), 1.49 (tdd, J=13.3, 11.5, 4.1 Hz, 1H). HRMS (ESI) m/z: [M+H]⁺ Calcd for C₂₃H₂₁Cl₂F₃N₃O, 482.1008. Found 482. 1012.

### Example 66: (±)-(4aR,13bS)-10,11-dichloro-4-(2,4-dichlorobenzyl)-1,2,3,4,4a,5,6,13b-octahydro-8H-[1,6]naphthyridino[5,6-b]quinazolin-8-one (7v)

Prepared according to Scheme 2.

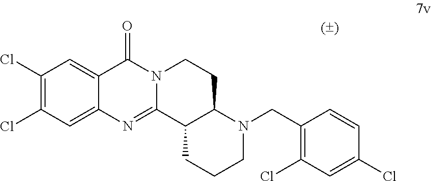 [SMILES: O=c1c2cc(Cl)c(Cl)cc2nc2n1CC[C@@H]1[C@@H]2CCCN1Cc1ccc(Cl)cc1Cl]

Steps 1-5: See Example 43—Synthesis of 6b.

Step 6: Synthesis of (±)-(4aR,13bS)-10,11-dichloro-4-(2,4-dichlorobenzyl)-1,2,3,4,4a,5,6,13b-octahydro-8H-[1,6]naphthyridino[5,6-b]quinazolin-8-one (7v). Compound 7v was prepared according to general procedure 8.1 by suspending 6b (1 eq.), 1-(bromomethyl)-2,4-dichlorobenzene (4 eq.), and Et₃N (6 eq.) in DMF (0.25 M) and heating to 50° C. for 20 h. Purification by flash chromatography 0.5-8% X/hexanes (X=2% NEt₃ 3:1 EtOAc/EtOH) afforded 7v as an off-white solid (90.5 mg, 87%). ¹H NMR (400 MHz, CDCl₃) δ 8.30 (s, 1H), 7.77 (s, 1H), 7.52 (d, J=8.3 Hz, 1H), 7.37 (d, J=2.1 Hz, 1H), 7.24 (dd, J=8.3, 2.1 Hz, 1H), 4.21 (ddd, J=14.3, 8.5, 5.8 Hz, 1H), 4.13-4.03 (m, 2H), 3.35 (d, J=15.1 Hz, 1H), 2.91 (dq, J=11.8, 3.5, 2.6 Hz, 1H), 2.76 (td, J=10.9, 3.7 Hz, 1H), 2.71-2.64 (m, 1H), 2.48-2.35 (m, 2H), 2.12 (td, J=12.0, 2.8 Hz, 1H), 1.95-1.78 (m, 2H), 1.74-1.63 (m, 1H), 1.56-1.45 (m, 1H). HRMS (ESI) m/z: [M+H]⁺ Calcd for C₂₂H₂₀Cl₄N₃O, 482.0355. Found 482.0360.

### Example 67: (±)-(4aR,13bS)-4-benzyl-10,11-dichloro-1,2,3,4,4a,5,6,13b-octahydro-8H-[1,6]naphthyridino[5,6-b]quinazolin-8-one (7w)

Prepared according to Scheme 2.

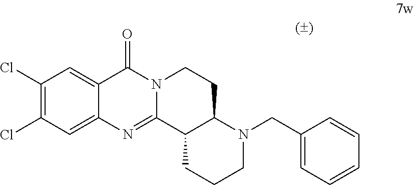 [SMILES: O=c1c2cc(Cl)c(Cl)cc2nc2n1CC[C@@H]1[C@@H]2CCCN1Cc1ccccc1]

Steps 1-5: See Example 43—Synthesis of 6b.

Step 6: Synthesis of (±)-(4aR,13bS)-4-benzyl-10,11-dichloro-1,2,3,4,4a,5,6,13b-octahydro-8H-[1,6]naphthyridino[5,6-b]quinazolin-8-one (7w). Compound 7w was prepared according to general procedure 8.1 by suspending 6b (1 eq.), benzyl bromide (4 eq.), and Et₃N (6 eq.) in DMF (0.25 M) and heating to 50° C. for 20 h. Purification by flash chromatography 0.5-8% X/hexanes (X=2% NEt₃ 3:1 EtOAc/EtOH) afforded 7w as an off-white solid (74.5 mg, 74%). ¹H NMR (400 MHz, CDCl₃) δ 8.30 (s, 1H), 7.76 (s, 1H), 7.33 (d, J=4.4 Hz, 4H), 7.30-7.24 (m, 1H), 4.29 (ddd, J=14.4, 8.0, 6.3 Hz, 1H), 4.17-4.04 (m, 2H), 3.24 (d, J=13.7 Hz, 1H), 2.95 (dq, J=13.3, 3.5, 2.7 Hz, 1H), 2.77 (td, J=11.1, 3.7 Hz, 1H), 2.67-2.60 (m, 1H), 2.55 (dq, J=13.3, 6.1 Hz, 1H), 2.30 (ddd, J=10.6, 9.1, 5.4 Hz, 1H), 2.05-1.91 (m, 2H), 1.82-1.75 (m, 1H), 1.66 (qt, J=12.9, 3.7 Hz, 1H), 1.47 (tdd, J=13.2, 11.5, 4.1 Hz, 1H). HRMS (ESI) m/z: [M+H]⁺ Calcd for C₂₂H₂₂Cl₂N₃O, 414.1134. Found 414.1136.

### Example 68: Synthesis of (±)-(4aR,13bS)-10,11-dichloro-4-(pyrazin-2-ylmethyl)-1,2,3,4,4a,5,6,13b-octahydro-8H-[1,6]naphthyridino[5,6-b]quinazolin-8-one (7x)

Prepared according to Scheme 2.

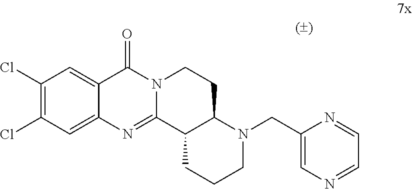 [SMILES: O=c1c2cc(Cl)c(Cl)cc2nc2n1CC[C@@H]1[C@@H]2CCCN1Cc1cnccn1]

Steps 1-5: See Example 43—Synthesis of 6b.

Step 6: Synthesis of (±)-(4aR,13bS)-10,11-dichloro-4-(pyrazin-2-ylmethyl)-1,2,3,4,4a,5,6,13b-octahydro-8H-[1,6]naphthyridino[5,6-b]quinazolin-8-one (7x). Compound 7x was prepared according to general procedure 8.1 by suspending 6b (1 eq.), 2-(bromomethyl)-pyrazine hydrobromide (4 eq.), and Et₃N (6 eq.) in DMF (0.25 M) and heating to 50° C. for 20 h. Purification by flash chromatography 1-25% X/hexanes (X=2% NEt₃ 3:1 EtOAc/EtOH) afforded 7w as an off-white solid (75.5 mg, 84%). ¹H NMR (400 MHz, CDCl₃) δ 8.69 (d, J=1.5 Hz, 1H), 8.53 (dd, J=2.6, 1.5 Hz, 1H), 8.47 (d, J=2.5 Hz, 1H), 8.28 (s, 1H), 7.74 (s, 1H), 4.26-4.07 (m, 3H), 3.64 (d, J=14.8 Hz, 1H), 2.94 (ddt, J=11.4, 4.0, 2.0 Hz, 1H), 2.77 (td, J=11.2, 3.7 Hz, 1H), 2.70-2.57 (m, 2H), 2.40 (ddd, J=10.7, 9.4, 5.1 Hz, 1H), 2.21 (td, J=11.9, 3.1 Hz, 1H), 1.92 (dtd, J=13.5, 8.9, 6.3 Hz, 1H), 1.84-1.68 (m, 2H), 1.51-1.39 (m, 1H). HRMS (ESI) m/z: [M+H]⁺ Calcd for C₂₀H₂₀Cl₂N₅O, 416.1039. Found 416.1040.

### Example 69: Synthesis of (±)-(4aR,13bS)-10,11-dichloro-4-(2-morpholinoethyl)-1,2,3,4,4a,5,6,13b-octahydro-8H-[1,6]naphthyridino[5,6-b]quinazolin-8-one (7y)

Prepared according to Scheme 2.

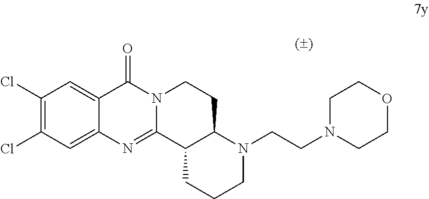 [SMILES: O=c1c2cc(Cl)c(Cl)cc2nc2n1CC[C@@H]1[C@@H]2CCCN1CCN1CCOCC1]

Steps 1-5: See Example 43—Synthesis of 6b.

Step 6: Synthesis of (±)-(4aR,13bS)-10,11-dichloro-4-(2-morpholinoethyl)-1,2,3,4,4a,5,6,13b-octahydro-8H-[1,6]naphthyridino[5,6-b]quinazolin-8-one (7z). Compound 7z was prepared according to general procedure 8.1 by suspending 6b (1 eq.), 4-(2-chloroethyl)morpholine (8 eq.), and Et₃N (6 eq.) in DMF (0.25 M) and heating to 80° C. for 24 h. Purification by flash chromatography 30-80% X/hexanes (X=2% NEt₃ 3:1 EtOAc/EtOH) afforded 7w as a tan solid (55.8 mg, 59%). ¹H NMR (400 MHz, CDCl₃) δ 8.27 (s, 1H), 7.74 (s, 1H), 4.19 (ddd, J=14.5, 8.4, 6.1 Hz, 1H), 4.07 (dt, J=14.4, 6.0 Hz, 1H), 3.70 (t, J=4.7 Hz, 4H), 3.10-3.04 (m, 1H), 3.02-2.91 (m, 1H), 2.65 (ddt, J=12.0, 9.5, 3.6 Hz, 2H), 2.55-2.41 (m, 8H), 2.28 (td, J=10.5, 5.2 Hz, 1H), 2.21 (td, J=12.0, 2.9 Hz, 1H), 1.89-1.80 (m, 2H), 1.70 (qt, J=13.1, 3.8 Hz, 1H), 1.45 (tdd, J=14.0, 10.8, 6.6 Hz, 1H). HRMS (ESI) m/z: [M+H]⁺ Calcd for C₂₁H₂₇Cl₂N₄O₂, 437.1506. Found 437.1509.

### Example 70: (±)-(4aR,13bS)-10,11-dichloro-4-(2-isopropoxyethyl)-1,2,3,4,4a,5,6,13b-octahydro-8H-[1,6]naphthyridino[5,6-b]quinazolin-8-one (7z)

Prepared according to Scheme 2.

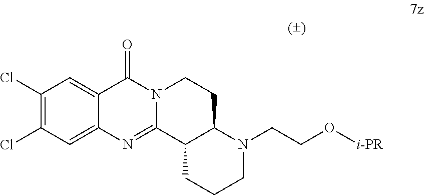

Steps 1-5: See Example 43-Synthesis of 6b.

Step 6: Synthesis of (±)-(4aR,13bS)-10,11-dichloro-4-(2-isopropoxyethyl)-1,2,3,4,4a,5,6,13b-octahydro-8H-[1,6]naphthyridino[5,6-b]quinazolin-8-one (7z). Compound 7z was prepared according to general procedure 8.1 by suspending 6b (1 eq.), 2-(2-bromoethoxy)propane (4 eq.), and Et₃N (6 eq.) in DMF (0.25 M) and heating to 50° C. for 20 h. Purification by flash chromatography 3-20% X/hexanes (X=2% NEt₃ 3:1 EtOAc/EtOH) afforded 7z as a tan solid (67.9 mg, 77%). ¹H NMR (400 MHz, CDCl₃) δ 8.28 (s, 1H), 7.74 (s, 1H), 4.18-4.08 (m, 2H), 3.65-3.46 (m, 3H), 3.07-3.00 (m, 1H), 2.96 (dt, J=13.9, 5.8 Hz, 1H), 2.75 (dt, J=13.5, 6.4 Hz, 1H), 2.70-2.61 (m, 2H), 2.55 (dq, J=13.3, 5.5 Hz, 1H), 2.38 (dtd, J=23.7, 12.0, 10.9, 3.9 Hz, 2H), 1.90-1.65 (m, 3H), 1.50-1.38 (m, 1H), 1.15 (dd, J=6.1, 1.2 Hz, 6H). FIRMS (ESI) m/z: [M+H]⁺ Calcd for C₂₀H₂₆Cl₂N₃O₂, 410.1397. Found 410.1397.

### Example 71: Synthesis of (±)-(4aR,13bS)-10,11-dichloro-4-(2-(isopropylamino)ethyl)-1,2,3,4,4a,5,6,13b-octahydro-8H-[1,6]naphthyridino[5,6-b]quinazolin-8-one (7aa)

Prepared according to Scheme 2.

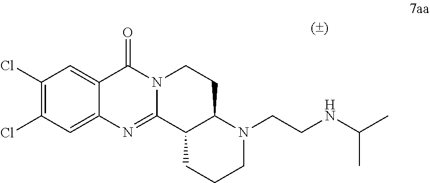 [SMILES: CC(C)NCCN1CCC[C@@H]2c3nc4cc(Cl)c(Cl)cc4c(=O)n3CC[C@H]21]

Steps 1-5: Example 43—Synthesis of 6b.

Step 6: Synthesis of (±)-(4aR,13bS)-10,11-dichloro-4-(2-(isopropylamino)ethyl)-1,2,3,4,4a,5,6,13b-octahydro-8H-[1,6]naphthyridino[5,6-b]quinazolin-8-one (7aa). Compound 7aa was prepared according to procedure 8.3 by suspending 7j (1 eq.) and Et₃N (2 eq.) in anhydrous DCE (0.25 M) and cooled to 0° C. At 0° C., MsCl (1.2 eq.) was added dropwise. Then allowed to warm to room temperature and stir for 2 h. After 2 h, isopropylamine (6 eq.) was added dropwise. The reaction was allowed to stir at room temperature for 22 h. Purification by flash chromatography 75-100% X/hexanes (X=2% NEt₃ 3:1 EtOAc/EtOH) afforded 7aa as an orange solid (38.1 mg, 49%). ¹H NMR (400 MHz, CDCl₃) δ 8.28 (s, 1H), 7.75 (s, 1H), 4.20 (ddd, J=14.4, 8.4, 6.3 Hz, 1H), 4.05 (ddd, J=14.9, 6.8, 5.4 Hz, 1H), 3.12-2.98 (m, 2H), 2.86-2.60 (m, 5H), 2.48-2.40 (m, 1H), 2.34-2.21 (m, 2H), 2.08 (td, J=12.1, 2.7 Hz, 1H), 1.91-1.77 (m, 3H), 1.70 (ddt, J=16.6, 12.9, 6.6 Hz, 1H), 1.52-1.40 (m, 1H), 1.09 (dd, J=6.4, 3.4 Hz, 6H). HRMS (ESI) m/z: [M+H]⁺ Calcd for C₂₀H₂₇Cl₂N₄O, 409.1556. Found 409.1576.

### Example 72: Synthesis of (±)-(4aR,13bS)-10,11-dichloro-4-(pyridin-3-ylmethyl)-1,2,3,4,4a,5,6,13b-octahydro-8H-[1,6]naphthyridino[5,6-b]quinazolin-8-one (7bb)

Prepared according to Scheme 2.

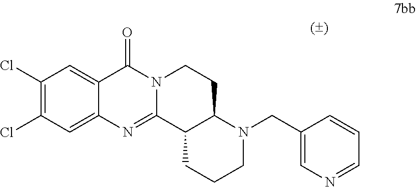 [SMILES: O=c1c2cc(Cl)c(Cl)cc2nc2n1CC[C@@H]1[C@@H]2CCCN1Cc1cccnc1]

Steps 1-5: Example 43—Synthesis of 6b.

Step 6: Synthesis of (±)-(4aR,13bS)-10,11-dichloro-4-(pyridin-3-ylmethyl)-1,2,3,4,4a,5,6,13b-octahydro-8H-[1,6]naphthyridino[5,6-b]quinazolin-8-one (7bb). Compound 7bb was prepared according to general procedure 8.1 by suspending 6b (1 eq.), 3-(bromomethyl)-pyridine hydrobromide (4 eq.), and Et₃N (6 eq.) in DMF (0.25 M) and heating to 50° C. for 20 h. Purification by flash chromatography 5-40% X/hexanes (X=2% NEt₃ 3:1 EtOAc/EtOH) afforded 7bb as a tan solid (28.1 mg, 31%). ¹H NMR (400 MHz, CDCl₃) δ 8.57 (d, J=2.2 Hz, 1H), 8.52 (dd, J=4.8, 1.6 Hz, 1H), 8.30 (s, 1H), 7.76 (s, 1H), 7.68 (dt, J=7.8, 2.0 Hz, 1H), 7.27 (dd, J=7.8, 4.8 Hz, 1H), 4.32-4.24 (m, 1H), 4.17-4.05 (m, 2H), 3.22 (d, J=14.0 Hz, 1H), 2.89 (dt, J=11.6, 3.5 Hz, 1H), 2.76 (td, J=11.1, 3.7 Hz, 1H), 2.68-2.61 (m, 1H), 2.58-2.48 (m, 1H), 2.33 (ddd, J=10.7, 9.1, 5.4 Hz, 1H), 2.07-1.90 (m, 2H), 1.79 (dt, J=13.3, 3.4 Hz, 1H), 1.66 (dddd, J=16.7, 13.0, 8.4, 3.8 Hz, 1H), 1.48 (qd, J=13.1, 3.9 Hz, 1H). HRMS (ESI) m/z: [M+H]⁺ Calcd for C₂₁H₂₁Cl₂N₄O, 415.1087.

### Example 73: Synthesis of (±)-(4aR,13bS)-10,11-dichloro-4-(2-oxopropyl)-1,2,3,4,4a,5,6,13b-octahydro-8H-[1,6]naphthyridino[5,6-b]quinazolin-8-one (7cc)

Prepared according to Scheme 2.

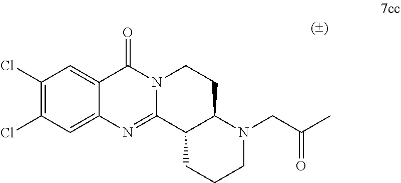 [SMILES: CC(=O)CN1CCC[C@@H]2c3nc4cc(Cl)c(Cl)cc4c(=O)n3CC[C@H]21]

Steps 1-5: Example 43—Synthesis of 6b.

Step 6: Synthesis of (±)-(4aR,13bS)-10,11-dichloro-4-(2-oxopropyl)-1,2,3,4,4a,5,6,13b-octahydro-8H-[1,6]naphthyridino[5,6-b]quinazolin-8-one (7cc). Compound 7cc was prepared according to general procedure 8.1 by suspending 6b (1 eq.), bromoacetone (4 eq.), and Et₃N (6 eq.) in DMF (0.25 M) and heating to 50° C. for 24 h. Purification by flash chromatography 3-25% X/hexanes (X=2% NEt₃ 3:1 EtOAc/EtOH) afforded 7cc as an off-white solid (202 mg, 86%). ¹H NMR (400 MHz, CDCl₃) δ 8.28 (s, 1H), 7.74 (s, 1H), 4.19-4.03 (m, 2H), 3.57 (d, J=17.3 Hz, 1H), 3.18 (d, J=17.4 Hz, 1H), 2.96-2.90 (m, 1H), 2.74-2.65 (m, 2H), 2.52 (td, J=10.1, 4.9 Hz, 1H), 2.36 (td, J=11.6, 3.5 Hz, 1H), 2.27 (dq, J=13.3, 5.4 Hz, 1H), 2.18 (s, 3H), 1.87-1.71 (m, 3H), 1.52-1.40 (m, 1H). HRMS (ESI) m/z: [M+H]⁺ Calcd for C₁₈H₂₀Cl₂N₃O₂, 380.0927.

### Example 74: Synthesis of (±)-(4aR,13bS)-10-chloro-11-methoxy-4-(oxetan-3-ylmethyl)-1,2,3,4,4a,5,6,13b-octahydro-8H-[1,6]naphthyridino[5,6-b]quinazolin-8-one (7dd)

Prepared according to Scheme 2.

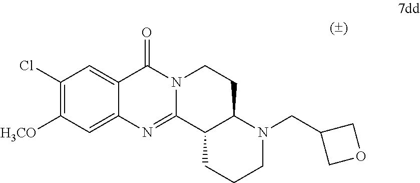 [SMILES: COc1cc2nc3n(c(=O)c2cc1Cl)CC[C@@H]1[C@@H]3CCCN1CC1COC1]

Steps 1-5: Example 44—Synthesis of 6c.

Step 6: Synthesis of (±)-(4aR,13bS)-10-chloro-11-methoxy-4-(oxetan-3-ylmethyl)-1,2,3,4,4a,5,6,13b-octahydro-8H-[1,6]naphthyridino[5,6-b]quinazolin-8-one (7dd). Compound 7dd was prepared according to general procedure 8.1 by suspending 6c (1 eq.), 3-(bromomethyl)-oxetane (4 eq.), and Et₃N (6 eq.) in DMF (0.25 M) and heating to 80° C. for 20 h. Purification by flash chromatography 20-40% X/hexanes (X=2% NEt₃ 3:1 EtOAc/EtOH) afforded 7dd as a white solid (48.1 mg, 57%). ¹H NMR (400 MHz, CDCl₃) δ 8.21 (s, 1H), 7.05 (s, 1H), 4.81 (ddd, J=9.1, 7.4, 6.0 Hz, 2H), 4.44 (t, J=6.0 Hz, 1H), 4.38 (t, J=5.9 Hz, 1H), 4.20 (ddd, J=14.5, 8.4, 6.1 Hz, 1H), 4.08 (dt, J=14.4, 6.1 Hz, 1H), 3.99 (s, 3H), 3.31-3.19 (m, 2H), 2.88 (dt, J=11.4, 3.8 Hz, 1H), 2.70-2.59 (m, 2H), 2.53-2.40 (m, 2H), 2.19 (td, J=9.9, 5.2 Hz, 1H), 2.00 (td, J=11.9, 2.8 Hz, 1H), 1.88-1.77 (m, 2H), 1.67 (qt, J=13.0, 3.6 Hz, 1H), 1.46 (dd, J=12.2, 2.8 Hz, 1H). HRMS (ESI) m/z: [M+H]⁺ Calcd for C₂₀H₂₅ClN₃O₃, 390.1579. Found 390.1589.

### Example 75: Synthesis of (±)-(4aR,13bS)-10-chloro-4-isobutyl-11-methoxy-1,2,3,4,4a,5,6,13b-octahydro-8H-[1,6]naphthyridino[5,6-b]quinazolin-8-one (7ee)

Prepared according to Scheme 2.

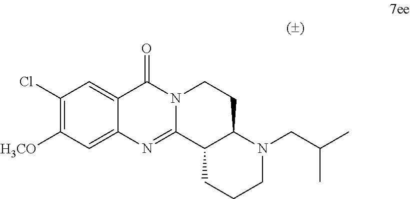 [SMILES: COc1cc2nc3n(c(=O)c2cc1Cl)CC[C@@H]1[C@@H]3CCCN1CC(C)C]

Steps 1-5: Example 44—Synthesis of 6c.

Step 6: Synthesis of (±)-(4aR,13bS)-10-chloro-4-isobutyl-11-methoxy-1,2,3,4,4a,5,6,13b-octahydro-8H-[1,6]naphthyridino[5,6-b]quinazolin-8-one (7ee). Compound 7ee was prepared according to general procedure 8.1 by suspending 6c (1 eq.), 1-bromo-2-methylpropane (4 eq.), and Et₃N (6 eq.) in DMF (0.25 M) and heating to 80° C. for 20 h. Purification by flash chromatography 0.5-10% X/hexanes (X=2% NEt₃ 3:1 EtOAc/EtOH) afforded 7ee as an off-white solid (205 mg, 70%). ¹H NMR (400 MHz, CDCl₃) δ 8.21 (s, 1H), 7.06 (s, 1H), 4.20 (ddd, J=14.4, 8.3, 6.2 Hz, 1H), 4.05 (dt, J=14.3, 6.1 Hz, 1H), 4.00 (s, 3H), 3.06 (d, J=11.5 Hz, 1H), 2.70-2.60 (m, 2H), 2.47 (dd, J=12.6, 9.2 Hz, 1H), 2.37 (dq, J=13.4, 5.9 Hz, 1H), 2.15 (td, J=9.9, 5.3 Hz, 1H), 2.02-1.87 (m, 2H), 1.87-1.61 (m, 4H), 1.47 (qd, J=13.7, 13.2, 4.3 Hz, 1H), 0.91 (dd, J=10.9, 6.5 Hz, 6H). HRMS (ESI) m/z: [M+H]⁺ Calcd for C₂₀H₂₇ClN₃O₂, 376.1786. Found 376.1784.

### Example 76: (±)-(4aR,13bS)-10-chloro-4-(2-isopropoxyethyl)-11-methoxy-1,2,3,4,4a,5,6,13b-octahydro-8H-[1,6]naphthyridino[5,6-b]quinazolin-8-one (7ff)

Prepared according to Scheme 2.

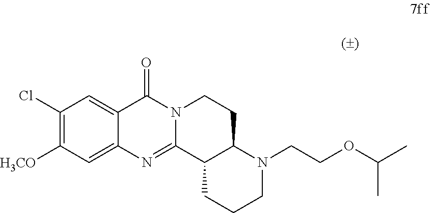 [SMILES: COc1cc2nc3n(c(=O)c2cc1Cl)CC[C@@H]1[C@@H]3CCCN1CCOC(C)C]

Steps 1-5: Example 44—Synthesis of 6c.

Step 6: Synthesis of (±)-(4aR,13bS)-10-chloro-4-(2-isopropoxyethyl)-11-methoxy-1,2,3,4,4a,5,6,13b-octahydro-8H-[1,6]naphthyridino[5,6-b]quinazolin-8-one (7ff). Compound 7ff was prepared according to general procedure 8.1 by suspending 6c (1 eq.), 1-bromo-2-methylpropane (4 eq.), and Et₃N (6 eq.) in DMF (0.25 M) and heating to 50° C. for 20 h. Purification by flash chromatography 0.5-10% X/hexanes (X=2% NEt₃ 3:1 EtOAc/EtOH) afforded 7ff as yellow solid (193 mg, 61%). ¹H NMR (400 MHz, CDCl₃) δ 8.19 (s, 1H), 7.04 (s, 1H), 4.17-4.06 (m, 2H), 3.98 (s, 3H), 3.65-3.45 (m, 3H), 3.07-3.01 (m, 1H), 3.01-2.90 (m, 1H), 2.78-2.60 (m, 3H), 2.53 (dq, J=13.2, 5.5 Hz, 1H), 2.37 (dtd, J=19.6, 11.9, 10.9, 3.9 Hz, 2H), 1.89-1.65 (m, 3H), 1.45 (qd, J=13.0, 4.2 Hz, 1H), 1.14 (dd, J=6.1, 1.0 Hz, 6H). HRMS (ESI) m/z: [M+H]⁺ Calcd for C₂₁H₂₉ClN₃O₃, 406.1892. Found 406.1886.

### Example 77: (±)-(4aR,13bS)-10-chloro-4-(cyclopropylmethyl)-11-methoxy-1,2,3,4,4a,5,6,13b-octahydro-8H-[1,6]naphthyridino[5,6-b]quinazolin-8-one (7gg)

Prepared according to Scheme 2.

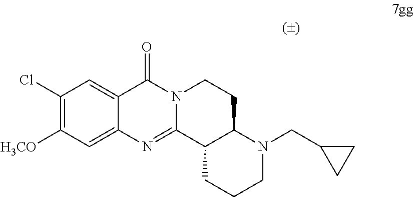 [SMILES: COc1cc2nc3n(c(=O)c2cc1Cl)CC[C@@H]1[C@@H]3CCCN1CC1CC1]

Steps 1-5: Example 44—Synthesis of 6c.

Step 6: Synthesis of (±)-(4aR,13bS)-10-chloro-4-(cyclopropylmethyl)-11-methoxy-1,2,3,4,4a,5,6,13b-octahydro-8H-[1,6]naphthyridino[5,6-b]quinazolin-8-one (7gg). Compound 7gg was prepared according to general procedure 8.1 by suspending 6c (1 eq.), (bromomethyl)-cyclopropane (4 eq.), and Et₃N (6 eq.) in DMF (0.25 M) and heating to 80° C. for 20 h. Purification by flash chromatography 2-20% X/hexanes (X=2% NEt₃ 3:1 EtOAc/EtOH) afforded 7gg as a yellow solid (358 mg, 70%). ¹H NMR (400 MHz, CDCl₃) δ 8.20 (s, 1H), 7.05 (s, 1H), 4.19 (ddd, J=14.5, 8.4, 6.1 Hz, 1H), 4.07 (dt, J=14.4, 6.1 Hz, 1H), 3.99 (s, 3H), 3.29 (dt, J=11.9, 2.8 Hz, 1H), 2.77 (dd, J=13.4, 6.1 Hz, 1H), 2.69 (td, J=12.3, 11.6, 3.7 Hz, 2H), 2.42 (dq, J=13.3, 5.9 Hz, 1H), 2.33-2.20 (m, 3H), 1.95-1.67 (m, 3H), 1.48 (qd, J=13.6, 13.1, 3.8 Hz, 1H), 0.95-0.83 (m, 1H), 0.54 (dpt, J=8.7, 6.2, 3.0 Hz, 2H), 0.12 (dhept, J=5.2, 1.7 Hz, 2H). HRMS (ESI) m/z: [M+H]⁺ Calcd for C₂₀H₂₅ClN₃O₂, 374.1611. Found 374.1622.

### Example 78: Synthesis of (±)-(4aR,13bS)-10-chloro-4-(cyclobutylmethyl)-11-methoxy-1,2,3,4,4a,5,6,13b-octahydro-8H-[1,6]naphthyridino[5,6-b]quinazolin-8-one (7hh)

Prepared according to Scheme 2.

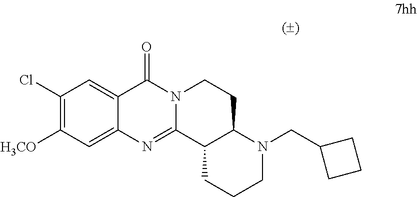 [SMILES: COc1cc2nc3n(c(=O)c2cc1Cl)CC[C@@H]1[C@@H]3CCCN1CC1CCC1]

Steps 1-5: Example 44—Synthesis of 6c.

Step 6: Synthesis of (±)-(4aR,13bS)-10-chloro-4-(cyclobutylmethyl)-11-methoxy-1,2,3,4,4a,5,6,13b-octahydro-8H-[1,6]naphthyridino[5,6-b]quinazolin-8-one (7hh). Compound 7hh was prepared according to general procedure 8.1 by suspending 6c (1 eq.), (bromomethyl)cyclobutane (4 eq.), and Et₃N (6 eq.) in DMF (0.25 M) and heating to 80° C. for 20 h. Purification by flash chromatography 0.5-15% X/hexanes (X=2% NEt₃ 3:1 EtOAc/EtOH) afforded 7hh as a yellow-white solid (217 mg, 72%). ¹H NMR (400 MHz, CDCl₃) δ 8.19 (s, 1H), 7.04 (s, 1H), 4.18 (ddd, J=14.5, 8.4, 6.1 Hz, 1H), 4.06 (dt, J=14.3, 6.0 Hz, 1H), 3.98 (s, 3H), 3.00 (dt, J=11.6, 3.6 Hz, 1H), 2.84 (dd, J=13.1, 7.0 Hz, 1H), 2.70-2.50 (m, 3H), 2.47-2.29 (m, 2H), 2.19 (td, J=9.9, 5.2 Hz, 1H), 2.06 (qdd, J=12.8, 11.7, 6.8, 3.5 Hz, 3H), 1.98-1.60 (m, 7H), 1.45 (dd, J=13.3, 3.4 Hz, 1H). HRMS (ESI) m/z: [M+H]⁺ Calcd for C₂₁H₂₇ClN₃O₂, 388.1786.

### Example 79: Synthesis of (±)-(4aR,13bS)-10-chloro-11-methoxy-4-(pyridin-3-ylmethyl)-1,2,3,4,4a,5,6,13b-octahydro-8H-[1,6]naphthyridino[5,6-b]quinazolin-8-one (7ii)

Prepared according to scheme 2.

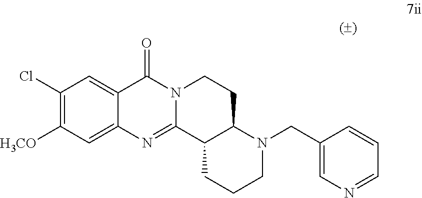 [SMILES: COc1cc2nc3n(c(=O)c2cc1Cl)CC[C@@H]1[C@@H]3CCCN1Cc1cccnc1]

Steps 1-5: Example 44—Synthesis of 6c.

Step 6: Synthesis of (±)-(4aR,13bS)-10-chloro-11-methoxy-4-(pyridin-3-ylmethyl)-1,2,3,4,4a,5,6,13b-octahydro-8H-[1,6]naphthyridino[5,6-b]quinazolin-8-one (7ii). Compound 7ii was prepared according to general procedure 8.1 by suspending 6c (1 eq.), 3-(bromomethyl)pyridine hydrobromide (1.2 eq.), and Et₃N (3 eq.) in DMF (0.25 M) and heating to 50° C. for 24 h. Purification by flash chromatography 20-40% X/hexanes (X=2% NEt₃ 3:1 EtOAc/EtOH) afforded 7ii as an off-white solid (143 mg, 37%). ¹H NMR (400 MHz, CDCl₃) δ 8.58 (d, J=2.2 Hz, 1H), 8.53 (dd, J=4.8, 1.6 Hz, 1H), 8.23 (s, 1H), 7.69 (dt, J=7.9, 2.0 Hz, 1H), 7.32-7.24 (m, 1H), 7.07 (s, 1H), 4.30 (ddd, J=14.3, 8.1, 6.3 Hz, 1H), 4.20-4.04 (m, 2H), 4.01 (s, 3H), 3.22 (d, J=13.9 Hz, 1H), 2.90 (dt, J=11.4, 3.7 Hz, 1H), 2.76 (td, J=11.1, 3.7 Hz, 1H), 2.72-2.63 (m, 1H), 2.52 (dq, J=12.3, 6.0 Hz, 1H), 2.33 (ddd, J=10.7, 9.1, 5.4 Hz, 1H), 2.08-1.87 (m, 2H), 1.86-1.75 (m, 1H), 1.67 (qt, J=12.8, 3.7 Hz, 1H), 1.52 (qd, J=13.1, 3.9 Hz, 1H). HRMS (ESI) m/z: [M+H]⁺ Calcd for C₂₂H₂₄ClN₄O₂, 411.1561. Found 411.1567.

### Example 80: Synthesis of (±)-(4aR,13bS)-11-methoxy-4-methyl-8-oxo-2,3,4,4a,5,6,8,13b-octahydro-1H-[1,6]naphthyridino[5,6-b]quinazoline-10-carbonitrile (7jj)

Prepared according to scheme 2.

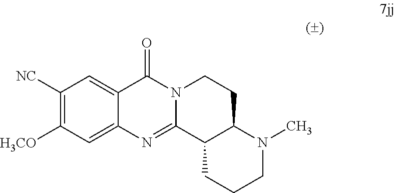 [SMILES: COc1cc2nc3n(c(=O)c2cc1C#N)CC[C@@H]1[C@@H]3CCCN1C]

Steps 1-4: See Example 40—Synthesis of (±)-trans-5gg.

Step 5: Synthesis of (±)-(4aR,13bS)-11-methoxy-4-methyl-8-oxo-2,3,4,4a,5,6,8,13b-octahydro-1H-[1,6]naphthyridino[5,6-b]quinazoline-10-carbonitrile (7jj). Compound 7jj was prepared from (±)-trans-5gg. (±)-trans-5gg (1 eq.), Pd₂(dba)₃ (10 mol %), S-Phos (20 mol %), and Zn(CN)₂ (1.2 eq.) place in a 2-5 mL MWI tube. The tube was then sealed, evacuated, and backfilled with Ar(g) two times. Then DMF/H₂O (99:1, 0.11 M) were added and the tube was heated MWI 120° C. for 1 h. After the reaction was concentrated, suspended in 20 mL MeOH. Absrobed to an agilent 10 g HSCX column, rinsed with an additional 80 mL of MeOH. The crude material was then collected with 60 mL 7N NH₃/MeOH. Purification by flash chromatography 15-60% X/hexanes (X=2% NEt₃ 3:1 EtOAc/EtOH) afforded 7jj as an off-white solid (42.7 mg, 44%). ¹H NMR (400 MHz, CDCl₃) δ 8.44 (s, 1H), 7.03 (s, 1H), 4.22-4.03 (m, 2H), 4.01 (s, 3H), 2.98 (dq, J=12.0, 3.1, 2.4 Hz, 1H), 2.74-2.63 (m, 2H), 2.43 (dq, J=13.5, 5.3 Hz, 1H), 2.36 (s, 3H), 2.11 (td, J=12.0, 3.1 Hz, 1H), 1.99 (td, J=10.2, 4.7 Hz, 1H), 1.92-1.73 (m, 3H), 1.45 (qd, J=12.7, 3.2 Hz, 1H). HRMS (ESI) m/z: [M+H]⁺ Calcd for C₁₈H₂₁ClN₄O₂, 325.1659. Found 325.1655.

### Example 81: (±)-(4aR,13bS)-11-methoxy-4-(oxetan-3-ylmethyl)-8-oxo-2,3,4,4a,5,6,8,13b-octahydro-1H-[1,6]naphthyridino[5,6-b]quinazoline-10-carbonitrile (7kk)

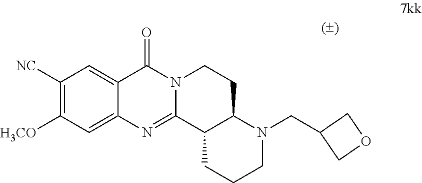 [SMILES: COc1cc2nc3n(c(=O)c2cc1C#N)CC[C@@H]1[C@@H]3CCCN1CC1COC1]

Steps 1-6: See Example 38—Synthesis of 5ee.

Step 7: Synthesis of (±)-(4aR,13bS)-11-methoxy-4-(oxetan-3-ylmethyl)-8-oxo-2,3,4,4a,5,6,8,13b-octahydro-1H-[1,6]naphthyridino[5,6-b]quinazoline-10-carbonitrile (7kk). Compound 7kk was prepared from 5ee. 5ee (1 eq.), Pd₂(dba)₃ (10 mol %), S-Phos (20 mol %), and Zn(CN)₂ (1.2 eq.) place in a 2-5 mL MWI tube. The tube was then sealed, evacuated, and backfilled with Ar(g) two times. Then DMF/H₂O (99:1, 0.11 M) were added and the tube was heated MWI 120° C. for 1.5 h. After the reaction was concentrated, suspended in 20 mL MeOH. Absorbed to an agilent 10 g HSCX column, rinsed with an additional 80 mL of MeOH. The crude material was then collected with 60 mL 7N NH₃/MeOH. Purification by flash chromatography 0.5-10% X/hexanes (X=2% NEt₃ 3:1 EtOAc/EtOH) afforded 7kk as a yellow-white solid (72.9 mg, 50%). ¹H NMR (400 MHz, CDCl₃) δ 8.43 (s, 1H), 7.03 (s, 1H), 4.81 (dt, J=9.5, 6.5 Hz, 2H), 4.43 (t, J=5.8 Hz, 1H), 4.37 (t, J=5.8 Hz, 1H), 4.18 (ddd, J=14.5, 8.6, 6.2 Hz, 1H), 4.13-4.05 (m, 1H), 4.01 (s, 3H), 3.29-3.18 (m, 2H), 2.88 (d, J=11.3 Hz, 1H), 2.72-2.61 (m, 2H), 2.55-2.41 (m, 2H), 2.22 (q, J=8.9 Hz, 1H), 2.01 (t, J=11.8 Hz, 1H), 1.91-1.78 (m, 2H), 1.67 (q, J=13.7 Hz, 1H), 1.46 (qd, J=15.5, 14.4, 5.4 Hz, 1H). HRMS (ESI) m/z: [M+H]⁺ Calcd for C₂₁H₂₅ClN₄O₃, 381.1921. Found 381.1911.

### Example 82: Synthesis of (±)-(4aR,13bS)-10-chloro-4-(2-hydroxyethyl)-11-methoxy-1,2,3,4,4a,5,6,13b-octahydro-8H-[1,6]naphthyridino[5,6-b]quinazolin-8-one (7ll)

Prepared according to Scheme 2.

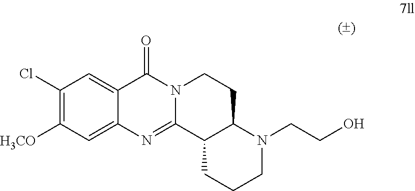 [SMILES: COc1cc2nc3n(c(=O)c2cc1Cl)CC[C@@H]1[C@@H]3CCCN1CCO]

Steps 1-5: See Example 44—Synthesis of 6c.

Step 6: Synthesis of (±)-(4aR,13bS)-10-chloro-4-(2-hydroxyethyl)-11-methoxy-1,2,3,4,4a,5,6,13b-octahydro-8H-[1,6]naphthyridino[5,6-b]quinazolin-8-one (7ll). Compound 7ll was prepared according to general procedure 8.1 by suspending 6c (1 eq.), 2-bromoethanol (4 eq.), and Et₃N (6 eq.) in DMF (0.25 M) and heating to 50° C. for 24 h. Purification by flash chromatography 0.5-10% X/hexanes (X=2% NEt₃ 3:1 EtOAc/EtOH) afforded 7ll as a tan solid (484 mg, 73%). ¹H NMR (400 MHz, CDCl₃) δ 8.19 (s, 1H), 7.04 (s, 1H), 4.20 (ddd, J=14.3, 8.2, 5.9 Hz, 1H), 4.05 (dt, J=14.4, 5.9 Hz, 1H), 3.99 (s, 3H), 3.73 (ddd, J=10.9, 9.2, 3.8 Hz, 1H), 3.59 (dt, J=10.9, 4.4 Hz, 1H), 3.12 (ddt, J=18.2, 9.2, 4.2 Hz, 2H), 2.71-2.60 (m, 3H), 2.46-2.33 (m, 2H), 2.29 (dt, J=13.2, 3.7 Hz, 1H), 2.15 (td, J=12.1, 2.8 Hz, 1H), 1.92-1.77 (m, 2H), 1.70 (qt, J=13.0, 3.7 Hz, 1H), 1.51 (qd, J=12.7, 3.5 Hz, 1H). HRMS (ESI) m/z: [M+H]⁺ Calcd for C₁₈H₂₃ClN₃O₃, 364.1422. Found 364.1414.

### Example 83: Synthesis of (±)-(4aR,13bS)-10-chloro-11-methoxy-4-(2-morpholinoethyl)-1,2,3,4,4a,5,6,13b-octahydro-8H-[1,6]naphthyridino[5,6-b]quinazolin-8-one (7 mm)

Prepared according to Scheme 2.

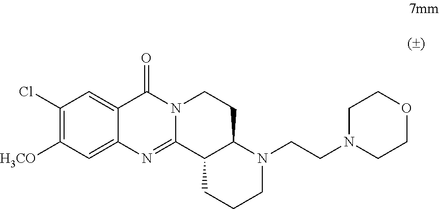 [SMILES: COc1cc2nc3n(c(=O)c2cc1Cl)CC[C@@H]1[C@@H]3CCCN1CCN1CCOCC1]

Steps 1-6: See Example 82—Synthesis of 7ll.

Step 7: Synthesis of (±)-(4aR,13bS)-10-chloro-11-methoxy-4-(2-morpholinoethyl)-1,2,3,4,4a,5,6,13b-octahydro-8H-[1,6]naphthyridino[5,6-b]quinazolin-8-one (7 mm). Compound 7 mm was prepared according to procedure 8.3 by suspending 7ll (1 eq.) and Et₃N (2 eq.) in anhydrous DCE (0.25 M) and cooled to 0° C. At 0° C., MsCl (1.2 eq.) was added dropwise. Then allowed to warm to room temperature and stir for 2 h. After 2 h, morpholine (8 eq.) was added dropwise. The reaction was allowed to stir at room temperature for 23 h and then heated to reflux for 7 h. Purification by flash chromatography 50-75% X/hexanes (X=2% NEt₃ 3:1 EtOAc/EtOH) afforded 7 mm as a tan solid (143 mg, 40%). ¹H NMR (400 MHz, CDCl₃) δ 8.19 (s, 1H), 7.03 (s, 1H), 4.17 (ddd, J=14.4, 8.4, 6.0 Hz, 1H), 4.07 (dt, J=14.3, 6.0 Hz, 1H), 3.98 (s, 3H), 3.69 (q, J=3.9, 3.2 Hz, 4H), 3.08 (dt, J=11.7, 3.4 Hz, 1H), 3.02-2.92 (m, 1H), 2.70-2.60 (m, 2H), 2.55-2.39 (m, 8H), 2.25 (dtd, J=27.4, 11.9, 10.8, 4.0 Hz, 2H), 1.89-1.79 (m, 2H), 1.71 (qt, J=13.1, 3.7 Hz, 1H), 1.46 (qd, J=13.4, 3.9 Hz, 1H). HRMS (ESI) m/z: [M+H]⁺ Calcd for C₂₂H₃₀ClN₄O₃, 433.2001. Found 433.1998.

### Example 84: Synthesis of (±)-(4aR,13bS)-10-chloro-11-methoxy-4-(2-(pyrrolidin-1-yl)ethyl)-1,2,3,4,4a,5,6,13b-octahydro-8H-[1,6]naphthyridino[5,6-b]quinazolin-8-one (7nn)

Prepared according to Scheme 2.

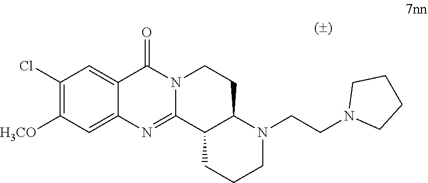 [SMILES: COc1cc2nc3n(c(=O)c2cc1Cl)CC[C@@H]1[C@@H]3CCCN1CCN1CCCC1]

Steps 1-6: See Example 82—Synthesis of 7ll.

Step 7: Synthesis of (±)-(4aR,13bS)-10-chloro-11-methoxy-4-(2-(pyrrolidin-1-yl)ethyl)-1,2,3,4,4a,5,6,13b-octahydro-8H-[1,6]naphthyridino[5,6-b]quinazolin-8-one (7nn). Compound 7nn was prepared according to general procedure 8.3 by suspending 7ll (1 eq.) and Et₃N (2 eq.) in anhydrous DCE (0.25 M) and cooled to 0° C. At 0° C., MsCl (1.2 eq.) was added dropwise, and then the reaction was allowed to warm to room temperature and stirred for 2 h. After 2 h, pyrrolidine (8 eq.) was added dropwise. The reaction was allowed to stir at room temperature for 23 h and then heated to reflux for 7 h. Purification by flash chromatography 50-80% X/hexanes (X=2% NEt₃ 3:1 EtOAc/EtOH) afforded 7nn as a yellow-orange solid (24.7 mg, 17%). ¹H NMR (400 MHz, CDCl₃) δ 8.19 (s, 1H), 7.03 (s, 1H), 4.22-4.02 (m, 2H), 3.98 (s, 3H), 3.12-2.97 (m, 2H), 2.73-2.40 (m, 10H), 2.32-2.17 (m, 2H), 1.91-1.63 (m, 7H), 1.46 (qd, J=13.2, 4.1 Hz, 1H). FIRMS (ESI) m/z: [M+H]⁺ Calcd for C₂₂H₃₉ClN₄O₂, 417.2052. Found 417.2053.

### Example 85: Synthesis of (±)-(4aR,13bS)-10,11-dichloro-4-(2-(dimethylamino)ethyl)-1,2,3,4,4a,5,6,13b-octahydro-8H-[1,6]naphthyridino[5,6-b]quinazolin-8-one (700)

Prepared according to Scheme 2.

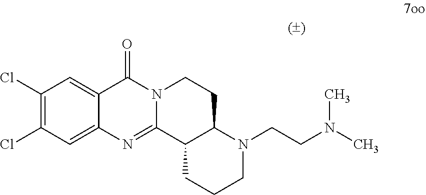 [SMILES: CN(C)CCN1CCC[C@@H]2c3nc4cc(Cl)c(Cl)cc4c(=O)n3CC[C@H]21]

Steps 1-5: See Example 43—Synthesis of 6b.

Step 6: Synthesis of (±)-(4aR,13bS)-10,11-dichloro-4-(2-(dimethylamino)ethyl)-1,2,3,4,4a,5,6,13b-octahydro-8H-[1,6]naphthyridino[5,6-b]quinazolin-8-one (700). Compound 7oo was prepared according to general procedure 8.3 by suspending 7j (1 eq.) and Et₃N (2 eq.) in anhydrous DCE (0.25 M) and cooled to 0° C. At 0° C., MsCl (1.2 eq.) was added dropwise. Then allowed to warm to room temperature and stir for 2 h. After 2 h, dimethylamine (2 M in THF, 6 eq.) was added dropwise, and then the reaction was allowed to warm to room temperature and stirred at room temperature for 22 h. Purification by flash chromatography 3-25% X/hexanes (X=2% NEt₃ 3:1 EtOAc/EtOH) afforded 7oo as an orange solid (33.2 mg, 44%). ¹H NMR (400 MHz, CDCl₃) δ 8.27 (s, 1H), 7.74 (s, 1H), 4.24-4.04 (m, 2H), 3.10 (ddd, J=12.1, 5.8, 3.4 Hz, 2H), 2.72-2.53 (m, 4H), 2.50-2.41 (m, 2H), 2.38 (s, 6H), 2.27 (td, J=10.0, 5.1 Hz, 1H), 2.17 (td, J=12.0, 2.9 Hz, 1H), 1.87 (ddq, J=17.9, 6.9, 4.7, 3.1 Hz, 2H), 1.72 (qt, J=13.0, 3.8 Hz, 1H), 1.50-1.37 (m, 1H). HRMS (ESI) m/z: [M+H]⁺ Calcd for C₁₉H₂₅Cl₂N₄O, 395.1400. Found 395.1415.

### Example 86: Synthesis of (4aR,13bS)-10-chloro-4-methyl-1,2,3,4,4a,5,6,13b-octahydro-8H-[1,6]naphthyridino[5,6-b]quinazolin-8-one (10a) and (4aS,13bR)-10-chloro-4-methyl-1,2,3,4,4a,5,6,13b-octahydro-8H-[1,6]naphthyridino[5,6-b]quinazolin-8-one (10b)

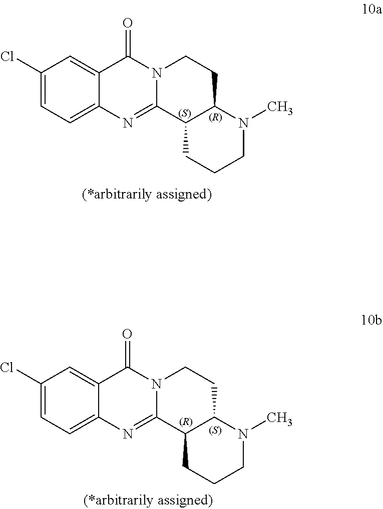

10a and 10b was prepared from the chiral separation of trans-5d (Example 4) on a Regis 25 cm×21.1 mm Reflect I-Cellulose 5 μM (3,5-dimethylphenylcarbamate) column 2-15% IPA/Hexanes. Chiral separation afforded 10a (14 mg, white solid, 99% ee) and 10b (14 mg, white solid, 99% ee).

(4aR,13bS)-10-chloro-4-methyl-1,2,3,4,4a,5,6,13b-octahydro-8H-[1,6]naphthyridino[5,6-b]quinazolin-8-one (10a). ¹H NMR (400 MHz, CDCl₃) δ 8.21 (d, J=2.4 Hz, 1H), 7.63 (dd, J=8.8, 2.4 Hz, 1H), 7.57 (d, J=8.7 Hz, 1H), 4.21-4.09 (m, 2H), 2.98 (ddt, J=11.6, 4.1, 1.9 Hz, 1H), 2.71 (ddd, J=12.5, 7.4, 3.8 Hz, 2H), 2.43 (dq, J=13.3, 5.5 Hz, 1H), 2.37 (s, 3H), 2.12 (td, J=12.0, 3.2 Hz, 1H), 1.98 (td, J=10.3, 5.0 Hz, 1H), 1.88-1.73 (m, 3H), 1.53-1.41 (m, 1H). HRMS (ESI) m/z: [M+H]⁺ Calcd for C₁₆H₁₉ClN₃O, 304.1211. Found 304.1209.

(4aS,13bR)-10-chloro-4-methyl-1,2,3,4,4a,5,6,13b-octahydro-8H-[1,6]naphthyridino[5,6-b]quinazolin-8-one (10b). ¹H NMR (400 MHz, CDCl₃) δ 8.21 (d, J=2.4 Hz, 1H), 7.63 (dd, J=8.8, 2.4 Hz, 1H), 7.57 (d, J=8.7 Hz, 1H), 4.21-4.09 (m, 2H), 2.98 (ddt, J=11.6, 4.1, 1.9 Hz, 1H), 2.71 (ddd, J=12.5, 7.4, 3.8 Hz, 2H), 2.43 (dq, J=13.3, 5.5 Hz, 1H), 2.37 (s, 3H), 2.12 (td, J=12.0, 3.2 Hz, 1H), 1.98 (dt, J=10.3, 5.0 Hz, 1H), 1.88-1.73 (m, 3H), 1.47 (tdd, J=12.8, 11.2, 4.0 Hz, 1H). HRMS (ESI) m/z: [M+H]⁺ Calcd for C₁₆H₁₉ClN₃O, 304.1211. Found 304.1209.

### Example 87: Synthesis of (4aR,13bS)-4-methyl-10-(trifluoromethyl)-1,2,3,4,4a,5,6,13b-octahydro-8H-[1,6]naphthyridino[5,6-b]quinazolin-8-one (10c) and (4aS,13bR)-4-methyl-10-(trifluoromethyl)-1,2,3,4,4a,5,6,13b-octahydro-8H-[1,6]naphthyridino[5,6-b]quinazolin-8-one (10d)

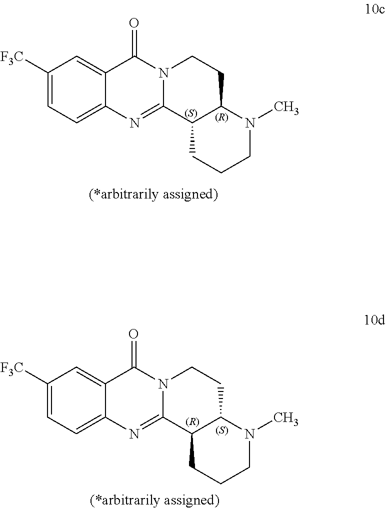

10c and 10d was prepared from the chiral separation of trans-5j (Example 10) on a Regis 25 cm×21.1 mm Reflect I-Cellulose 5 μM (3,5-dimethylphenylcarbamate) column 2-15% IPA/Hexanes. Chiral separation afforded 10c (22 mg, tan-white solid, 97% ee) and 10d (20 mg, tan-white solid, 98% ee).

(4aR,13bS)-4-methyl-10-(trifluoromethyl)-1,2,3,4,4a,5,6,13b-octahydro-8H-[1,6]naphthyridino[5,6-b]quinazolin-8-one (10c). ¹H NMR (400 MHz, CDCl₃) δ 8.53 (d, J=2.1 Hz, 1H), 7.88 (dd, J=8.6, 2.2 Hz, 1H), 7.72 (d, J=8.6 Hz, 1H), 4.23-4.10 (m, 2H), 2.98 (dq, J=11.5, 2.2 Hz, 1H), 2.79-2.66 (m, 2H), 2.45 (dq, J=13.4, 5.4 Hz, 1H), 2.37 (s, 3H), 2.12 (td, J=12.0, 3.1 Hz, 1H), 2.00 (td, J=10.1, 4.7 Hz, 1H), 1.91-1.74 (m, 3H), 1.48 (qd, J=12.6, 3.7, 1.6 Hz, 1H). HRMS (ESI) m/z: [M+H]⁺ Calcd for C₁₇H₁₉F₃N₃O, 338.1375. Found 338.1471.

(4aS,13bR)-4-methyl-10-(trifluoromethyl)-1,2,3,4,4a,5,6,13b-octahydro-8H-[1,6]naphthyridino[5,6-b]quinazolin-8-one (10d). ¹H NMR (400 MHz, CDCl₃) δ 8.53 (d, J=2.2 Hz, 1H), 7.88 (dd, J=8.7, 2.2 Hz, 1H), 7.72 (d, J=8.6 Hz, 1H), 4.26-4.09 (m, 2H), 2.98 (dt, J=11.9, 3.3 Hz, 1H), 2.80-2.66 (m, 2H), 2.37 (s, 4H), 2.12 (td, J=11.9, 3.1 Hz, 1H), 2.00 (td, J=10.2, 4.8 Hz, 1H), 1.91-1.76 (m, 3H), 1.48 (qd, J=13.0, 12.4, 4.1 Hz, 1H). HRMS (ESI) m/z: [M+H]⁺ Calcd for C₁₇H₁₉F₃N₃O, 338.1375. Found 338.1471.

### Example 88: Synthesis of (4aR,13bS)-10,11-dichloro-4-methyl-1,2,3,4,4a,5,6,13b-octahydro-8H-[1,6]naphthyridino[5,6-b]quinazolin-8-one (10e) and (4aS,13bR)-10,11-dichloro-4-methyl-1,2,3,4,4a,5,6,13b-octahydro-8H-[1,6]naphthyridino[5,6-b]quinazolin-8-one (10f)

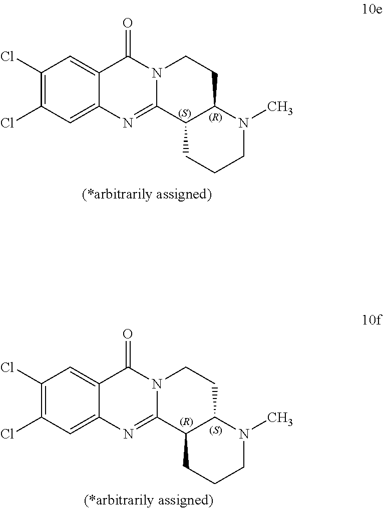

Compounds 10e and 10f were prepared from the chiral separation of trans-5q (Example 18) on a Regis 25 cm×21.1 mm Reflect I-Cellulose 5 μM (3,5-dimethylphenylcarbamate) column 2-15% IPA/Hexanes. Chiral separation afforded 10e (28 mg, tan-white solid, 99% ee) and 10f (19 mg, tan-white solid, 99% ee).

(4aR,13bS)-10,11-dichloro-4-methyl-1,2,3,4,4a,5,6,13b-octahydro-8H-[1,6]naphthyridino[5,6-b]quinazolin-8-one (10e). ¹H NMR (400 MHz, CDCl₃) δ 8.26 (s, 1H), 7.73 (s, 1H), 4.12 (t, J=6.1 Hz, 2H), 2.97 (d, J=11.4 Hz, 1H), 2.72-2.63 (m, 2H), 2.46-2.32 (m, 4H), 2.10 (td, J=12.0, 3.1 Hz, 1H), 1.97 (td, J=10.0, 4.8 Hz, 1H), 1.90-1.70 (m, 3H), 1.43 (qd, J=13.2, 12.6, 3.8 Hz, 1H). HRMS (ESI) m/z: [M+H]⁺ Calcd for C₁₆H₁₈Cl₂N₃O, 338.0821. Found 338.0817.

(4aS,13bR)-10,11-dichloro-4-methyl-1,2,3,4,4a,5,6,13b-octahydro-8H-[1,6]naphthyridino[5,6-b]quinazolin-8-one (10f). ¹H NMR (400 MHz, CDCl₃) δ 8.28 (s, 1H), 7.74 (s, 1H), 4.13 (t, J=6.4 Hz, 2H), 2.98 (d, J=11.6 Hz, 1H), 2.76-2.64 (m, 2H), 2.50-2.35 (m, 4H), 2.12 (td, J=11.9, 3.2 Hz, 1H), 1.99 (td, J=10.0, 4.9 Hz, 1H), 1.92-1.74 (m, 3H), 1.44 (qd, J=12.9, 4.4 Hz, 1H). HRMS (ESI) m/z: [M+H]⁺ Calcd for C₁₆H₁₈Cl₂N₃O, 338.0821. Found 338.0817.

### Example 89: Synthesis of (4aR,13bS)-10-chloro-4-isobutyl-11-methoxy-1,2,3,4,4a,5,6,13b-octahydro-8H-[1,6]naphthyridino[5,6-b]quinazolin-8-one (10g) and (4aS,13bR)-10-chloro-4-isobutyl-11-methoxy-1,2,3,4,4a,5,6,13b-octahydro-8H-[1,6]naphthyridino[5,6-b]quinazolin-8-one (10h)

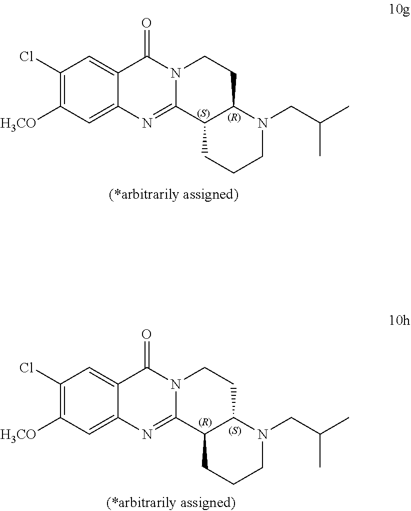

Compounds 10g and 10h were prepared from the chiral separation of trans-7ee (Example 75) on a Regis 25 cm×21.1 mm R,R-Welk-O 5 μM Kromasil column 5% B/A (B=2% IPA in DCM; A=25% B in Hexanes. Chiral separation afforded 10g (42 mg, grey-white solid, 99% ee) and 10h (34 mg, grey-white solid, 99% ee).

(4aR,13bS)-10-chloro-4-isobutyl-11-methoxy-1,2,3,4,4a,5,6,13b-octahydro-8H-[1,6]naphthyridino[5,6-b]quinazolin-8-one (10g). ¹H NMR (400 MHz, CDCl₃) δ 8.21 (s, 1H), 7.06 (s, 1H), 4.20 (ddd, J=14.4, 8.3, 6.2 Hz, 1H), 4.05 (dt, J=14.3, 6.1 Hz, 1H), 4.00 (s, 3H), 3.06 (d, J=11.5 Hz, 1H), 2.70-2.60 (m, 2H), 2.47 (dd, J=12.6, 9.2 Hz, 1H), 2.37 (dq, J=13.4, 5.9 Hz, 1H), 2.15 (td, J=9.9, 5.3 Hz, 1H), 2.02-1.87 (m, 2H), 1.87-1.61 (m, 4H), 1.47 (qd, J=13.7, 13.2, 4.3 Hz, 1H), 0.91 (dd, J=10.9, 6.5 Hz, 6H). HRMS (ESI) m/z: [M+H]⁺ Calcd for C₂₀H₂₇ClN₃O₂, 376.1786. Found 376.1784.

(4aS,13bR)-10-chloro-4-isobutyl-11-methoxy-1,2,3,4,4a,5,6,13b-octahydro-8H-[1,6]naphthyridino[5,6-b]quinazolin-8-one (10h). ¹H NMR (400 MHz, CDCl₃) δ 8.21 (s, 1H), 7.06 (s, 1H), 4.20 (ddd, J=14.4, 8.3, 6.2 Hz, 1H), 4.05 (dt, J=14.3, 6.1 Hz, 1H), 4.00 (s, 3H), 3.06 (d, J=11.5 Hz, 1H), 2.70-2.60 (m, 2H), 2.47 (dd, J=12.6, 9.2 Hz, 1H), 2.37 (dq, J=13.4, 5.9 Hz, 1H), 2.15 (td, J=9.9, 5.3 Hz, 1H), 2.02-1.87 (m, 2H), 1.87-1.61 (m, 4H), 1.47 (qd, J=13.7, 13.2, 4.3 Hz, 1H), 0.91 (dd, J=10.9, 6.5 Hz, 6H). HRMS (ESI) m/z: [M+H]⁺ Calcd for C₂₀H₂₇ClN₃O₂, 376.1786. Found 376.1784.

### Example 90. Synthesis of (4aR,13bS)-10-chloro-11-methoxy-4-(oxetan-3-ylmethyl)-1,2,3,4,4a,5,6,13b-octahydro-8H-[1,6]naphthyridino[5,6-b]quinazolin-8-one (10i) and (4aS,13bR)-10-chloro-11-methoxy-4-(oxetan-3-ylmethyl)-1,2,3,4,4a,5,6,13b-octahydro-8H-[1,6]naphthyridino[5,6-b]quinazolin-8-one (10j)

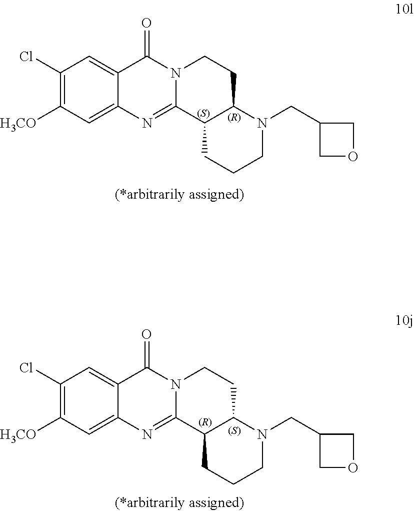

Compounds 10i and 10j were prepared from the chiral separation of trans-7dd (example 74) on a Chiral Tech Chiracel OJ-H, 250 mm×20 mm 25% EtOH with 0.1% dimethylamine in CO₂. Chiral separation afforded 10i (245 mg, yellow solid, 99% ee) and 10h (236 mg, yellow solid, 99% ee).

(4aR,13bS)-10-chloro-11-methoxy-4-(oxetan-3-ylmethyl)-1,2,3,4,4a,5,6,13b-octahydro-8H-[1,6]naphthyridino[5,6-b]quinazolin-8-one (10i). ¹H NMR (400 MHz, CDCl₃) δ 8.21 (s, 1H), 7.05 (s, 1H), 4.81 (ddd, J=9.1, 7.4, 6.0 Hz, 2H), 4.44 (t, J=6.0 Hz, 1H), 4.38 (t, J=5.9 Hz, 1H), 4.20 (ddd, J=14.5, 8.4, 6.1 Hz, 1H), 4.08 (dt, J=14.4, 6.1 Hz, 1H), 3.99 (s, 3H), 3.31-3.19 (m, 2H), 2.88 (dt, J=11.4, 3.8 Hz, 1H), 2.70-2.59 (m, 2H), 2.53-2.40 (m, 2H), 2.19 (td, J=9.9, 5.2 Hz, 1H), 2.00 (td, J=11.9, 2.8 Hz, 1H), 1.88-1.77 (m, 2H), 1.67 (qt, J=13.0, 3.6 Hz, 1H), 1.46 (dd, J=12.2, 2.8 Hz, 1H). HRMS (ESI) m/z: [M+H]⁺ Calcd for C₂₀H₂₅ClN₃O₃, 390.1579. Found 390.1589.

(4aS,13bR)-10-chloro-11-methoxy-4-(oxetan-3-ylmethyl)-1,2,3,4,4a,5,6,13b-octahydro-8H-[1,6]naphthyridino[5,6-b]quinazolin-8-one (10j). ¹H NMR (400 MHz, CDCl₃) δ 8.21 (s, 1H), 7.05 (s, 1H), 4.81 (ddd, J=9.1, 7.4, 6.0 Hz, 2H), 4.44 (t, J=6.0 Hz, 1H), 4.38 (t, J=5.9 Hz, 1H), 4.20 (ddd, J=14.5, 8.4, 6.1 Hz, 1H), 4.08 (dt, J=14.4, 6.1 Hz, 1H), 3.99 (s, 3H), 3.31-3.19 (m, 2H), 2.88 (dt, J=11.4, 3.8 Hz, 1H), 2.70-2.59 (m, 2H), 2.53-2.40 (m, 2H), 2.19 (td, J=9.9, 5.2 Hz, 1H), 2.00 (td, J=11.9, 2.8 Hz, 1H), 1.88-1.77 (m, 2H), 1.67 (qt, J=13.0, 3.6 Hz, 1H), 1.46 (dd, J=12.2, 2.8 Hz, 1H). HRMS (ESI) m/z: [M+H]⁺ Calcd for C₂₀H₂₅ClN₃O₃, 390.1579. Found 390.1589.

### Example 91: Synthesis of (S)-6,7-dichloro-2-(1-isopropylpiperidin-3-yl)-3-methylquinazolin-4(3H)-one (14a)

Prepared according to scheme 4.

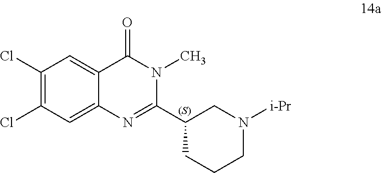

Step 1: Synthesis of 2-amino-4,5-dichloro-N-methylbenzamide (11a). Compound 11a was prepared according to general procedure 9a from 2-amino, 4,5-dichloro-benzoic acid and methylammonium chloride. After work-up, 11a was afforded as a light yellow solid (1.41 g, 88%). ¹H NMR (400 MHz, DMSO) δ 8.36 (q, J=4.4 Hz, 1H), 7.68 (s, 1H), 6.93 (s, 1H), 6.73 (s, 2H), 2.71 (d, J=4.5 Hz, 3H). HRMS (ESI) m/z: [M+H]⁺ Calcd for C₈H₉Cl₂N₂O, 219.1009. Found 219.0082.

Step 2: Synthesis of tert-butyl (S)-3-(6,7-dichloro-3-methyl-4-oxo-3,4-dihydroquinazolin-2-yl)piperidine-1-carboxylate (12a). Compound 12a was prepared according to general procedure 10 from (S)-1-(tert-butoxycarbonyl)piperidine-3-carboxylic acid and 11a. Purification by flash chromatography (5-25% X/hexanes, X=4:1 EtOAc/DCM) afforded 12a as an off-white solid (0.77 g, 88%). ¹H NMR (400 MHz, CDCl₃) δ 8.28 (s, 1H), 7.73 (s, 1H), 4.22 (d, J=49.5 Hz, 2H), 3.67 (s, 3H), 3.12-2.98 (m, 1H), 2.90 (tt, J=10.7, 3.3 Hz, 1H), 2.84-2.76 (m, 1H), 2.13-2.03 (m, 1H), 1.97-1.88 (m, 1H), 1.88-1.79 (m, 1H), 1.59 (t, J=12.5 Hz, 1H), 1.46 (s, 9H). HRMS (ESI) m/z: [M+H]⁺ Calcd for C₁₉H₂₄Cl₂N₃O₃, 412.1189. Found 412.1183.

Step 3: Synthesis of (S)-6,7-dichloro-3-methyl-2-(piperidin-3-yl)quinazolin-4(3H)-one trifluoroacetic acid salt (13a). Compound 13a was prepared according to general procedure 11 from 12a. Isolation by vacuum filtration afforded 13a as a white solid (0.26 g, 66%). 13a was used directly without analysis.

Step 4: Synthesis of (S)-6,7-dichloro-2-(1-isopropylpiperidin-3-yl)-3-methylquinazolin-4(3H)-one (14a) (14a). Compound 14a was prepared according to general procedure 12 from 13a and isopropyl iodide. Purification by flash chromatography (3-25% X/hexanes; X=2% NEt₃ 3:1 EtOAc/EtOH) afforded 14a as a tan solid (64 mg, 97%, 98.2% ee). ¹H NMR (400 MHz, CDCl₃) δ 8.28 (d, J=1.7 Hz, 1H), 7.75 (d, J=1.2 Hz, 1H), 3.64 (s, 3H), 3.11-2.99 (m, 2H), 2.95-2.88 (m, 1H), 2.82 (h, J=6.6 Hz, 1H), 2.51 (t, J=10.6 Hz, 1H), 2.23 (td, J=11.5, 3.0 Hz, 1H), 2.03-1.95 (m, 1H), 1.86 (dq, J=11.8, 3.0, 2.4 Hz, 1H), 1.78-1.57 (m, 2H), 1.07 (d, J=6.5 Hz, 6H). HRMS (ESI) m/z: [M+H]⁺ Calcd for C₁₇H₂₂Cl₂N₃O, 354.1134. Found 354.1129.

### Example 92: Synthesis of (R)-6,7-dichloro-2-(1-isopropylpiperidin-3-yl)-3-methylquinazolin-4(3H)-one (14b)

Prepared according to Scheme 4.

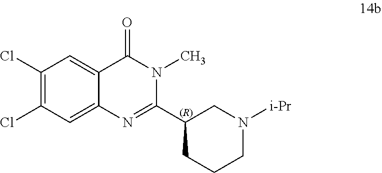

Step 1: See Example 91—synthesis of 11a.

Step 2: Synthesis of tert-butyl (R)-3-(6,7-dichloro-3-methyl-4-oxo-3,4-dihydroquinazolin-2-yl)piperidine-1-carboxylate (12b). Compound 12b was prepared according to general procedure 10 from (R)-1-(tert-butoxycarbonyl)piperidine-3-carboxylic acid and 11a. Purification by flash chromatography (5-25% X/hexanes, X=4:1 EtOAc/DCM) afforded 12b as an off-white solid (0.46 g, 49%). ¹H NMR (400 MHz, CDCl₃) δ 8.28 (s, 1H), 7.73 (s, 1H), 4.22 (d, J=47.7 Hz, 2H), 3.67 (s, 3H), 3.14-2.97 (m, 1H), 2.90 (tt, J=10.7, 3.3 Hz, 1H), 2.86-2.74 (m, 1H), 2.12-2.05 (m, 1H), 1.98-1.88 (m, 1H), 1.88-1.79 (m, 1H), 1.59 (t, J=12.5 Hz, 1H), 1.46 (s, 9H). HRMS (ESI) m/z: [M+H]⁺ Calcd for C₁₉H₂₄Cl₂N₃O₃, 412.1189. Found 412.1185.

Step 3: Synthesis of (R)-6,7-dichloro-3-methyl-2-(piperidin-3-yl)quinazolin-4(3H)-one trifluoroacetic acid salt (13b). Compound 13b was prepared according to general procedure 11 from 12a. Isolation by vacuum filtration afforded 13b as a white solid (0.33 g, 66%). 13b was used directly without analysis.

Step 4: Synthesis of (R)-6,7-dichloro-2-(1-isopropylpiperidin-3-yl)-3-methylquinazolin-4(3H)-one (14b). Compound 14b was prepared according to general procedure 12 from 13b and isopropyl iodide. Purification by flash chromatography (3-25% X/hexanes; X=2% NEt₃ 3:1 EtOAc/EtOH) afforded 14b as a tan solid (64 mg, 98%, 96.5% ee). ¹H NMR (400 MHz, CDCl₃) δ 8.27 (d, J=2.0 Hz, 1H), 7.75 (d, J=1.4 Hz, 1H), 3.64 (s, 3H), 3.10-2.97 (m, 2H), 2.91 (d, J=11.0 Hz, 1H), 2.80 (h, J=6.6 Hz, 1H), 2.51 (t, J=10.6 Hz, 1H), 2.23 (td, J=11.5, 2.9 Hz, 1H), 2.03-1.94 (m, 1H), 1.86 (dq, J=11.8, 2.9, 2.4 Hz, 1H), 1.78-1.57 (m, 2H), 1.07 (d, J=6.5 Hz, 6H). HRMS (ESI) m/z: [M+H]⁺ Calcd for C₁₇H₂₂Cl₂N₃O, 354.1134. Found 354.1128.

### Example 93: Synthesis of (S)-6,7-dichloro-3-methyl-2-(1-((tetrahydro-2H-pyran-4-yl)methyl)piperidin-3-yl)quinazolin-4(3H)-one (14c)

Prepared according to Scheme 4.

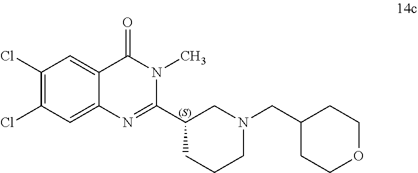

Steps 1-3: See Example 91—Synthesis of 13a.

Step 4: Synthesis of (S)-6,7-dichloro-3-methyl-2-(1-((tetrahydro-2H-pyran-4-yl)methyl)piperidin-3-yl)quinazolin-4(3H)-one (14c). Compound 14c was prepared according to general procedure 12 from 13a and 4-bromomethyl-tetrahydropyran. Purification by flash chromatography (3-30% X/hexanes; X=2% NEt₃ 3:1 EtOAc/EtOH) afforded 14a as a tan, orange solid (43 mg, 87%, 97.4% ee). ¹H NMR (400 MHz, CDCl₃) δ 8.29 (s, 1H), 7.74 (s, 1H), 3.96 (ddd, J=11.5, 4.5, 1.8 Hz, 2H), 3.65 (s, 3H), 3.37 (td, J=11.8, 2.1 Hz, 2H), 3.19-2.89 (m, 3H), 2.46-2.37 (m, 1H), 2.30 (d, J=6.7 Hz, 2H), 2.14-1.96 (m, 2H), 1.88-1.74 (m, 3H), 1.73-1.56 (m, 3H), 1.33-1.24 (m, 2H). HRMS (ESI) m/z: [M+H]⁺ Calcd for C₂₀H₂₆Cl₂N₃O₂, 410.1397. Found 410.1416.

### Example 94: Synthesis of (R)-6,7-dichloro-3-methyl-2-(1-((tetrahydro-2H-pyran-4-yl)methyl)piperidin-3-yl)quinazolin-4(3H)-one (14d)

Prepared according to Scheme 4.

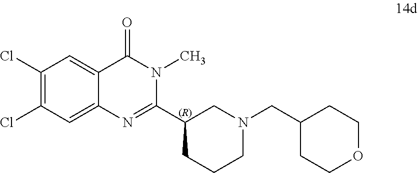

Steps 1-3: See Example 92—Synthesis of 13b.

Step 4: (R)-6,7-dichloro-3-methyl-2-(1-((tetrahydro-2H-pyran-4-yl)methyl)piperidin-3-yl)quinazolin-4(3H)-one (14d). Compound 14d was prepared according to general procedure 12 from 13b and 4-bromomethyl-tetrahydropyran. Purification by flash chromatography (3-30% X/hexanes; X=2% NEt₃ 3:1 EtOAc/EtOH) afforded 14d as a tan, orange solid (61 mg, 81%, 95.2% ee). ¹H NMR (400 MHz, CDCl₃) δ 8.28 (d, J=1.3 Hz, 1H), 7.74 (d, J=0.9 Hz, 1H), 3.95 (ddd, J=11.4, 4.5, 1.8 Hz, 2H), 3.64 (s, 3H), 3.37 (td, J=11.8, 2.1 Hz, 2H), 3.18-2.99 (m, 2H), 2.95 (d, J=11.2 Hz, 1H), 2.39 (t, J=10.9 Hz, 1H), 2.27 (d, J=7.1 Hz, 2H), 2.07-1.96 (m, 2H), 1.86-1.74 (m, 3H), 1.70-1.58 (m, 3H), 1.28 (tdd, J=14.3, 6.5, 3.7 Hz, 2H). HRMS (ESI) m/z: [M+H]⁺ Calcd for C₂₀H₂₆Cl₂N₃O₂, 410.1397. Found 410.1411.

### Example 95: Synthesis of (S)-6,7-dichloro-3-methyl-2-(1-(oxetan-3-ylmethyl)piperidin-3-yl)quinazolin-4(3H)-one (14e)

Prepared according to Scheme 4.

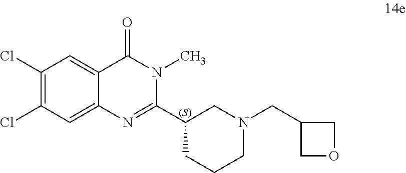

Steps 1-3: See Example 91—Synthesis of 13a.

Step 4: Synthesis of (S)-6,7-dichloro-3-methyl-2-(1-(oxetan-3-ylmethyl)piperidin-3-yl)quinazolin-4(3H)-one (14e). Compound 14e was prepared according to general procedure 12 from 13a and 4-bromomethyl-oxetane. Purification by flash chromatography (15-60% X/hexanes; X=2% NEt₃ 3:1 EtOAc/EtOH) afforded 14e as a tan, orange solid (81.8 mg, 96%, 97% ee). ¹H NMR (400 MHz, CDCl₃) δ 8.28 (s, 1H), 7.74 (s, 1H), 4.80 (dt, J=7.8, 5.7 Hz, 2H), 4.42 (td, J=6.2, 1.0 Hz, 2H), 3.63 (s, 3H), 3.26 (hept, J=7.1 Hz, 1H), 3.05 (tt, J=10.8, 3.3 Hz, 1H), 2.94 (ddt, J=11.4, 3.7, 1.8 Hz, 1H), 2.84 (d, J=11.4 Hz, 1H), 2.78 (d, J=7.2 Hz, 2H), 2.43 (t, J=10.9 Hz, 1H), 2.02 (qd, J=11.6, 2.9 Hz, 2H), 1.82 (dt, J=12.5, 3.0 Hz, 1H), 1.75-1.55 (m, 2H). HRMS (ESI) m/z: [M+H]⁺ Calcd for C₁₈H₂₂Cl₂N₃O₂, 382.1084. Found 382.1089.

### Example 96: Synthesis of (R)-6,7-dichloro-3-methyl-2-(1-(oxetan-3-ylmethyl)piperidin-3-yl)quinazolin-4(3H)-one (14f)

Prepared according to Scheme 4.

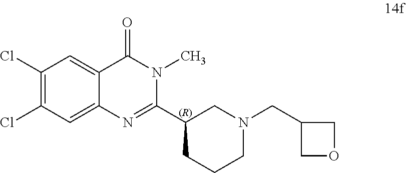

Steps 1-3: See Example 92—Synthesis of 13b.

Step 4: Synthesis of (R)-6,7-dichloro-3-methyl-2-(1-(oxetan-3-ylmethyl)piperidin-3-yl)quinazolin-4(3H)-one (14f). Compound 14f was prepared according to general procedure 12 from 13b and 4-bromomethyl-oxetane. Purification by flash chromatography (15-60% X/hexanes; X=2% NEt₃ 3:1 EtOAc/EtOH) afforded 14f as a tan, orange solid (43.7 mg, 98%, 95.7% ee). ¹H NMR (400 MHz, CDCl₃) δ 8.28 (d, J=2.7 Hz, 1H), 7.74 (d, J=1.9 Hz, 1H), 4.80 (dtd, J=7.2, 5.9, 1.2 Hz, 2H), 4.42 (t, J=6.3 Hz, 2H), 3.63 (d, J=1.3 Hz, 3H), 3.27 (dq, J=14.3, 7.2 Hz, 1H), 3.07 (tt, J=10.9, 3.3 Hz, 1H), 3.00-2.89 (m, 1H), 2.85 (d, J=11.4 Hz, 1H), 2.79 (d, J=7.2 Hz, 2H), 2.44 (t, J=10.9 Hz, 1H), 2.03 (dt, J=17.0, 11.1 Hz, 2H), 1.83 (dt, J=12.7, 3.1 Hz, 1H), 1.78-1.57 (m, 2H). HRMS (ESI) m/z: the [M+H]⁺ Cald for C₁₈H₂₂Cl₂N₃O₂, 382.1084. Found 382.1094.

### Example 97: Synthesis of (R)-6,7-dichloro-2-(1-isobutylpiperidin-3-yl)-3 methylquinazolin-4(3H)-one (14g)

Prepared according to scheme 4.

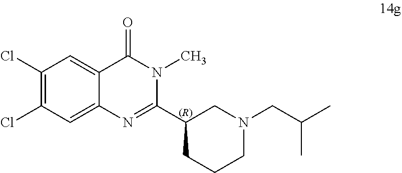

Steps 1-3: See Example 92—Synthesis of 13b.

Step 4: Synthesis of (R)-6,7-dichloro-2-(1-isobutylpiperidin-3-yl)-3-methylquinazolin-4(3H)-one (14g). Compound 14g was prepared according to general procedure 12 from 13b and isobutyl bromide. Purification by flash chromatography (1-15% X/hexanes; X=2% NEt₃ 3:1 EtOAc/EtOH) afforded 14g as a tan solid (78 mg, 85%, 97.1% ee). ¹H NMR (400 MHz, CDCl₃) δ 8.28 (s, 1H), 7.75 (s, 1H), 3.65 (s, 3H), 3.14-2.97 (m, 2H), 2.96-2.86 (m, 1H), 2.30 (t, J=10.8 Hz, 1H), 2.15 (d, J=7.3 Hz, 2H), 1.98 (ddd, J=11.6, 6.8, 4.0 Hz, 2H), 1.86-1.58 (m, 4H), 0.90 (dd, J=6.6, 3.2 Hz, 6H). HRMS (ESI) m/z: [M+H]⁺ Calcd for C₁₈H₂₄Cl₂N₃O, 368.1291. Found 368.1291.

### Example 98: Synthesis of (R)-6,7-dichloro-2-(1-(2-hydroxy-2-methylpropyl)piperidin-3-yl)-3-methylquinazolin-4(3H)-one (14h)

Prepared according to scheme 4.

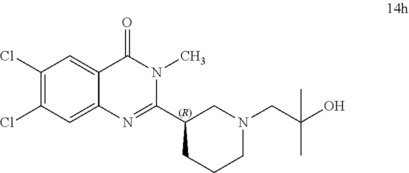

Steps 1-3: See Example 92—Synthesis of 13b.

Step 4: Synthesis of (R)-6,7-dichloro-2-(1-(2-hydroxy-2-methylpropyl)piperidin-3-yl)-3-methylquinazolin-4(3H)-one (14h). Compound 14h was prepared according to general procedure 12 from 13b and 1-bromo-2-methyl-propan-2-ol. Purification by flash chromatography (1-25% X/hexanes; X=2% NEt₃ 3:1 EtOAc/EtOH) afforded 14h as a yellow solid (65 mg, 77%, 99.1% ee). ¹H NMR (400 MHz, CDCl₃) δ 8.28 (s, 1H), 7.75 (s, 1H), 3.65 (s, 3H), 3.02 (dd, J=51.4, 11.1 Hz, 4H), 2.80 (t, J=10.7 Hz, 1H), 2.51-2.35 (m, 3H), 1.98 (d, J=12.0 Hz, 1H), 1.85-1.61 (m, 3H), 1.18 (s, 6H). HRMS (ESI) m/z: [M+H]⁺ Calcd for C₁₈H₂₄Cl₂N₃O₂, 384.1240. Found 384.1231.

### Example 99: Synthesis of 6,7-dichloro-3-methyl-2-(1-(oxetan-3-ylmethyl)azetidin-3-yl)quinazolin-4(3H)-one (14i)

Prepared according to scheme 4.

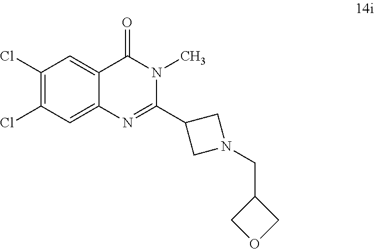 [SMILES: Cn1c(C2CN(CC3COC3)C2)nc2cc(Cl)c(Cl)cc2c1=O]

Step 1: See example 91—Synthesis of 11a.

Step 2: Synthesis of tert-butyl 3-(6,7-dichloro-3-methyl-4-oxo-3,4-dihydroquinazolin-2-yl)azetidine-1-carboxylate (12c). Compound 12c was prepared according to general procedure 10 from 1-(tert-butoxycarbonyl)azetidine-3-carboxylic acid and 11a. Purification by flash chromatography (7-45% X/hexanes, X=4:1 EtOAc/DCM) afforded 12c as a yellow solid (0.87 g, 41%). ¹H NMR (400 MHz, CDCl₃) δ 8.27 (s, 1H), 7.78 (s, 1H), 4.41 (dd, J=8.4, 6.3 Hz, 2H), 4.27 (t, J=8.5 Hz, 2H), 3.90 (tt, J=8.7, 6.3 Hz, 1H), 3.44 (s, 3H), 1.45 (s, 9H). HRMS (ESI) m/z: [M+H]⁺ Calcd for C₁₇H₂₀Cl₂N₃O₃, 384.0876. Found 384.0872.

Step 3: Synthesis of 2-(azetidin-3-yl)-6,7-dichloro-3-methylquinazolin-4(3H)-one trifluoroacetic acid salt (13c). Compound 13c was prepared according to general procedure 11 from 12c. Isolation by vacuum filtration afforded 13c as a white solid (0.67 g, 75%). 13c was used directly without analysis.

Step 4: Synthesis of (6,7-dichloro-3-methyl-2-(1-(oxetan-3-ylmethyl)azetidin-3-yl)quinazolin-4(3H)-one (14i). Compound 14i was prepared according to general procedure 12 from 13c and (bromomethyl)oxetane. Purification by flash chromatography (1-25% X/hexanes; X=2% NEt₃ 3:1 EtOAc/EtOH) afforded 14i as a tan solid (59 mg, 67%). ¹H NMR (400 MHz, CDCl₃) δ 8.28 (s, 1H), 7.77 (s, 1H), 4.79 (dd, J=7.8, 6.1 Hz, 2H), 4.42 (t, J=6.1 Hz, 2H), 3.89 (p, J=7.7 Hz, 1H), 3.79 (t, J=7.4 Hz, 2H), 3.51 (dd, J=8.6, 6.4 Hz, 2H), 3.45 (s, 3H), 3.11-3.01 (m, 1H), 2.84 (d, J=7.5 Hz, 2H). HRMS (ESI) m/z: [M+H]⁺ Calcd for C₁₆H₁₈Cl₂N₃O₂, 354.0771. Found 354.0773.

### Example 100: Synthesis of (R)-6,7-dichloro-3-methyl-2-(1-(oxetan-3-ylmethyl)pyrrolidin-3-yl)quinazolin-4(3H)-one (14j)

Prepared according to scheme 4.

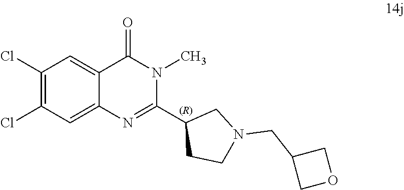

Step 1: See Example 91—Synthesis of 11a.

Step 2: Synthesis tert-butyl (R)-3-(6,7-dichloro-3-methyl-4-oxo-3,4-dihydroquinazolin-2-yl)pyrrolidine-1-carboxylate (12d). Compound 12d was prepared according to general procedure 10 from 1-(tert-butoxycarbonyl)azetidine-3-carboxylic acid and 11a. Purification by flash chromatography (15-40% X/hexanes, X=4:1 EtOAc/DCM) afforded 12d as a light-tan solid (1.31 g, 72%). ¹H NMR (400 MHz, CDCl₃) δ 8.26 (s, 1H), 7.71 (s, 1H), 3.89-3.70 (m, 2H), 3.70-3.52 (m, 5H), 3.48 (d, J=9.5 Hz, 1H), 2.47-2.22 (m, 2H), 1.47 (s, 9H). HRMS (ESI) m/z: [M+H]⁺ Calcd for C₁₈H₂₂Cl₂N₃O₃, 398.1033. Found 398.1030.

Step 3: Synthesis of (R)-6,7-dichloro-3-methyl-2-(pyrrolidin-3-yl)quinazolin-4(3H)-one trifluoroacetic acid salt (13d). Compound 13d was prepared according to general procedure 11 from 12d. Isolation by vacuum filtration afforded 13d as a white solid (0.86 g, 71%). 13d was used directly without analysis.

Step 4: Synthesis of (R)-6,7-dichloro-3-methyl-2-(1-(oxetan-3-ylmethyl)pyrrolidin-3-yl)quinazolin-4(3H)-one (14j). Compound 14j was prepared according to general procedure 12 from 13d and (bromomethyl)oxetane. Purification by flash chromatography (50-80% X/hexanes; X=2% NEt₃ 3:1 EtOAc/EtOH) afforded 14j (R)-6,7-dichloro-3-methyl-2-(1-(oxetan-3-ylmethyl)pyrrolidin-3-yl)quinazolin-4(3H)-one (14j) as a waxy orange solid (87 mg, 95%, 88.8% ee). ¹H NMR (400 MHz, CDCl₃) δ 8.28 (s, 1H), 7.75 (s, 1H), 4.83 (dd, J=7.8, 6.1 Hz, 2H), 4.46 (td, J=6.1, 4.8 Hz, 2H), 3.68-3.54 (m, 4H), 3.26 (dq, J=8.0, 6.6 Hz, 1H), 3.12-2.84 (m, 5H), 2.61 (q, J=8.1 Hz, 1H), 2.33 (ddt, J=12.5, 9.9, 7.4 Hz, 1H), 2.27-2.17 (m, 1H). HRMS (ESI) m/z: [M+H]⁺ Calcd for C₁₇H₂₀Cl₂N₃O₂, 368.0927. Found 368.0932.

### Example 101: Synthesis of (R)-6,7-dichloro-3-(2-methoxyethyl)-2-(1-(oxetan-3-ylmethyl)piperidin-3-yl)quinazolin-4(3H)-one (14k)

Prepared according to scheme 4.

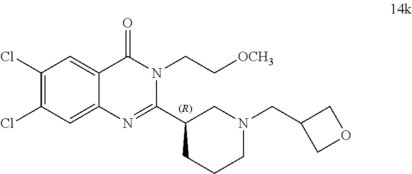

Step 1: Synthesis of 2-amino-4,5-dichloro-N-(2-methoxyethyl)benzamide (11b). Compound 11b was prepared according to general procedure 9b from 2-amino, 4,5-dichloro-benzoic acid and 2-methoxyethan-1-amine. After work-up, 11b was afforded as a yellow solid (1.67 g, 80%). ¹H NMR (400 MHz, CDCl₃) δ 7.36 (s, 1H), 6.74 (s, 1H), 6.49 (d, J=6.4 Hz, 1H), 5.60 (s, 2H), 3.61-3.50 (m, 4H), 3.38 (s, 3H). HRMS (ESI) m/z: [M+H]⁺ Calcd for C₁₀H₁₃Cl₂N₂O₂, 263.0349. Found 263.0341.

Step 2: Synthesis of tert-butyl (R)-3-(6,7-dichloro-3-(2-methoxyethyl)-4-oxo-3,4-dihydroquinazolin-2-yl)piperidine-1-carboxylate (12e). Compound 12e was prepared according to general procedure 10 from (R)-1-(tert-butoxycarbonyl)piperidine-3-carboxylic acid and 11b. Purification by flash chromatography (3-20% X/hexanes, X=4:1 EtOAc/DCM) afforded 12e as an off-white solid (1.23 g, 71%). ¹H NMR (400 MHz, CDCl₃) δ 8.28 (s, 1H), 7.74 (s, 1H), 4.43 (dt, J=14.4, 5.2 Hz, 1H), 4.35-4.07 (m, 3H), 3.66 (t, J=5.2 Hz, 2H), 3.29 (s, 3H), 3.21 (tt, J=10.8, 3.5 Hz, 1H), 3.12 (s, 1H), 2.78 (s, 1H), 2.06-1.99 (m, 1H), 1.91 (s, 1H), 1.78 (dt, J=13.5, 3.1 Hz, 1H), 1.61 (d, J=11.7 Hz, 1H), 1.46 (s, 9H). HRMS (ESI) m/z: [M+H]⁺ Calcd for C₂₁H₂₈Cl₂N₃O₄, 456.1451. Found 456.1455.

Step 3: Synthesis of (R)-6,7-dichloro-3-(2-methoxyethyl)-2-(piperidin-3-yl)quinazolin-4(3H)-one trifluoroacetic acid salt (13e). Compound 13e was prepared according to general procedure 11 from 12e. Isolation by vacuum filtration afforded 13e as a white solid (0.88 g, 65%). 13e was used directly without analysis.

Step 4: Synthesis of (R)-6,7-dichloro-3-(2-methoxyethyl)-2-(1-(oxetan-3-ylmethyl)piperidin-3-yl)quinazolin-4(3H)-one (14k). Compound 14k was prepared according to general procedure 12 from 13e and (bromomethyl)oxetane. Purification by flash chromatography (20-35% X/hexanes; X=2% NEt₃ 3:1 EtOAc/EtOH) afforded 14k as a tan solid (92 mg, 87%, 97.9% ee). ¹H NMR (400 MHz, CDCl₃) δ 8.28 (s, 1H), 7.75 (s, 1H), 4.79 (ddd, J=8.0, 6.0, 4.1 Hz, 2H), 4.48-4.36 (m, 3H), 4.26 (dt, J=14.3, 5.6 Hz, 1H), 3.64 (t, J=5.3 Hz, 2H), 3.36-3.22 (m, 5H), 2.89 (d, J=11.1 Hz, 1H), 2.83 (d, J=11.1 Hz, 1H), 2.77 (d, J=7.2 Hz, 2H), 2.39 (t, J=10.8 Hz, 1H), 2.10-2.01 (m, 1H), 1.92 (d, J=9.7 Hz, 1H), 1.82-1.74 (m, 1H), 1.66 (qd, J=11.1, 10.4, 3.8 Hz, 2H). HRMS (ESI) m/z: [M+H]⁺ Calcd for C₂₀H₂₆Cl₂N₃O₃, 426.1246. Found 426.1250.

### Example 102: Synthesis of (R)-6,7-dichloro-3-(2-(dimethylamino)ethyl)-2-(1-(oxetan-3-ylmethyl)piperidin-3-yl)quinazolin-4(3H)-one (141)

Prepared according to scheme 4.

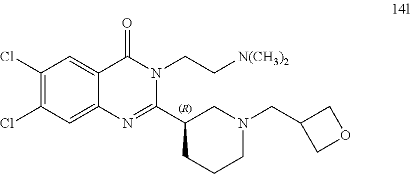

Step 1: Synthesis of 2-amino-4,5-dichloro-N-(2-(dimethylamino)ethyl)benzamide (11c). Compound 11c was prepared according to general procedure 9b from 2-amino, 4,5-dichloro-benzoic acid and 2-dimethylamino-ethan-1-amine. After work-up, 11c was afforded as an orange solid (1.78 g, 81%). ¹H NMR (400 MHz, CD₃OD_SPE) δ 7.62 (s, 1H), 6.88 (s, 1H), 4.86 (s, 3H), 3.47 (t, J=6.6 Hz, 2H), 2.60 (t, J=6.6 Hz, 2H), 2.35 (s, 6H). HRMS (ESI) m/z: [M+H]⁺ Calcd for C₁₁H₁₆Cl₂N₃O, 276.0665. Found 276.0667.

Step 2: Synthesis of tert-butyl (R)-3-(6,7-dichloro-3-(2-(dimethylamino)ethyl)-4-oxo-3,4-dihydroquinazolin-2-yl)piperidine-1-carboxylate (12f). Compound 12f was prepared according to general procedure 10 from (R)-1-(tert-butoxycarbonyl)piperidine-3-carboxylic acid and 11c. Purification by flash chromatography (3-10% X/hexanes, X=4:1 EtOAc/DCM) afforded 12f as a light-yellow solid (1.08 g, 63%). ¹H NMR (400 MHz, CDCl₃) δ 8.27 (s, 1H), 7.73 (s, 1H), 4.38-4.07 (m, 4H), 3.04 (d, J=49.9 Hz, 2H), 2.80 (s, 1H), 2.68-2.48 (m, 2H), 2.30 (s, 6H), 2.05 (d, J=14.4 Hz, 2H), 1.80 (d, J=13.6 Hz, 1H), 1.58 (d, J=30.6 Hz, 1H), 1.46 (s, 9H). HRMS (ESI) m/z: [M+H]⁺ Calcd for C₂₂H₃₁Cl₂N₄O₃, 469.1768. Found 469.1771.

Step 3: Synthesis of (R)-6,7-dichloro-3-(2-(dimethylamino)ethyl)-2-(piperidin-3-yl)quinazolin-4(3H)-one trifluoroacetic acid salt (13f). Compound 13f was prepared according to general procedure 11 from 12f. Isolation by vacuum filtration afforded 13f as a white solid (1.01 g, 75%). 13f was used directly without analysis.

Step 4: Synthesis of (R)-6,7-dichloro-3-(2-(dimethylamino)ethyl)-2-(1-(oxetan-3-ylmethyl)piperidin-3-yl)quinazolin-4(3H)-one (141). Compound 141 was prepared according to general procedure 12 from 13f and (bromomethyl)oxetane. Purification by flash chromatography (80-100% X/hexanes; X=2% NEt₃ 3:1 EtOAc/EtOH) afforded 141 as a waxy orange solid (80 mg, 74%, 94.9% ee). ¹H NMR (400 MHz, CDCl₃) δ 8.28 (s, 1H), 7.74 (s, 1H), 4.80 (ddd, J=7.9, 6.1, 4.3 Hz, 2H), 4.41 (td, J=6.2, 2.8 Hz, 2H), 4.28 (ddd, J=14.0, 8.0, 6.2 Hz, 1H), 4.15 (ddd, J=13.9, 8.1, 6.3 Hz, 1H), 3.26 (p, J=7.0 Hz, 1H), 3.09 (s, 1H), 2.91 (d, J=11.3 Hz, 1H), 2.84 (d, J=11.1 Hz, 1H), 2.78 (d, J=7.3 Hz, 2H), 2.63-2.52 (m, 2H), 2.45 (t, J=10.8 Hz, 1H), 2.33 (s, 6H), 2.11-2.03 (m, 1H), 1.96 (d, J=8.5 Hz, 1H), 1.81 (dq, J=8.5, 2.8 Hz, 1H), 1.74-1.63 (m, 2H). HRMS (ESI) m/z: [M+H]⁺ Calcd for C₂₁H₂₉Cl₂N₄O₂, 439.1662. Found 439.1662.

### Example 103: Synthesis of (R)-6,7-dichloro-3-(2-morpholinoethyl)-2-(1-(oxetan-3-ylmethyl)piperidin-3-yl)quinazolin-4(3H)-one (14m)

Prepared according to scheme 4.

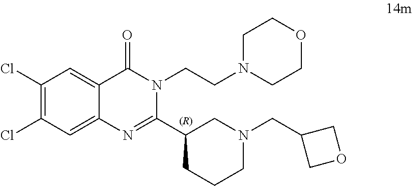

Step 1: Synthesis of 2-amino-4,5-dichloro-N-(2-morpholinoethyl)benzamide (11d). Compound 11d was prepared according to general procedure 9b from 2-amino, 4,5-dichloro-benzoic acid and 2-morpholinoethan-1-amine. After work-up, 11d was afforded as an off-white solid (2.1 g, 83%, 95.2% ee). ¹H NMR (400 MHz, CDCl₃) δ 7.35 (s, 1H), 6.76 (s, 1H), 6.60 (t, J=5.2 Hz, 1H), 5.60 (s, 2H), 3.77-3.68 (m, 4H), 3.55-3.45 (m, 2H), 2.58 (t, J=6.0 Hz, 2H), 2.50 (t, J=4.7 Hz, 4H). HRMS (ESI) m/z: [M+H]⁺ Calcd for C₁₃H₁₈Cl₂N₃O₂, 318.0771. Found 318.0776.

Step 2: Synthesis of tert-butyl (R)-3-(6,7-dichloro-3-(2-morpholinoethyl)-4-oxo-3,4-dihydroquinazolin-2-yl)piperidine-1-carboxylate (12g). Compound 12g was prepared according to general procedure 10 from (R)-1-(tert-butoxycarbonyl)piperidine-3-carboxylic acid and 11d. Purification by flash chromatography (1-10% X/hexanes, X=4:1 EtOAc/DCM) afforded 12g as a yellow solid (1.23 g, 76%). ¹H NMR (400 MHz, CDCl₃) δ 8.28 (s, 1H), 7.74 (s, 1H), 4.28 (d, J=41.9 Hz, 4H), 3.65 (t, J=4.6 Hz, 4H), 3.15-2.60 (m, 5H), 2.52 (t, J=4.6 Hz, 4H), 2.05 (d, J=10.0 Hz, 1H), 1.87-1.80 (m, 1H), 1.59-1.50 (m, 1H), 1.48-1.45 (m, 10H). HRMS (ESI) m/z: [M+H]⁺ Calcd for C₂₄H₃₃Cl₂N₄O₄, 511.1873. Found 511.1867.

Step 3: Synthesis of (R)-6,7-dichloro-3-(2-morpholinoethyl)-2-(piperidin-3-yl)quinazolin-4(3H)-one trifluoroacetic acid salt (13g). Compound 13g was prepared according to general procedure 11 from 12g. Isolation by vacuum filtration afforded 13g as a white solid (1.05 g, 66%). 13g was used directly without analysis.

Step 4: Synthesis of (R)-6,7-dichloro-3-(2-morpholinoethyl)-2-(1-(oxetan-3-ylmethyl)piperidin-3-yl)quinazolin-4(3H)-one (14m). Compound 14m was prepared according to general procedure 12 from 13g and (bromomethyl)oxetane. Purification by flash chromatography (40-75% X/hexanes; X=2% NEt₃ 3:1 EtOAc/EtOH) afforded 14m as a tan solid (96 mg, 80%). ¹H NMR (400 MHz, CDCl₃) δ 8.27 (s, 1H), 7.74 (s, 1H), 4.79 (ddd, J=7.9, 6.0, 4.6 Hz, 2H), 4.41 (dt, J=6.2, 3.1 Hz, 2H), 4.28 (dt, J=13.1, 6.3 Hz, 1H), 4.12 (dt, J=13.7, 6.8 Hz, 1H), 3.67 (dt, J=5.3, 3.5 Hz, 4H), 3.26 (p, J=7.5 Hz, 1H), 3.10 (d, J=14.1 Hz, 1H), 2.92 (d, J=11.1 Hz, 1H), 2.81 (dd, J=23.0, 9.1 Hz, 3H), 2.68-2.59 (m, 2H), 2.58-2.42 (m, 5H), 2.01 (dd, J=38.0, 9.7 Hz, 2H), 1.82 (dq, J=10.0, 4.0, 3.5 Hz, 1H), 1.68 (d, J=24.2 Hz, 2H). HRMS (ESI) m/z: [M+H]⁺ Calcd for C₂₃H₃₁Cl₂N₄O₃, 481.1768. Found 481.1764.

### Example 104: Synthesis of (R)-6-chloro-3-methyl-2-(1-(oxetan-3-ylmethyl)piperidin-3-yl)pyrido[3,4-d]pyrimidin-4(3H)-one (14n)

Prepared according to scheme 4.

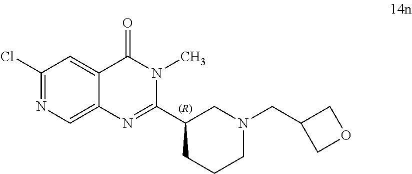

Step 1: Synthesis of 5-amino-2-chloro-N-methylisonicotinamide (11e). Compound 11e was prepared according to general procedure 9b from 5-amino-2-chloroisonicotinic acid and methylammonium chloride. After work-up, 11e was afforded as a yellow solid (1.67 g, 80%). ¹H NMR (400 MHz, DMSO) δ 8.59 (q, J=4.9, 4.5 Hz, 1H), 7.92 (s, 1H), 7.46 (s, 1H), 6.53 (s, 2H), 2.74 (d, J=4.6 Hz, 3H). ¹³C NMR (101 MHz, DMSO) δ 166.4, 143.9, 139.1, 135.1, 122.7, 120.9, 26.0. HRMS (ESI) m/z: [M+H]⁺ Calcd for C₇H₉ClN₃O, 186.0428. Found 186.0428.

Step 2: Synthesis of tert-butyl (R)-3-(6-chloro-3-methyl-4-oxo-3,4-dihydropyrido[3,4-d]pyrimidin-2-yl)piperidine-1-carboxylate (12h). Compound 12h was prepared according to general procedure 10 from (R)-1-(tert-butoxycarbonyl)piperidine-3-carboxylic acid and 11e. Purification by flash chromatography (3-20% X/hexanes, X=4:1 EtOAc/DCM) afforded 12h as an off-white solid (1.67 g, 82%). ¹H NMR (400 MHz, CDCl₃) δ 8.83 (s, 1H), 8.02 (s, 1H), 4.23 (d, J=58.0 Hz, 2H), 3.69 (s, 3H), 3.10 (d, J=14.9 Hz, 1H), 2.93 (tt, J=10.6, 3.2 Hz, 1H), 2.82 (s, 1H), 2.09 (dt, J=13.6, 3.3 Hz, 1H), 2.01-1.89 (m, 1H), 1.84 (dq, J=13.5, 3.1 Hz, 1H), 1.65-1.52 (m, 1H), 1.46 (s, 9H). ¹³C NMR (101 MHz, CDCl₃) δ 160.5, 159.5, 154.7, 151.2, 147.6, 140.8, 127.6, 119.2, 80.2, 47.7, 41.0, 30.7, 29.4, 28.6, 28.5, 24.9. HRMS (ESI) m/z: [M+H]⁺ Calcd for C₁₈H₂₄ClN₄O₃, 379.1531. Found 379.1527.

Step 3: Synthesis of (R)-6-chloro-3-methyl-2-(piperidin-3-yl)pyrido[3,4-d]pyrimidin-4(3H)-one trifluoroacetic acid salt (13h). Compound 13h was prepared according to general procedure 11 from 12h. Isolation by vacuum filtration afforded 13h as a white solid (1.05 g, 66%). 13h was used directly without analysis.

Step 4: Synthesis of (R)-6-chloro-3-methyl-2-(1-(oxetan-3-ylmethyl)piperidin-3-yl)pyrido[3,4-d]pyrimidin-4(3H)-one (14n). Compound 14n was prepared according to general procedure 12 from 13h and (bromomethyl)oxetane. Purification by flash chromatography (40-75% X/hexanes; X=2% NEt₃ 3:1 EtOAc/EtOH) afforded 14n as a waxy orange solid (137 mg, 99%, 95.1% ee). ¹H NMR (400 MHz, CDCl₃) δ 8.83 (s, 1H), 8.02 (s, 1H), 4.79 (dt, J=7.9, 5.7 Hz, 2H), 4.41 (t, J=6.2 Hz, 2H), 3.65 (s, 3H), 3.26 (dq, J=14.2, 6.7 Hz, 1H), 3.09 (tt, J=10.8, 3.1 Hz, 1H), 2.95 (ddt, J=11.3, 3.5, 1.7 Hz, 1H), 2.88-2.82 (m, 1H), 2.81-2.77 (m, 2H), 2.47 (t, J=10.9 Hz, 1H), 2.03 (qd, J=11.6, 3.2 Hz, 2H), 1.84 (dt, J=12.1, 4.4 Hz, 1H), 1.76-1.57 (m, 2H). ¹³C NMR (101 MHz, CDCl₃) δ 160.6, 160.4, 151.2, 147.5, 140.9, 127.6, 119.2, 76.7, 76.4, 62.5, 57.6, 53.7, 41.3, 33.3, 30.7, 29.5, 25.2. HRMS (ESI) m/z: [M+H]⁺ Calcd for C₁₇H₂₂ClN₄O₂, 349.1426. Found 349.1422.

### Example 105: Synthesis of (R)-3-methyl-2-(1-(oxetan-3-ylmethyl)piperidin-3-yl)-4-oxo-3,4-dihydroquinazoline-6-carbonitrile (140)

Prepared according to scheme 4.

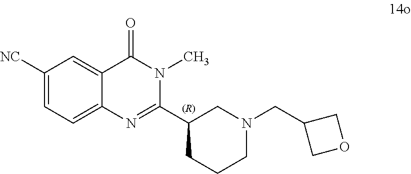

Step 1: Synthesis of 2-amino-5-cyano-N-methylbenzamide (11f). Compound 11f was prepared according to general procedure 9a from 2-amino-5-cyano-benzoic acid and methylammonium chloride. After work-up, 11f was afforded as a light yellow solid (2.48 g, 92%). ¹H NMR (400 MHz, DMSO) δ 8.40 (s, 1H), 7.91 (d, J=2.0 Hz, 1H), 7.47 (dd, J=8.7, 2.0 Hz, 1H), 7.33 (s, 2H), 6.78 (d, J=8.7 Hz, 1H), 2.73 (d, J=4.5 Hz, 3H).

Step 2: Synthesis of tert-butyl (R)-3-(6-cyano-3-methyl-4-oxo-3,4-dihydroquinazolin-2-yl)piperidine-1-carboxylate (12i). Compound 12i was prepared according to general procedure 10 from (R)-1-(tert-butoxycarbonyl)piperidine-3-carboxylic acid and 11f. Purification by flash chromatography (0-50% EtOAc/Hexanes) afforded 12i as a yellow solid (1.85 g, 39%). ¹H NMR (400 MHz, CDCl₃) δ 8.58 (d, J=2.0 Hz, 1H), 7.88 (dd, J=8.5, 2.0 Hz, 1H), 7.69 (d, J=8.5 Hz, 1H), 4.24 (d, J=51.1 Hz, 2H), 3.71 (s, 3H), 3.14 (s, 1H), 2.99-2.90 (m, 1H), 2.83 (s, 1H), 2.11 (d, J=13.2 Hz, 1H), 1.96 (tdd, J=13.2, 11.1, 3.8 Hz, 1H), 1.85 (d, J=13.7 Hz, 1H), 1.48 (s, 10H).

Step 3: Synthesis of (R)-3-methyl-4-oxo-2-(piperidin-3-yl)-3,4-dihydroquinazoline-6-carbonitrile trifluoroacetic acid salt (13i). Compound 13i was prepared according to general procedure 11 from 12i. Isolation by vacuum filtration afforded 13i as a white solid (1.16 g, 51%). 13i was used directly without analysis.

Step 4: Synthesis of (R)-3-methyl-2-(1-(oxetan-3-ylmethyl)piperidin-3-yl)-4-oxo-3,4-dihydroquinazoline-6-carbonitrile (14o). Compound 14o was prepared according to general procedure 12 from 13i and 3-(bromomethyl)oxetane. Purification by flash chromatography (25-50% X/hexanes; X=2% NEt₃ 3:1 EtOAc/EtOH) afforded 14o as a dark yellow solid (64.3 mg, 63%). ¹H NMR (400 MHz, CDCl₃) δ 8.57 (d, J=1.9 Hz, 1H), 7.88 (dd, J=8.5, 2.0 Hz, 1H), 7.68 (d, J=8.5 Hz, 1H), 4.81 (dt, J=7.8, 5.9 Hz, 2H), 4.43 (t, J=6.1 Hz, 2H), 3.67 (s, 3H), 3.27 (s, 1H), 3.10 (s, 1H), 2.97 (d, J=11.3 Hz, 1H), 2.83 (dd, J=21.3, 8.5 Hz, 3H), 2.49 (s, 1H), 2.06-1.98 (m, 2H), 1.86 (s, 1H), 1.69-1.56 (m, 2H). HRMS (ESI) m/z: [M+H]⁺ Calcd for C₁₉H₂₃N₄O₂, 339.1816. Found 339.1827.

### Example 106: Synthesis of (R)-6-chloro-3-methyl-2-(1-(oxetan-3-ylmethyl)piperidin-3-yl)quinazolin-4(3H)-one (14p)

Prepared according to scheme 4.

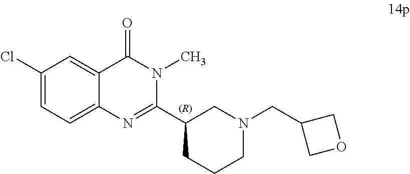

Step 1: Synthesis of 2-amino-5-chloro-N-methylbenzamide (11g). Compound 11g was prepared according to general procedure 9a from 2-amino-5-chloro-benzoic acid and methylammonium chloride. After work-up, 11g was afforded as a light yellow solid (2.58 g, 96%). ¹H NMR (400 MHz, CDCl₃) δ 7.26 (s, 1H), 7.14 (dd, J=8.7, 2.4 Hz, 1H), 6.62 (d, J=8.7 Hz, 1H), 6.04 (s, 1H), 5.48 (s, 2H), 2.96 (d, J=4.8 Hz, 3H).

Step 2: Synthesis of tert-butyl (R)-3-(6-chloro-3-methyl-4-oxo-3,4-dihydroquinazolin-2-yl)piperidine-1-carboxylate (12j). Compound 12j was prepared according to general procedure 10 from (R)-1-(tert-butoxycarbonyl)piperidine-3-carboxylic acid and 11g. Purification by flash chromatography (0-40% EtOAc/Hexanes) afforded 12j as a yellow solid (3.78 g, 81%). ¹H NMR (400 MHz, CDCl₃) δ 8.21 (d, J=2.4 Hz, 1H), 7.64 (dd, J=8.7, 2.4 Hz, 1H), 7.56 (d, J=8.7 Hz, 1H), 4.24 (d, J=41.2 Hz, 2H), 3.69 (s, 3H), 3.11 (d, J=17.3 Hz, 1H), 2.92 (tt, J=10.9, 3.4 Hz, 1H), 2.82 (s, 1H), 2.10 (dq, J=13.0, 3.2 Hz, 1H), 1.95 (tdd, J=13.2, 11.2, 3.8 Hz, 1H), 1.84 (d, J=13.8 Hz, 1H), 1.66-1.55 (m, 1H), 1.48 (s, 9H).

Step 3: Synthesis of (R)-6-chloro-3-methyl-2-(piperidin-3-yl)quinazolin-4(3H)-one trifluoroacetic acid salt (13j). Compound 13j was prepared according to general procedure 11 from 12j. Isolation by vacuum filtration afforded 13j as a white solid (3.35 g, 71%). 13j was used directly without analysis.

Step 4: Synthesis of (R)-6-chloro-3-methyl-2-(1-(oxetan-3-ylmethyl)piperidin-3-yl)quinazolin-4(3H)-one (14p). Compound 14p was prepared according to general procedure 12 from 13j and 3-(bromomethyl)oxetane. Purification by flash chromatography (30-50% X/hexanes; X=2% NEt₃ 3:1 EtOAc/EtOH) afforded 14p as a dark yellow solid (60.0 mg, 57%). ¹H NMR (400 MHz, CDCl₃) δ 8.21 (d, J=2.4 Hz, 1H), 7.63 (dd, J=8.7, 2.5 Hz, 1H), 7.56 (d, J=8.7 Hz, 1H), 4.81 (dt, J=7.9, 5.9 Hz, 2H), 4.46-4.40 (m, 2H), 3.66 (s, 3H), 3.29-3.25 (m, 1H), 3.06 (s, 1H), 2.96 (d, J=11.3 Hz, 1H), 2.86-2.79 (m, 3H), 2.46 (t, J=10.9 Hz, 1H), 2.08-2.00 (m, 2H), 1.83 (d, J=11.9 Hz, 1H), 1.68-1.59 (m, 2H). HRMS (ESI) m/z: [M+H]⁺ Calcd for C₁₈H₂₃ClN₃O₂, 348.1473. Found 348.1486.

### Example 107: Synthesis of (R)-6-fluoro-3-methyl-2-(1-(oxetan-3-ylmethyl)piperidin-3-yl)quinazolin-4(3H)-one (14q)

Prepared according to scheme 4.

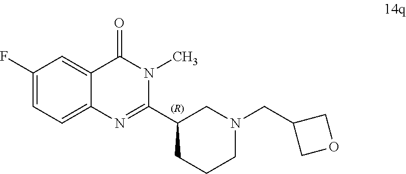

Step 1: Synthesis of 2-amino-5-fluoro-N-methylbenzamide (11h). Compound 11h was prepared according to general procedure 9a from 2-amino-5-fluoro-benzoic acid and methylammonium chloride. After work-up, 11h was afforded as a light yellow solid (2.02 g, 93%). ¹H NMR (400 MHz, DMSO) δ 8.23 (s, 1H), 7.30 (dd, J=10.3, 3.0 Hz, 1H), 7.03 (td, J=8.6, 3.0 Hz, 1H), 6.70 (dd, J=9.0, 5.0 Hz, 1H), 6.29 (s, 2H), 2.73 (d, J=4.5 Hz, 3H).

Step 2: Synthesis of tert-butyl (R)-3-(6-fluoro-3-methyl-4-oxo-3,4-dihydroquinazolin-2-yl)piperidine-1-carboxylate (12k). Compound 12k was prepared according to general procedure 10 from (R)-1-(tert-butoxycarbonyl)piperidine-3-carboxylic acid and 11h. Purification by flash chromatography (0-40% EtOAc/Hexanes) afforded 12k as a light yellow solid (2.66 g, 69%). ¹H NMR (400 MHz, CDCl₃) δ 7.83 (dd, J=8.5, 3.0 Hz, 1H), 7.61 (dd, J=9.0, 4.9 Hz, 1H), 7.40 (td, J=8.5, 3.0 Hz, 1H), 4.26 (d, J=56.6 Hz, 2H), 3.70 (s, 3H), 3.12-3.08 (m, 1H), 2.95 (tt, J=10.9, 3.4 Hz, 1H), 2.86-2.83 (m, 1H), 2.12 (dt, J=14.5, 3.5 Hz, 1H), 1.96 (tdd, J=13.2, 11.1, 3.8 Hz, 1H), 1.87-1.82 (m, 1H), 1.64 (dt, J=14.8, 6.5 Hz, 1H), 1.49 (s, 9H).

Step 3: Synthesis of (R)-6-fluoro-3-methyl-2-(piperidin-3-yl)quinazolin-4(3H)-one trifluoroacetic acid salt (13k). Compound 13k was prepared according to general procedure 11 from 12k. Isolation by vacuum filtration afforded 13k as an off-white solid (2.80 g, 83%). 13k was used directly without analysis.

Step 4: Synthesis of (R)-6-fluoro-3-methyl-2-(1-(oxetan-3-ylmethyl)piperidin-3-yl)quinazolin-4(3H)-one (14q). Compound 14q was prepared according to general procedure 12 from 13k and 3-(bromomethyl)oxetane. Purification by flash chromatography (20-50% X/hexanes; X=2% NEt₃ 3:1 EtOAc/EtOH) afforded 14q as an off-white solid (86.8 mg, 87%). ¹H NMR (400 MHz, CDCl₃) δ 7.87 (dd, J=8.5, 2.9 Hz, 1H), 7.62 (dd, J=8.9, 4.8 Hz, 1H), 7.42 (td, J=8.6, 3.0 Hz, 1H), 4.81 (dt, J=8.0, 5.9 Hz, 2H), 4.43 (td, J=6.2, 1.1 Hz, 2H), 3.66 (s, 3H), 3.27 (hept, J=7.2 Hz, 1H), 3.07 (tt, J=10.7, 3.4 Hz, 1H), 2.96 (ddt, J=11.4, 3.7, 1.8 Hz, 1H), 2.88-2.77 (m, 3H), 2.46 (t, J=10.9 Hz, 1H), 2.09-1.98 (m, 2H), 1.86-1.81 (m, 1H), 1.76-1.59 (m, 2H). HRMS (ESI) m/z: [M+H]⁺ Calcd for C₁₈H₂₃FN₃O₂, 332.1769. Found 332.1785.

### Example 108: Synthesis of (R)-7-fluoro-3-methyl-2-(1-(oxetan-3-ylmethyl)piperidin-3-yl)quinazolin-4(3H)-one (14r)

Prepared according to scheme 4.

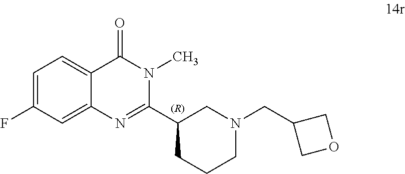

Step 1: Synthesis of 2-amino-4-fluoro-N-methylbenzamide (11i). Compound 11i was prepared according to general procedure 9a from 2-amino-4-fluoro-benzoic acid and methylammonium chloride. After work-up, 11i was afforded as a light yellow solid (2.02 g, 93%). ¹H NMR (400 MHz, CDCl₃) δ 7.32-7.28 (m, 1H), 6.78 (s, 1H), 6.32 (dd, J=11.0, 2.5 Hz, 1H), 6.22 (td, J=8.4, 2.5 Hz, 1H), 5.79 (s, 2H), 2.83 (d, J=4.9 Hz, 3H).

Step 2: Synthesis of tert-butyl (R)-3-(7-fluoro-3-methyl-4-oxo-3,4-dihydroquinazolin-2-yl)piperidine-1-carboxylate (121). Compound 121 was prepared according to general procedure 10 from (R)-1-(tert-butoxycarbonyl)piperidine-3-carboxylic acid and 11i. Purification by flash chromatography (5-40% EtOAc/Hexanes) afforded 121 as a yellow solid (3.70 g, 96%). ¹H NMR (400 MHz, CDCl₃) δ 8.19 (dd, J=8.9, 6.1 Hz, 1H), 7.22 (dd, J=9.7, 2.5 Hz, 1H), 7.11 (td, J=8.5, 2.5 Hz, 1H), 4.27 (d, J=58.0 Hz, 2H), 3.69 (s, 3H), 3.14-3.09 (m, 1H), 2.95 (tt, J=10.8, 3.3 Hz, 1H), 2.88-2.80 (m, 1H), 2.12 (dt, J=13.7, 3.3 Hz, 1H), 1.96 (tdd, J=13.2, 11.0, 3.8 Hz, 1H), 1.85 (dt, J=13.6, 3.0 Hz, 1H), 1.68-1.55 (m, 1H), 1.49 (s, 9H).

Step 3: Synthesis of (R)-7-fluoro-3-methyl-2-(piperidin-3-yl)quinazolin-4(3H)-one trifluoroacetic acid salt (131). Compound 131 was prepared according to general procedure 11 from 121. Isolation by vacuum filtration afforded 131 as a white solid (3.10 g, 65%). 131 was used directly without analysis.

Step 4: Synthesis of (R)-7-fluoro-3-methyl-2-(1-(oxetan-3-ylmethyl)piperidin-3-yl)quinazolin-4(3H)-one (14r). Compound 14r was prepared according to general procedure 12 from 131 and 3-(bromomethyl)oxetane. Purification by flash chromatography (30-50% X/hexanes; X=2% NEt₃ 3:1 EtOAc/EtOH) afforded 14r as a viscous dark yellow oil (93.5 mg, 94%). ¹H NMR (400 MHz, CDCl₃) δ 8.25 (dd, J=8.9, 6.2 Hz, 1H), 7.26 (dd, J=9.7, 2.5 Hz, 1H), 7.14 (td, J=8.6, 2.5 Hz, 1H), 4.81 (dt, J=7.8, 5.7 Hz, 2H), 4.43 (td, J=6.2, 1.2 Hz, 2H), 3.65 (s, 3H), 3.27 (p, J=7.1 Hz, 1H), 3.06 (s, 1H), 2.96 (d, J=11.4 Hz, 1H), 2.82 (dd, J=19.1, 9.2 Hz, 3H), 2.47 (t, J=10.9 Hz, 1H), 2.08-1.99 (m, 2H), 1.85-1.81 (m, 1H), 1.75-1.59 (m, 2H). HRMS (ESI) m/z: [M+H]⁺ Calcd for C₁₈H₂₃FN₃O₂, 332.1769. Found 332.1784.

### Example 109: Synthesis of (R)-6-methoxy-3-methyl-2-(1-(oxetan-3-ylmethyl)piperidin-3-yl)quinazolin-4(3H)-one (14s)

Prepared according to scheme 4.

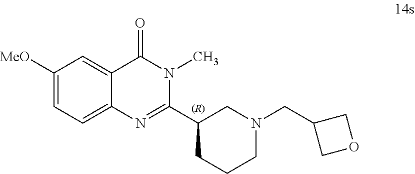

Step 1: Synthesis of 2-amino-5-methoxy-N-methylbenzamide (11j). Compound 11j was prepared according to general procedure 9a from 2-amino-5-methoxy-benzoic acid and methylammonium chloride. After work-up, 11j was afforded as a brown solid (2.03 g, 94%). ¹H NMR (400 MHz, CDCl₃) δ 6.88 (d, J=2.9 Hz, 1H), 6.83 (dd, J=8.7, 2.9 Hz, 1H), 6.63 (d, J=8.7 Hz, 1H), 6.46 (s, 1H), 5.01 (s, 2H), 3.71 (s, 3H), 2.92 (d, J=4.9 Hz, 3H).

Step 2: Synthesis of tert-butyl (R)-3-(6-methoxy-3-methyl-4-oxo-3,4-dihydroquinazolin-2-yl)piperidine-1-carboxylate (12m). Compound 12m was prepared according to general procedure 10 from (R)-1-(tert-butoxycarbonyl)piperidine-3-carboxylic acid and 11j. Purification by flash chromatography (10-40% EtOAc/Hexanes) afforded 12m as a yellow solid (2.00 g, 54%). ¹H NMR (400 MHz, CDCl₃) δ 7.61 (d, J=2.9 Hz, 1H), 7.55 (d, J=8.9 Hz, 1H), 7.30 (dd, J=8.9, 2.9 Hz, 1H), 4.32-4.09 (m, 2H), 3.91 (s, 3H), 3.71 (s, 3H), 3.19-2.99 (m, 1H), 2.92 (tt, J=10.8, 3.4 Hz, 1H), 2.85-2.80 (m, 1H), 2.14-2.06 (m, 2H), 1.96 (tdd, J=13.3, 11.2, 3.9 Hz, 1H), 1.87-1.80 (m, 1H), 1.48 (s, 9H).

Step 3: Synthesis of (R)-6-methoxy-3-methyl-2-(piperidin-3-yl)quinazolin-4(3H)-one trifluoroacetic acid salt (13m). Compound 13m was prepared according to general procedure 11 from 12m. Isolation by vacuum filtration afforded 13m as a light yellow solid (1.80 g, 75%). 13m was used directly without analysis.

Step 4: Synthesis of (R)-6-methoxy-3-methyl-2-(1-(oxetan-3-ylmethyl)piperidin-3-yl)quinazolin-4(3H)-one (14s). Compound 14s was prepared according to general procedure 12 from 13m and 3-(bromomethyl)oxetane. Purification by flash chromatography (20-50% X/hexanes; X=2% NEt₃ 3:1 EtOAc/EtOH) afforded 14s as a sticky yellow solid (90.8 mg, 88%). ¹H NMR (400 MHz, CDCl₃) δ 7.61 (d, J=2.9 Hz, 1H), 7.55 (d, J=8.9 Hz, 1H), 7.30 (dd, J=8.9, 3.0 Hz, 1H), 4.81 (dt, J=7.8, 5.9 Hz, 2H), 4.43 (td, J=6.2, 1.4 Hz, 2H), 3.91 (s, 3H), 3.67 (s, 3H), 3.34-3.24 (m, 1H), 3.06 (tt, J=10.7, 3.3 Hz, 1H), 2.96 (ddt, J=11.4, 3.5, 1.8 Hz, 1H), 2.85-2.77 (m, 3H), 2.46 (t, J=10.9 Hz, 1H), 2.04 (qd, J=11.9, 2.8 Hz, 2H), 1.85-1.80 (m, 1H), 1.73-1.59 (m, 2H). HRMS (ESI) m/z: [M+H]⁺ Calcd for C₁₉H₂₆N₃O₃, 344.1969. Found 344.1984.

### Example 110: Synthesis of (R)-3-methyl-2-(1-(oxetan-3-ylmethyl)piperidin-3-yl)-6-(trifluoromethyl)quinazolin-4(3H)-one (14t)

Prepared according to scheme 4.

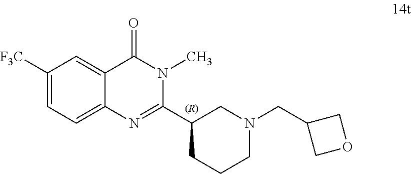

Step 1: Synthesis of 2-amino-N-methyl-5-(trifluoromethyl)benzamide (11k). Compound 11k was prepared according to general procedure 9a from 2-amino-5-(trifluoromethyl)-benzoic acid and methylammonium chloride. After work-up, 11k was afforded as a light yellow solid (1.36 g, 86%). ¹H NMR (400 MHz, CDCl₃) δ 7.55 (s, 1H), 7.40 (d, J=8.6 Hz, 1H), 6.70 (d, J=8.7 Hz, 1H), 6.20 (s, 1H), 5.91 (s, 2H), 2.97 (d, J=4.9 Hz, 3H).

Step 2: Synthesis of tert-butyl (R)-3-(3-methyl-4-oxo-6-(trifluoromethyl)-3,4-dihydroquinazolin-2-yl)piperidine-1-carboxylate (12n). Compound 12n was prepared according to general procedure 10 from (R)-1-(tert-butoxycarbonyl)piperidine-3-carboxylic acid and 11k. Purification by flash chromatography (15-40% EtOAc/Hexanes) afforded 12n as a white solid (1.86 g, 78%). ¹H NMR (400 MHz, CDCl₃) δ 8.53 (d, J=2.7 Hz, 1H), 7.89 (dd, J=8.7, 2.2 Hz, 1H), 7.72 (d, J=8.6 Hz, 1H), 4.33-4.11 (m, 2H), 3.72 (s, 3H), 3.24-3.05 (m, 1H), 2.97 (tt, J=10.9, 3.4 Hz, 1H), 2.88-2.78 (m, 1H), 2.13 (dt, J=13.4, 3.3 Hz, 1H), 1.97 (tdd, J=13.3, 11.0, 3.8 Hz, 1H), 1.86 (d, J=12.3 Hz, 1H), 1.67-1.55 (m, 1H), 1.47 (d, J=11.0 Hz, 9H).

Step 3: Synthesis of (R)-3-methyl-2-(piperidin-3-yl)-6-(trifluoromethyl)quinazolin-4(3H)-one trifluoroacetic acid salt (13n). Compound 13n was prepared according to general procedure 11 from 12n. Isolation by vacuum filtration afforded 13n as a light yellow solid (1.85 g, 79%). 13n was used directly without analysis.

Step 4: Synthesis of (R)-3-methyl-2-(1-(oxetan-3-ylmethyl)piperidin-3-yl)-6-(trifluoromethyl)quinazolin-4(3H)-one (14t). Compound 14t was prepared according to general procedure 12 from 13n and 3-(bromomethyl)oxetane. Purification by flash chromatography (20-50% X/hexanes; X=2% NEt₃ 3:1 EtOAc/EtOH) afforded 14t as a viscous yellow oil (89.1 mg, 78%). ¹H NMR (400 MHz, CDCl₃) δ 8.53-8.51 (m, 1H), 7.88 (dd, J=8.6, 2.2 Hz, 1H), 7.71 (d, J=8.6 Hz, 1H), 4.81 (dt, J=7.9, 5.9 Hz, 2H), 4.43 (t, J=6.2 Hz, 2H), 3.68 (s, 3H), 3.28 (hept, J=6.8 Hz, 1H), 3.12 (tt, J=9.3, 2.5 Hz, 1H), 2.99 (ddt, J=11.4, 3.6, 1.8 Hz, 1H), 2.88-2.80 (m, 3H), 2.50 (t, J=10.9 Hz, 1H), 2.06 (dt, J=12.5, 9.1 Hz, 2H), 1.88-1.83 (m, 1H), 1.78-1.61 (m, 2H). HRMS (ESI) m/z: [M+H]⁺ Calcd for C₁₉H₂₃F₃N₃O₂, 382.1737. Found 382.1748.

### Example 111: Synthesis of (R)-6,7-dichloro-3-methyl-2-(1-(pyridin-4-ylmethyl)piperidin-3-yl)quinazolin-4(3H)-one (14u)

Prepared according to scheme 4.

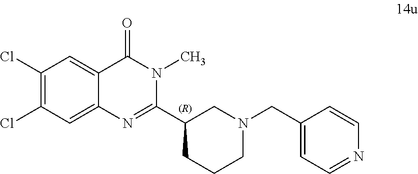

Steps 1-3: See Example 92—Synthesis of 13b.

Step 4: Synthesis of (R)-6,7-dichloro-3-methyl-2-(1-(pyridin-4-ylmethyl)piperidin-3-yl)quinazolin-4(3H)-one (14u). Compound 14u was prepared according to general procedure 12 from 13b and 4-(bromomethyl)pyridine hydrobromide. Purification by flash chromatography (20-60% X/hexanes; X=2% NEt₃ 3:1 EtOAc/EtOH) afforded 14u as a light yellow solid (101.4 mg, 84%). ¹H NMR (400 MHz, CDCl₃) δ 8.56-8.54 (m, 2H), 8.29 (s, 1H), 7.75 (s, 1H), 7.30-7.28 (m, 2H), 3.62 (s, 3H), 3.58 (d, J=5.4 Hz, 1H), 3.11 (tt, J=10.9, 3.3 Hz, 1H), 3.02-2.91 (m, 2H), 2.47 (t, J=10.8 Hz, 1H), 2.13 (td, J=11.5, 3.1 Hz, 1H), 2.05-2.02 (m, 1H), 1.89-1.83 (m, 1H), 1.82-1.60 (m, 3H). HRMS (ESI) m/z: [M+H]⁺ Calcd for C₂₀H₂₁Cl₂N₄O, 403.1087. Found 403.1102.

### Example 112: Synthesis of (R)-6,7-dichloro-3-methyl-2-(1-(pyridin-3-ylmethyl)piperidin-3-yl)quinazolin-4(3H)-one (14v)

Prepared according to scheme 4.

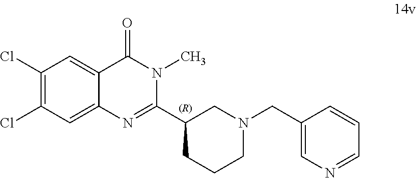

Steps 1-3: See Example 92—Synthesis of 13b.

Step 4: Synthesis of (R)-6,7-dichloro-3-methyl-2-(1-(pyridin-3-ylmethyl)piperidin-3-yl)quinazolin-4(3H)-one (14v). Compound 14v was prepared according to general procedure 12 from 13b and 3-(bromomethyl)pyridine hydrobromide. Purification by flash chromatography (20-60% X/hexanes; X=2% NEt₃ 3:1 EtOAc/EtOH) afforded 14v as an off-white solid (46.2 mg, 38%). ¹H NMR (400 MHz, CDCl₃) δ 8.59 (d, J=2.2 Hz, 1H), 8.51 (dd, J=4.9, 1.7 Hz, 1H), 8.29 (s, 1H), 7.75 (s, 1H), 7.67 (dt, J=7.8, 2.0 Hz, 1H), 7.28-7.25 (m, 1H), 3.64-3.55 (m, 4H), 3.12-3.04 (m, 1H), 2.99 (dd, J=21.8, 11.6 Hz, 2H), 2.44 (t, J=10.8 Hz, 1H), 2.12 (td, J=11.4, 3.0 Hz, 1H), 2.05-1.98 (m, 1H), 1.85 (dq, J=12.4, 3.1 Hz, 1H), 1.80-1.61 (m, 3H). HRMS (ESI) m/z: [M+H]⁺ Calcd for C₂₀H₂₁Cl₂N₄O, 403.1087. Found 403.1103.

### Example 113: Synthesis of (R)-6,7-dichloro-3-methyl-2-(1-(pyridin-2-ylmethyl)piperidin-3-yl)quinazolin-4(3H)-one (14w)

Prepared according to scheme 4.

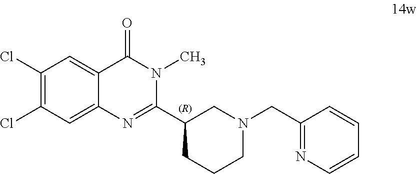

Steps 1-3: See Example 92—Synthesis of 13b.

Step 4: Synthesis of (R)-6,7-dichloro-3-methyl-2-(1-(pyridin-2-ylmethyl)piperidin-3-yl)quinazolin-4(3H)-one (14w). Compound 14w was prepared according to general procedure 12 from 13b and 2-(bromomethyl)pyridine hydrobromide. Purification by flash chromatography (10-30% X/hexanes; X=2% NEt₃ 3:1 EtOAc/EtOH) afforded 14w as an off-white solid (79.5 mg, 66%). ¹H NMR (400 MHz, CDCl₃) δ 8.60-8.58 (m, 1H), 8.29 (s, 1H), 7.75 (s, 1H), 7.66 (td, J=7.6, 1.8 Hz, 1H), 7.38 (d, J=7.8 Hz, 1H), 7.19-7.16 (m, 1H), 3.78-3.70 (m, 1H), 3.61 (s, 3H), 3.18 (tt, J=10.8, 3.3 Hz, 1H), 3.08 (d, J=11.5 Hz, 1H), 3.00 (d, J=11.4 Hz, 1H), 2.49 (t, J=10.9 Hz, 1H), 2.19 (td, J=11.0, 5.4 Hz, 1H), 2.03 (s, 1H), 1.82 (tt, J=10.7, 3.1 Hz, 1H), 1.76-1.63 (m, 3H). HRMS (ESI) m/z: [M+H]⁺ Calcd for C₂₀H₂₁Cl₂N₄O, 403.1087. Found 403.1098.

### Example 114: Synthesis of (R)-6,7-dichloro-3-methyl-2-(1-(pyrazin-2-ylmethyl)piperidin-3-yl)quinazolin-4(3H)-one (14x)

Prepared according to scheme 4.

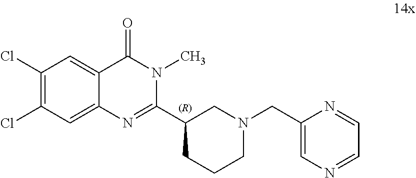

Steps 1-3: See Example 92—Synthesis of 13b.

Step 4: Synthesis of (R)-6,7-dichloro-3-methyl-2-(1-(pyrazin-2-ylmethyl)piperidin-3-yl)quinazolin-4(3H)-one (14x). Compound 14x was prepared according to general procedure 12 from 13b and 2-(bromomethyl)pyrazine hydrobromide. Purification by flash chromatography (5-50% X/hexanes; X=2% NEt₃ 3:1 EtOAc/EtOH) afforded 14x as a light yellow solid (51.3 mg, 42%). ¹H NMR (400 MHz, CDCl₃) δ 8.69 (d, J=1.5 Hz, 1H), 8.55 (dd, J=2.5, 1.6 Hz, 1H), 8.48 (d, J=2.6 Hz, 1H), 8.29 (s, 1H), 7.75 (s, 1H), 3.79 (s, 2H), 3.62 (s, 3H), 3.17 (tt, J=10.8, 3.3 Hz, 1H), 3.09-3.06 (m, 1H), 2.99 (d, J=11.4 Hz, 1H), 2.58 (t, J=10.9 Hz, 1H), 2.22 (td, J=11.2, 3.8 Hz, 1H), 2.04 (d, J=13.0 Hz, 1H), 1.90-1.61 (m, 3H). HRMS (ESI) m/z: [M+H]⁺ Calcd for C₁₉H₂₀Cl₂N₅O, 404.1039. Found 404.1056.

### Example 115: Synthesis of (S)-6,7-dichloro-2-(1-(2-hydroxyethyl)piperidin-3-yl)-3-methylquinazolin-4(3H)-one (14y)

Prepared according to scheme 4.

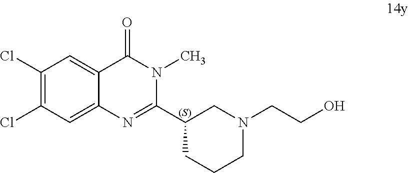

Steps 1-3: See Example 91—Synthesis of 13a.

Step 4: Synthesis of (S)-6,7-dichloro-2-(1-(2-hydroxyethyl)piperidin-3-yl)-3-methylquinazolin-4(3H)-one (14y). Compound 14y was prepared according to general procedure 12 from 13a and 2-bromoethanol. Purification by flash chromatography (40-100% X/hexanes; X=2% NEt₃ 3:1 EtOAc/EtOH) afforded 14y as a white solid (101.6 mg, 95%). ¹H NMR (400 MHz, CDCl₃) δ 8.31 (s, 1H), 7.76 (s, 1H), 3.66 (s, 5H), 3.12-3.04 (m, 2H), 2.99 (d, J=11.1 Hz, 1H), 2.67-2.61 (m, 2H), 2.60-2.55 (m, 1H), 2.19 (td, J=11.5, 3.0 Hz, 1H), 2.03 (d, J=11.6 Hz, 1H), 1.87 (dq, J=12.1, 3.1 Hz, 1H), 1.77-1.62 (m, 3H). HRMS (ESI) m/z: [M+H]⁺ Calcd for C₁₆H₂₀Cl₂N₃O₂, 356.0927. Found 356.0920.

### Example 116: Synthesis of (R)-6,7-dichloro-2-(1-(2-hydroxyethyl)piperidin-3-yl)-3-methylquinazolin-4(3H)-one (14z)

Prepared according to scheme 4.

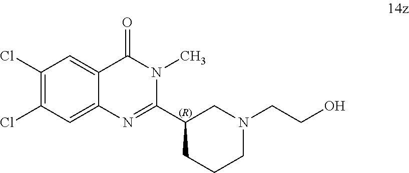

Steps 1-3: See Example 92—Synthesis of 13b.

Step 4: Synthesis of (R)-6,7-dichloro-2-(1-(2-hydroxyethyl)piperidin-3-yl)-3-methylquinazolin-4(3H)-one (14z). Compound 14z was prepared according to general procedure 12 from 13b and 2-bromoethanol. Purification by flash chromatography (10-100% X/hexanes; X=2% NEt₃ 3:1 EtOAc/EtOH) afforded 14z as a white solid (383.0 mg, 97%). ¹H NMR (400 MHz, CDCl₃) δ 8.31 (s, 1H), 7.76 (s, 1H), 3.69-3.64 (m, 5H), 3.10 (ddt, J=14.4, 10.2, 3.4 Hz, 2H), 3.01 (d, J=11.5 Hz, 1H), 2.67-2.62 (m, 2H), 2.61-2.57 (m, 1H), 2.20 (td, J=11.4, 3.0 Hz, 1H), 2.03 (d, J=11.6 Hz, 1H), 1.87 (dt, J=12.2, 2.9 Hz, 1H), 1.79-1.62 (m, 3H). HRMS (ESI) m/z: [M+H]⁺ Calcd for C₁₆H₂₀Cl₂N₃O₂, 356.0927. Found 356.0941.

### Example 117: Synthesis of (R)-6,7-dichloro-2-(1-(2-isopropoxyethyl)piperidin-3-yl)-3-methylquinazolin-4(3H)-one (14aa)

Prepared according to scheme 4.

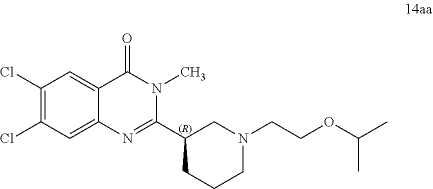

Steps 1-3: See Example 92—Synthesis of 13b.

Step 4: Synthesis of (R)-6,7-dichloro-2-(1-(2-isopropoxyethyl)piperidin-3-yl)-3-methylquinazolin-4(3H)-one (14aa). Compound 14aa was prepared according to general procedure 12 from 13b and 2-(2-bromoethoxy)propane. Purification by flash chromatography (5-50% X/hexanes; X=2% NEt₃ 3:1 EtOAc/EtOH) afforded 14aa as a white solid (96.7 mg, 81%). ¹H NMR (400 MHz, CDCl₃) δ 8.30 (s, 1H), 7.76 (s, 1H), 3.65 (s, 3H), 3.58 (t, J=6.1 Hz, 2H), 3.13 (ddt, J=11.4, 6.6, 3.4 Hz, 2H), 3.03 (d, J=11.5 Hz, 1H), 2.67 (t, J=6.2 Hz, 2H), 2.46 (t, J=11.7 Hz, 1H), 2.14 (td, J=11.5, 3.2 Hz, 1H), 2.00-1.97 (m, 1H), 1.86-1.80 (m, 1H), 1.78-1.62 (m, 3H), 1.16 (d, J=6.1 Hz, 6H). HRMS (ESI) m/z: [M+H]⁺ Calcd for C₁₉H₂₆Cl₂N₃O₂, 398.1397. Found 398.1406.

### Example 118: Synthesis of (R)-6,7-dichloro-3-methyl-2-(1-(2-morpholinoethyl)piperidin-3-yl)quinazolin-4(3H)-one (14bb)

Prepared according to scheme 4.

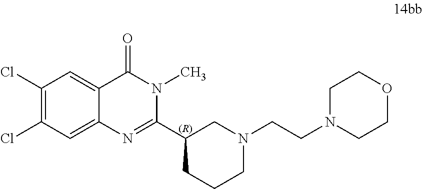

Steps 1-4: See Example 116—Synthesis of 14z.

Step 5: Synthesis of (R)-6,7-dichloro-3-methyl-2-(1-(2-morpholinoethyl)piperidin-3-yl)quinazolin-4(3H)-one (14bb). Compound 14bb was prepared according to general procedure 12b from 14z and morpholine. Purification by flash chromatography (20-80% X/hexanes; X=2% NEt₃ 3:1 EtOAc/EtOH) afforded 14bb as an off-white solid (115.6 mg, 65%). ¹H NMR (400 MHz, CDCl₃) δ 8.30 (s, 1H), 7.76 (s, 1H), 3.72-3.70 (m, 4H), 3.65 (s, 3H), 3.11 (ddt, J=11.1, 7.3, 3.2 Hz, 2H), 3.02 (d, J=11.4 Hz, 1H), 2.64-2.42 (m, 8H), 2.11 (td, J=11.5, 3.0 Hz, 1H), 2.04-1.97 (m, 1H), 1.90-1.60 (m, 4H). HRMS (ESI) m/z: [M+H]⁺ Calcd for C₂₀H₂₇Cl₂N₄O₂, 425.1506. Found 425.1522.

### Example 119: Synthesis of (S)-6,7-dichloro-3-methyl-2-(1-(2-(pyrrolidin-1-yl)ethyl)piperidin-3-yl)quinazolin-4(3H)-one (14cc)

Prepared according to scheme 4.

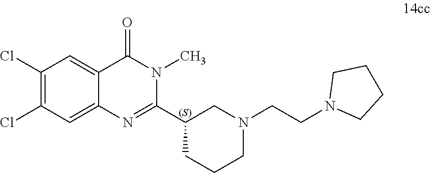

Steps 1-4: See Example 115—Synthesis of 14y.

Step 5: Synthesis of (S)-6,7-dichloro-3-methyl-2-(1-(2-(pyrrolidin-1-yl)ethyl)piperidin-3-yl)quinazolin-4(3H)-one (14cc). Compound 14cc was prepared according to general procedure 12b from 14y and pyrrolidine. Purification by flash chromatography (20-80% X/hexanes; X=2% NEt₃ 3:1 EtOAc/EtOH) afforded 14cc as a yellow solid (38.6 mg, 47%). ¹H NMR (400 MHz, CDCl₃) δ 8.27 (s, 1H), 7.75 (s, 1H), 3.65 (s, 3H), 3.12 (dt, J=10.9, 5.6 Hz, 2H), 3.05-3.02 (m, 1H), 2.72-2.60 (m, 4H), 2.54 (tt, J=4.1, 1.7 Hz, 4H), 2.45 (t, J=11.6 Hz, 1H), 2.10 (td, J=11.5, 3.1 Hz, 1H), 2.00 (dt, J=12.5, 2.8 Hz, 1H), 1.88-1.72 (m, 5H) 1.71-1.61 (m, 2H). HRMS (ESI) m/z: [M+H]⁺ Calcd for C₂₀H₂₇Cl₂N₄O, 409.1556. Found 409.1548.

### Example 120: Synthesis of (R)-6,7-dichloro-3-methyl-2-(1-(2-(pyrrolidin-1-yl)ethyl)piperidin-3-yl)quinazolin-4(3H)-one (14dd)

Prepared according to scheme 4.

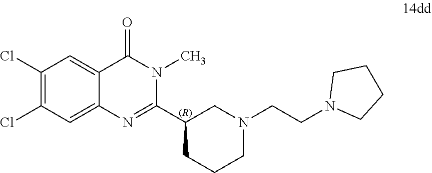

Steps 1-4: See Example 116—Synthesis of 14z.

Step 5: Synthesis of (R)-6,7-dichloro-3-methyl-2-(1-(2-(pyrrolidin-1-yl)ethyl) piperidin-3-yl)quinazolin-4(3H)-one (14dd). Compound 14dd was prepared according to general procedure 12b from 14z and pyrrolidine. Purification by flash chromatography (20-100% X/hexanes; X=2% NEt₃ 3:1 EtOAc/EtOH) afforded 14dd as a yellow solid (36.3 mg, 21%). ¹H NMR (400 MHz, CDCl₃) δ 8.30 (s, 1H), 7.76 (s, 1H), 3.65 (s, 3H), 3.17-3.09 (m, 2H), 3.05-3.00 (m, 1H), 2.74-2.60 (m, 4H), 2.59-2.56 (m, 4H), 2.48-2.40 (m, 1H), 2.10 (td, J=11.5, 3.1 Hz, 1H), 2.02-1.97 (m, 1H), 1.87-1.76 (m, 5H), 1.74-1.60 (m, 2H). HRMS (ESI) m/z: [M+H]⁺ Calcd for C₂₀H₂₇Cl₂N₄O, 409.1556. Found 409.1573.

### Example 121: Synthesis of (R)-6,7-dichloro-2-(1-(cyclopropylmethyl)piperidin-3-yl)-3-methylquinazolin-4(3H)-one (14ee)

Prepared according to scheme 4.

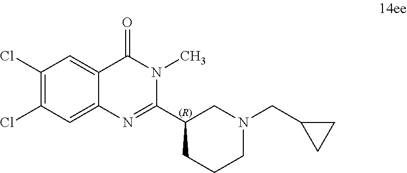

Steps 1-3: See Example 92—Synthesis of 13b.

Step 4: Synthesis of (R)-6,7-dichloro-2-(1-(cyclopropylmethyl)piperidin-3-yl)-3-methylquinazolin-4(3H)-one (14ee). Compound 14ee was prepared according to general procedure 12b from 14z and (bromomethyl)cyclopropane. Purification by flash chromatography (2-25% X/hexanes; X=2% NEt₃ 3:1 EtOAc/EtOH) afforded 14ee as an off white solid (65.2 mg, 89%). ¹H NMR (400 MHz, CDCl₃) δ 8.30 (s, 1H), 7.76 (s, 1H), 3.67 (s, 3H), 3.24 (ddt, J=11.4, 3.6, 1.8 Hz, 1H), 3.15 (ddq, J=10.4, 6.6, 3.4 Hz, 2H), 2.42-2.32 (m, 3H), 2.11-1.97 (m, 2H), 1.90-1.61 (m, 3H), 0.97-0.84 (m, 1H), 0.59-0.49 (m, 2H), 0.17-0.10 (m, 2H). HRMS (ESI) m/z: [M+H]⁺ Calcd for C₁₈H₂₂Cl₂N₃O, 366.1134. Found 366.1118.

### Example 122: Synthesis of (R)-6-chloro-8-fluoro-2-(1-(2-hydroxyethyl)piperidin-3-yl)-3-methylquinazolin-4(3H)-one (14ff)

Prepared according to scheme 4.

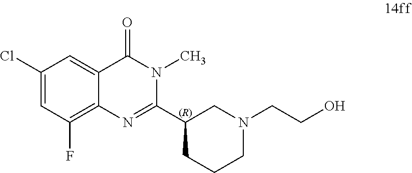

Step 1: Synthesis of 2-amino-5-chloro-3-fluoro-N-methylbenzamide (11l). Compound 11l was prepared according to general procedure 9a from 2-amino-5-chloro-3-fluorobenzoic acid and methylammonium chloride. After work-up, 11l was afforded as a tan solid (501.3 mg, 94%). ¹H NMR (400 MHz, DMSO) δ 8.51-8.35 (m, 1H), 7.44 (t, J=1.9 Hz, 1H), 7.34 (dd, J=11.1, 2.3 Hz, 1H), 6.48 (s, 2H), 2.74 (d, J=4.5 Hz, 3H). HRMS (ESI) m/z: [M+H]⁺ Calcd for C₈H₉ClFN₂O, 203.0381. Found 203.0381.

Step 2: Synthesis of tert-butyl (R)-3-(6-chloro-8-fluoro-3-methyl-4-oxo-3,4-dihydroquinazolin-2-yl)piperidine-1-carboxylate (120). Compound 12o was prepared according to general procedure 10 from (R)-1-(tert-butoxycarbonyl)piperidine-3-carboxylic acid and 11l. Purification by reverse phase flash chromatography (10-100% MeOH/H₂O) afforded 12o as a white solid (212.7 mg, 24%). ¹H NMR (400 MHz, CDCl₃) δ 8.02 (t, J=1.8 Hz, 1H), 7.43 (dd, J=9.5, 2.3 Hz, 1H), 4.23 (d, J=64.2 Hz, 2H), 3.71 (s, 3H), 3.12 (s, 1H), 2.95 (d, J=11.0 Hz, 1H), 2.83 (s, 1H), 2.11 (d, J=13.5 Hz, 1H), 1.99 (td, J=12.2, 3.8 Hz, 1H), 1.89-1.79 (m, 1H), 1.48 (s, 10H). HRMS (ESI) m/z: [M+H]⁺ Calcd for C₁₉H₂₄ClFN₃O₃, 396.1484. Found 396.1485.

Step 3: Synthesis of (R)-6-chloro-8-fluoro-3-methyl-2-(piperidin-3-yl)quinazolin-4(3H)-one trifluoroacetic acid salt (130). Compound 13o was prepared according to general procedure 11 from 12o. Isolation by vacuum filtration afforded 13o as a white solid (156.2 mg, 66%). 13o was used directly without analysis.

Step 4: Synthesis of (R)-6-chloro-8-fluoro-2-(1-(2-hydroxyethyl)piperidin-3-yl)-3-methylquinazolin-4(3H)-one (14ff). Compound 14ff was prepared according to general procedure 12 from 13o and 2-bromoethanol. Purification by flash chromatography (40-100% X/hexanes; X=2% NEt₃ 3:1 EtOAc/EtOH) afforded 14ff as an off white solid (81.2 mg, 96%). ¹H NMR (400 MHz, CDCl₃) δ 8.02 (dd, J=2.3, 1.4 Hz, 1H), 7.42 (dd, J=9.5, 2.3 Hz, 1H), 3.72-3.61 (m, 5H), 3.16-3.06 (m, 2H), 3.00 (d, J=11.3 Hz, 1H), 2.71-2.58 (m, 3H), 2.21 (td, J=11.5, 3.0 Hz, 1H), 2.05 (dq, J=6.2, 3.0 Hz, 2H), 1.88 (dq, J=8.4, 3.0 Hz, 1H), 1.80-1.67 (m, 2H). HRMS (ESI) m/z: [M+H]⁺ Calcd for C₁₆H₂₀ClFN₃O₂, 340.1222. Found 340.1220.

### Example 123: Synthesis of (R)-6-chloro-8-fluoro-3-methyl-2-(1-(2-(pyrrolidin-1-yl)ethyl)piperidin-3-yl)quinazolin-4(3H)-one (14gg)

Prepared according to scheme 4.

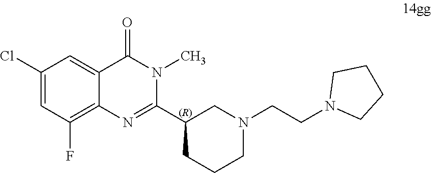

Steps 1-4: See Example 122—Synthesis of 14ff.

Step 5: Synthesis of (R)-6-chloro-8-fluoro-3-methyl-2-(1-(2-(pyrrolidin-1-yl)ethyl)piperidin-3-yl)quinazolin-4(3H)-one (14gg). Compound 14gg was prepared according to general procedure 12b from 14ff and pyrrolidine. Purification by flash chromatography (30-70% X/hexanes; X=2% NEt₃ 3:1 EtOAc/EtOH) afforded 14gg as a viscous yellow oil (27.1 mg, 46%). ¹H NMR (400 MHz, CDCl₃) δ 8.03-7.98 (m, 1H), 7.41 (dd, J=9.6, 2.4 Hz, 1H), 3.68 (s, 3H), 3.15 (tdd, J=10.5, 5.3, 2.4 Hz, 2H), 3.02 (d, J=10.9 Hz, 1H), 2.79-2.69 (m, 2H), 2.69-2.61 (m, 6H), 2.53 (t, J=11.1 Hz, 1H), 2.14 (td, J=11.4, 3.2 Hz, 1H), 2.06-1.97 (m, 1H), 1.88-1.76 (m, 5H), 1.76-1.66 (m, 2H). HRMS (ESI) m/z: [M+H]⁺ Calcd for C₂₀H₂₇ClFN₄O, 393.1851. Found 393.1848.

### Example 124: Assay for Determination of EC₅₀ Values Against Naegleria fowleri

N. fowleri strain Nf69 (ATCC 30215) trophozoites were cultured axenically. Amoeba were seeded at 1×10\hat{ } cells/mL in 100 μL of the indicated medium in white TC-treated 96-well plates (Thermo Scientific Nunc Microwell 136101). Cells were seeded into Nelson's Complete Media (NCM, 0.17% liver infusion broth (BD Difco, Franklin Lakes, NJ), 0.17% glucose, 0.012% sodium chloride, 0.0136% potassium phosphate monobasic, 0.0142% sodium phosphate dibasic, 0.0004% calcium chloride, 0.0002% magnesium sulfate, 10% heat-inactivated fetal bovine serum (FBS), 1% penicillin-streptomycin) at 37° C. in 96-well tissue culture plates. Vehicle or compound at concentrations initially ranging from 20 to 0.375 μM were added to cells, followed by culture for 48 h (37° C., 5% CO₂). Plates were equilibrated to rt for 15 min and CellTiter Glo reagent was added followed by orbital shaking for 2 min. After an additional incubation (rt, 10 min), luminescence was scored on a BioTek Synergy H1 microplate reader. Luminescence values were used to calculate percent cell growth inhibition (based on controls) for each concentration of compound tested. Averages of triplicates were calculated and fit to dose-response curves for the determination of EC₅₀ values, using Prism 9.0 (GraphPad Software, San Diego, CA). Miltefosine (Sigma Aldrich) was used a positive control (N. fowleri EC₅₀=35.7±9.10 mM). Other controls included media alone, cells and media, and vehicle and media. For very potent compounds, concentration of reagent in the dose-response assays were reduced to provide an appropriate range for EC₅₀ value resolution.

Compounds of the present technology were tested for growth inhibition of N. fowleri per General Procedure 13, above. Results are shown in Table 1.

TABLE 1
In vitro bioactivity of compounds.

| | | | | |
|---|---|---|---|---|
| EC₅₀ range | 0.0001-0.10 μM | >0.10 to 1 μM | >1 to 10 μM | > 10 μM |
| notation | XXXX | XXX | XX | X |

| Structure | Compound | EC₅₀ uM against N. fowleri |
|---|---|---|
| 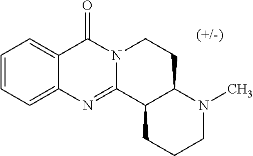 [SMILES: CN1CCC[C@H]2c3nc4ccccc4c(=O)n3CC[C@H]21] | cis-5a | X |
| 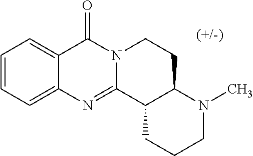 [SMILES: CN1CCC[C@@H]2c3nc4ccccc4c(=O)n3CC[C@H]21] | trans-5a | X |
| 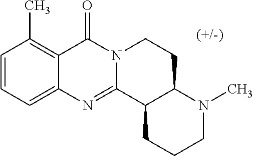 [SMILES: Cc1cccc2nc3n(c(=O)c12)CC[C@@H]1[C@H]3CCCN1C] | cis-5b | X |
| 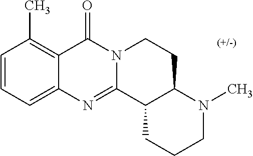 [SMILES: Cc1cccc2nc3n(c(=O)c12)CC[C@@H]1[C@@H]3CCCN1C] | trans-5b | X |
| 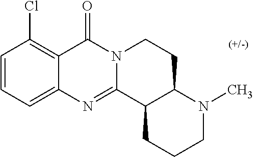 [SMILES: CN1CCC[C@H]2c3nc4cccc(Cl)c4c(=O)n3CC[C@H]21] | cis-5c | X |
| 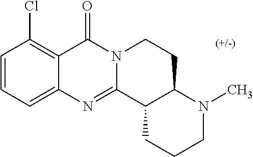 [SMILES: CN1CCC[C@@H]2c3nc4cccc(Cl)c4c(=O)n3CC[C@H]21] | trans-5c | X |
| 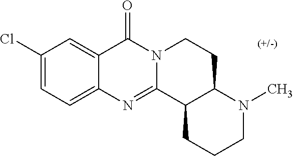 [SMILES: CN1CCC[C@H]2c3nc4ccc(Cl)cc4c(=O)n3CC[C@H]21] | cis-5d | XX |
| 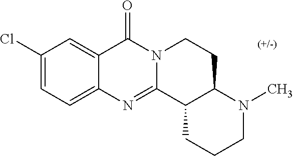 [SMILES: CN1CCC[C@@H]2c3nc4ccc(Cl)cc4c(=O)n3CC[C@H]21] | trans-5d | XXX |
| 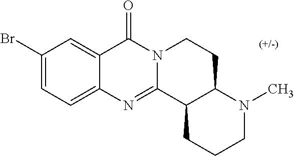 [SMILES: CN1CCC[C@H]2c3nc4ccc(Br)cc4c(=O)n3CC[C@H]21] | cis-5e | XXX |
| 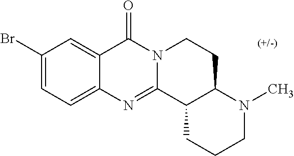 [SMILES: CN1CCC[C@@H]2c3nc4ccc(Br)cc4c(=O)n3CC[C@H]21] | trans-5e | XX |
| 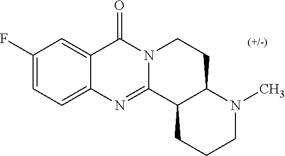 [SMILES: CN1CCC[C@H]2c3nc4ccc(F)cc4c(=O)n3CC[C@H]21] | cis-5f | X |
| 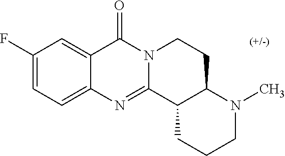 [SMILES: CN1CCC[C@@H]2c3nc4ccc(F)cc4c(=O)n3CC[C@H]21] | trans-5f | X |
| 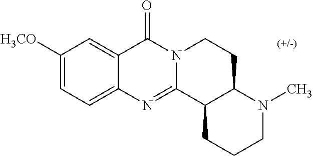 [SMILES: COc1ccc2nc3n(c(=O)c2c1)CC[C@@H]1[C@H]3CCCN1C] | cis-5g | X |
| 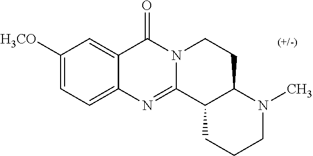 [SMILES: COc1ccc2nc3n(c(=O)c2c1)CC[C@@H]1[C@@H]3CCCN1C] | trans-5g | XX |
| 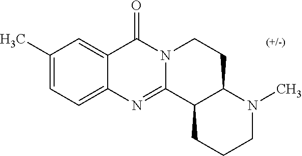 | cis-5h | XX |
| 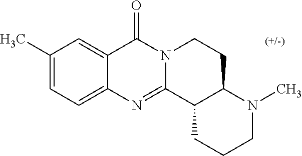 | trans-5h | XX |
| 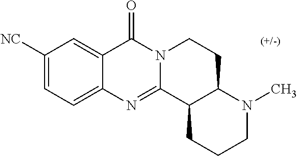 [SMILES: CN1CCC[C@H]2c3nc4ccc(C#N)cc4c(=O)n3CC[C@H]21] | cis-5i | XX |
| 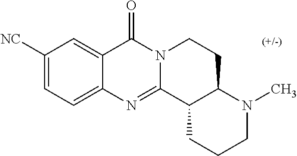 [SMILES: CN1CCC[C@@H]2c3nc4ccc(C#N)cc4c(=O)n3CC[C@H]21] | trans-5i | XXX |
| 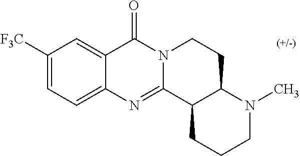 [SMILES: CN1CCC[C@H]2c3nc4ccc(C(F)(F)F)cc4c(=O)n3CC[C@H]21] | cis-5j | XXX |
| 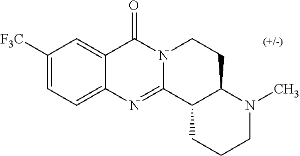 [SMILES: CN1CCC[C@@H]2c3nc4ccc(C(F)(F)F)cc4c(=O)n3CC[C@H]21] | trans-5j | XX |
| 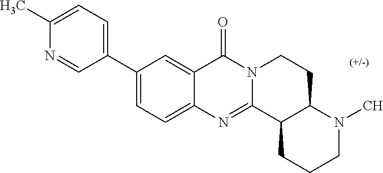 | cis-9a | X |
| 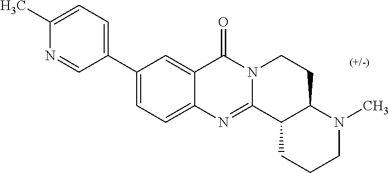 | trans-9a | X |
| 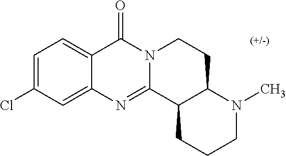 [SMILES: CN1CCC[C@H]2c3nc4cc(Cl)ccc4c(=O)n3CC[C@H]21] | cis-5k | X |
| 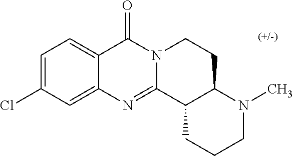 [SMILES: CN1CCC[C@@H]2c3nc4cc(Cl)ccc4c(=O)n3CC[C@H]21] | trans-5k | XX |
| 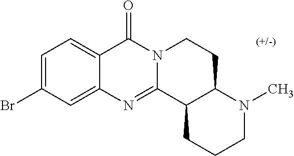 [SMILES: CN1CCC[C@H]2c3nc4cc(Br)ccc4c(=O)n3CC[C@H]21] | cis-5l | XX |
| 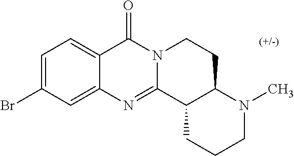 [SMILES: CN1CCC[C@@H]2c3nc4cc(Br)ccc4c(=O)n3CC[C@H]21] | trans-5l | XX |
| 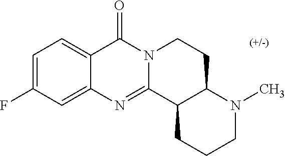 [SMILES: CN1CCC[C@H]2c3nc4cc(F)ccc4c(=O)n3CC[C@H]21] | cis-5m | X |
| 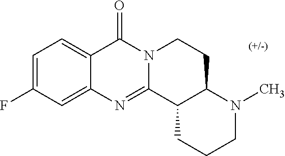 [SMILES: CN1CCC[C@@H]2c3nc4cc(F)ccc4c(=O)n3CC[C@H]21] | trans-5m | X |
| 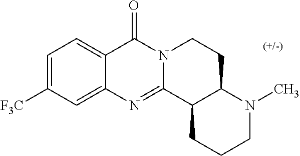 [SMILES: CN1CCC[C@H]2c3nc4cc(C(F)(F)F)ccc4c(=O)n3CC[C@H]21] | cis-5n | XX |
| 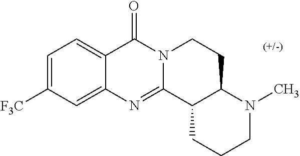 [SMILES: CN1CCC[C@@H]2c3nc4cc(C(F)(F)F)ccc4c(=O)n3CC[C@H]21] | trans-5n | XXX |
| 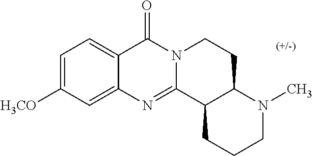 [SMILES: COc1ccc2c(=O)n3c(nc2c1)[C@@H]1CCCN(C)[C@@H]1CC3] | cis-50 | XX |
| 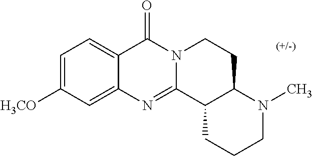 [SMILES: COc1ccc2c(=O)n3c(nc2c1)[C@H]1CCCN(C)[C@@H]1CC3] | trans-50 | XX |
| 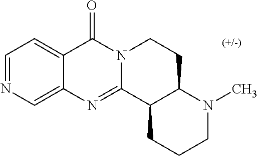 [SMILES: CN1CCC[C@H]2c3nc4cnccc4c(=O)n3CC[C@H]21] | cis-5p | X |
| 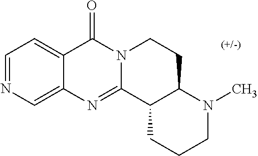 [SMILES: CN1CCC[C@@H]2c3nc4cnccc4c(=O)n3CC[C@H]21] | trans-5p | X |
| 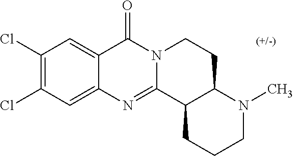 [SMILES: CN1CCC[C@H]2c3nc4cc(Cl)c(Cl)cc4c(=O)n3CC[C@H]21] | cis-5q | XXX |
| 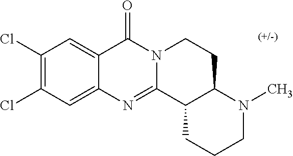 [SMILES: CN1CCC[C@@H]2c3nc4cc(Cl)c(Cl)cc4c(=O)n3CC[C@H]21] | trans-5q | XXX |
| 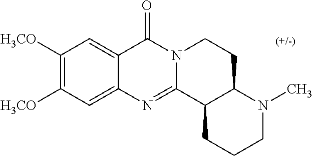 [SMILES: COc1cc2nc3n(c(=O)c2cc1OC)CC[C@@H]1[C@H]3CCCN1C] | cis-5r | X |
| 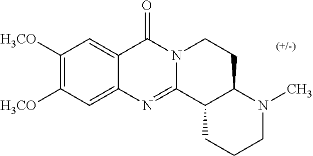 [SMILES: COc1cc2nc3n(c(=O)c2cc1OC)CC[C@@H]1[C@@H]3CCCN1C] | trans-5r | X |
| 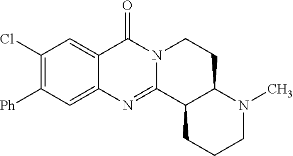 [SMILES: CN1CCC[C@H]2c3nc4cc(-c5ccccc5)c(Cl)cc4c(=O)n3CC[C@H]21] | cis-9b | XX |
| 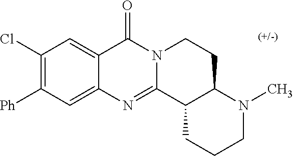 [SMILES: CN1CCC[C@@H]2c3nc4cc(-c5ccccc5)c(Cl)cc4c(=O)n3CC[C@H]21] | trans-9b | XX |
| 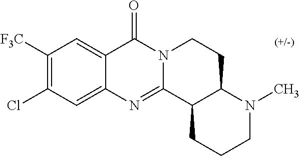 [SMILES: CN1CCC[C@H]2c3nc4cc(Cl)c(C(F)(F)F)cc4c(=O)n3CC[C@H]21] | cis-5s | XXX |
| 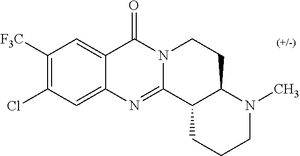 [SMILES: CN1CCC[C@@H]2c3nc4cc(Cl)c(C(F)(F)F)cc4c(=O)n3CC[C@H]21] | trans-5s | XXX |
| 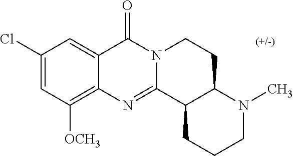 [SMILES: COc1cc(Cl)cc2c(=O)n3c(nc12)[C@@H]1CCCN(C)[C@@H]1CC3] | cis-5t | X |
| 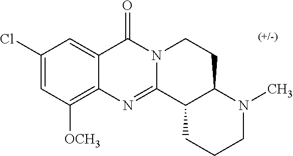 [SMILES: COc1cc(Cl)cc2c(=O)n3c(nc12)[C@H]1CCCN(C)[C@@H]1CC3] | trans-5t | X |
| 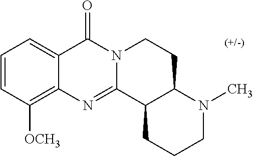 [SMILES: COc1cccc2c(=O)n3c(nc12)[C@@H]1CCCN(C)[C@@H]1CC3] | cis-5u | Not tested |
| 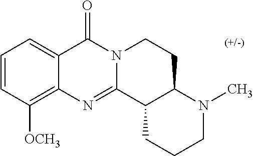 [SMILES: COc1cccc2c(=O)n3c(nc12)[C@H]1CCCN(C)[C@@H]1CC3] | trans-5u | X |
| 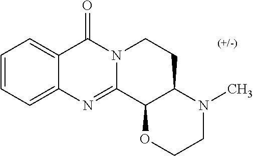 [SMILES: CN1CCO[C@H]2c3nc4ccccc4c(=O)n3CC[C@H]21] | cis-5v | X |
| 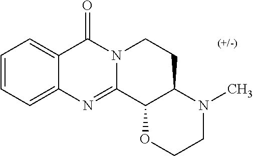 [SMILES: CN1CCO[C@@H]2c3nc4ccccc4c(=O)n3CC[C@H]21] | trans-5v | X |
| 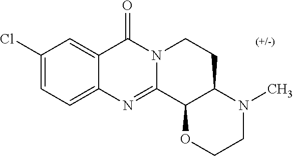 [SMILES: CN1CCO[C@H]2c3nc4ccc(Cl)cc4c(=O)n3CC[C@H]21] | cis-5w | X |
| 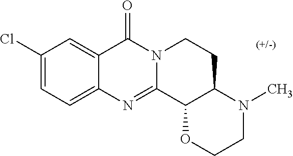 [SMILES: CN1CCO[C@@H]2c3nc4ccc(Cl)cc4c(=O)n3CC[C@H]21] | trans-5w | X |
| 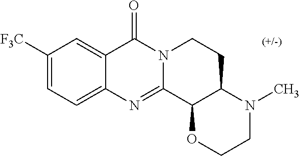 [SMILES: CN1CCO[C@H]2c3nc4ccc(C(F)(F)F)cc4c(=O)n3CC[C@H]21] | cis-5x | X |
| 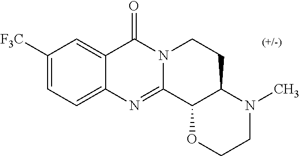 [SMILES: CN1CCO[C@@H]2c3nc4ccc(C(F)(F)F)cc4c(=O)n3CC[C@H]21] | trans-5x | X |
| 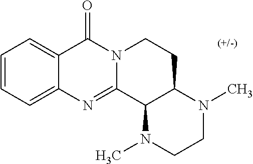 [SMILES: CN1CCN(C)[C@H]2c3nc4ccccc4c(=O)n3CC[C@H]21] | cis-5y | X |
| 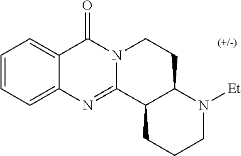 [SMILES: CCN1CCC[C@H]2c3nc4ccccc4c(=O)n3CC[C@H]21] | cis-5z | X |
| 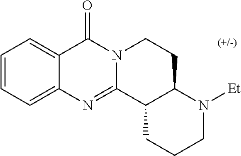 [SMILES: CCN1CCC[C@@H]2c3nc4ccccc4c(=O)n3CC[C@H]21] | trans-5z | X |
| 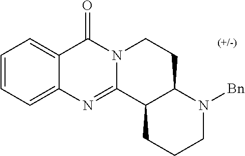 [SMILES: O=c1c2ccccc2nc2n1CC[C@@H]1[C@H]2CCCN1Cc1ccccc1] | cis-5aa | Not tested |
| 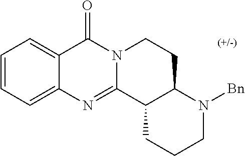 [SMILES: O=c1c2ccccc2nc2n1CC[C@@H]1[C@@H]2CCCN1Cc1ccccc1] | trans-5aa | X |
| 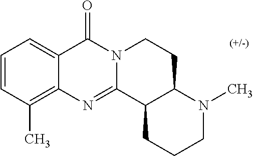 [SMILES: Cc1cccc2c(=O)n3c(nc12)[C@@H]1CCCN(C)[C@@H]1CC3] | cis-5bb | Not tested |
| 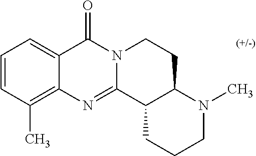 [SMILES: Cc1cccc2c(=O)n3c(nc12)[C@H]1CCCN(C)[C@@H]1CC3] | trans-5bb | Not tested |
| 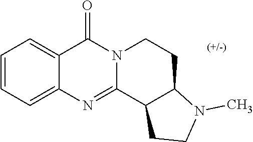 [SMILES: CN1CC[C@H]2c3nc4ccccc4c(=O)n3CC[C@H]21] | cis-5cc | X |
| 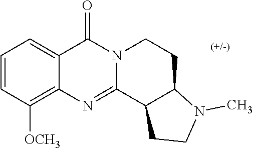 [SMILES: COc1cccc2c(=O)n3c(nc12)[C@@H]1CCN(C)[C@@H]1CC3] | cis-5dd | X |
| 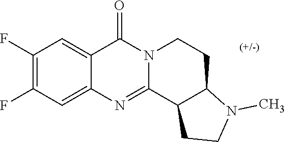 [SMILES: CN1CC[C@H]2c3nc4cc(F)c(F)cc4c(=O)n3CC[C@H]21] | cis-5ee | X |
| 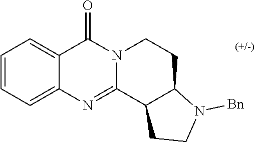 [SMILES: O=c1c2ccccc2nc2n1CC[C@@H]1[C@H]2CCN1Cc1ccccc1] | cis-5ff | X |
| 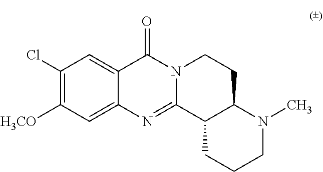 [SMILES: COc1cc2nc3n(c(=O)c2cc1Cl)CC[C@@H]1[C@@H]3CCCN1C] | trans-5gg | XXX |
| 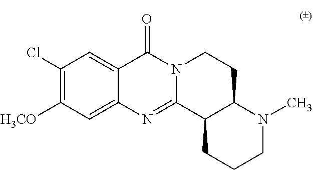 [SMILES: COc1cc2nc3n(c(=O)c2cc1Cl)CC[C@@H]1[C@H]3CCCN1C] | cis-5gg | XXX |
| 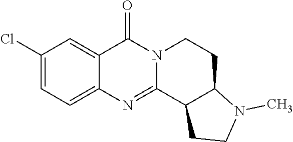 [SMILES: CN1CC[C@H]2c3nc4ccc(Cl)cc4c(=O)n3CC[C@H]21] | cis-5hh | X |
| 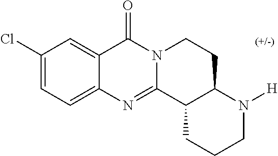 [SMILES: O=c1c2cc(Cl)ccc2nc2n1CC[C@H]1NCCC[C@H]21] | trans-6a | XX |
| 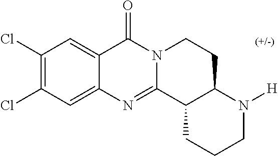 [SMILES: O=c1c2cc(Cl)c(Cl)cc2nc2n1CC[C@H]1NCCC[C@H]21] | trans-6b | XX |
| 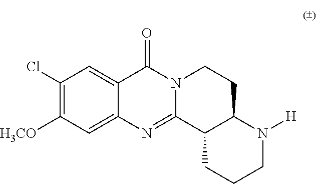 [SMILES: COc1cc2nc3n(c(=O)c2cc1Cl)CC[C@H]1NCCC[C@H]31] | trans-6c | XX |
| 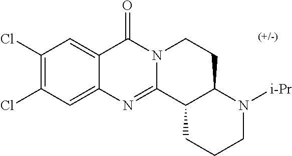 [SMILES: CC(C)N1CCC[C@@H]2c3nc4cc(Cl)c(Cl)cc4c(=O)n3CC[C@H]21] | trans-7a | XXXX |
| 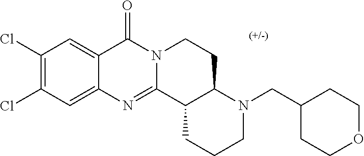 [SMILES: O=c1c2cc(Cl)c(Cl)cc2nc2n1CC[C@@H]1[C@@H]2CCCN1CC1CCOCC1] | trans-7b | XXXX |
| 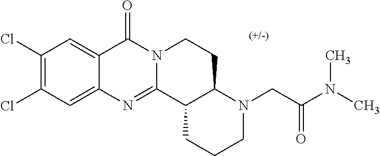 [SMILES: CN(C)C(=O)CN1CCC[C@@H]2c3nc4cc(Cl)c(Cl)cc4c(=O)n3CC[C@H]21] | trans-7c | XXX |
| 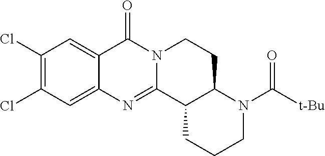 [SMILES: CC(C)(C)C(=O)N1CCC[C@@H]2c3nc4cc(Cl)c(Cl)cc4c(=O)n3CC[C@H]21] | trans-7d | X |
| 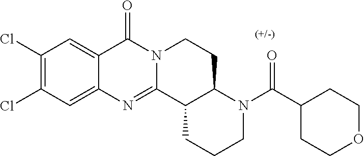 [SMILES: O=C(C1CCOCC1)N1CCC[C@@H]2c3nc4cc(Cl)c(Cl)cc4c(=O)n3CC[C@H]21] | trans-7e | X |
| 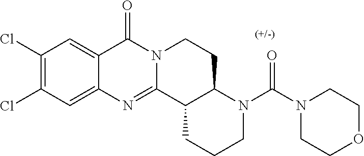 [SMILES: O=C(N1CCOCC1)N1CCC[C@@H]2c3nc4cc(Cl)c(Cl)cc4c(=O)n3CC[C@H]21] | trans-7f | X |
| 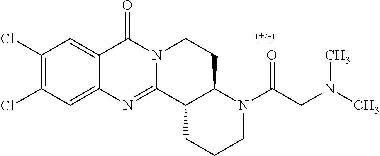 [SMILES: CN(C)CC(=O)N1CCC[C@@H]2c3nc4cc(Cl)c(Cl)cc4c(=O)n3CC[C@H]21] | trans-7g | XXX |
| 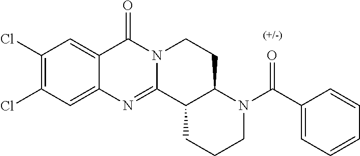 [SMILES: O=C(c1ccccc1)N1CCC[C@@H]2c3nc4cc(Cl)c(Cl)cc4c(=O)n3CC[C@H]21] | trans-7h | X |
| 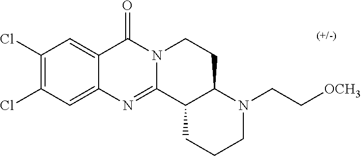 [SMILES: COCCN1CCC[C@@H]2c3nc4cc(Cl)c(Cl)cc4c(=O)n3CC[C@H]21] | trans-7i | XXXX |
| 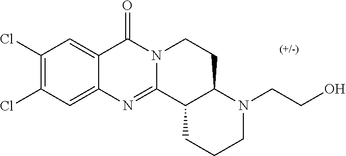 [SMILES: O=c1c2cc(Cl)c(Cl)cc2nc2n1CC[C@@H]1[C@@H]2CCCN1CCO] | trans-7j | XXXX |
| 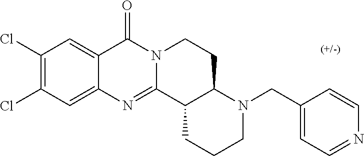 [SMILES: O=c1c2cc(Cl)c(Cl)cc2nc2n1CC[C@@H]1[C@@H]2CCCN1Cc1ccncc1] | trans-7k | XXX |
| 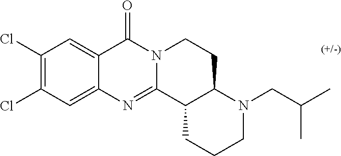 [SMILES: CC(C)CN1CCC[C@@H]2c3nc4cc(Cl)c(Cl)cc4c(=O)n3CC[C@H]21] | trans-7l | XXXX |
| 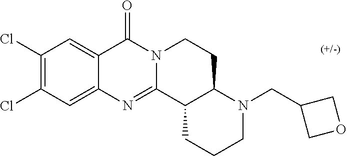 [SMILES: O=c1c2cc(Cl)c(Cl)cc2nc2n1CC[C@@H]1[C@@H]2CCCN1CC1COC1] | trans-7m | XXXX |
| 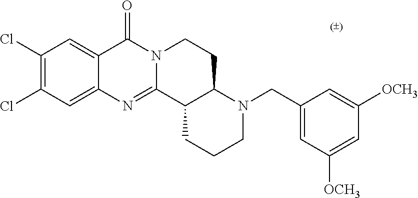 [SMILES: COc1cc(CN2CCC[C@@H]3c4nc5cc(Cl)c(Cl)cc5c(=O)n4CC[C@H]32)cc(OC)c1] | trans-7n | XXXX |
| 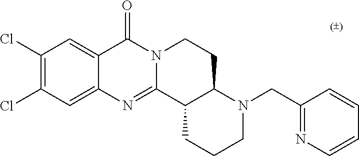 [SMILES: O=c1c2cc(Cl)c(Cl)cc2nc2n1CC[C@@H]1[C@@H]2CCCN1Cc1ccccn1] | trans-7o | XX |
| 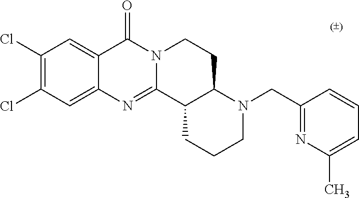 [SMILES: Cc1cccc(CN2CCC[C@@H]3c4nc5cc(Cl)c(Cl)cc5c(=O)n4CC[C@H]32)n1] | trans-7p | XXX |
| 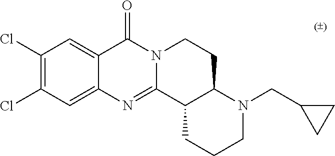 [SMILES: O=c1c2cc(Cl)c(Cl)cc2nc2n1CC[C@@H]1[C@@H]2CCCN1CC1CC1] | trans-7q | XXXX |
| 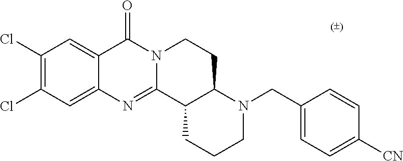 [SMILES: N#Cc1ccc(CN2CCC[C@@H]3c4nc5cc(Cl)c(Cl)cc5c(=O)n4CC[C@H]32)cc1] | trans-7r | X |
| 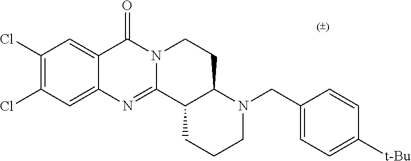 [SMILES: CC(C)(C)c1ccc(CN2CCC[C@@H]3c4nc5cc(Cl)c(Cl)cc5c(=O)n4CC[C@H]32)cc1] | trans-7s | X |
| 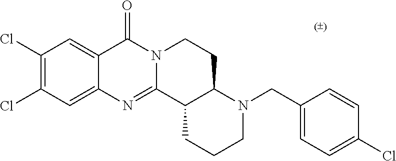 [SMILES: O=c1c2cc(Cl)c(Cl)cc2nc2n1CC[C@@H]1[C@@H]2CCCN1Cc1ccc(Cl)cc1] | 7t | X |
| 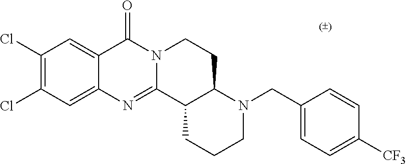 [SMILES: O=c1c2cc(Cl)c(Cl)cc2nc2n1CC[C@@H]1[C@@H]2CCCN1Cc1ccc(C(F)(F)F)cc1] | 7u | X |
| 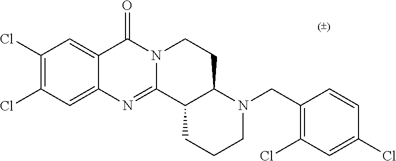 [SMILES: O=c1c2cc(Cl)c(Cl)cc2nc2n1CC[C@@H]1[C@@H]2CCCN1Cc1ccc(Cl)cc1Cl] | 7v | X |
| 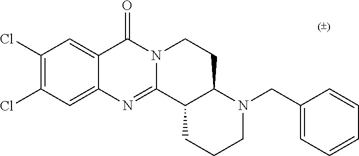 [SMILES: O=c1c2cc(Cl)c(Cl)cc2nc2n1CC[C@@H]1[C@@H]2CCCN1Cc1ccccc1] | trans-7w | XXX |
| 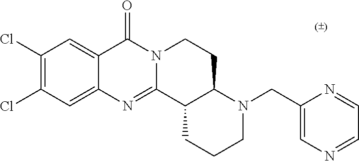 [SMILES: O=c1c2cc(Cl)c(Cl)cc2nc2n1CC[C@@H]1[C@@H]2CCCN1Cc1cnccn1] | trans-7x | XXXX |
| 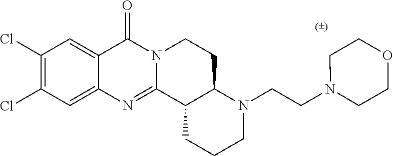 [SMILES: O=c1c2cc(Cl)c(Cl)cc2nc2n1CC[C@@H]1[C@@H]2CCCN1CCN1CCOCC1] | trans-7y | XXXX |
| 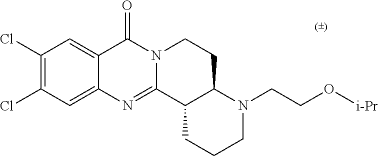 [SMILES: CC(C)OCCN1CCC[C@@H]2c3nc4cc(Cl)c(Cl)cc4c(=O)n3CC[C@H]21] | trans-7z | XXXX |
| 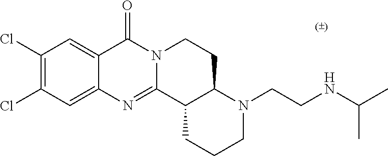 [SMILES: CC(C)NCCN1CCC[C@@H]2c3nc4cc(Cl)c(Cl)cc4c(=O)n3CC[C@H]21] | trans-7aa | XXX |
| 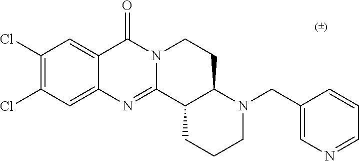 [SMILES: O=c1c2cc(Cl)c(Cl)cc2nc2n1CC[C@@H]1[C@@H]2CCCN1Cc1cccnc1] | trans-7bb | XXXX |
| 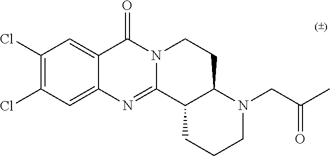 [SMILES: CC(=O)CN1CCC[C@@H]2c3nc4cc(Cl)c(Cl)cc4c(=O)n3CC[C@H]21] | trans-7cc | XXXX |
| 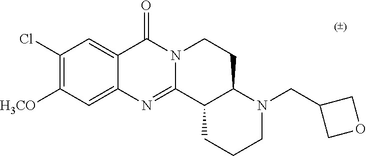 [SMILES: COc1cc2nc3n(c(=O)c2cc1Cl)CC[C@@H]1[C@@H]3CCCN1CC1COC1] | trans-7dd | XXXX |
| 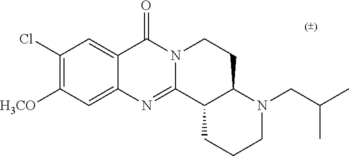 [SMILES: COc1cc2nc3n(c(=O)c2cc1Cl)CC[C@@H]1[C@@H]3CCCN1CC(C)C] | trans-7ee | XXX |
| 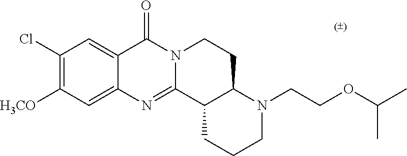 [SMILES: COc1cc2nc3n(c(=O)c2cc1Cl)CC[C@@H]1[C@@H]3CCCN1CCOC(C)C] | trans-7ff | XXX |
| 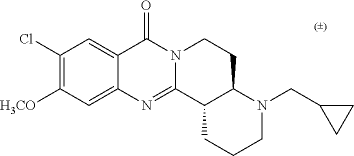 [SMILES: COc1cc2nc3n(c(=O)c2cc1Cl)CC[C@@H]1[C@@H]3CCCN1CC1CC1] | trans-7gg | XXXX |
| 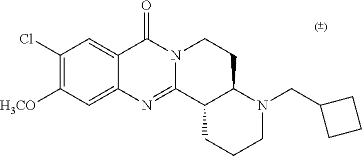 [SMILES: COc1cc2nc3n(c(=O)c2cc1Cl)CC[C@@H]1[C@@H]3CCCN1CC1CCC1] | trans-7hh | XXXX |
| 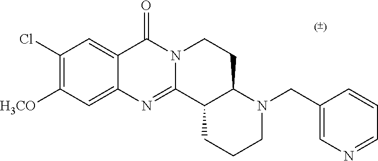 [SMILES: COc1cc2nc3n(c(=O)c2cc1Cl)CC[C@@H]1[C@@H]3CCCN1Cc1cccnc1] | trans-7ii | XXX |
| 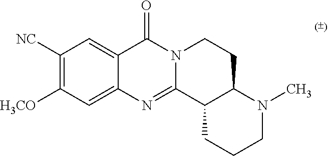 [SMILES: COc1cc2nc3n(c(=O)c2cc1C#N)CC[C@@H]1[C@@H]3CCCN1C] | trans-7jj | XX |
| 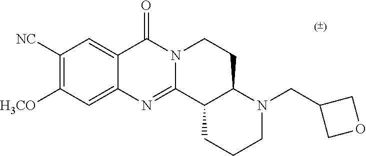 [SMILES: COc1cc2nc3n(c(=O)c2cc1C#N)CC[C@@H]1[C@@H]3CCCN1CC1COC1] | trans-7kk | XXX |
| 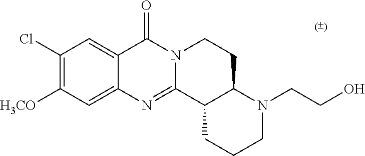 [SMILES: COc1cc2nc3n(c(=O)c2cc1Cl)CC[C@@H]1[C@@H]3CCCN1CCO] | trans-7ll | Not tested |
| 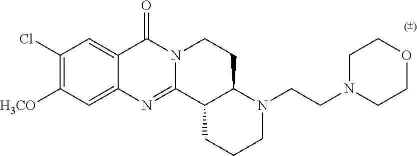 [SMILES: COc1cc2nc3n(c(=O)c2cc1Cl)CC[C@@H]1[C@@H]3CCCN1CCN1CCOCC1] | trans-7mm | XXX |
| 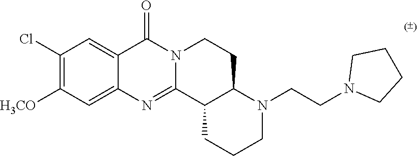 [SMILES: COc1cc2nc3n(c(=O)c2cc1Cl)CC[C@@H]1[C@@H]3CCCN1CCN1CCCC1] | trans-7nn | XXX |
| 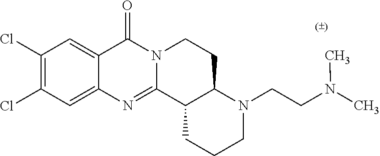 [SMILES: CN(C)CCN1CCC[C@@H]2c3nc4cc(Cl)c(Cl)cc4c(=O)n3CC[C@H]21] | trans-7oo | XXX |
| 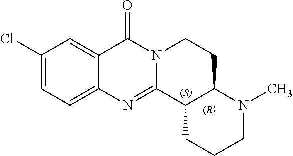 (*arbitrarily assigned) | trans-10a- ent 1 | XXX |
| 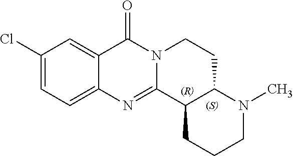 (*arbitrarily assigned) | trans-10b- ent-2 | XX |
| 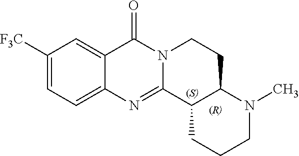 (*arbitrarily assigned) | trans-10c- ent-1 | XXX |
| 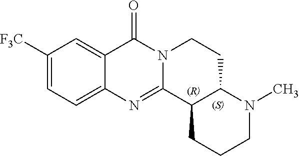 (*arbitrarily assigned) | trans-10d- ent-2 | XX |
| 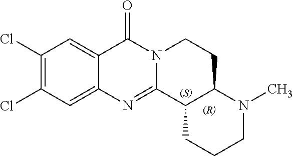 (*arbitrarily assigned) | trans-10e- ent-1 | XXXX |
| 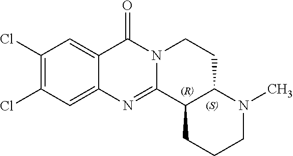 (*arbitrarily assigned) | trans-10f- ent-2 | XX |
| 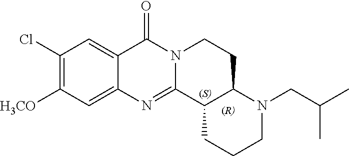 (*arbitrarily assigned) | trans-10g- ent-1 | XXX |
| 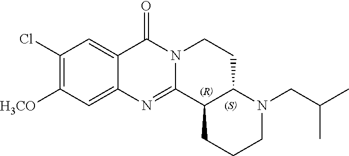 (*arbitrarily assigned) | trans-10h- ent-2 | XXXX |
| 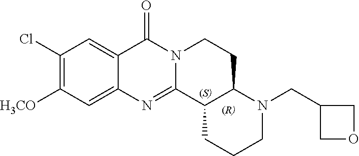 (*arbitrarily assigned) | trans-10i- ent-1 | XXX |
| 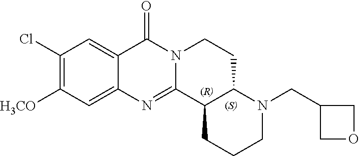 (*arbitrarily assigned) | trans-10j- ent-2 | XXXX |
| 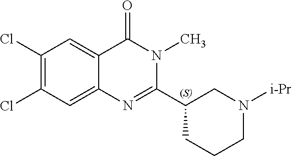 | S-14a | X |
| 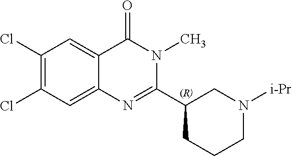 | R-14b | X |
| 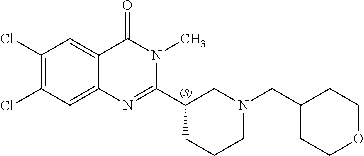 | S-14c | X |
| 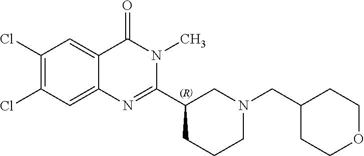 | R-14d | X |
| 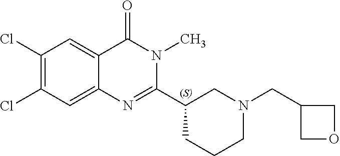 | S-14e | X |
| 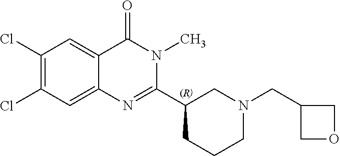 | R-14f | XXX |
| 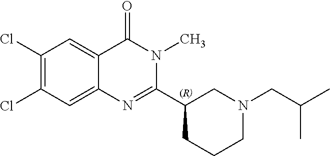 | R-14g | X |
| 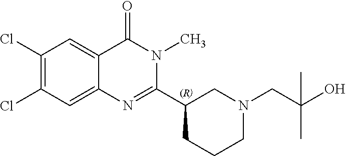 | R-14h | X |
| 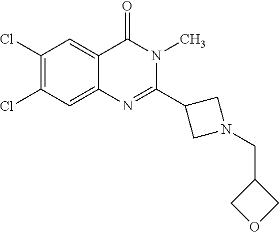 [SMILES: Cn1c(C2CN(CC3COC3)C2)nc2cc(Cl)c(Cl)cc2c1=O] | 14i | X |
| 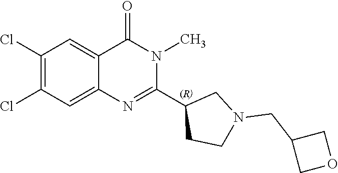 | R-14j | XX |
| 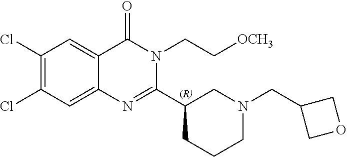 | R-14k | X |
| 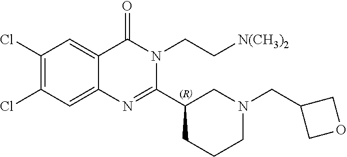 | R-14l | X |
| 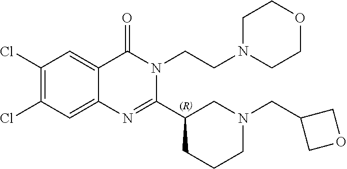 | R-14m | X |
| 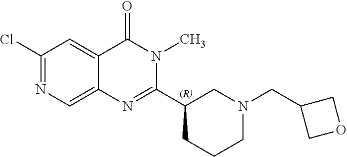 | R-14n | X |
| 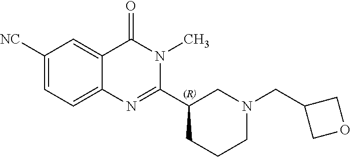 | R-14o | X |
| 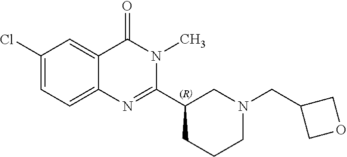 | R-14p | X |
| 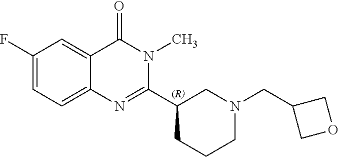 | R-14q | X |
| 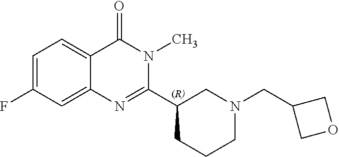 | R-14r | X |
| 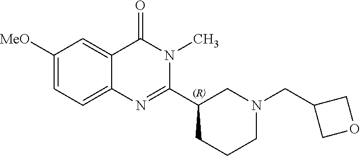 | R-14s | X |
| 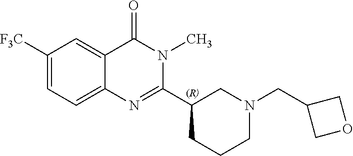 | R-14t | X |
| 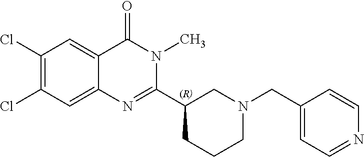 | R-14u | X |
| 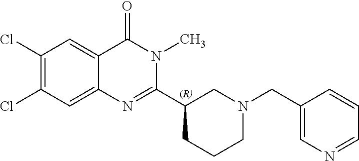 | R-14v | X |
| 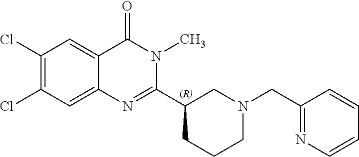 | R-14w | X |
| 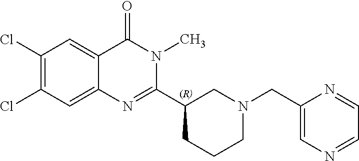 | R-14x | X |
| 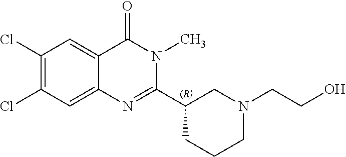 | S-14y | X |
| 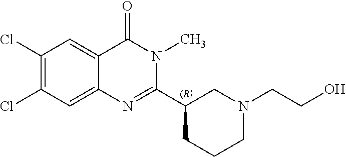 | R-14z | X |
| 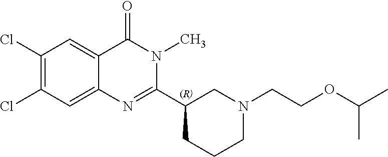 | R-14aa | XX |
| 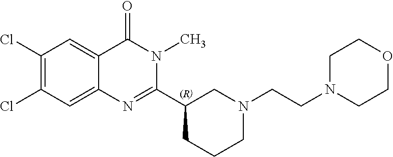 | R-14bb | XX |
| 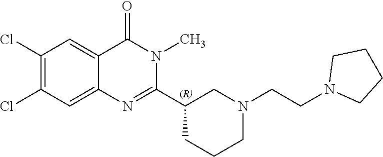 | S-14cc | X |
| 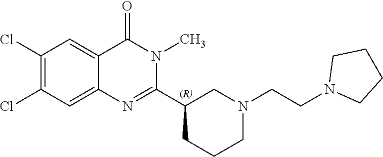 | R-14dd | XXX |
| 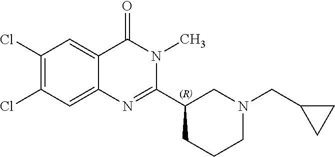 | R-14ee | Not tested |
| 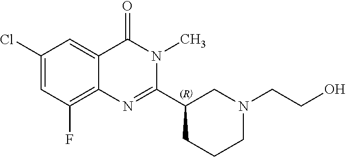 | R-14ff | Not tested |
| 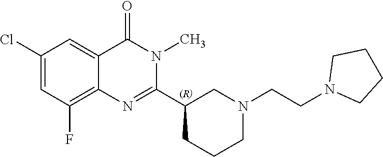 | R-14gg | Not tested |

### EQUIVALENTS

While certain embodiments have been illustrated and described, a person with ordinary skill in the art, after reading the foregoing specification, can effect changes, substitutions of equivalents and other types of alterations to the compounds and compositions of the present technology or derivatives, prodrugs, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, conjugates, reagents, compounds, compositions, labeled compounds or biological systems, which can, of course, vary. It is also to be understood that the terminology used herein is for the purpose of describing particular aspects only and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A compound of Formula I

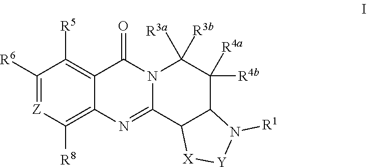

a stereoisomer of thereof, a pharmaceutically acceptable salt of the compound, or a pharmaceutically acceptable salt of the stereoisomer;
wherein,
   X is CH₂, O, or NR²;
   Y is CH₂ or CH₂CH₂, provided that when Y is CH₂, X is CH₂;
   Z is N or CR⁷;
   R¹ is H, C(O)R⁹, or a substituted or unsubstituted alkyl, cycloalkyl, cycloalkylalkyl, alkenyl, aralkyl, heterocyclylalkyl, or heteroarylalkyl group;
   R² is H or an unsubstituted alkyl group;
   R^{3a}, R^{3b}, R^{4a}, and R⁴ are independently selected from the group consisting of H and a substituted or unsubstituted alkyl group;
   R⁵ and R⁸ are independently selected from the group consisting of H, OR¹⁰, halogen and a substituted or unsubstituted alkyl, alkenyl, aryl, aralkyl, or heteroaryl group;
   R⁶ and R⁷ are independently selected from the group consisting of H, halogen, CN, NO₂, C(O)R¹¹, C(O)OR¹¹, C(O)NR¹¹R¹², OR¹², and a substituted or unsubstituted alkyl, aryl or heteroaryl group;
   R⁹ is substituted or unsubstituted alkyl, cycloalkyl, cycloalkylalkyl, alkenyl, aryl, aralkyl, heterocyclyl, heterocyclylalkyl, heteroaryl, or heteroarylalkyl group; and
   R¹⁰, R¹¹, and R¹² at each occurrence are independently H or an unsubstituted alkyl group.
B. The compound of Paragraph A, wherein X is CH₂.
C. The compound of Paragraph A, wherein X is O.
D. The compound of Paragraph A, wherein X is NR².
E. The compound of any one of Paragraphs A-D, wherein Y is CH₂CH₂.
F. The compound of any one of Paragraphs A-D, wherein Y is CH₂.
G. The compound of any one of Paragraphs A-F, wherein Z is N.
H. The compound of any one of Paragraphs A-F, wherein Z is CR⁷ and the compound is of Formula IA

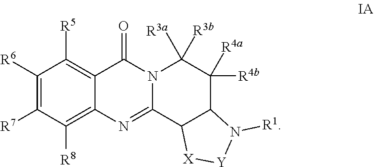

I. The compound of any one of Paragraphs A-H, wherein substituted alkyl, cycloalkyl, cycloalkylalkyl, alkenyl, aryl, aralkyl, heterocyclyl, heterocyclylalkyl, heteroaryl, and heteroarylalkyl groups are substituted with one or more substituents selected from the group consisting of halogen, hydroxyl, alkoxy, alkenoxy, aryloxy, aralkyloxy, heterocyclyl, heterocyclylalkyl, heterocyclyloxy, heterocyclylalkoxy, oxo, carboxyl, ester, urethane, oximes, hydroxylamine, alkoxyamine, aralkoxyamine, thiol, sulfide, sulfoxide, sulfone, sulfonyl, sulfonamide, sulfate, phosphate, amine, N-oxide, hydrazine, hydrazide, hydrazone, azide, amide, urea, amidine, guanidine, enamine, imide, imine, nitro, and nitrile groups, and further wherein the cycloalkylalkyl, aralkyl, heterocyclylalkyl, and heteroarylalkyl groups are also optionally substituted with one or more of an unsubstituted alkyl or alkenyl group, a haloalkyl group, or a haloalkenyl group.
J. The compound of Paragraph I, wherein the one or more substituents are selected from the group consisting of F, Cl, Br, OH, NR₂, oxo, C₁₋₆ alkoxy, C₁₋₆ alkyl, CN, halogen, and C₁₋₆ haloalkyl group, wherein R is H or an unsubstituted C₁₋₃ alkyl group.
K. The compound of any one of Paragraphs A-J, wherein R¹ is selected from H, C(O)R⁹, or a substituted or unsubstituted alkyl, cycloalkylalkyl, aralkyl, heterocyclylalkyl, or heteroarylalkyl group.
L. The compound of any one of Paragraphs A-K, wherein R¹ is selected from H or an unsubstituted alkyl, cycloalkylalkyl, aralkyl, or heterocyclylalkyl group.
M. The compound of any one of Paragraphs A-K, wherein R¹ is selected from H, C(O)R⁹, or a methyl, ethyl, isopropyl, s-butyl, t-butyl, n-pentyl, neopentyl, cyclopropylmethyl, cyclobutylmethyl, cyclopentylmethyl, cyclohexylmethyl, benzyl, pyridinylmethyl, pyridazinylmethyl, morpholinylethyl, oxetanylmethyl, tetrahydropyranylmethyl, 3,5-dimethoxybenzyl, 4-cyano-benzyl, tetrahydropyranylmethyl, pyridin-2-yl-methyl, 6-methylpyridin-2-yl-methyl, 4-t-butyl-benzyl, 4-chlorobenzyl, 4-trifluoromethylbenzyl, or 2,4-dichlorobenzyl group, wherein R⁹ is selected from a substituted or unsubstituted alkyl, cycloalkyl, aryl, heterocyclyl, or heteroaryl group.
N. The compound of Paragraph M, wherein R¹ is C(O)R⁹, and R⁹ is selected from a C₁₋₆ alkyl group optionally substituted with a N(CH₃)₂, phenyl, tetrahydropyranyl, or morpholinyl group.
O. The compound of any one of Paragraphs A-N, wherein R⁵ and R⁸ are independently selected from the group consisting of H, halogen, an unsubstituted C₁₋₆ alkyl group, and OR¹⁰, wherein R¹⁰ is an unsubstituted C₁₋₆ alkyl group.
P. The compound of Paragraph O, wherein R⁵ and R⁸ are independently selected from the group consisting of H, Cl, methyl and methoxy.
Q. The compound of any one of Paragraphs A-P, wherein R⁶ and R⁷ are not both H.
R. The compound of any one of Paragraphs A-Q, wherein R⁶ and R⁷ are independently selected from the group consisting of H, halogen, CN, NO₂, C(O)R¹¹, C(O)OR¹¹, C(O)NR⁹, OR¹², and a substituted or unsubstituted aryl or heteroaryl group.
S. The compound of Paragraph R, wherein R⁶ and R⁷ are independently selected from the group consisting of H, F, Cl, Br, CN, CH₃, CF₃, OCH₃, phenyl, and 2-methylpyridin-4-yl.
T. A compound of Formula II

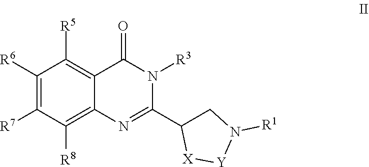

a stereoisomer of thereof, a pharmaceutically acceptable salt of the compound, or a pharmaceutically acceptable salt of the stereoisomer;
wherein,
   X is CH₂, O, or NR²;
   Y is CH₂ or CH₂CH₂, provided that when Y is CH₂, X is CH₂;
   R¹ is H, C(O)R⁹, or a substituted or unsubstituted alkyl, cycloalkyl, cycloalkylalkyl, alkenyl, aralkyl, heterocyclylalkyl, or heteroarylalkyl group;
   R² is H or an unsubstituted alkyl group;
   R³ is selected from the group consisting of H and a substituted or unsubstituted alkyl, alkenyl, aryl or heteroaryl group;
   R⁵ and R⁸ are independently selected from the group consisting of H, OR¹⁰, and a substituted or unsubstituted alkyl, alkenyl, aryl, heteroaryl, or aralkyl group;
   R⁶ and R⁷ are independently selected from the group consisting of H, halogen, CN, OR¹⁰, and a substituted or unsubstituted aryl or heteroaryl group;
   R⁹ is substituted or unsubstituted alkyl, cycloalkyl, cycloalkylalkyl, alkenyl, aryl, aralkyl, heterocyclyl, heterocyclylalkyl, heteroaryl, or heteroarylalkyl group; and
   R¹⁰ at each occurrence is independently H or an unsubstituted alkyl group.
U. The compound Paragraph T, wherein X is CH₂.
V. The compound of Paragraph T or Paragraph U, wherein R¹ is selected from an unsubstituted C₁₋₆ alkyl, oxetanylmethyl, or tetrahydropyranylmethyl group.
W. The compound of any one of Paragraphs T-V, wherein R³ is CH₃.
X. The compound of any one of Paragraphs T-W, wherein R⁶ and R⁷ are independently selected from H or halogen.
Y. A pharmaceutical composition comprising
   a compound of any one of Paragraphs A-X; and
   a pharmaceutically acceptable excipient and/or carrier.
Z. A method of treatment comprising administering a therapeutically effective amount of the compound of any one of Paragraphs A-X or the composition of Paragraph Y to a subject suffering from or at risk of suffering of suffering from an infection of Naegleria fowleri.
AA. The method of Paragraph Z, wherein the subject is a human.
AB. The method of Paragraph Z or Paragraph AA, wherein the effective amount of the compound is 0.001 mg/kg to 1000 mg/kg.
AC. The method of any one of Paragraphs Z-AB, wherein the effective amount of the compound is 0.01 mg/kg to 500 mg/kg.
AD. The method of any one of Paragraphs Z-AC, wherein the effective amount of the compound is 0.1 mg/kg to 100 mg/kg.
AE. The method of any one of Paragraphs Z-AD, wherein the effective amount of the compound is administered orally.

Other embodiments are set forth in the following claims, along with the full scope of equivalents to which such claims are entitled.

What is claimed is:

1. A compound of Formula I 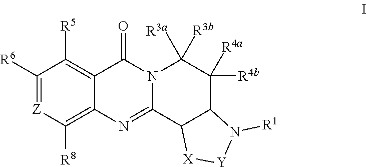
a stereoisomer of thereof, a pharmaceutically acceptable salt of the compound, or a pharmaceutically acceptable salt of the stereoisomer;
wherein,
X is CH₂, O, or NR²;
Y is CH₂ or CH₂CH₂, provided that when Y is CH₂, X is CH₂;
Z is N or CR⁷;
R¹ is H, C(O)R⁹, or a substituted or unsubstituted alkyl, cycloalkyl, cycloalkylalkyl, alkenyl, aralkyl, heterocyclylalkyl, or heteroarylalkyl group;
R² is H or an unsubstituted alkyl group;
R^{3a}, R^{3b}, R^{4a}, and R^{4b} are independently selected from the group consisting of H and a substituted or unsubstituted alkyl group;
R⁵ and R⁸ are independently selected from the group consisting of H, OR¹⁰, halogen and a substituted or unsubstituted alkyl, alkenyl, aryl, aralkyl, or heteroaryl group;
R⁶ and R⁷ are independently selected from the group consisting of H, halogen, CN, NO₂, C(O)R¹¹, C(O)OR¹¹, C(O)NR¹¹R¹², OR¹², and a substituted or unsubstituted alkyl, aryl or heteroaryl group;
R⁹ is substituted or unsubstituted alkyl, cycloalkyl, cycloalkylalkyl, alkenyl, aryl, aralkyl, heterocyclyl, heterocyclylalkyl, heteroaryl, or heteroarylalkyl group; and
R¹⁰, R¹¹, and R¹² at each occurrence are independently H or an unsubstituted alkyl group.

2. The compound of claim 1, wherein substituted alkyl, cycloalkyl, cycloalkylalkyl, alkenyl, aryl, aralkyl, heterocyclyl, heterocyclylalkyl, heteroaryl, and heteroarylalkyl groups are substituted with one or more substituents selected from the group consisting of halogen, hydroxyl, alkoxy, alkenoxy, aryloxy, aralkyloxy, heterocyclyl, heterocyclylalkyl, heterocyclyloxy, heterocyclylalkoxy, oxo, carboxyl, ester, urethane, oximes, hydroxylamine, alkoxyamine, aralkoxyamine, thiol, sulfide, sulfoxide, sulfone, sulfonyl, sulfonamide, sulfate, phosphate, amine, N-oxide, hydrazine, hydrazide, hydrazone, azide, amide, urea, amidine, guanidine, enamine, imide, imine, nitro, and nitrile groups, and further wherein the cycloalkylalkyl, aralkyl, heterocyclylalkyl, and heteroarylalkyl groups are also optionally substituted with one or more of an unsubstituted alkyl or alkenyl group, a haloalkyl group, or a haloalkenyl group.

3. The compound of claim 2, wherein the one or more substituents are selected from the group consisting of F, Cl, Br, OH, NR₂, oxo, C₁₋₆ alkoxy, C₁₋₆ alkyl, CN, halogen, and C₁₋₆ haloalkyl group, wherein R is H or an unsubstituted C₁₋₃ alkyl group.

4. The compound of claim 1, wherein R¹ is selected from H, C(O)R⁹, or a substituted or unsubstituted alkyl, cycloalkylalkyl, aralkyl, heterocyclylalkyl, or heteroarylalkyl group.

5. The compound of claim 1, wherein R¹ is selected from H, C(O)R⁹, or a methyl, ethyl, isopropyl, s-butyl, t-butyl, n-pentyl, neopentyl, cyclopropylmethyl, cyclobutylmethyl, cyclopentylmethyl, cyclohexylmethyl, benzyl, pyridinylmethyl, pyridazinylmethyl, morpholinylethyl, oxetanylmethyl, tetrahydropyranylmethyl, 3,5-dimethoxybenzyl, 4-cyano-benzyl, tetrahydropyranylmethyl, pyridin-2-yl-methyl, 6-methylpyridin-2-yl-methyl, 4-t-butyl-benzyl, 4-chlorobenzyl, 4-trifluoromethylbenzyl, or 2,4-dichlorobenzyl group, wherein R⁹ is selected from a substituted or unsubstituted alkyl, cycloalkyl, aryl, heterocyclyl, or heteroaryl group.

6. The compound of claim 5, wherein R¹ is C(O)R⁹, and R⁹ is selected from a C₁₋₆ alkyl group optionally substituted with a N(CH₃)₂, phenyl, tetrahydropyranyl, or morpholinyl group.

7. The compound of claim 1, wherein R⁵ and R⁸ are independently selected from the group consisting of H, halogen, an unsubstituted C₁₋₆ alkyl group, and OR¹⁰, wherein R¹⁰ is an unsubstituted C₁₋₆ alkyl group.

8. The compound of claim 1, wherein R⁶ and R⁷ are not both H.

9. The compound of claim 1, wherein R⁶ and R⁷ are independently selected from the group consisting of H, halogen, CN, NO₂, C(O)R¹¹, C(O)OR¹¹, C(O)NR⁹, OR¹², and a substituted or unsubstituted aryl or heteroaryl group.

10. The compound of claim 9, wherein R⁶ and R⁷ are independently selected from the group consisting of H, F, Cl, Br, CN, CH₃, CF₃, OCH₃, phenyl, and 2-methylpyridin-4-yl.

11. A compound of Formula II 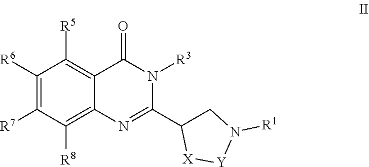
a stereoisomer of thereof, a pharmaceutically acceptable salt of the compound, or a pharmaceutically acceptable salt of the stereoisomer;
wherein,
X is CH₂, O, or NR²;
Y is CH₂ or CH₂CH₂, provided that when Y is CH₂, X is CH₂;
R¹ is H, C(O)R⁹, or a substituted or unsubstituted alkyl, cycloalkyl, cycloalkylalkyl, alkenyl, aralkyl, heterocyclylalkyl, or heteroarylalkyl group;
R² is H or an unsubstituted alkyl group;
R³ is selected from the group consisting of H and a substituted or unsubstituted alkyl, alkenyl, aryl or heteroaryl group;
R⁵ and R⁸ are independently selected from the group consisting of H, OR¹⁰, and a substituted or unsubstituted alkyl, alkenyl, aryl, heteroaryl, or aralkyl group;
R⁶ and R⁷ are independently selected from the group consisting of H, halogen, CN, OR¹⁰, and a substituted or unsubstituted aryl or heteroaryl group;
R⁹ is substituted or unsubstituted alkyl, cycloalkyl, cycloalkylalkyl, alkenyl, aryl, aralkyl, heterocyclyl, heterocyclylalkyl, heteroaryl, or heteroarylalkyl group; and
R¹⁰ at each occurrence is independently H or an unsubstituted alkyl group.

12. The compound claim 11, wherein X is CH₂.

13. The compound of claim 11, wherein R¹ is selected from an unsubstituted C₁₋₆ alkyl, oxetanylmethyl, or tetrahydropyranylmethyl group.

14. The compound of claim 11, wherein R³ is CH₃.

15. The compound of claim 11, wherein R⁶ and R⁷ are independently selected from H or halogen.

16. A pharmaceutical composition comprising
a compound of claim 1; and
a pharmaceutically acceptable excipient and/or carrier.

17. A pharmaceutical composition comprising
a compound of claim 11; and
a pharmaceutically acceptable excipient and/or carrier.

18. A method of treatment comprising administering a therapeutically effective amount of a compound of claim 1 to a subject suffering from or at risk of suffering of suffering from an infection of Naegleria fowleri.

19. A method of treatment comprising administering a therapeutically effective amount of a compound of claim 11 to a subject suffering from or at risk of suffering of suffering from an infection of Naegleria fowleri.

20. A method of treatment comprising administering a therapeutically effective amount of a composition of claim 1 to a subject suffering from or at risk of suffering of suffering from an infection of Naegleria fowleri.
